(19) United States
(12) Patent Application Publication
(10) Pub. No.: US 2025/0311527 A1
(43) Pub. Date: Oct. 2, 2025

(54) ORGANIC COMPOUND, ELECTRONIC ELEMENT AND ELECTRONIC APPARATUS INCLUDING SAME

(71) Applicant: Shaanxi Lighte Optoelectronics Material Co., Ltd., Xi'an (CN)

(72) Inventors: Fumin YUE, Xi'an (CN); Yun LIU, Xi'an (CN)

(21) Appl. No.: 18/865,420

(22) PCT Filed: Aug. 25, 2023
(86) PCT No.: PCT/CN2023/115095

§ 371 Date: Nov. 13, 2024

(30) Foreign Application Priority Data
Jan. 17, 2023 (CN) 202310073444.7

(51) Int. Cl. H10K 50/11; C07D 209/82; C07D 251/12; C09K 11/06; H10K 85/60

(52) U.S. Cl. CPC H10K 50/11; C07D 209/82; C07D 251/12; C09K 11/06; H10K 85/626; H10K 85/633; H10K 85/636; H10K 85/654; H10K 85/6572; H10K 85/6574; H10K 85/6576; C09K 2211/1029

(57) ABSTRACT

The present disclosure relates to an organic compound, and an electronic element and an electronic apparatus including the same. A structural formula of the organic compound of the present disclosure is shown in a formula 1, and when the organic compound is applied to an organic electroluminescent device, the performance of the device can be significantly improved.

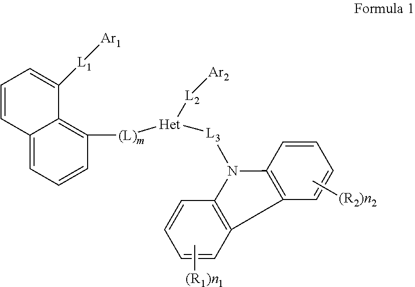

### CROSS REFERENCE TO RELATED APPLICATIONS

This disclosure claims priority to Chinese patent application No. CN202310073444.7, filed on Jan. 17, 2023, the content of which is incorporated herein by reference in its entirety.

### TECHNICAL FIELD

The present disclosure belongs to the technical field of organic materials, and particularly relates to an organic compound, and an electronic element and an electronic apparatus including the same.

### BACKGROUND

An organic electroluminescent device (OLED), also referred to as an organic light-emitting diode, generally includes a cathode and an anode which are oppositely disposed, and a functional layer disposed between the cathode and the anode. The functional layer consists of a plurality of organic or inorganic film layers, typically including an organic light-emitting layer, a hole transport layer, an electron transport layer, and the like. When a voltage is applied to the cathode and the anode, an electric field is generated between the two electrodes, electrons on the cathode side move toward the organic light-emitting layer and holes on the anode side also move toward the organic light-emitting layer under the action of the electric field. The electrons and the holes are combined in the organic light-emitting layer to form excitons, and the excitons are in an excited state and release energy outwards, thus causing the organic light-emitting layer to emit light outwards.

In general, in a host material/dopant system, the choice of a host material is critical because the host material has an important impact on the efficiency and service life of a light-emitting device. Host materials with excellent performance have suitable molecular weights, high glass transition temperatures and thermal decomposition temperatures, high electrochemical stability, and good interfacial contact with adjacent functional layer materials. For a red light host, a material is required to have good carrier transport ability and an appropriate triplet energy level, ensuring that energy can be efficiently transferred from the host material to a guest material during light emission, thus achieving higher device efficiency.

In the currently reported red light host materials, in order to ensure the carrier mobility of molecules, an aromatic structure containing a large conjugated system is generally selected so that the T1 energy level of the molecules is low, and the carrier injection barrier is high, resulting in low exciton recombination efficiency; and in addition, a single large conjugated aromatic structure will also cause defects such as high evaporation temperature and crystallization of materials, and the like, making it difficult to obtain OLED devices with long service life.

Thus, providing a light-emitting host material to improve the efficiency and service life of the device has become an urgent problem to be solved at present.

### SUMMARY

An object of the present disclosure is to provide an organic compound, and an electronic element and an electronic apparatus including the same. When the organic compound is applied to an organic electroluminescent device, the performance of the device can be improved.

A first aspect of the present disclosure provides an organic compound, having a structure represented by a Formula 1:

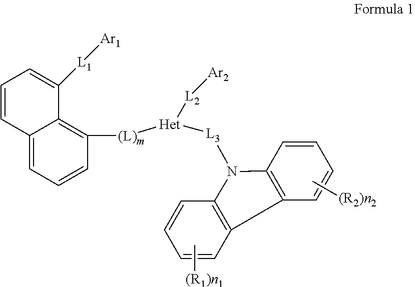

where Het is a 6- to 18-membered nitrogen-containing heteroarylene;
Ar₁ is selected from a substituted or unsubstituted aryl with 6 to 40 carbon atoms;
Ar₂ is selected from hydrogen, a substituted or unsubstituted aryl with 6 to 40 carbon atoms, or a substituted or unsubstituted heteroaryl with 3 to 40 carbon atoms;
L, L₁, L₂ and L₃ are the same or different, and are each independently selected from a single bond, or a substituted or unsubstituted arylene with 6 to 30 carbon atoms;
m is selected from 1 or 2;
substituent(s) of L, L₁, L₂, L₃, Ar₁ and Ar₂ are the same or different, and are each independently selected from a deuterium, a halogen group, a cyano, an alkyl with 1 to 10 carbon atoms, a trialkylsilyl with 3 to 12 carbon atoms, a haloalkyl with 1 to 10 carbon atoms, a cycloalkyl with 3 to 10 carbon atoms, an aryl with 6 to 20 carbon atoms, or a heteroaryl with 3 to 20 carbon atoms;
R₁ and R₂ are the same or different, and are each independently selected from a deuterium, a halogen group, a cyano, an alkyl with 1 to 10 carbon atoms, a trialkylsilyl with 3 to 12 carbon atoms, a cycloalkyl with 3 to 10 carbon atoms, a substituted or unsubstituted aryl with 6 to 20 carbon atoms, or a substituted or unsubstituted heteroaryl with 3 to 20 carbon atoms;
n₁ represents the number of R₁, n₁ is selected from 0, 1, 2, 3 or 4, when n₁ is greater than 1, any two R₁ are the same or different, and optionally, any two adjacent R₁ form an aromatic ring having 6 to 14 carbon atoms;
n₂ represents the number of R₂, n₂ is selected from 0, 1, 2, 3 or 4, when n₂ is greater than 1, any two R₂ are the same or different, and optionally, any two adjacent R₂ form an aromatic ring having 6 to 14 carbon atoms; and
substituent(s) of R₁ and R₂ are the same or different, and are each independently selected from a deuterium, a halogen group, a cyano, an alkyl with 1 to 5 carbon atoms, an aryl with 6 to 12 carbon atoms, or a heteroaryl with 3 to 12 carbon atoms.

A second aspect of the present disclosure provides an electronic element, including an anode and a cathode which are oppositely disposed, and a functional layer disposed between the anode and the cathode, where the functional layer includes the organic compound in the first aspect of the present disclosure.

A third aspect of the present disclosure provides an electronic apparatus, including the electronic element in the second aspect.

The structure of the organic compound of the present disclosure includes a carbazole-derived group, nitrogen-containing heteroarylene, and 1,8-substituted naphthyl which are connected to each other by a single bond or arylene, and compounds formed by directly connecting nitrogen-containing heteroarylene or connecting to a 8-position of naphthyl through arylene have a greater degree of steric distortion, which can improve the glass transition temperature of materials, thus ensuring the formation of a stable amorphous film during evaporation, and improving the service life of the device. In addition, carbazole and naphthalene are electron-rich groups and can serve as an electron donor (D: donor), while nitrogen-containing heteroarylene is an electron-deficient group that is suitable as an electron accepting group (A: Acceptor), the three groups are combined with each other to form a structure of D-A-D, which contributes to energy transfer of light-emitting excitons, thus improving the optical coupling output efficiency of the OLED device; in particular, when 1,8-substituted naphthalene used in the present disclosure is used as an electron donor, the T1 energy level of the molecule can be lowered due to its fused ring properties, and the energy transfer efficiency between excitons and a light-emitting guest material can be improved. Thus, using the organic compound of the present disclosure as a host material can significantly improve the luminous efficiency and service life of the device.

### BRIEF DESCRIPTION OF THE DRAWINGS

The accompanying drawings are used to provide a further understanding of the present disclosure and constitute a part of the specification, and together with the detailed description below, serve to explain the present disclosure, but do not constitute limitations on the present disclosure.

FIG. 1 is a structural schematic diagram of an organic electroluminescent device according to an embodiment of the present disclosure.

FIG. 2 is a schematic diagram of a first electronic device according to an embodiment of the present disclosure.

### REFERENCE SIGNS

100, anode; 200, cathode; 300, functional layer; 310, hole injection layer; 320, hole transport layer; 330, electron blocking layer; 340, organic light-emitting layer; 350, electron transport layer; 360, electron injection layer; and 400, first electronic apparatus.

### DETAILED DESCRIPTION

Examples will now be described more fully with reference to the accompanying drawings. However, the examples can be implemented in various forms and should not be construed as limited to the instances set forth here; rather, these examples are provided so that the present disclosure will be thorough and complete, and the concept of the examples will be fully conveyed to those skilled in the art. The described features, structures, or characteristics may be combined in any suitable manner in one or more embodiments. In the following description, numerous specific details are provided to provide a thorough understanding of the examples of the present disclosure.

In a first aspect, the present disclosure provides an organic compound, having a structure represented by a Formula 1:

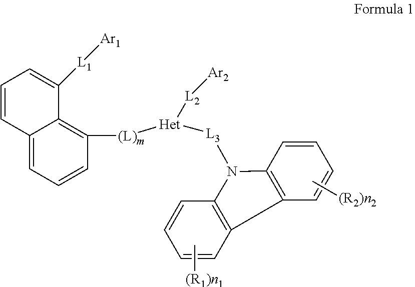

where Het is a 6- to 18-membered nitrogen-containing heteroarylene;
Ar₁ is selected from a substituted or unsubstituted aryl with 6 to 40 carbon atoms;
Ar₂ is selected from hydrogen, a substituted or unsubstituted aryl with 6 to 40 carbon atoms, or a substituted or unsubstituted heteroaryl with 3 to 40 carbon atoms;
L, L₁, L₂ and L₃ are the same or different, and are each independently selected from a single bond, or a substituted or unsubstituted arylene with 6 to 30 carbon atoms;
m is selected from 1 or 2;
substituent(s) of L, L₁, L₂, L₃, Ar₁ and Ar₂ are the same or different, and are each independently selected from a deuterium, a halogen group, a cyano, an alkyl with 1 to 10 carbon atoms, a trialkylsilyl with 3 to 12 carbon atoms, a haloalkyl with 1 to 10 carbon atoms, a cycloalkyl with 3 to 10 carbon atoms, an aryl with 6 to 20 carbon atoms, or a heteroaryl with 3 to 20 carbon atoms;
R₁ and R₂ are the same or different, and are each independently selected from a deuterium, a halogen group, a cyano, an alkyl with 1 to 10 carbon atoms, a trialkylsilyl with 3 to 12 carbon atoms, a cycloalkyl with 3 to 10 carbon atoms, a substituted or unsubstituted aryl with 6 to 20 carbon atoms, or a substituted or unsubstituted heteroaryl with 3 to 20 carbon atoms;
n₁ represents the number of R₁, n₁ is selected from 0, 1, 2, 3 or 4, when n₁ is greater than 1, any two R₁ are the same or different, and optionally, any two adjacent R₁ form an aromatic ring having 6 to 14 carbon atoms;
n₂ represents the number of R₂, n₂ is selected from 0, 1, 2, 3 or 4, when n₂ is greater than 1, any two R₂ are the same or different, and optionally, any two adjacent R₂ form an aromatic ring having 6 to 14 carbon atoms; and
substituent(s) of R₁ and R₂ are the same or different, and are each independently selected from a deuterium, a halogen group, a cyano, an alkyl with 1 to 5 carbon atoms, an aryl with 6 to 12 carbon atoms, or a heteroaryl with 3 to 12 carbon atoms.

In the present disclosure, the organic compound is selected from structures represented by a Formula 2-1, a Formula 2-2, a Formula 2-3, a Formula 2-4, a Formula 2-5, a Formula 2-6, a Formula 2-7, a Formula 2-8, a Formula 2-9, or a Formula 2-10:

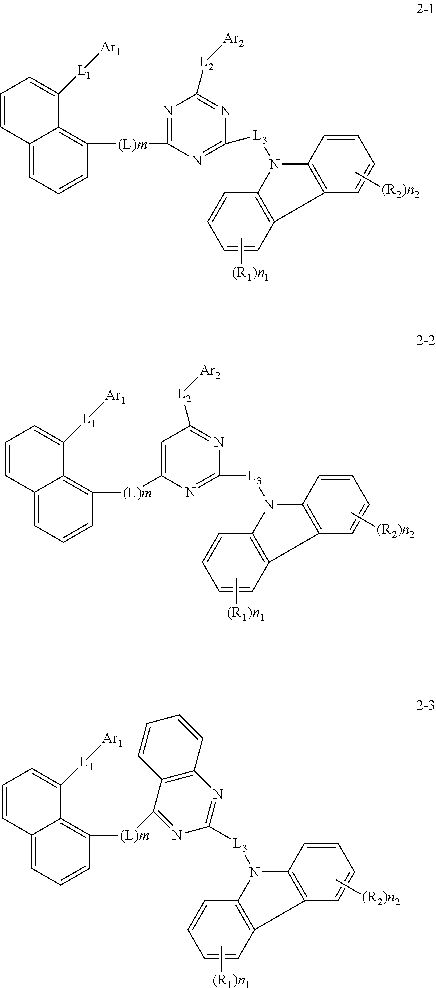

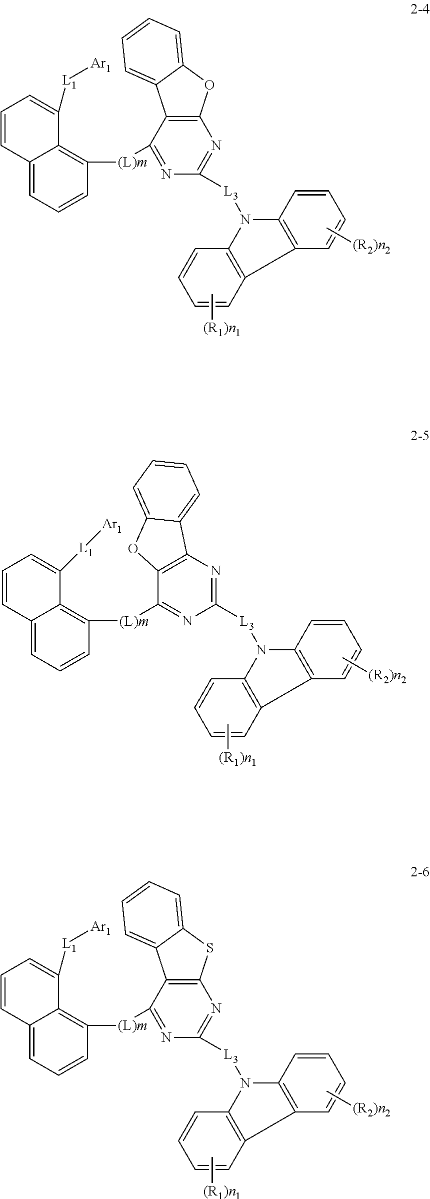

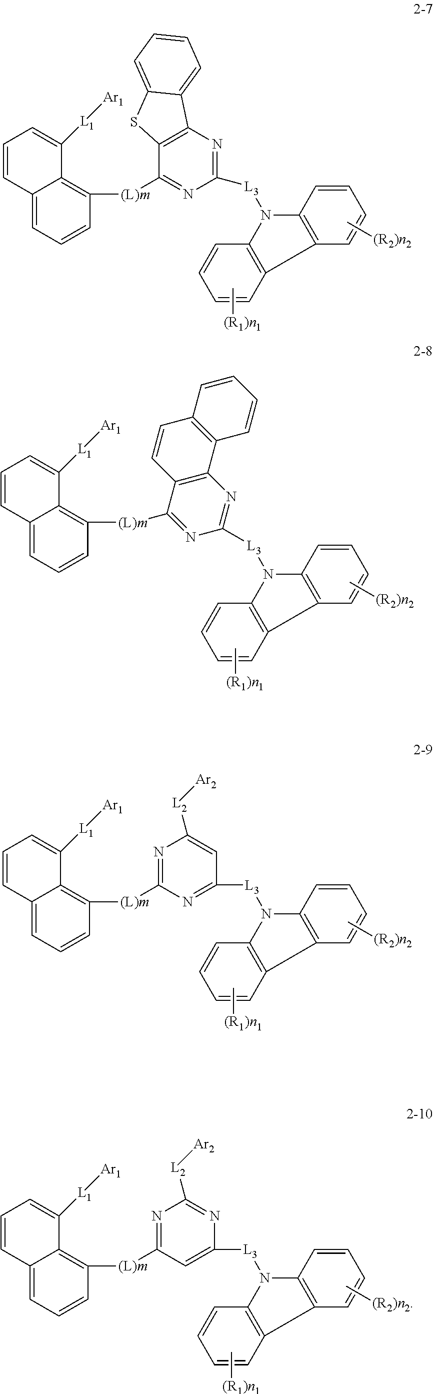

In the present disclosure, the terms “optional” and “optionally” mean that the subsequently described event or circumstance may but need not occur, and that the description includes instances where the event or circumstance occurs and instances where the event or circumstance does not occur. For example, “optionally, any two adjacent substituents xx form a ring”, which means that the two substituents can form a ring but do not necessarily form a ring, including the scenario where two adjacent substituents form a ring and the scenario where two adjacent substituents do not form a ring.

In the present disclosure, the adopted description modes “each . . . is independently”, “ . . . is respectively and independently” and “ . . . is independently selected from” can be interchanged, and should be understood in a broad sense, which means that in different groups, specific options expressed between the same symbols do not influence each other, or in a same group, specific options expressed between the same symbols do not influence each other. For example, the meaning of “

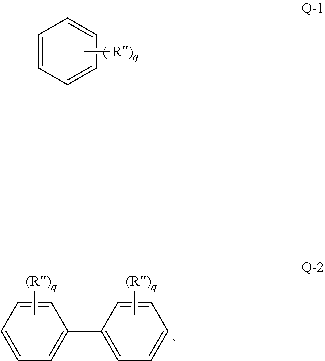

where each q is independently 0, 1, 2 or 3, and each R″ is independently selected from hydrogen, deuterium, fluorine and chlorine” is as follows: a formula Q-1 represents that q substituents R″ exist on a benzene ring, each R″ can be the same or different, and options of each R″ do not influence each other; and a formula Q-2 represents that each benzene ring of biphenyl has q substituents R″, the number q of the substituents R″ on the two benzene rings can be the same or different, each R″ can be the same or different, and options of each R″ do not influence each other.

In the present disclosure, the term such as “substituted or unsubstituted” means that a functional group described behind the term may have or may not have a substituent (in the below, the substituent is collectively referred to as Re in order to facilitate description). For example, the “substituted or unsubstituted aryl” refers to aryl having the substituent Rc or unsubstituted aryl. Where the above substituent, i.e., Rc, for example, can be a deuterium, a halogen group, a cyano, a heteroaryl, an aryl, a trialkylsilyl, an alkyl, a haloalkyl, a cycloalkyl, or the like.

In the present disclosure, the number of carbon atoms of a substituted or unsubstituted functional group refers to the number of all carbon atoms. For example, if L is substituted arylene with 12 carbon atoms, then the number of all carbon atoms of the arylene and substituents on the arylene is 12.

In the present disclosure, aryl refers to an optional functional group or substituent derived from an aromatic carbocyclic ring. The aryl may be monocyclic aryl (e.g., phenyl) or polycyclic aryl, in other words, the aryl may be monocyclic aryl, fused aryl, two or more monocyclic aryl conjugatedly linked by carbon-carbon bonds, monocyclic aryl and fused aryl which are conjugatedly linked by a carbon-carbon bond, or two or more fused aryl conjugatedly linked by carbon-carbon bonds. That is, unless otherwise indicated, two or more aromatic groups conjugatedly linked by carbon-carbon bonds may also be considered as the aryl in the present disclosure. The fused aryl may include, for example, bicyclic fused aryl (e.g., naphthyl), tricyclic fused aryl (e.g., phenanthryl, fluorenyl, and anthryl), and the like. The aryl does not contain heteroatoms such as B, N, O, S, P, Se, and Si. For example, in the present disclosure, biphenyl, terphenyl, and the like are the aryl. Examples of the aryl can include, but are not limited to, phenyl, naphthyl, fluorenyl, anthryl, phenanthryl, biphenyl, terphenyl, benzo[9,10]phenanthryl, pyrenyl, benzofluoranthenyl, chrysenyl, and the like. In the present disclosure, the arylene involved refers to a divalent group formed by the further loss of one hydrogen atom of the aryl.

In the present disclosure, the substituted aryl may be that one or two or more hydrogen atoms in the aryl are substituted by groups such as a deuterium atom, a halogen group, a cyano, an aryl, a heteroaryl, a trialkylsilyl, an alkyl, a cycloalkyl and a haloalkyl. It should be understood that the number of carbon atoms of the substituted aryl refers to the total number of carbon atoms of the aryl and the substituents on the aryl, e.g., substituted aryl with carbon atoms of 18 means that the total number of carbon atoms of the aryl and substituents is 18.

In the present disclosure, heteroaryl refers to a monovalent aromatic ring containing at least one heteroatom in the ring or its derivative, and the heteroatom may be one or more of B, O, N, P, Si, Se, and S. The heteroaryl may be monocyclic heteroaryl or polycyclic heteroaryl, in other words, the heteroaryl may be a single aromatic ring system or a plurality of aromatic ring systems conjugatedly linked by carbon-carbon bonds, and any one aromatic ring system is a monocyclic aromatic ring or a fused aromatic ring. For example, the heteroaryl may include thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silafluorenyl, dibenzofuranyl, and N-phenylcarbazolyl, N-pyridylcarbazolyl, N-methylcarbazolyl, and the like, but is not limited to this. In the present disclosure, the heteroarylene involved refers to a divalent group formed by the further loss of one hydrogen atom of the heteroaryl.

In the present disclosure, the substituted heteroaryl can be that may be that one or two or more hydrogen atoms in the heteroaryl are substituted by groups such as deuterium, halogen group, cyano, aryl, heteroaryl, trialkylsilyl, alkyl, cycloalkyl, and haloalkyl. It should be understood that the number of carbon atoms of the substituted heteroaryl refers to the total number of carbon atoms of the heteroaryl and the substituents on the heteroaryl.

In the present disclosure, the number of carbon atoms of the aryl as a substituent in L, L₁, L₂, L₃, Ar₁ and Ar₂ may be 6 to 20, for example, the number of carbon atoms may be 6, 7, 8, 9, 10, 11, 12, 13, 14, 15, 16, 17, 18, 19, or 20, and specific examples of the aryl as the substituent include, but are not limited to, phenyl, biphenyl, naphthyl, fluorenyl, phenanthryl, anthryl, and chrysenyl.

In the present disclosure, the number of carbon atoms of the heteroaryl as a substituent in L, L₁, L₂, L₃, Ar₁ and Ar₂ may be 3 to 20, for example, the number of carbon atoms may be 3, 4, 5, 6, 7, 8, 9, 10, 11, 12, 13, 14, 15, 16, 17, 18, 19, or 20, and specific examples of the heteroaryl as the substituent include, but are not limited to, pyridyl, pyrimidinyl, carbazolyl, dibenzofuranyl, dibenzothienyl, quinolyl, quinazolinyl, quinoxalinyl, and isoquinolyl.

In the present disclosure, the number of carbon atoms of the alkyl with 1 to 10 carbon atoms may be 1, 2, 3, 4, 5, 6, 7, 8, 9, or 10, and specific examples of the alkyl include, but are not limited to, methyl, ethyl, n-propyl, isopropyl, n-butyl, isobutyl, tert-butyl, n-pentyl, isopentyl, neopentyl, n-hexyl, n-octyl, 2-ethylhexyl, nonyl, decyl, 3,7-dimethyloctyl, and the like.

In the present disclosure, the halogen group may be, for example, fluorine, chlorine, bromine, or iodine.

In the present disclosure, specific examples of trialkylsilyl include, but are not limited to, trimethylsilyl, triethylsilyl, and the like.

In the present disclosure, specific examples of haloalkyl include, but are not limited to, trifluoromethyl.

In the present disclosure, the number of carbon atoms of cycloalkyl with 3 to 10 carbon atoms may be, for example, 3, 4, 5, 6, 7, 8, or 10. Specific examples of cycloalkyl include, but are not limited to, cyclopentyl, cyclohexyl, and adamantyl.

In the present disclosure, n atoms form a ring system, i.e., an n-membered ring. For example, phenyl is 6-membered aryl. 6- to 18-membered nitrogen-containing heteroarylene refers to heteroarylene with 6 to 18 ring atoms including a nitrogen atom. The number of ring atoms of the 6- to 18-membered nitrogen-containing heteroarylene may be, for example, 6, 7, 8, 9, 10, 11, 12, 13, 14, 15, 16, 17, or 18.

In the present disclosure, an unpositioned connecting bond refers to a single bond “ [symbol] ” and [symbol] extending from a ring system, which means that one end of the connecting bond can be connected with any position in the ring system through which the bond penetrates, and the other end of the connecting bond is connected with the remaining part of a compound molecule. For example, as shown in the following formula (f), naphthyl represented by the formula (f) is connected to other positions of a molecule through two unpositioned connecting bonds penetrating a dicyclic ring, and its meaning includes any one possible connecting mode represented by Formulae (f-1) to (f-10).

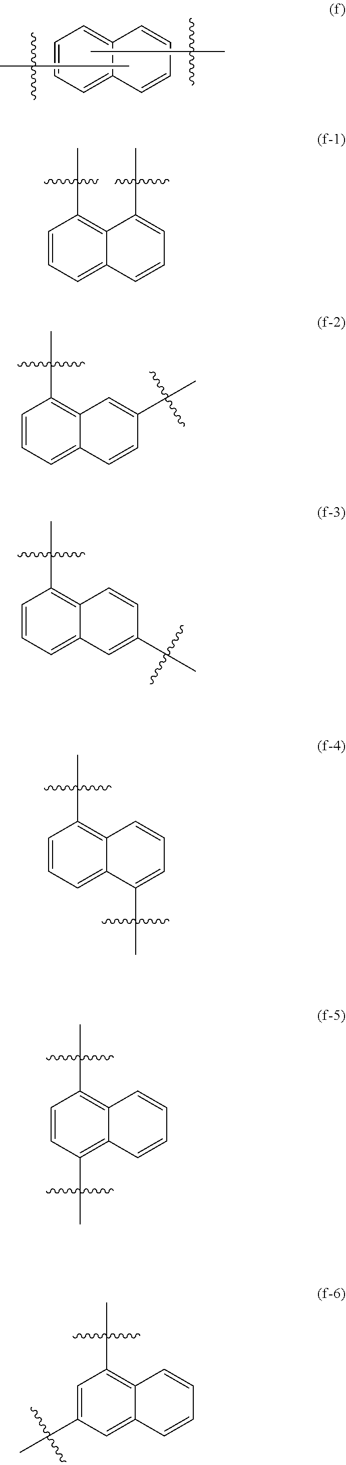

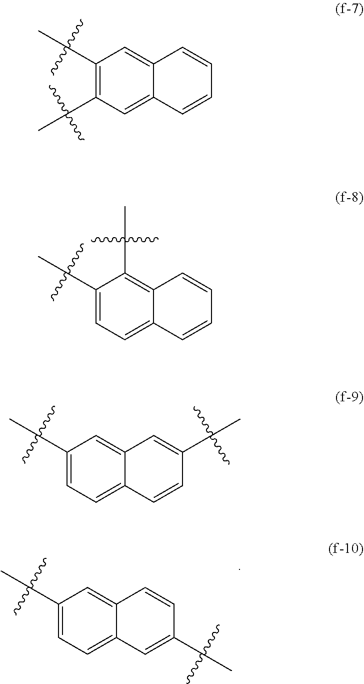 [SMILES: *c1cc2ccccc2cc1* ; *c1ccc2ccccc2c1* ; *c1ccc2ccc(*)cc2c1 ; *c1ccc2cc(*)ccc2c1]

For another example, as shown in the following formula (X′), dibenzofuranyl represented by the formula (X′) is connected with other positions of a molecule through one unpositioned connecting bond extending from the middle of a benzene ring on one side, and its meaning includes any one possible connecting mode represented by Formulae (X′-1) to (X′-4).

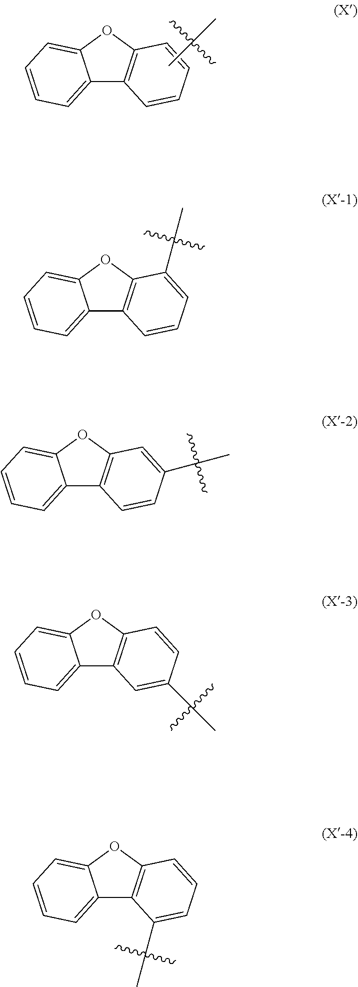

In the present disclosure, -(L)ₘ- indicates that m unit(s) of group L are linked in sequence.

In some embodiments, Het in the Formula 1 is a 6- to 14-membered nitrogen-containing heteroarylene.

In other embodiments of the present disclosure, Het is a 6-membered nitrogen-containing heteroarylene, a 10-membered nitrogen-containing a heteroarylene, a 13-membered nitrogen-containing heteroarylene, or a 14-membered nitrogen-containing heteroarylene.

In some embodiments, Het in the Formula 1 is selected from:

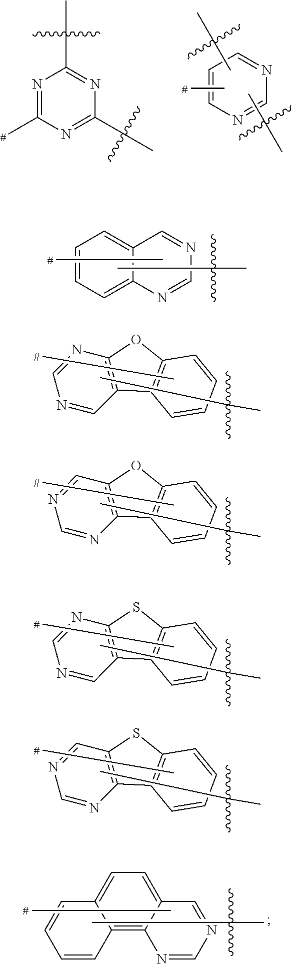

and

[symbol] represents a bond connected to L₃, and [symbol] represents a bond connected to L or L₂; and when only one [symbol] is present in the Het group, [symbol] represents a bond connected to L, and at this time, L₂ is a single bond and Ar₂ is hydrogen, i.e.,

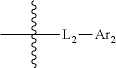

absent.

In some more specific embodiments, Het is selected from:

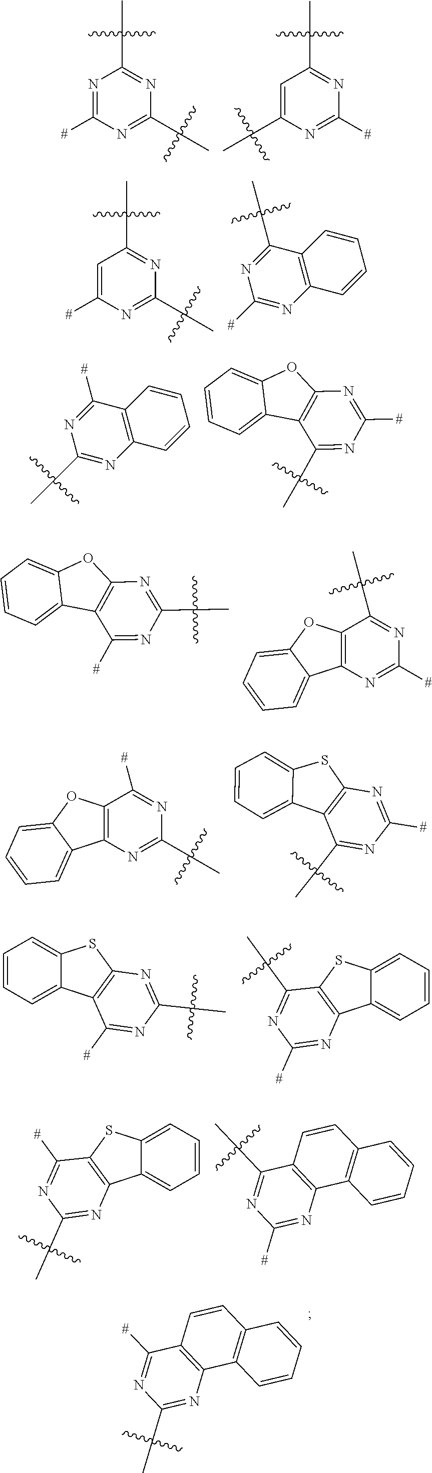

and

[symbol] represents a bond connected to L₃, and [symbol] represents a bond connected to L or L₂; and when only one [symbol] is present in the Het group, [symbol] represents a bond connected to L, and at this time, L₂ is a single bond and Ar₂ is hydrogen.

In some embodiments, Ar₁ is selected from a substituted or unsubstituted aryl with 6 to 20 carbon atoms. For example, Ar₁ is selected from a substituted or unsubstituted aryl with 6, 7, 8, 9, 10, 11, 12, 13, 14, 15, 16, 17, 18, 19, or 20 carbon atoms.

Preferably, substituent(s) of Ar₁ are each independently selected from deuterium, a fluorine, a cyano, an alkyl with 1 to 5 carbon atoms, a trimethylsilyl, a trifluoromethyl, a cycloalkyl with 5 to 10 carbon atoms, or an aryl with 6 to 12 carbon atoms.

Optionally, Ar₁ is selected from a substituted or unsubstituted phenyl, a substituted or unsubstituted naphthyl, a substituted or unsubstituted biphenyl, a substituted or unsubstituted fluorenyl, a substituted or unsubstituted phenanthryl, or a substituted or unsubstituted terphenyl.

Preferably, substituent(s) of Ar₁ are each independently selected from a deuterium, a fluorine, a cyano, a methyl, an ethyl, an isopropyl, a tert-butyl, a trimethylsilyl, a trifluoromethyl, a cyclopentyl, a cyclohexyl, an adamantyl, a phenyl, a naphthyl or a biphenyl.

Optionally, Ar₁ is selected from a substituted or unsubstituted group W, where the unsubstituted group W is selected from:

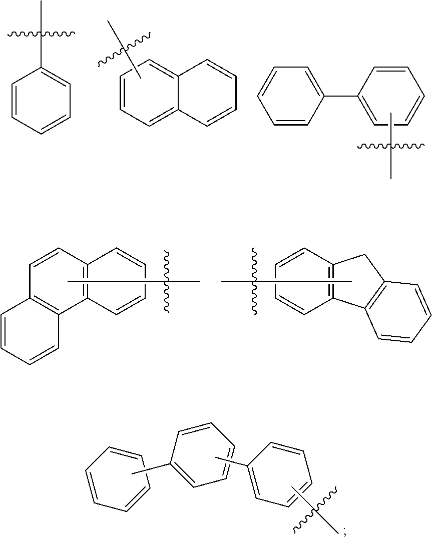

where the substituted group W has one or two or more substituents independently selected from a deuterium, a fluorine, a cyano, a methyl, an ethyl, an isopropyl, a tert-butyl, a trimethylsilyl, a trifluoromethyl, a cyclopentyl, a cyclohexyl, an adamantyl, a phenyl, a naphthyl or a biphenyl, and when the number of the substituents is greater than 1, the substituents are the same or different.

Optionally, Ar₁ is selected from:

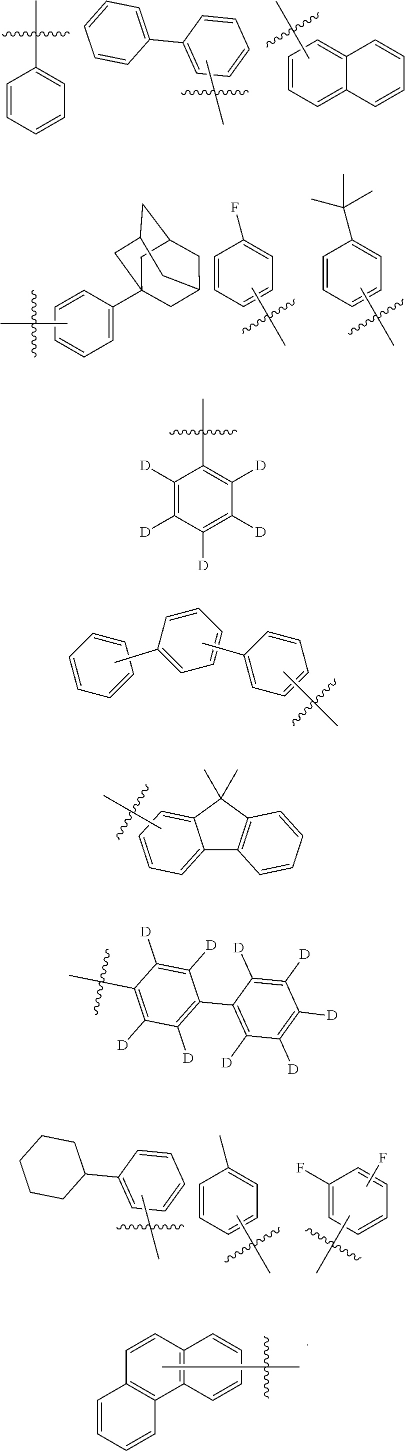

In some specific embodiments, Ar₁ is selected from:

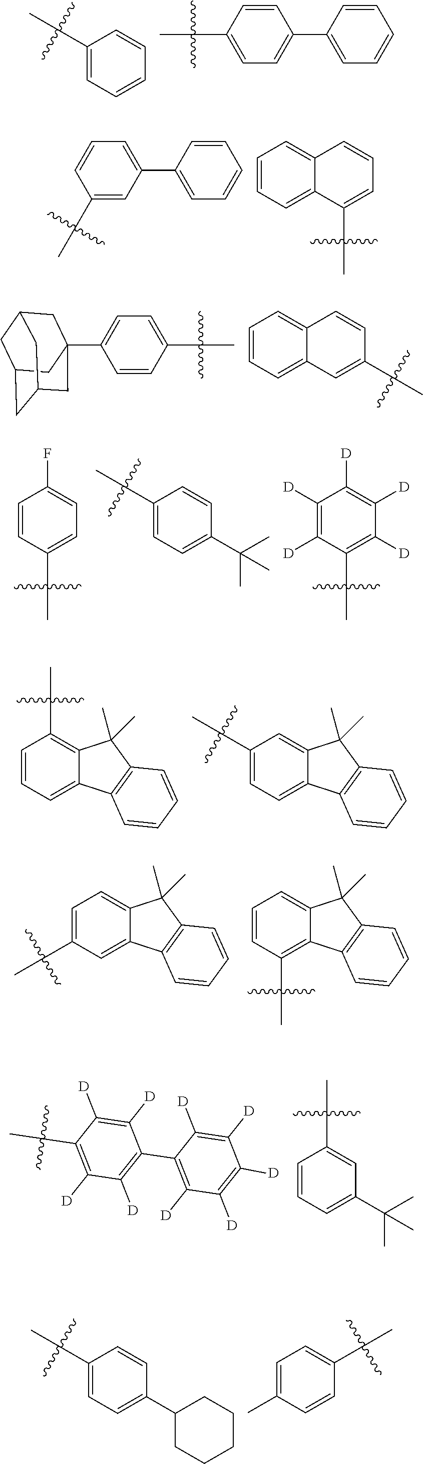

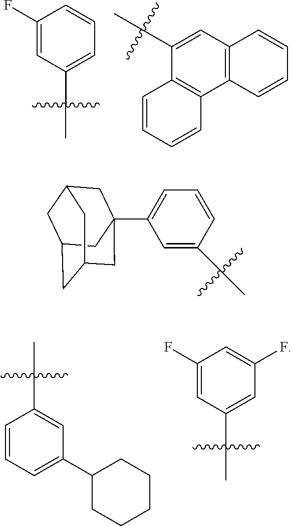

In some embodiments, Ar₂ is selected from hydrogen, a substituted or unsubstituted aryl with 6 to 25 carbon atoms, or a substituted or unsubstituted heteroaryl with 5 to 18 carbon atoms. For example, Ar₂ is selected from hydrogen, or a substituted or unsubstituted aryl with 6, 7, 8, 9, 10, 11, 12, 13, 14, 15, 16, 17, 18, 19, 20, 21, 22, 23, 24, or 25; or a substituted or unsubstituted heteroaryl with 5, 6, 7, 8, 9, 10, 11, 12, 13, 14, 15, 16, 17, or 18 carbon atoms.

Preferably, substituent(s) of Ar₂ are each independently selected from a deuterium, a fluorine, a cyano, an alkyl with 1 to 5 carbon atoms, a trimethylsilyl, a trifluoromethyl, a cycloalkyl with 5 to 10 carbon atoms, an aryl with 6 to 12 carbon atoms, or a heteroaryl with 5 to 12 carbon atoms.

Optionally, Ar₂ is selected from hydrogen, a substituted or unsubstituted phenyl, a substituted or unsubstituted naphthyl, a substituted or unsubstituted biphenyl, a substituted or unsubstituted fluorenyl, a substituted or unsubstituted phenanthryl, a substituted or unsubstituted terphenyl, a substituted or unsubstituted pyridyl, a substituted or unsubstituted quinolyl, a substituted or unsubstituted dibenzofuranyl, a substituted or unsubstituted dibenzothienyl, or a substituted or unsubstituted carbazolyl.

Preferably, substituent(s) of Ar₂ are each independently selected from deuterium, a fluorine, a cyano, a methyl, an ethyl, an isopropyl, a tert-butyl, a trimethylsilyl, a trifluoromethyl, a cyclopentyl, a cyclohexyl, an adamantyl, a phenyl, a naphthyl, a biphenyl, a dibenzofuranyl, a dibenzothienyl or a carbazolyl.

Optionally, Ar₂ is selected from hydrogen or a substituted or unsubstituted group V, where the unsubstituted group V is selected from:

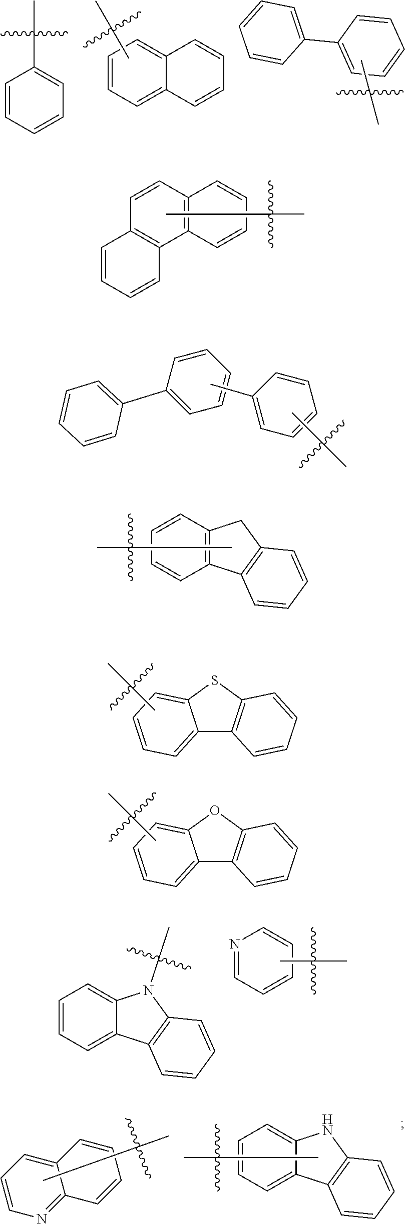

where the substituted group V has one or two or more substituents independently selected from a deuterium, a fluorine, a cyano, a methyl, an ethyl, an isopropyl, a tert-butyl, a trimethylsilyl, a trifluoromethyl, a cyclopentyl, a cyclohexyl, an adamantyl, a phenyl, a naphthyl, a biphenyl, a dibenzofuranyl, a dibenzothienyl or a carbazolyl, and when the number of the substituents is greater than 1, the substituents are the same or different.

Optionally, Ar₂ is selected from hydrogen or the following groups:

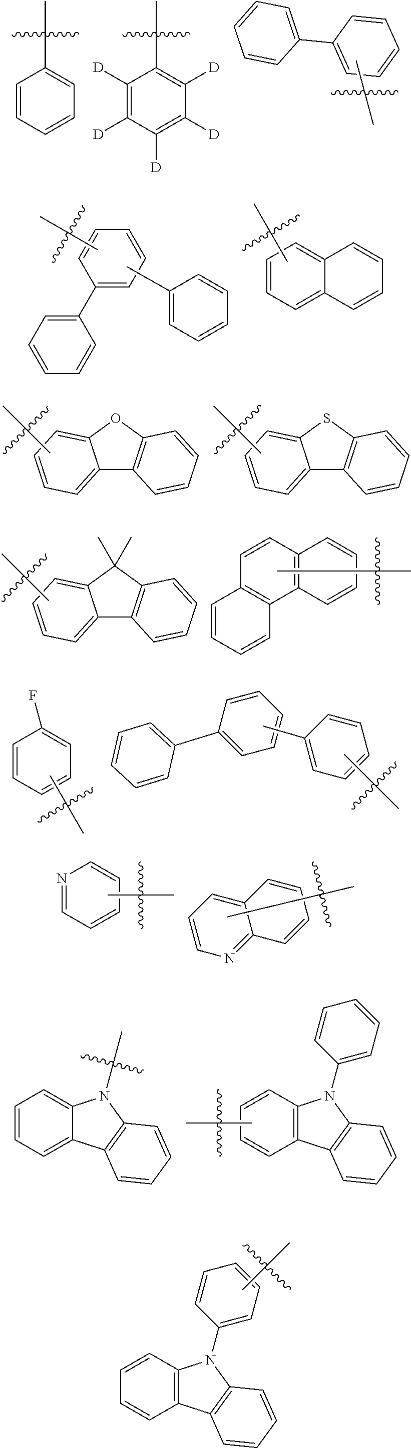

In some specific embodiments, Ar₂ is selected from hydrogen or the following groups:

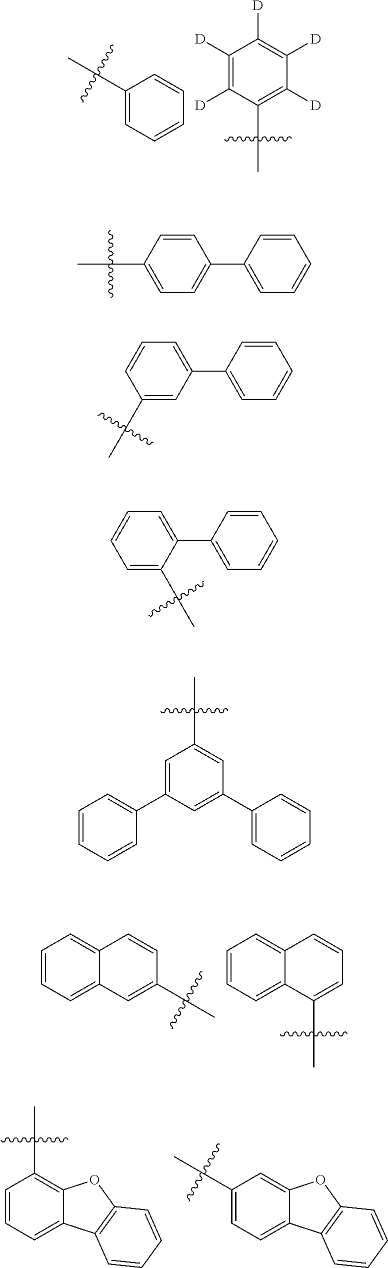

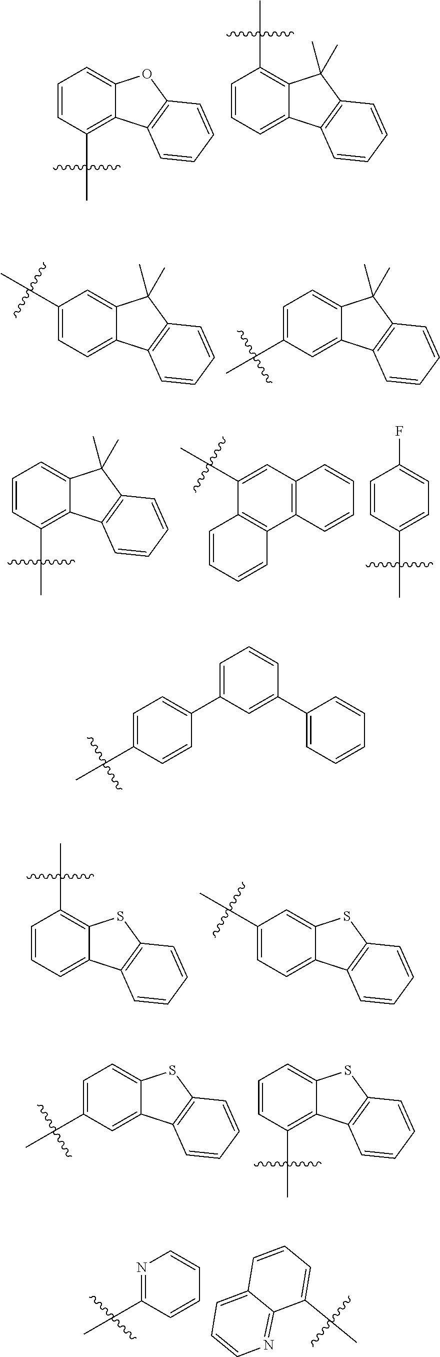

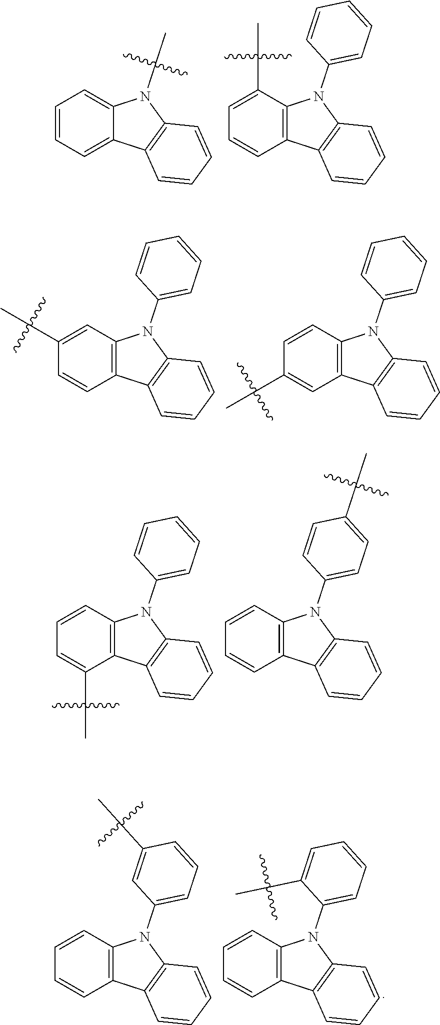

In some embodiments, L, L₁, L₂ and L₃ are each independently selected from a single bond or a substituted or unsubstituted arylene with 6 to 20 carbon atoms. For example, L, L₁, L₂ and L₃ are each independently selected from a single bond; or a substituted or unsubstituted arylene with 6, 7, 8, 9, 10, 11, 12, 13, 14, 15, 16, 17, 18, 19, or 20 carbon atoms.

Preferably, substituent(s) of L, L₁, L₂, and L₃ are each independently selected from a deuterium, a fluorine, a cyano, an alkyl with 1 to 5 carbon atoms or an aryl with 6 to 12 carbon atoms.

Optionally, L, L₁, L₂ and L₃ are each independently selected from a single bond, a substituted or unsubstituted phenylene, a substituted or unsubstituted naphthylene, or a substituted or unsubstituted biphenylene.

Preferably, substituent(s) of L, L₁, L₂, and L₃ are each independently selected from a deuterium, a fluorine, a cyano, a methyl, an ethyl, an isopropyl, a tert-butyl, a phenyl, a naphthyl or a biphenyl.

Optionally, L₁, L₂ and L₃ are each independently selected from a single bond or the following groups:

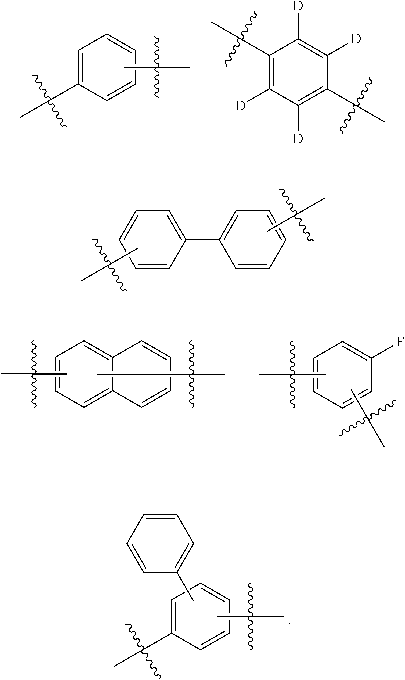

Further optionally, L₁, L₂ and L₃ are each independently selected from a single bond or the following groups:

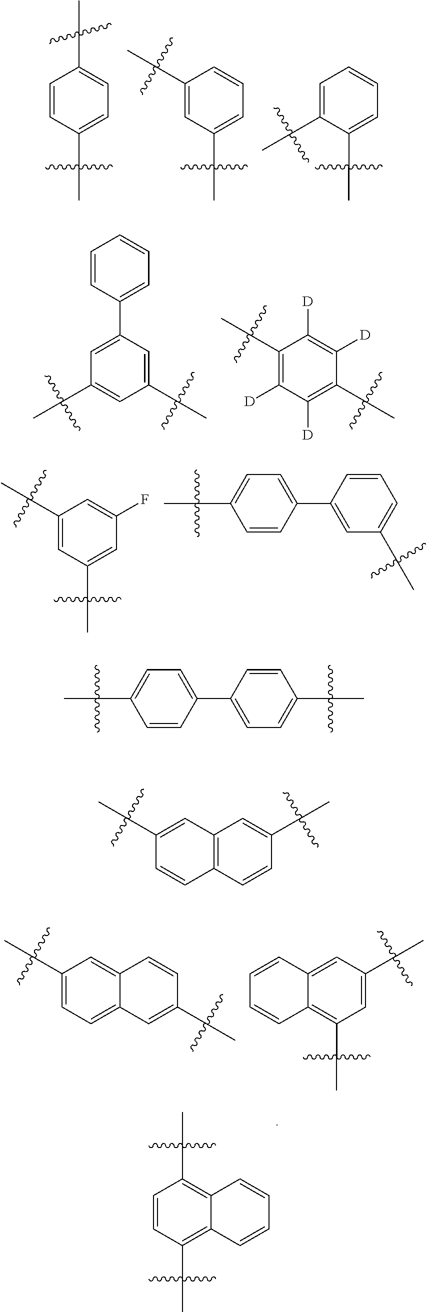

Optionally, -(L)ₘ- is selected from a single bond or the following groups:

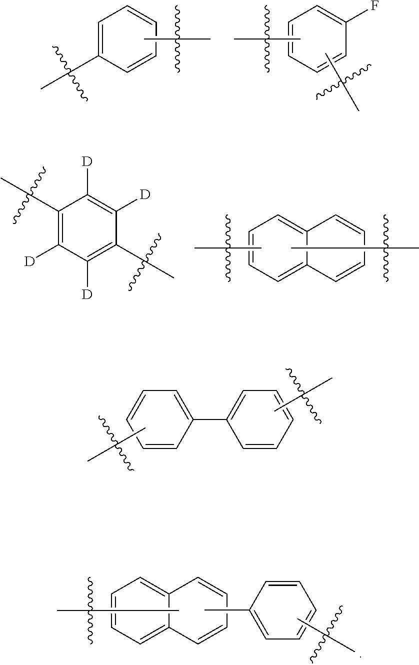

Further optionally, -(L)ₘ- is selected from a single bond or the following groups:

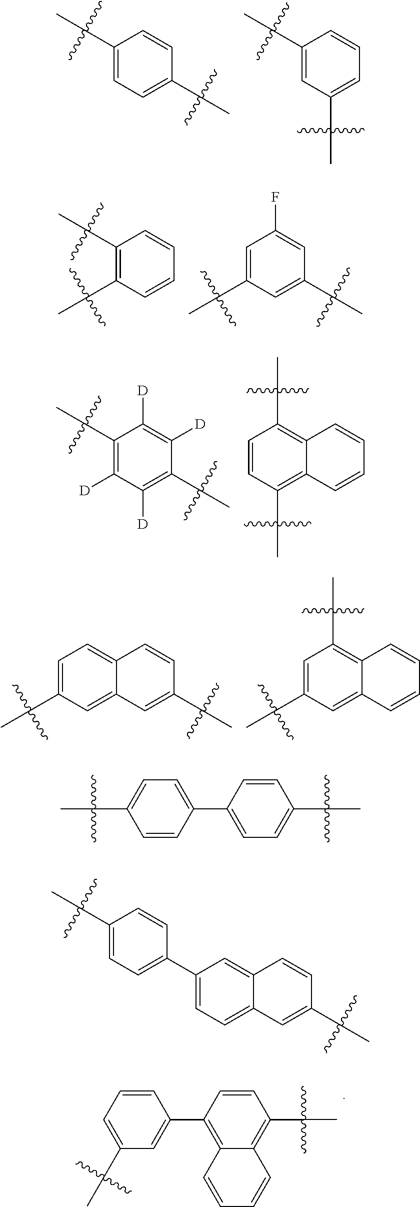

In one embodiment of the present disclosure, R₁ and R₂ are each independently selected from deuterium, a fluorine, a cyano, a methyl, an ethyl, an isopropyl, a tert-butyl, a trimethylsilyl, a substituted or unsubstituted phenyl, a substituted or unsubstituted naphthyl, a substituted or unsubstituted biphenyl, a substituted or unsubstituted fluorenyl, a substituted or unsubstituted phenanthryl, a substituted or unsubstituted pyridyl, a substituted or unsubstituted quinolyl, a substituted or unsubstituted dibenzofuranyl, a substituted or unsubstituted dibenzothienyl, or a substituted or unsubstituted carbazolyl; or any two adjacent R₁ form a benzene ring or a naphthalene ring; or any two adjacent R₂ form a benzene ring or a naphthalene ring.

Preferably, substituent(s) of R₁ and R₂ are each independently selected from deuterium, a fluorine, a cyano, a methyl, an ethyl, an isopropyl, a tert-butyl, a phenyl, a naphthyl, a biphenyl, a dibenzofuranyl, a dibenzothienyl or a carbazolyl.

Optionally, R₁ and R₂ are each independently selected from deuterium, a fluorine, a cyano, a methyl, an ethyl, an isopropyl, a tert-butyl, a trimethylsilyl, a deuterium-substituted phenyl, a phenyl, a naphthyl, a biphenyl, a phenanthryl, a pyridyl, a quinolyl, a 9,9-dimethylfluorenyl, a dibenzofuranyl, a dibenzothienyl, a N-carbazolyl, or a N-phenylcarbazolyl; or any two adjacent R₁ form a benzene ring or a naphthalene ring; or any two adjacent R₂ form a benzene ring or a naphthalene ring.

Optionally, R₁ and R₂ are each independently selected from deuterium, a fluorine, a methyl, an ethyl, an isopropyl, a tert-butyl, a trimethylsilyl, or the following groups:

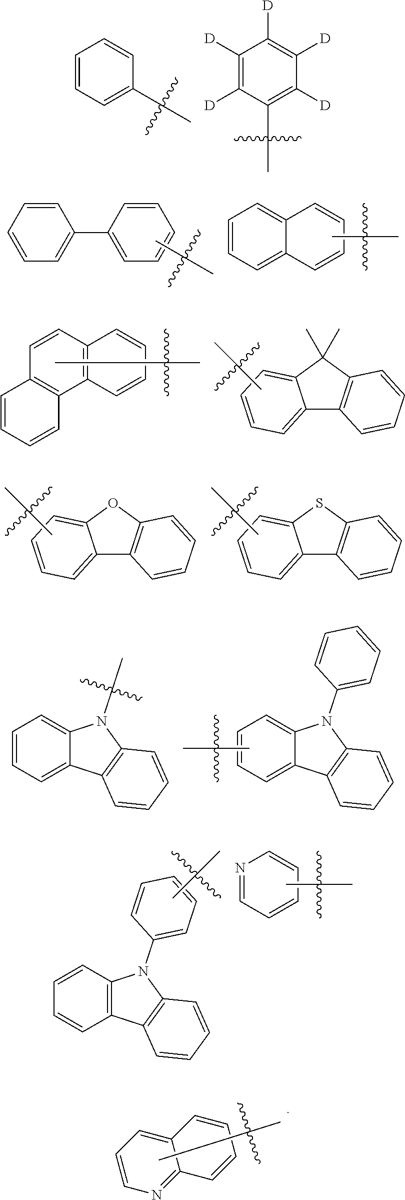

Further optionally, R₁ and R₂ are each independently selected from deuterium, a fluorine, a methyl, an ethyl, an isopropyl, a tert-butyl, a trimethylsilyl, or the following groups:

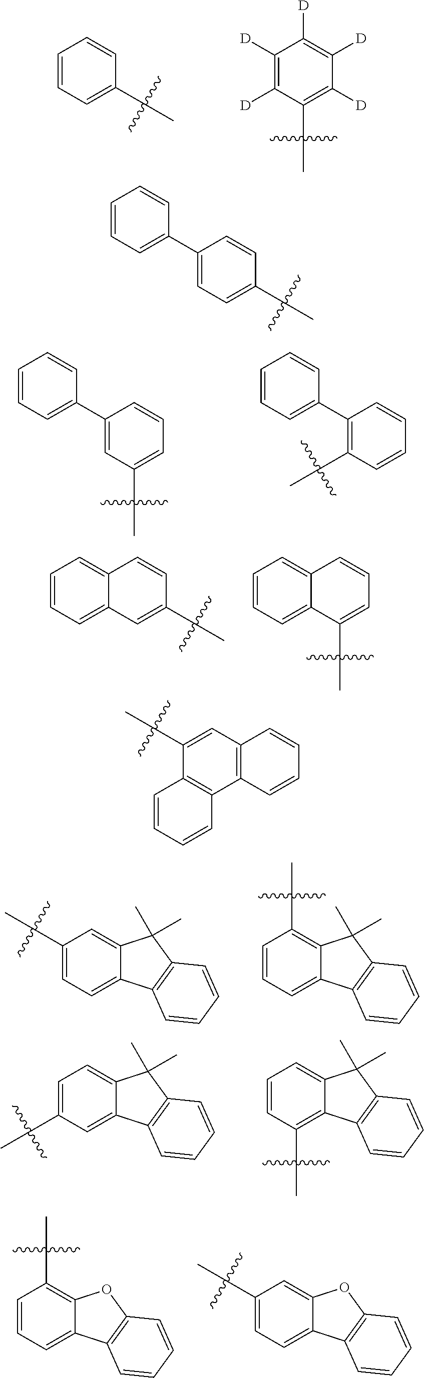

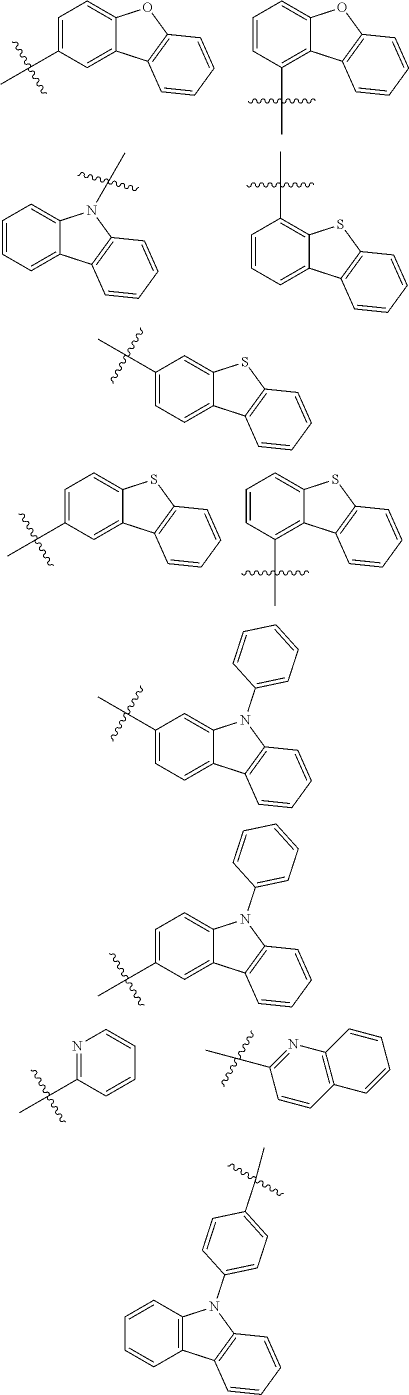

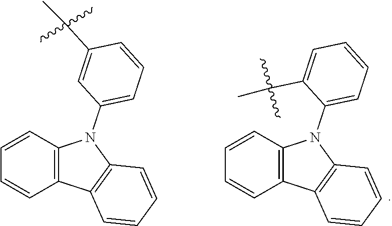 [SMILES: *c1cccc(-n2c3ccccc3c3ccccc32)c1 ; *c1ccccc1-n1c2ccccc2c2ccccc21]

In one embodiment of the present disclosure,

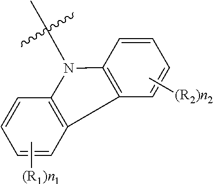

in the formula 1 is selected from the following structures:

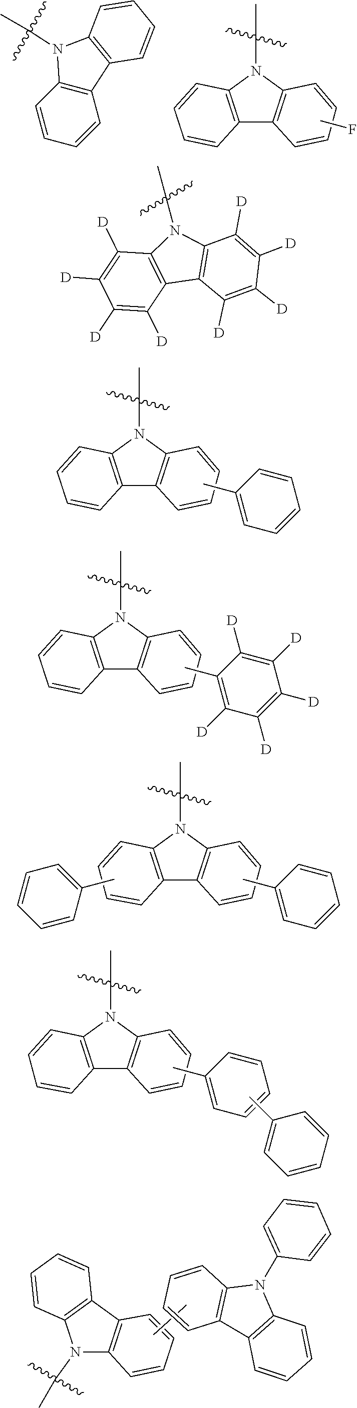

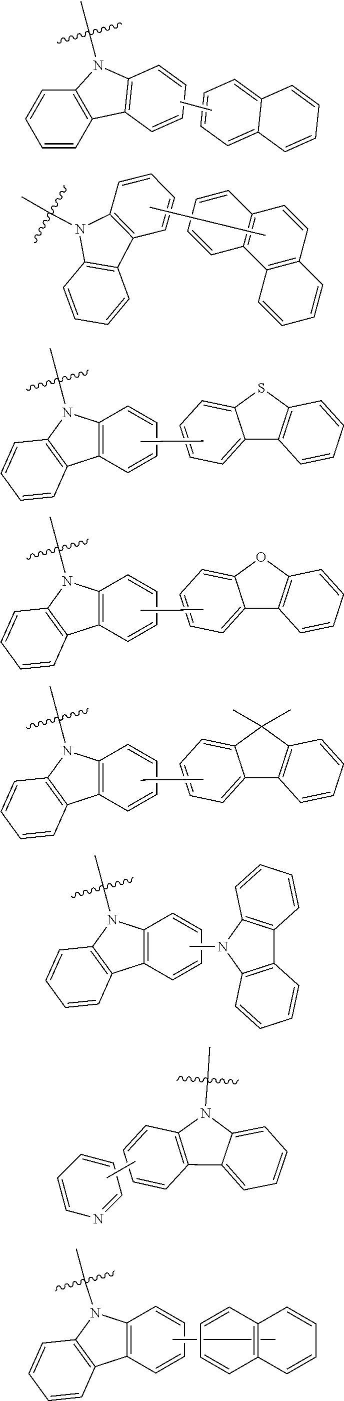

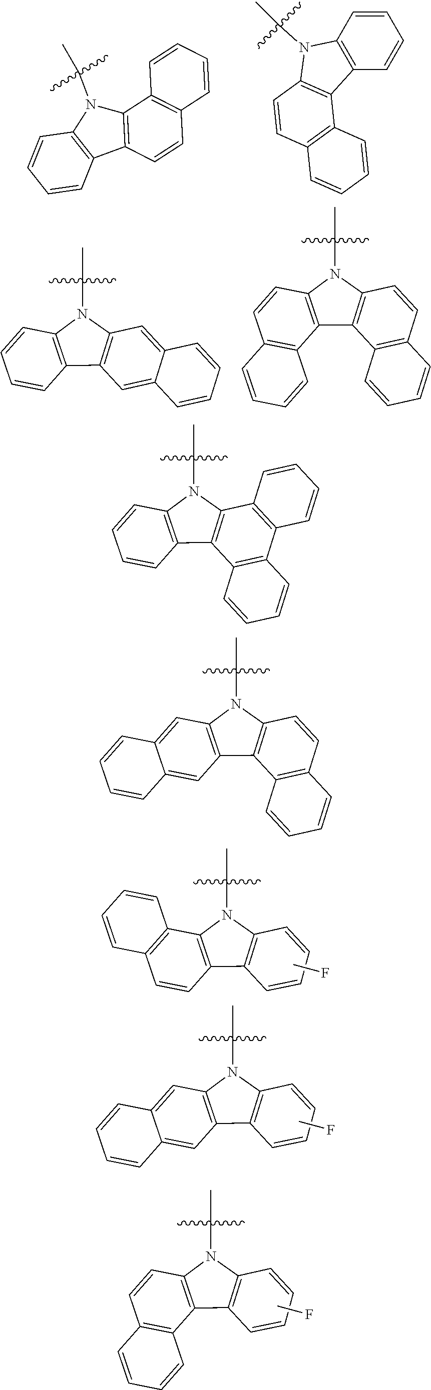

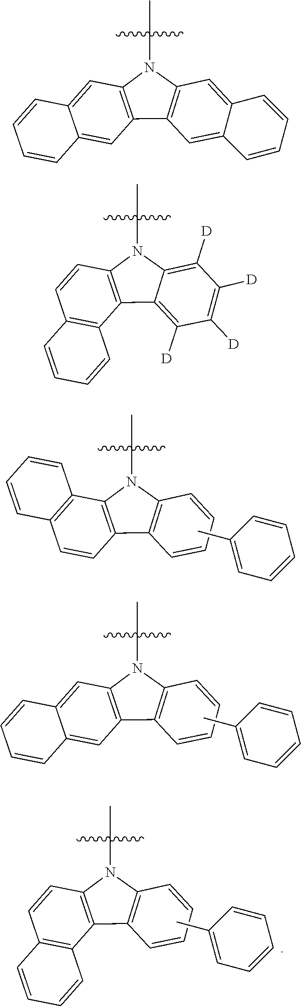

Optionally,

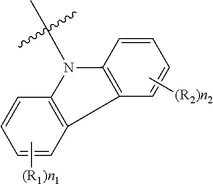

in the formula 1 is selected from the following structures:

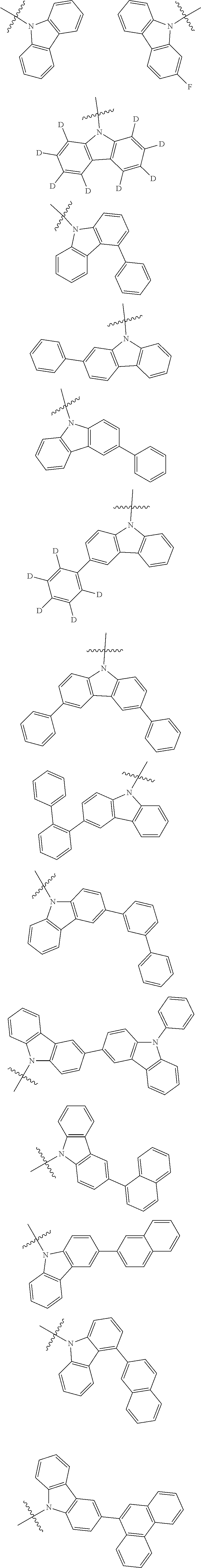

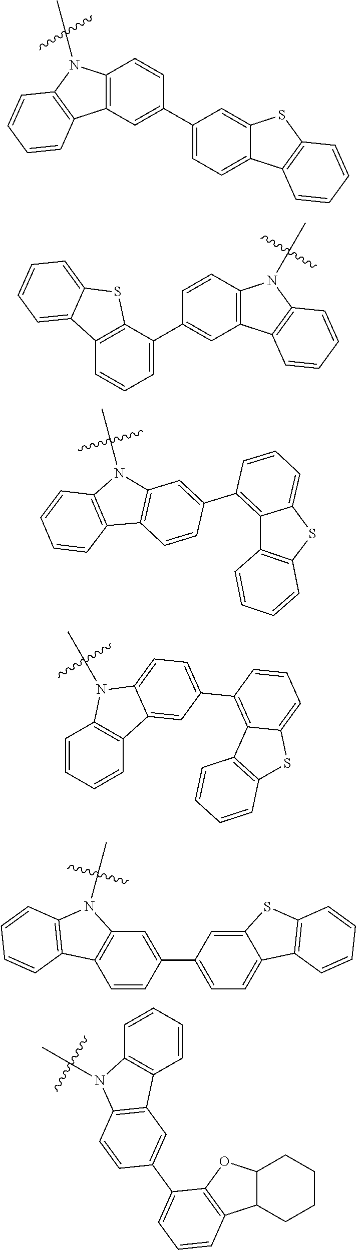

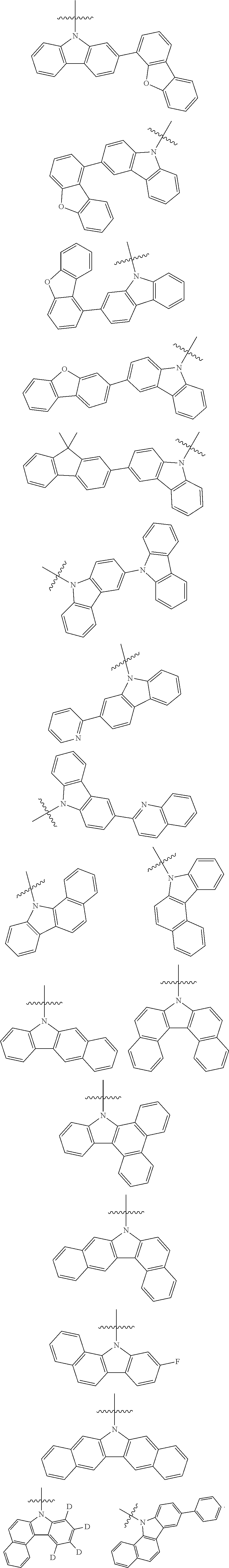

Optionally, the organic compound is selected from the group consisting of the following compounds:

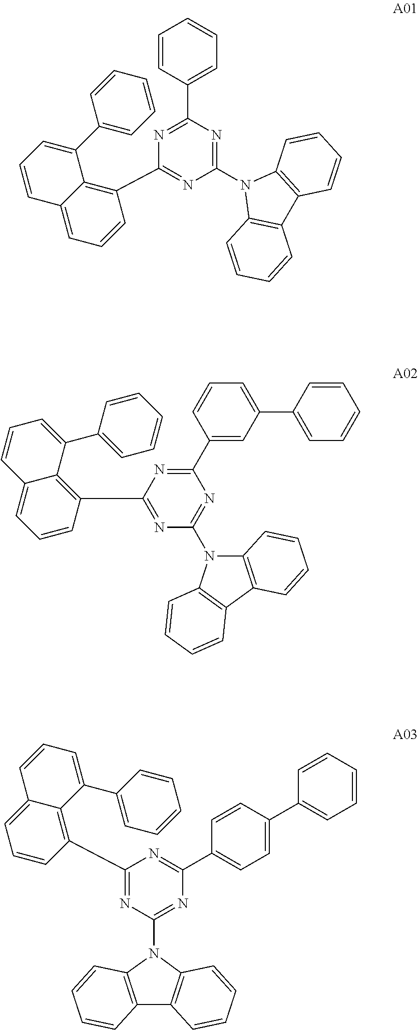 [SMILES: c1ccc(-c2nc(-c3cccc4cccc(-c5ccccc5)c34)nc(-n3c4ccccc4c4ccccc43)n2)cc1 ; c1ccc(-c2cccc(-c3nc(-c4cccc5cccc(-c6ccccc6)c45)nc(-n4c5ccccc5c5ccccc54)n3)c2)cc1 ; c1ccc(-c2ccc(-c3nc(-c4cccc5cccc(-c6ccccc6)c45)nc(-n4c5ccccc5c5ccccc54)n3)cc2)cc1]

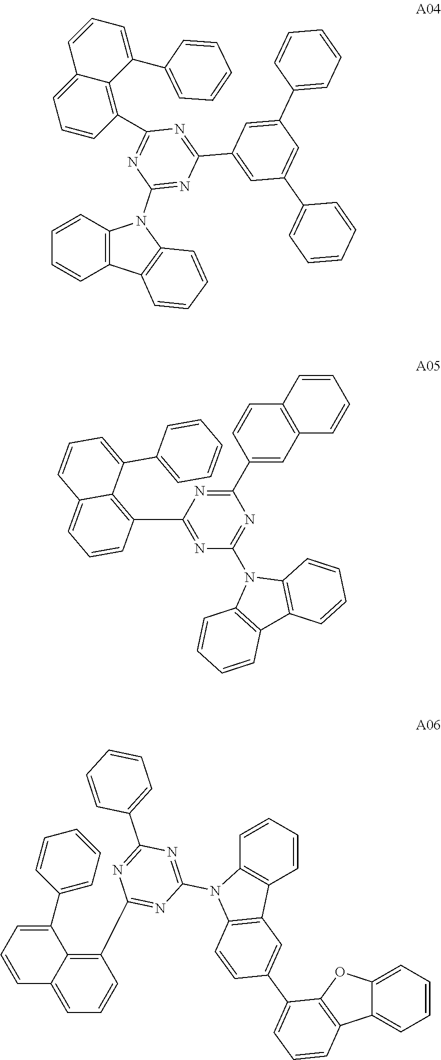 [SMILES: c1ccc(-c2cc(-c3ccccc3)cc(-c3nc(-c4cccc5cccc(-c6ccccc6)c45)nc(-n4c5ccccc5c5ccccc54)n3)c2)cc1 ; c1ccc(-c2cccc3cccc(-c4nc(-c5ccc6ccccc6c5)nc(-n5c6ccccc6c6ccccc65)n4)c23)cc1 ; c1ccc(-c2nc(-c3cccc4cccc(-c5ccccc5)c34)nc(-n3c4ccccc4c4cc(-c5cccc6c5oc5ccccc56)ccc43)n2)cc1]

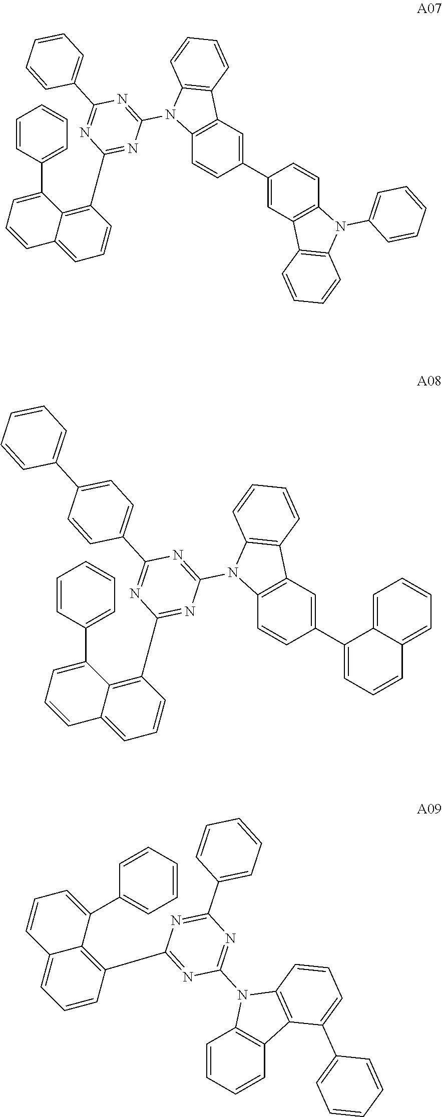 [SMILES: c1ccc(-c2nc(-c3cccc4cccc(-c5ccccc5)c34)nc(-n3c4ccccc4c4cc(-c5ccc6c(c5)c5ccccc5n6-c5ccccc5)ccc43)n2)cc1 ; c1ccc(-c2ccc(-c3nc(-c4cccc5cccc(-c6ccccc6)c45)nc(-n4c5ccccc5c5cc(-c6cccc7ccccc67)ccc54)n3)cc2)cc1 ; c1ccc(-c2nc(-c3cccc4cccc(-c5ccccc5)c34)nc(-n3c4ccccc4c4c(-c5ccccc5)cccc43)n2)cc1]

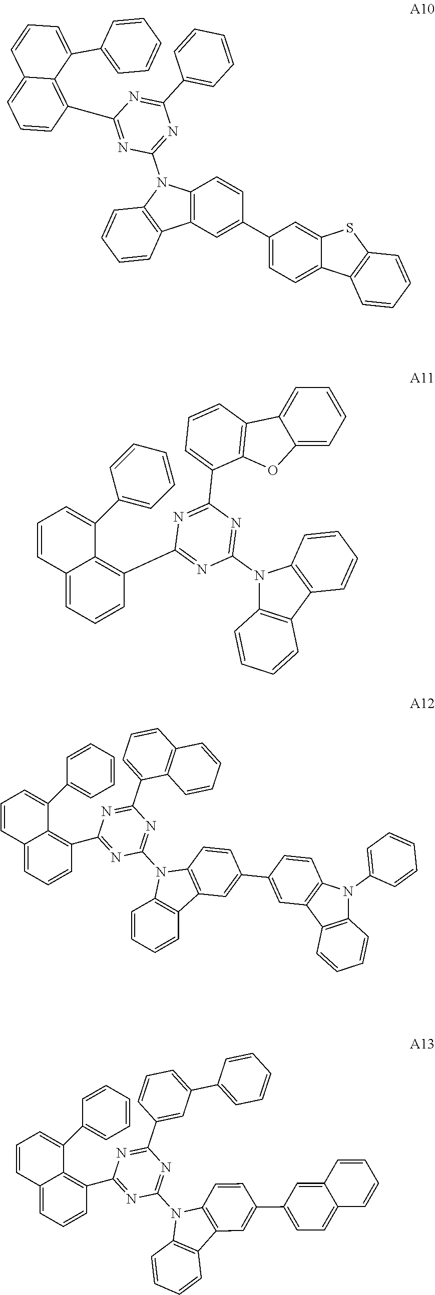 [SMILES: c1ccc(-c2nc(-c3cccc4cccc(-c5ccccc5)c34)nc(-n3c4ccccc4c4cc(-c5ccc6c(c5)sc5ccccc56)ccc43)n2)cc1 ; c1ccc(-c2cccc3cccc(-c4nc(-c5cccc6c5oc5ccccc56)nc(-n5c6ccccc6c6ccccc65)n4)c23)cc1 ; c1ccc(-c2cccc3cccc(-c4nc(-c5cccc6ccccc56)nc(-n5c6ccccc6c6cc(-c7ccc8c(c7)c7ccccc7n8-c7ccccc7)ccc65)n4)c23)cc1 ; c1ccc(-c2cccc(-c3nc(-c4cccc5cccc(-c6ccccc6)c45)nc(-n4c5ccccc5c5cc(-c6ccc7ccccc7c6)ccc54)n3)c2)cc1]

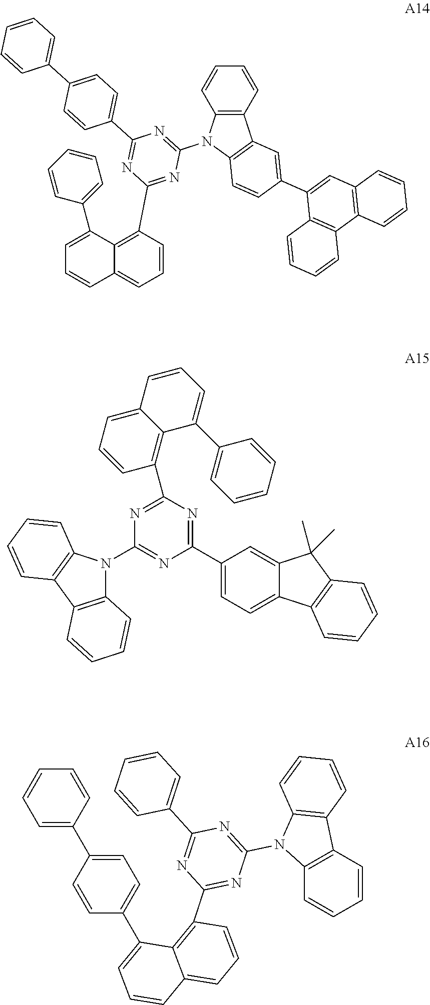 [SMILES: c1ccc(-c2ccc(-c3nc(-c4cccc5cccc(-c6ccccc6)c45)nc(-n4c5ccccc5c5cc(-c6cc7ccccc7c7ccccc67)ccc54)n3)cc2)cc1 ; CC1(C)c2ccccc2-c2ccc(-c3nc(-c4cccc5cccc(-c6ccccc6)c45)nc(-n4c5ccccc5c5ccccc54)n3)cc21 ; c1ccc(-c2ccc(-c3cccc4cccc(-c5nc(-c6ccccc6)nc(-n6c7ccccc7c7ccccc76)n5)c34)cc2)cc1]

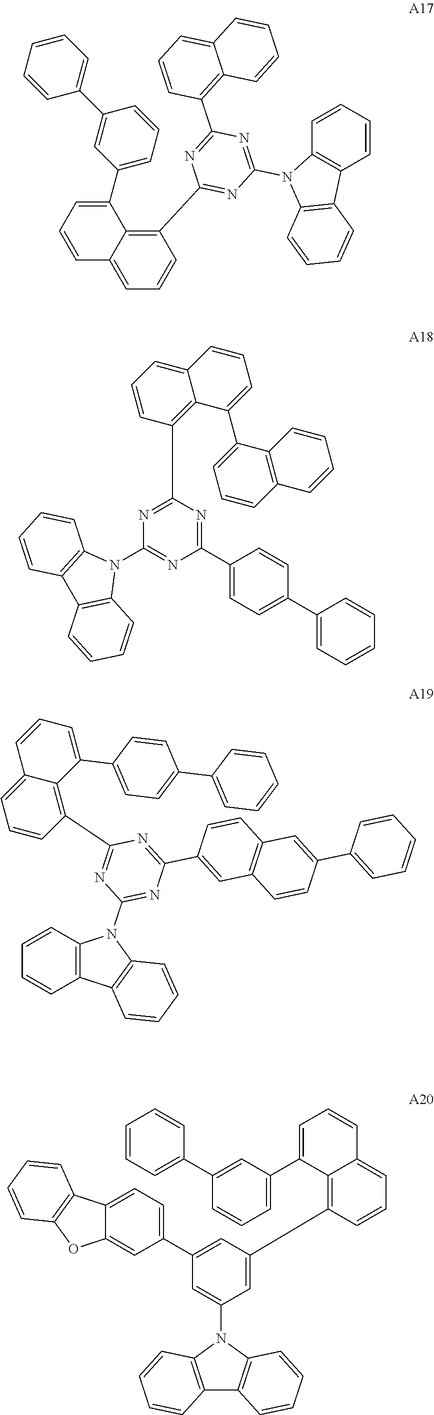 [SMILES: c1ccc(-c2cccc(-c3cccc4cccc(-c5nc(-c6cccc7ccccc67)nc(-n6c7ccccc7c7ccccc76)n5)c34)c2)cc1 ; c1ccc(-c2ccc(-c3nc(-c4cccc5cccc(-c6cccc7ccccc67)c45)nc(-n4c5ccccc5c5ccccc54)n3)cc2)cc1 ; c1ccc(-c2ccc(-c3cccc4cccc(-c5nc(-c6ccc7cc(-c8ccccc8)ccc7c6)nc(-n6c7ccccc7c7ccccc76)n5)c34)cc2)cc1 ; c1ccc(-c2cccc(-c3cccc4cccc(-c5cc(-c6ccc7c(c6)oc6ccccc67)cc(-n6c7ccccc7c7ccccc76)c5)c34)c2)cc1]

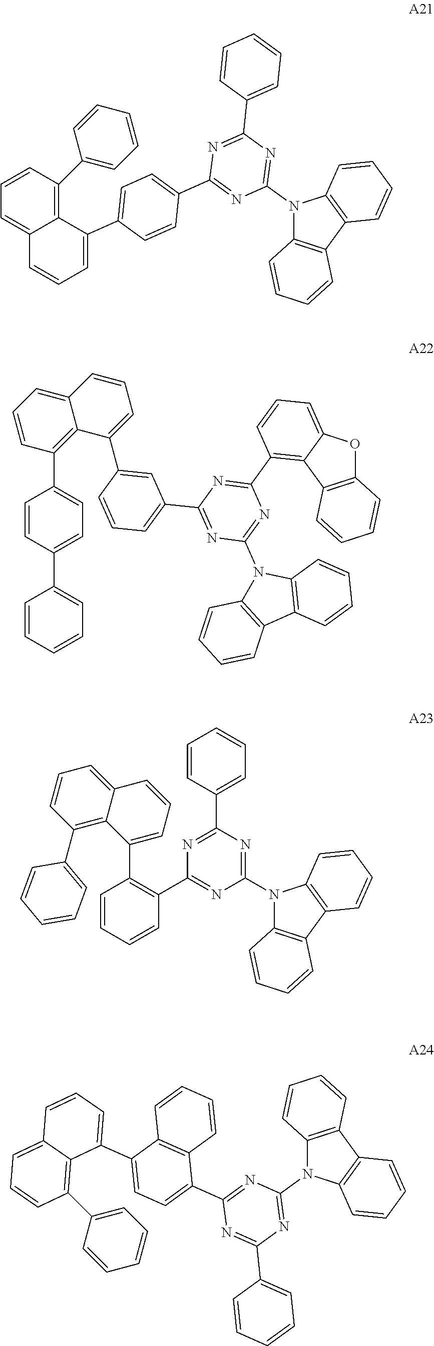 [SMILES: c1ccc(-c2nc(-c3ccc(-c4cccc5cccc(-c6ccccc6)c45)cc3)nc(-n3c4ccccc4c4ccccc43)n2)cc1 ; c1ccc(-c2ccc(-c3cccc4cccc(-c5cccc(-c6nc(-c7cccc8oc9ccccc9c78)nc(-n7c8ccccc8c8ccccc87)n6)c5)c34)cc2)cc1 ; c1ccc(-c2nc(-c3ccccc3-c3cccc4cccc(-c5ccccc5)c34)nc(-n3c4ccccc4c4ccccc43)n2)cc1 ; c1ccc(-c2nc(-c3ccc(-c4cccc5cccc(-c6ccccc6)c45)c4ccccc34)nc(-n3c4ccccc4c4ccccc43)n2)cc1]

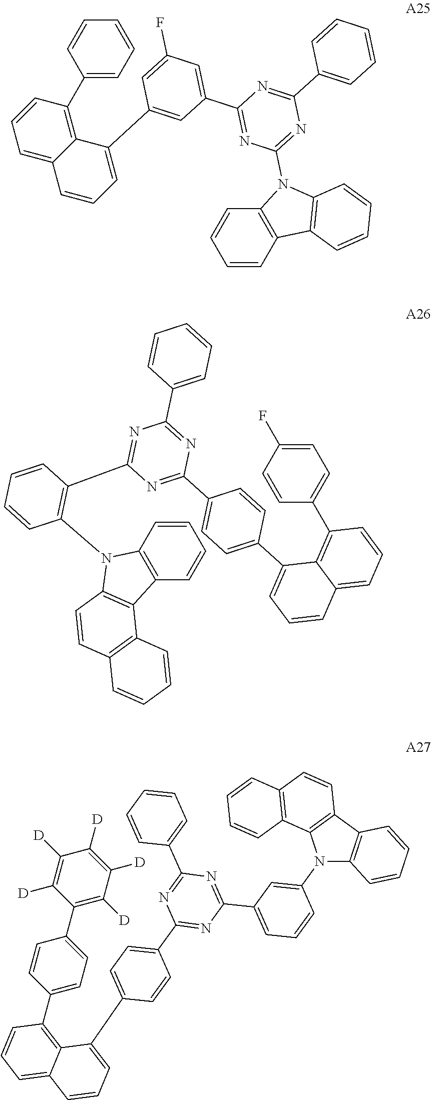 [SMILES: Fc1cc(-c2nc(-c3ccccc3)nc(-n3c4ccccc4c4ccccc43)n2)cc(-c2cccc3cccc(-c4ccccc4)c23)c1 ; Fc1ccc(-c2cccc3cccc(-c4ccc(-c5nc(-c6ccccc6)nc(-c6ccccc6-n6c7ccccc7c7c8ccccc8ccc76)n5)cc4)c23)cc1 ; [2H]c1c([2H])c([2H])c(-c2ccc(-c3cccc4cccc(-c5ccc(-c6nc(-c7ccccc7)nc(-c7cccc(-n8c9ccccc9c9ccc%10ccccc%10c98)c7)n6)cc5)c34)cc2)c([2H])c1[2H]]

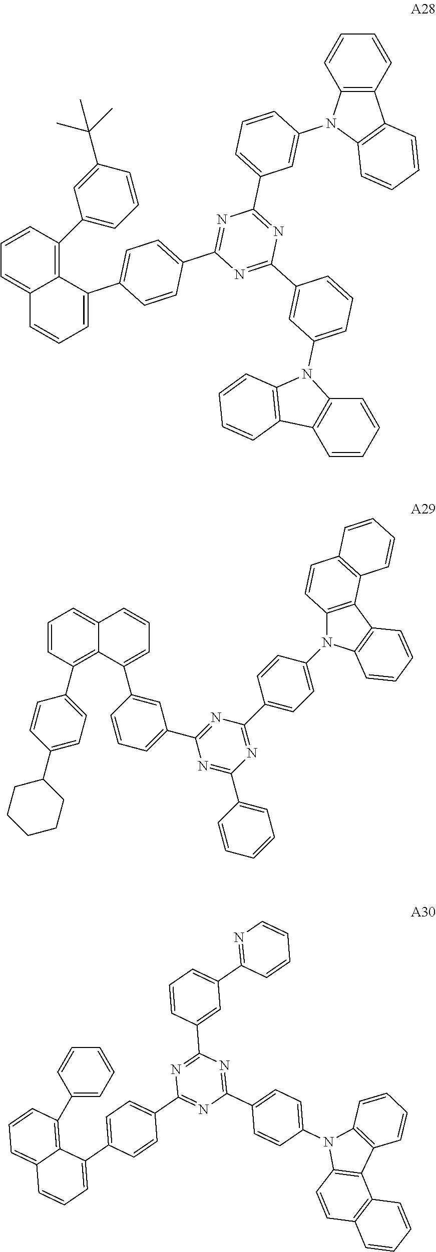 [SMILES: CC(C)(C)c1cccc(-c2cccc3cccc(-c4ccc(-c5nc(-c6cccc(-n7c8ccccc8c8ccccc87)c6)nc(-c6cccc(-n7c8ccccc8c8ccccc87)c6)n5)cc4)c23)c1 ; c1ccc(-c2nc(-c3ccc(-n4c5ccccc5c5c6ccccc6ccc54)cc3)nc(-c3cccc(-c4cccc5cccc(-c6ccc(C7CCCCC7)cc6)c45)c3)n2)cc1 ; c1ccc(-c2cccc3cccc(-c4ccc(-c5nc(-c6ccc(-n7c8ccccc8c8c9ccccc9ccc87)cc6)nc(-c6cccc(-c7ccccn7)c6)n5)cc4)c23)cc1]

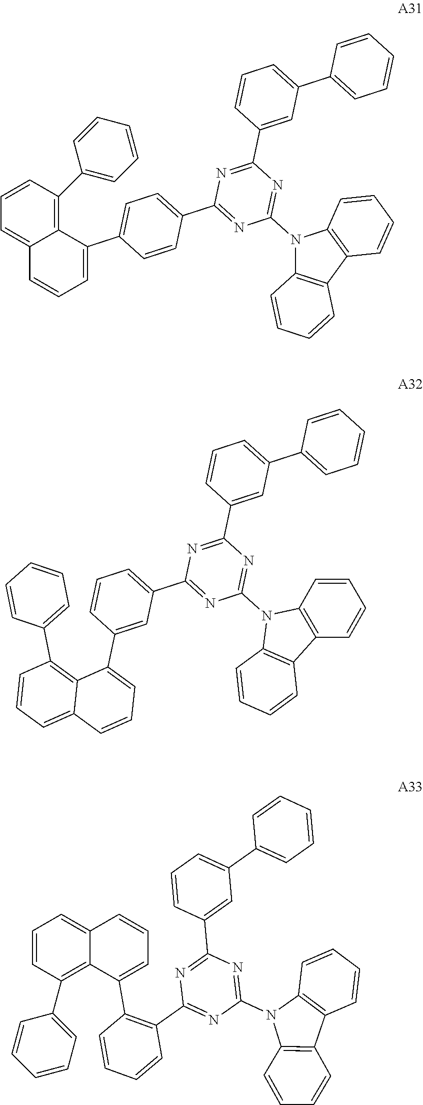 [SMILES: c1ccc(-c2cccc(-c3nc(-c4ccc(-c5cccc6cccc(-c7ccccc7)c56)cc4)nc(-n4c5ccccc5c5ccccc54)n3)c2)cc1 ; c1ccc(-c2cccc(-c3nc(-c4cccc(-c5cccc6cccc(-c7ccccc7)c56)c4)nc(-n4c5ccccc5c5ccccc54)n3)c2)cc1 ; c1ccc(-c2cccc(-c3nc(-c4ccccc4-c4cccc5cccc(-c6ccccc6)c45)nc(-n4c5ccccc5c5ccccc54)n3)c2)cc1]

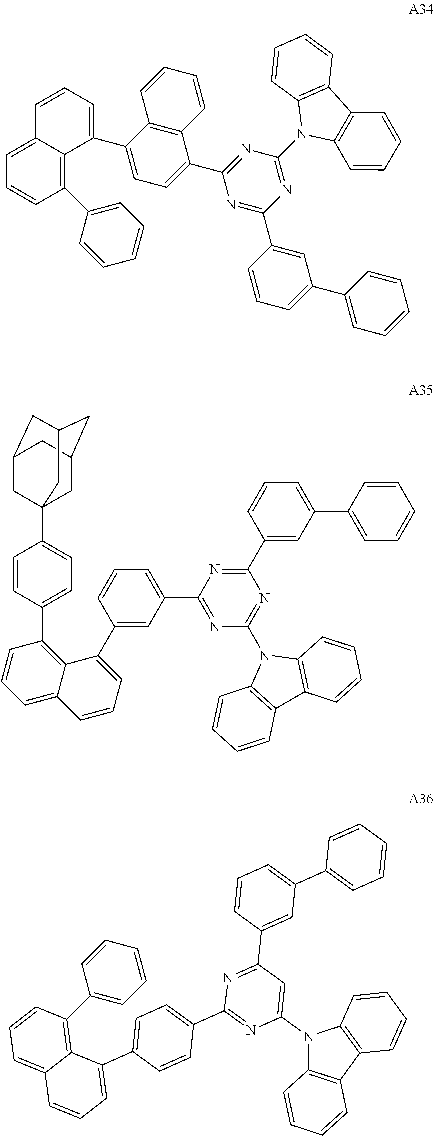 [SMILES: c1ccc(-c2cccc(-c3nc(-c4ccc(-c5cccc6cccc(-c7ccccc7)c56)c5ccccc45)nc(-n4c5ccccc5c5ccccc54)n3)c2)cc1 ; c1ccc(-c2cccc(-c3nc(-c4cccc(-c5cccc6cccc(-c7ccc(C89CC%10CC(CC(C%10)C8)C9)cc7)c56)c4)nc(-n4c5ccccc5c5ccccc54)n3)c2)cc1 ; c1ccc(-c2cccc(-c3cc(-n4c5ccccc5c5ccccc54)nc(-c4ccc(-c5cccc6cccc(-c7ccccc7)c56)cc4)n3)c2)cc1]

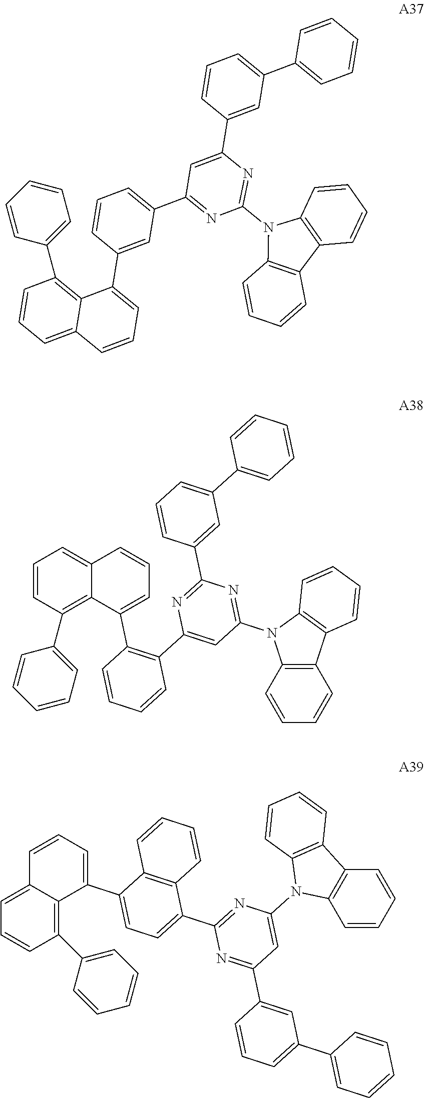 [SMILES: c1ccc(-c2cccc(-c3cc(-c4cccc(-c5cccc6cccc(-c7ccccc7)c56)c4)nc(-n4c5ccccc5c5ccccc54)n3)c2)cc1 ; c1ccc(-c2cccc(-c3nc(-c4ccccc4-c4cccc5cccc(-c6ccccc6)c45)cc(-n4c5ccccc5c5ccccc54)n3)c2)cc1 ; c1ccc(-c2cccc(-c3cc(-n4c5ccccc5c5ccccc54)nc(-c4ccc(-c5cccc6cccc(-c7ccccc7)c56)c5ccccc45)n3)c2)cc1]

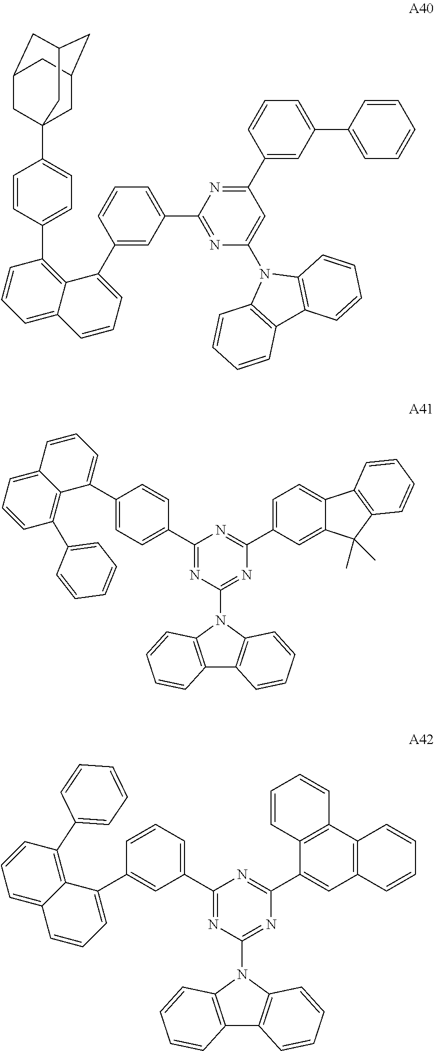 [SMILES: c1ccc(-c2cccc(-c3cc(-n4c5ccccc5c5ccccc54)nc(-c4cccc(-c5cccc6cccc(-c7ccc(C89CC%10CC(CC(C%10)C8)C9)cc7)c56)c4)n3)c2)cc1 ; CC1(C)c2ccccc2-c2ccc(-c3nc(-c4ccc(-c5cccc6cccc(-c7ccccc7)c56)cc4)nc(-n4c5ccccc5c5ccccc54)n3)cc21 ; c1ccc(-c2cccc3cccc(-c4cccc(-c5nc(-c6cc7ccccc7c7ccccc67)nc(-n6c7ccccc7c7ccccc76)n5)c4)c23)cc1]

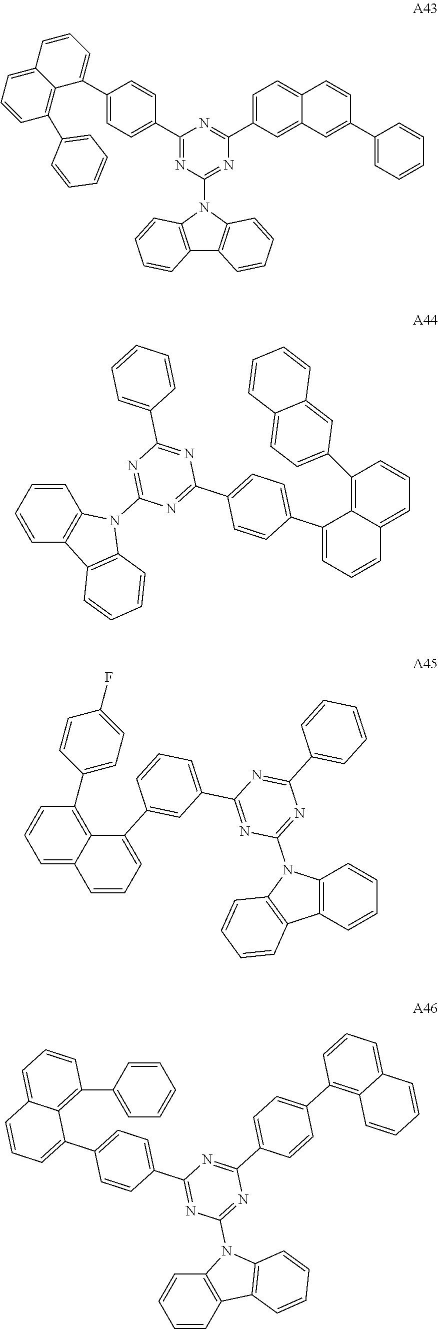 [SMILES: c1ccc(-c2ccc3ccc(-c4nc(-c5ccc(-c6cccc7cccc(-c8ccccc8)c67)cc5)nc(-n5c6ccccc6c6ccccc65)n4)cc3c2)cc1 ; c1ccc(-c2nc(-c3ccc(-c4cccc5cccc(-c6ccc7ccccc7c6)c45)cc3)nc(-n3c4ccccc4c4ccccc43)n2)cc1 ; Fc1ccc(-c2cccc3cccc(-c4cccc(-c5nc(-c6ccccc6)nc(-n6c7ccccc7c7ccccc76)n5)c4)c23)cc1 ; c1ccc(-c2cccc3cccc(-c4ccc(-c5nc(-c6ccc(-c7cccc8ccccc78)cc6)nc(-n6c7ccccc7c7ccccc76)n5)cc4)c23)cc1]

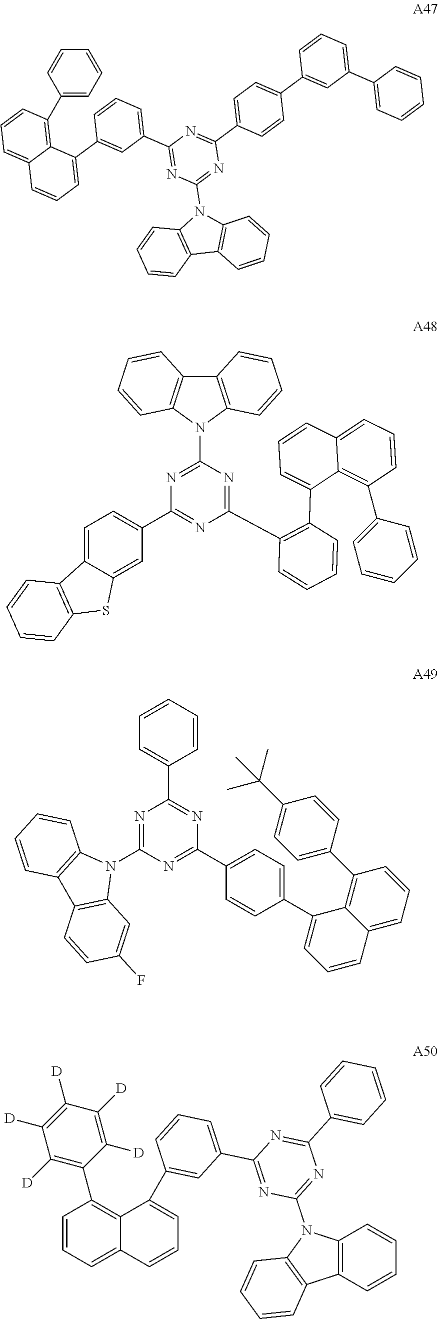 [SMILES: c1ccc(-c2cccc(-c3ccc(-c4nc(-c5cccc(-c6cccc7cccc(-c8ccccc8)c67)c5)nc(-n5c6ccccc6c6ccccc65)n4)cc3)c2)cc1 ; c1ccc(-c2cccc3cccc(-c4ccccc4-c4nc(-c5ccc6c(c5)sc5ccccc56)nc(-n5c6ccccc6c6ccccc65)n4)c23)cc1 ; CC(C)(C)c1ccc(-c2cccc3cccc(-c4ccc(-c5nc(-c6ccccc6)nc(-n6c7ccccc7c7ccc(F)cc76)n5)cc4)c23)cc1 ; [2H]c1c([2H])c([2H])c(-c2cccc3cccc(-c4cccc(-c5nc(-c6ccccc6)nc(-n6c7ccccc7c7ccccc76)n5)c4)c23)c([2H])c1[2H]]

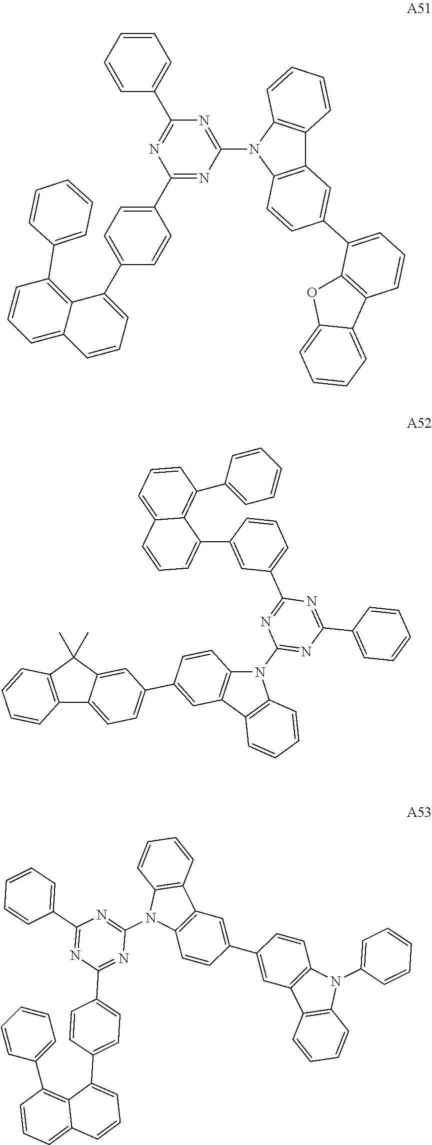 [SMILES: c1ccc(-c2nc(-c3ccc(-c4cccc5cccc(-c6ccccc6)c45)cc3)nc(-n3c4ccccc4c4cc(-c5cccc6c5oc5ccccc56)ccc43)n2)cc1 ; CC1(C)c2ccccc2-c2ccc(-c3ccc4c(c3)c3ccccc3n4-c3nc(-c4ccccc4)nc(-c4cccc(-c5cccc6cccc(-c7ccccc7)c56)c4)n3)cc21 ; c1ccc(-c2nc(-c3ccc(-c4cccc5cccc(-c6ccccc6)c45)cc3)nc(-n3c4ccccc4c4cc(-c5ccc6c(c5)c5ccccc5n6-c5ccccc5)ccc43)n2)cc1]

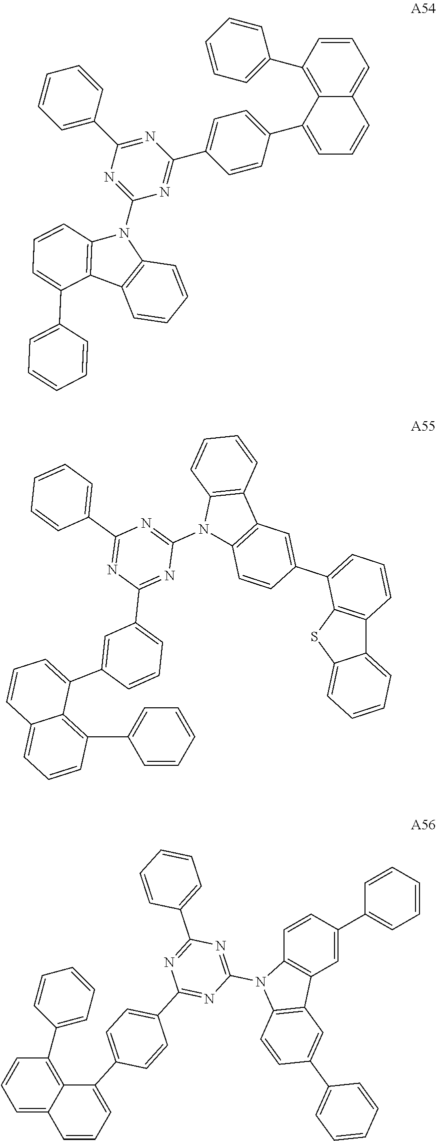 [SMILES: c1ccc(-c2nc(-c3ccc(-c4cccc5cccc(-c6ccccc6)c45)cc3)nc(-n3c4ccccc4c4c(-c5ccccc5)cccc43)n2)cc1 ; c1ccc(-c2nc(-c3cccc(-c4cccc5cccc(-c6ccccc6)c45)c3)nc(-n3c4ccccc4c4cc(-c5cccc6c5sc5ccccc56)ccc43)n2)cc1 ; c1ccc(-c2ccc3c(c2)c2cc(-c4ccccc4)ccc2n3-c2nc(-c3ccccc3)nc(-c3ccc(-c4cccc5cccc(-c6ccccc6)c45)cc3)n2)cc1]

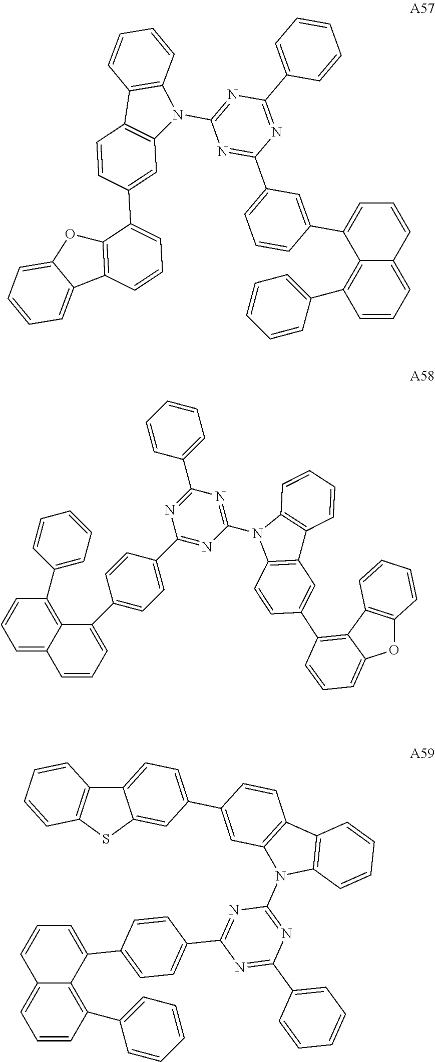 [SMILES: c1ccc(-c2nc(-c3cccc(-c4cccc5cccc(-c6ccccc6)c45)c3)nc(-n3c4ccccc4c4ccc(-c5cccc6c5oc5ccccc56)cc43)n2)cc1 ; c1ccc(-c2nc(-c3ccc(-c4cccc5cccc(-c6ccccc6)c45)cc3)nc(-n3c4ccccc4c4cc(-c5cccc6oc7ccccc7c56)ccc43)n2)cc1 ; c1ccc(-c2nc(-c3ccc(-c4cccc5cccc(-c6ccccc6)c45)cc3)nc(-n3c4ccccc4c4ccc(-c5ccc6c(c5)sc5ccccc56)cc43)n2)cc1]

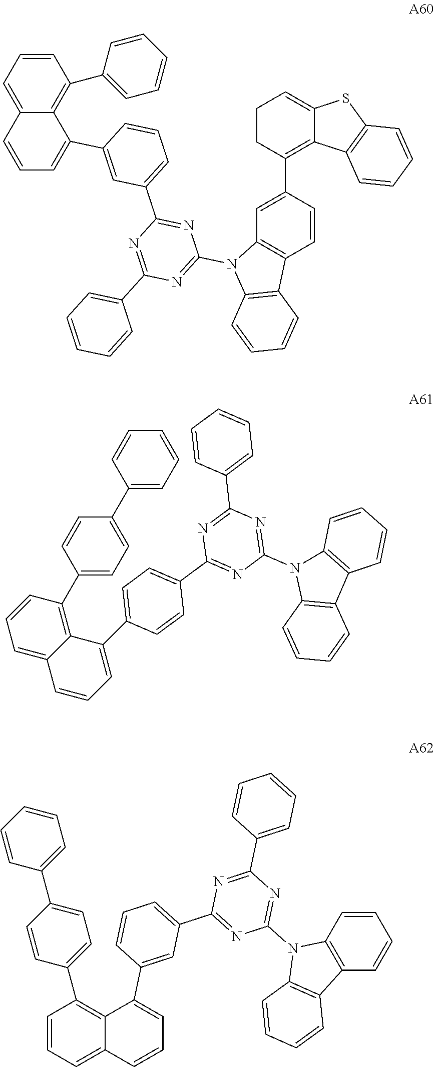 [SMILES: C1=c2sc3ccccc3c2=C(c2ccc3c4ccccc4n(-c4nc(-c5ccccc5)nc(-c5cccc(-c6cccc7cccc(-c8ccccc8)c67)c5)n4)c3c2)CC1 ; c1ccc(-c2ccc(-c3cccc4cccc(-c5ccc(-c6nc(-c7ccccc7)nc(-n7c8ccccc8c8ccccc87)n6)cc5)c34)cc2)cc1 ; c1ccc(-c2ccc(-c3cccc4cccc(-c5cccc(-c6nc(-c7ccccc7)nc(-n7c8ccccc8c8ccccc87)n6)c5)c34)cc2)cc1]

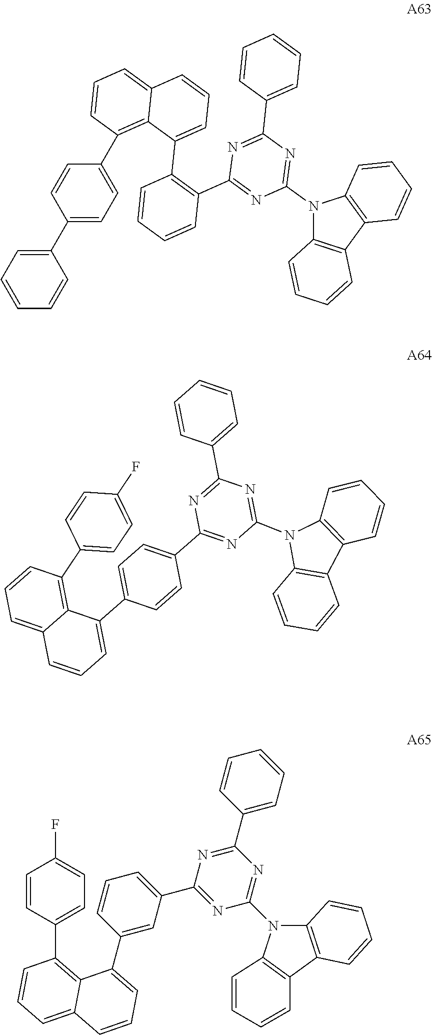 [SMILES: c1ccc(-c2ccc(-c3cccc4cccc(-c5ccccc5-c5nc(-c6ccccc6)nc(-n6c7ccccc7c7ccccc76)n5)c34)cc2)cc1 ; Fc1ccc(-c2cccc3cccc(-c4ccc(-c5nc(-c6ccccc6)nc(-n6c7ccccc7c7ccccc76)n5)cc4)c23)cc1 ; Fc1ccc(-c2cccc3cccc(-c4cccc(-c5nc(-c6ccccc6)nc(-n6c7ccccc7c7ccccc76)n5)c4)c23)cc1]

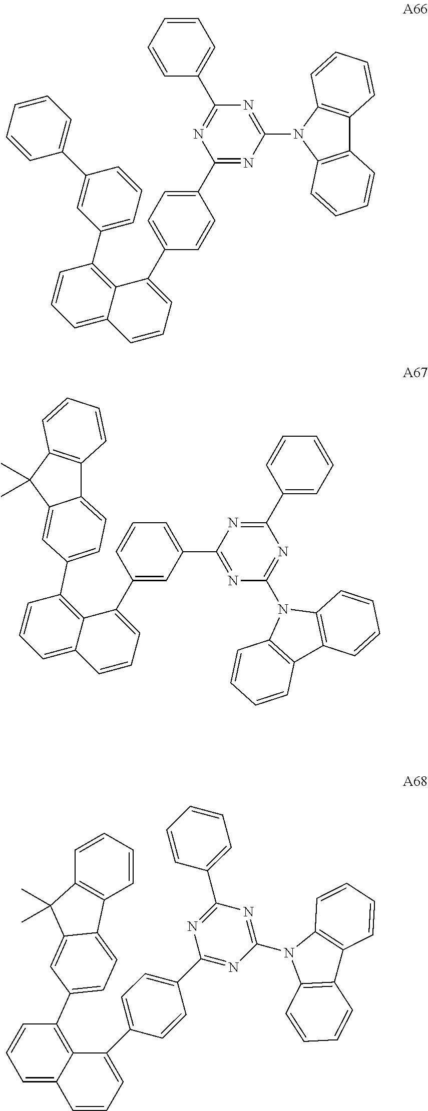 [SMILES: c1ccc(-c2cccc(-c3cccc4cccc(-c5ccc(-c6nc(-c7ccccc7)nc(-n7c8ccccc8c8ccccc87)n6)cc5)c34)c2)cc1 ; CC1(C)c2ccccc2-c2ccc(-c3cccc4cccc(-c5cccc(-c6nc(-c7ccccc7)nc(-n7c8ccccc8c8ccccc87)n6)c5)c34)cc21 ; CC1(C)c2ccccc2-c2ccc(-c3cccc4cccc(-c5ccc(-c6nc(-c7ccccc7)nc(-n7c8ccccc8c8ccccc87)n6)cc5)c34)cc21]

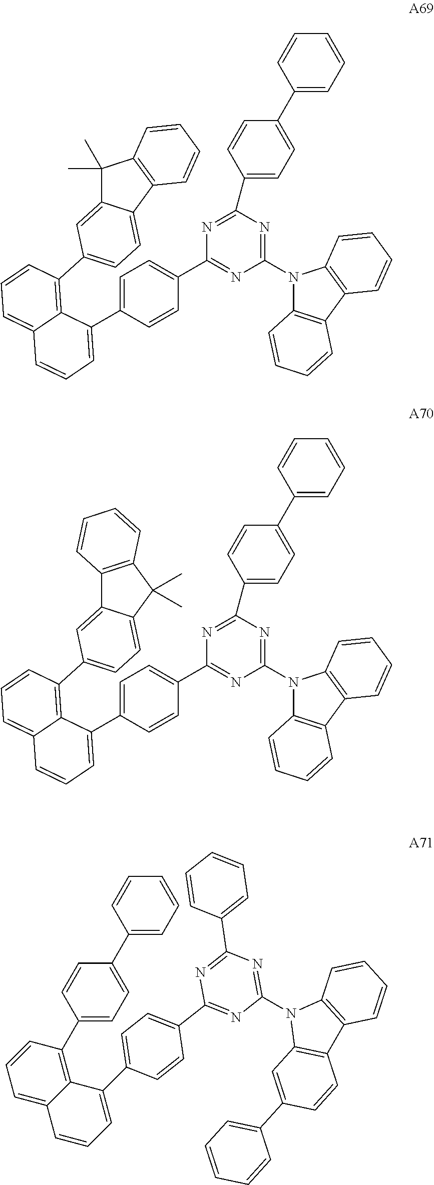 [SMILES: CC1(C)c2ccccc2-c2ccc(-c3cccc4cccc(-c5ccc(-c6nc(-c7ccc(-c8ccccc8)cc7)nc(-n7c8ccccc8c8ccccc87)n6)cc5)c34)cc21 ; CC1(C)c2ccccc2-c2cc(-c3cccc4cccc(-c5ccc(-c6nc(-c7ccc(-c8ccccc8)cc7)nc(-n7c8ccccc8c8ccccc87)n6)cc5)c34)ccc21 ; c1ccc(-c2ccc(-c3cccc4cccc(-c5ccc(-c6nc(-c7ccccc7)nc(-n7c8ccccc8c8ccc(-c9ccccc9)cc87)n6)cc5)c34)cc2)cc1]

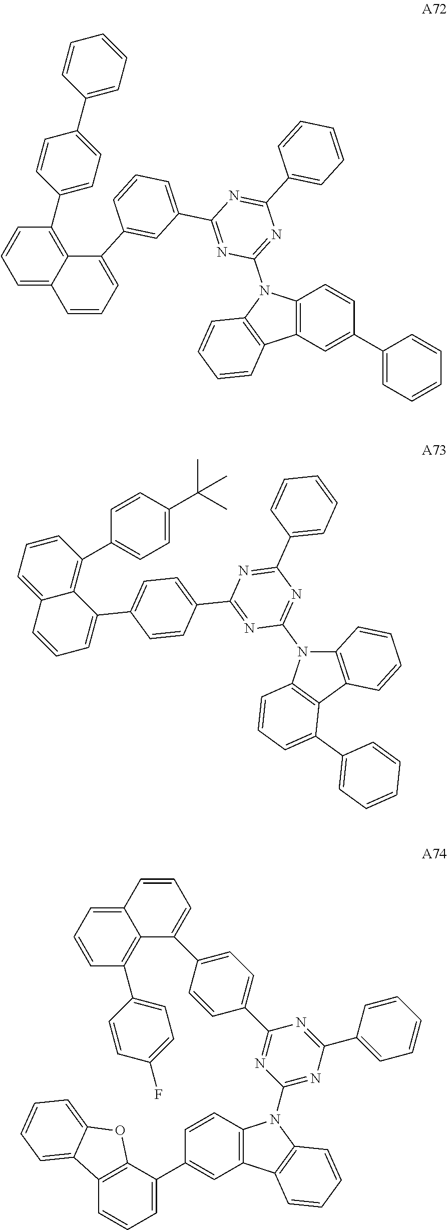 [SMILES: c1ccc(-c2ccc(-c3cccc4cccc(-c5cccc(-c6nc(-c7ccccc7)nc(-n7c8ccccc8c8cc(-c9ccccc9)ccc87)n6)c5)c34)cc2)cc1 ; CC(C)(C)c1ccc(-c2cccc3cccc(-c4ccc(-c5nc(-c6ccccc6)nc(-n6c7ccccc7c7c(-c8ccccc8)cccc76)n5)cc4)c23)cc1 ; Fc1ccc(-c2cccc3cccc(-c4ccc(-c5nc(-c6ccccc6)nc(-n6c7ccccc7c7cc(-c8cccc9c8oc8ccccc89)ccc76)n5)cc4)c23)cc1]

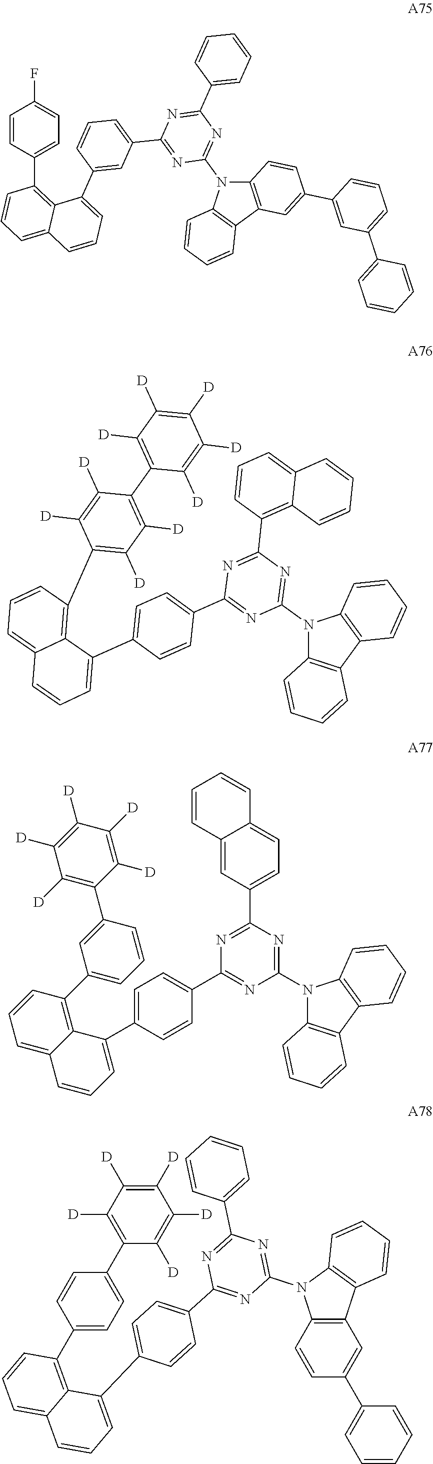

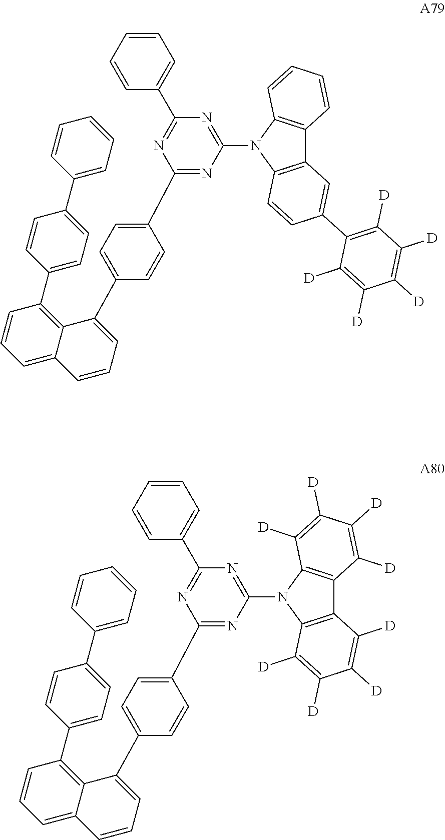 [SMILES: [2H]c1c([2H])c([2H])c(-c2ccc3c(c2)c2ccccc2n3-c2nc(-c3ccccc3)nc(-c3ccc(-c4cccc5cccc(-c6ccc(-c7ccccc7)cc6)c45)cc3)n2)c([2H])c1[2H] ; [2H]c1c([2H])c([2H])c2c(c1[2H])c1c([2H])c([2H])c([2H])c([2H])c1n2-c1nc(-c2ccccc2)nc(-c2ccc(-c3cccc4cccc(-c5ccc(-c6ccccc6)cc5)c34)cc2)n1]

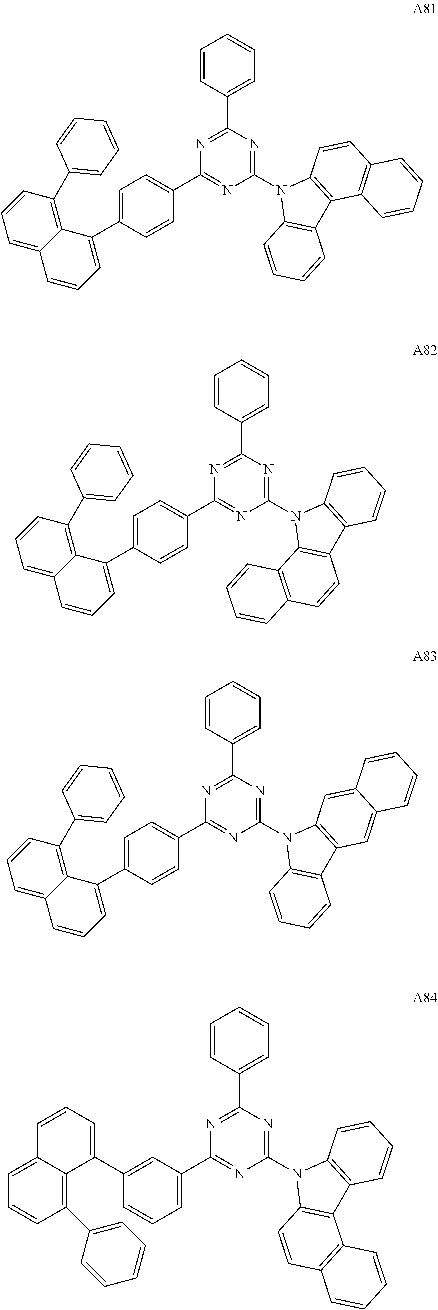 [SMILES: c1ccc(-c2nc(-c3ccc(-c4cccc5cccc(-c6ccccc6)c45)cc3)nc(-n3c4ccccc4c4c5ccccc5ccc43)n2)cc1 ; c1ccc(-c2nc(-c3ccc(-c4cccc5cccc(-c6ccccc6)c45)cc3)nc(-n3c4ccccc4c4ccc5ccccc5c43)n2)cc1 ; c1ccc(-c2nc(-c3ccc(-c4cccc5cccc(-c6ccccc6)c45)cc3)nc(-n3c4ccccc4c4cc5ccccc5cc43)n2)cc1 ; c1ccc(-c2nc(-c3cccc(-c4cccc5cccc(-c6ccccc6)c45)c3)nc(-n3c4ccccc4c4c5ccccc5ccc43)n2)cc1]

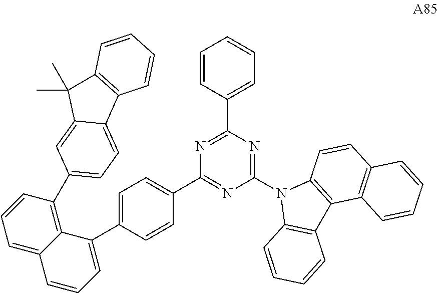 [SMILES: CC1(C)c2ccccc2-c2ccc(-c3cccc4cccc(-c5ccc(-c6nc(-c7ccccc7)nc(-n7c8ccccc8c8c9ccccc9ccc87)n6)cc5)c34)cc21]

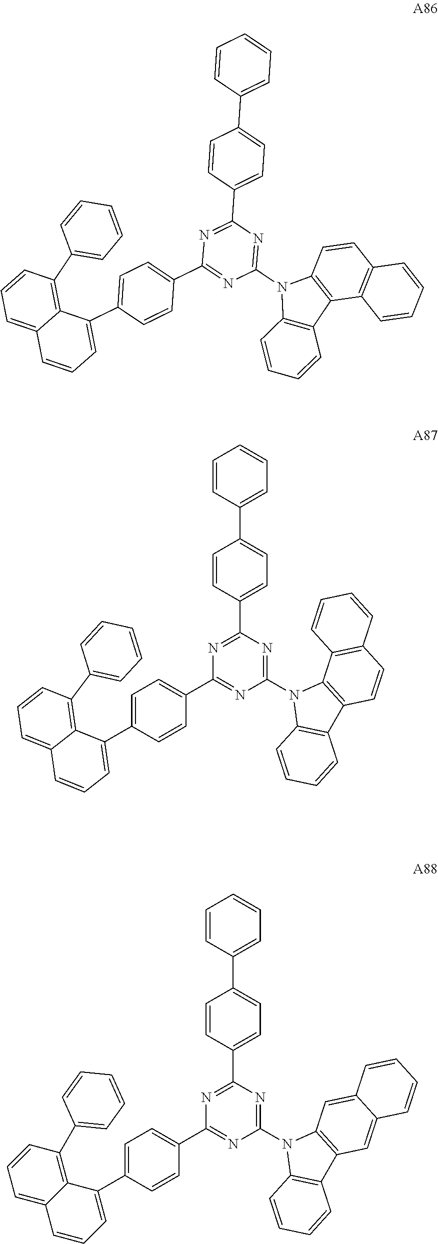 [SMILES: c1ccc(-c2ccc(-c3nc(-c4ccc(-c5cccc6cccc(-c7ccccc7)c56)cc4)nc(-n4c5ccccc5c5c6ccccc6ccc54)n3)cc2)cc1 ; c1ccc(-c2ccc(-c3nc(-c4ccc(-c5cccc6cccc(-c7ccccc7)c56)cc4)nc(-n4c5ccccc5c5ccc6ccccc6c54)n3)cc2)cc1 ; c1ccc(-c2ccc(-c3nc(-c4ccc(-c5cccc6cccc(-c7ccccc7)c56)cc4)nc(-n4c5ccccc5c5cc6ccccc6cc54)n3)cc2)cc1]

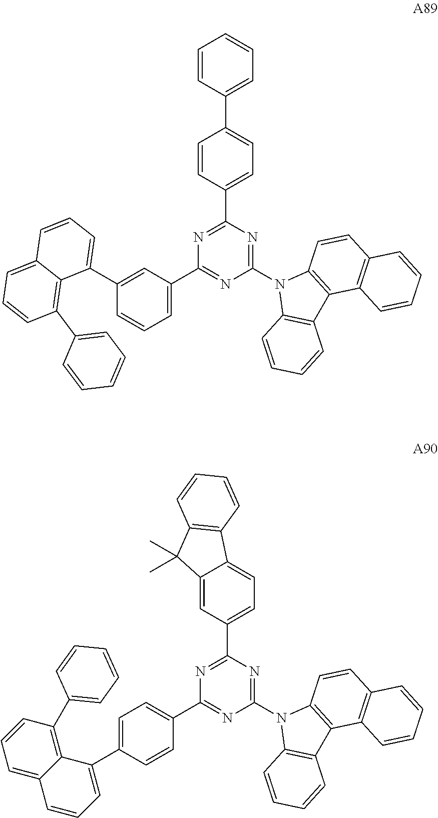 [SMILES: c1ccc(-c2ccc(-c3nc(-c4cccc(-c5cccc6cccc(-c7ccccc7)c56)c4)nc(-n4c5ccccc5c5c6ccccc6ccc54)n3)cc2)cc1 ; CC1(C)c2ccccc2-c2ccc(-c3nc(-c4ccc(-c5cccc6cccc(-c7ccccc7)c56)cc4)nc(-n4c5ccccc5c5c6ccccc6ccc54)n3)cc21]

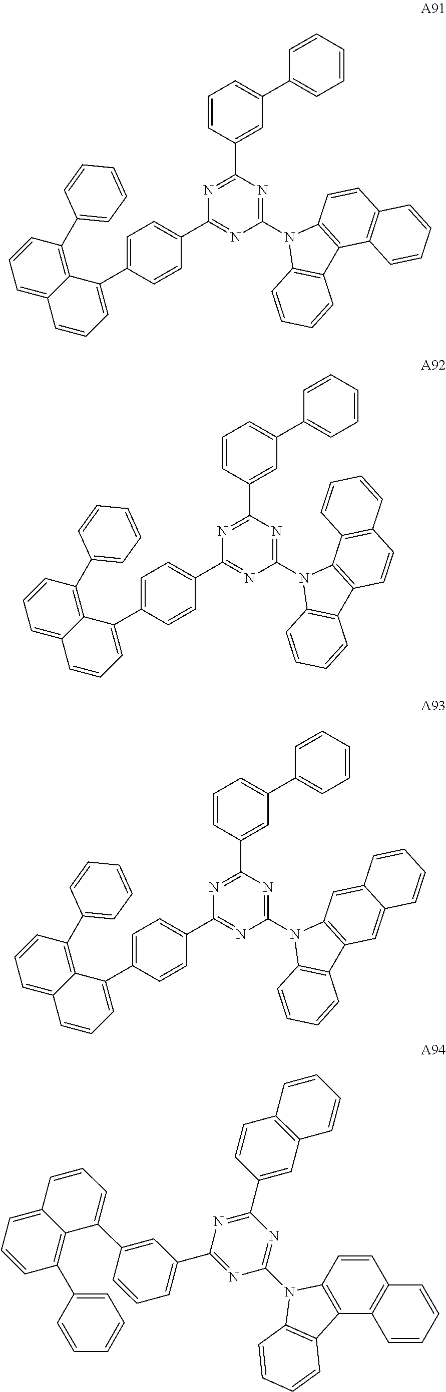 [SMILES: c1ccc(-c2cccc(-c3nc(-c4ccc(-c5cccc6cccc(-c7ccccc7)c56)cc4)nc(-n4c5ccccc5c5c6ccccc6ccc54)n3)c2)cc1 ; c1ccc(-c2cccc(-c3nc(-c4ccc(-c5cccc6cccc(-c7ccccc7)c56)cc4)nc(-n4c5ccccc5c5ccc6ccccc6c54)n3)c2)cc1 ; c1ccc(-c2cccc(-c3nc(-c4ccc(-c5cccc6cccc(-c7ccccc7)c56)cc4)nc(-n4c5ccccc5c5cc6ccccc6cc54)n3)c2)cc1 ; c1ccc(-c2cccc3cccc(-c4cccc(-c5nc(-c6ccc7ccccc7c6)nc(-n6c7ccccc7c7c8ccccc8ccc76)n5)c4)c23)cc1]

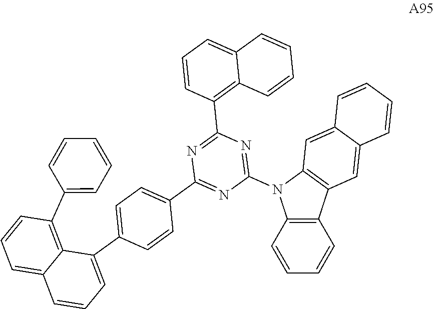 [SMILES: c1ccc(-c2cccc3cccc(-c4ccc(-c5nc(-c6cccc7ccccc67)nc(-n6c7ccccc7c7cc8ccccc8cc76)n5)cc4)c23)cc1]

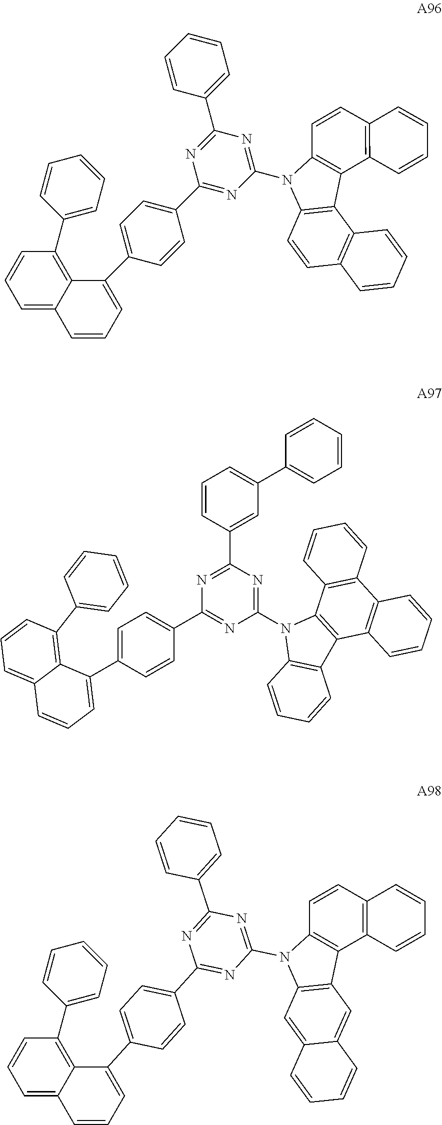 [SMILES: c1ccc(-c2nc(-c3ccc(-c4cccc5cccc(-c6ccccc6)c45)cc3)nc(-n3c4ccc5ccccc5c4c4c5ccccc5ccc43)n2)cc1 ; c1ccc(-c2cccc(-c3nc(-c4ccc(-c5cccc6cccc(-c7ccccc7)c56)cc4)nc(-n4c5ccccc5c5c6ccccc6c6ccccc6c54)n3)c2)cc1 ; c1ccc(-c2nc(-c3ccc(-c4cccc5cccc(-c6ccccc6)c45)cc3)nc(-n3c4cc5ccccc5cc4c4c5ccccc5ccc43)n2)cc1]

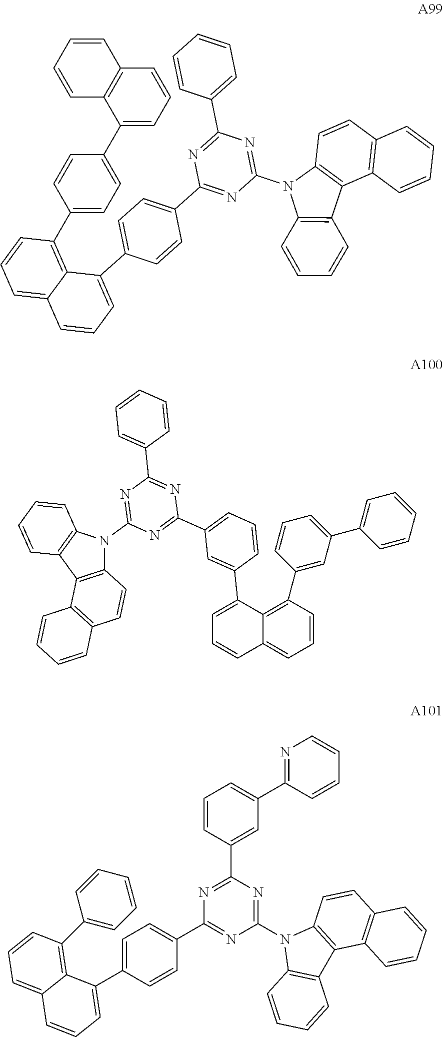 [SMILES: c1ccc(-c2nc(-c3ccc(-c4cccc5cccc(-c6ccc(-c7cccc8ccccc78)cc6)c45)cc3)nc(-n3c4ccccc4c4c5ccccc5ccc43)n2)cc1 ; c1ccc(-c2cccc(-c3cccc4cccc(-c5cccc(-c6nc(-c7ccccc7)nc(-n7c8ccccc8c8c9ccccc9ccc87)n6)c5)c34)c2)cc1 ; c1ccc(-c2cccc3cccc(-c4ccc(-c5nc(-c6cccc(-c7ccccn7)c6)nc(-n6c7ccccc7c7c8ccccc8ccc76)n5)cc4)c23)cc1]

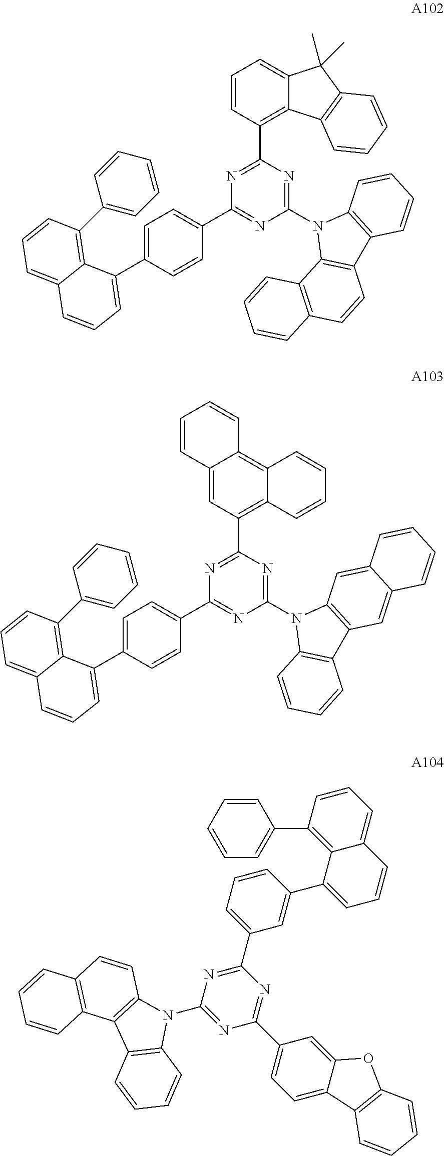 [SMILES: CC1(C)c2ccccc2-c2c(-c3nc(-c4ccc(-c5cccc6cccc(-c7ccccc7)c56)cc4)nc(-n4c5ccccc5c5ccc6ccccc6c54)n3)cccc21 ; c1ccc(-c2cccc3cccc(-c4ccc(-c5nc(-c6cc7ccccc7c7ccccc67)nc(-n6c7ccccc7c7cc8ccccc8cc76)n5)cc4)c23)cc1 ; c1ccc(-c2cccc3cccc(-c4cccc(-c5nc(-c6ccc7c(c6)oc6ccccc67)nc(-n6c7ccccc7c7c8ccccc8ccc76)n5)c4)c23)cc1]

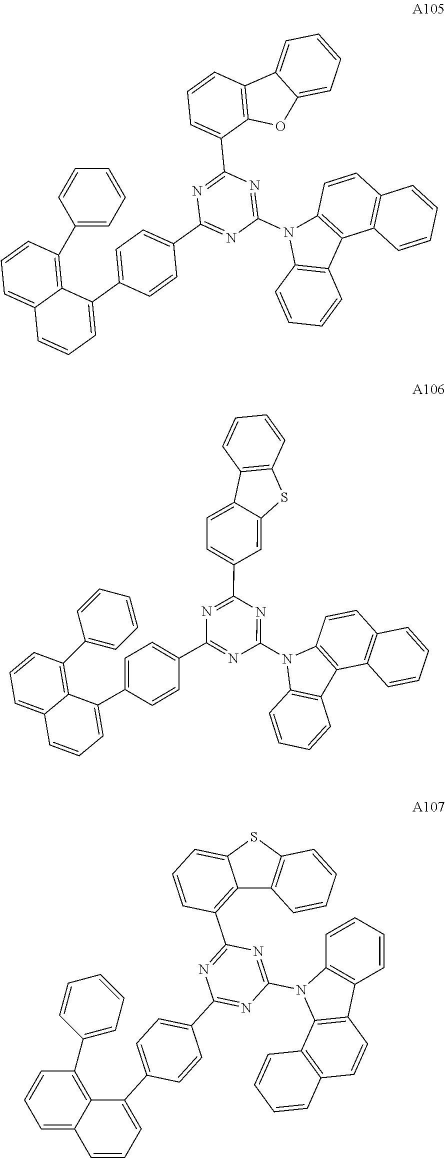 [SMILES: c1ccc(-c2cccc3cccc(-c4ccc(-c5nc(-c6cccc7c6oc6ccccc67)nc(-n6c7ccccc7c7c8ccccc8ccc76)n5)cc4)c23)cc1 ; c1ccc(-c2cccc3cccc(-c4ccc(-c5nc(-c6ccc7c(c6)sc6ccccc67)nc(-n6c7ccccc7c7c8ccccc8ccc76)n5)cc4)c23)cc1 ; c1ccc(-c2cccc3cccc(-c4ccc(-c5nc(-c6cccc7sc8ccccc8c67)nc(-n6c7ccccc7c7ccc8ccccc8c76)n5)cc4)c23)cc1]

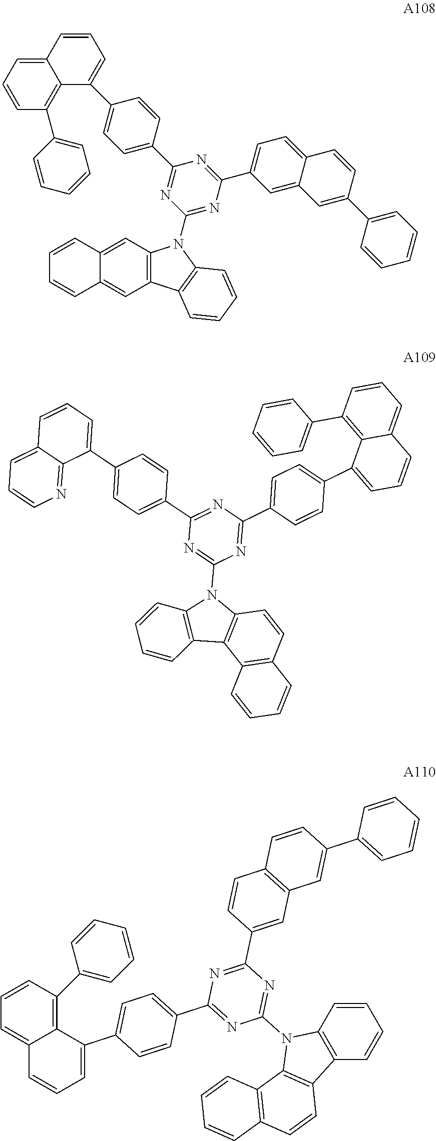 [SMILES: c1ccc(-c2ccc3ccc(-c4nc(-c5ccc(-c6cccc7cccc(-c8ccccc8)c67)cc5)nc(-n5c6ccccc6c6cc7ccccc7cc65)n4)cc3c2)cc1 ; c1ccc(-c2cccc3cccc(-c4ccc(-c5nc(-c6ccc(-c7cccc8cccnc78)cc6)nc(-n6c7ccccc7c7c8ccccc8ccc76)n5)cc4)c23)cc1 ; c1ccc(-c2ccc3ccc(-c4nc(-c5ccc(-c6cccc7cccc(-c8ccccc8)c67)cc5)nc(-n5c6ccccc6c6ccc7ccccc7c65)n4)cc3c2)cc1]

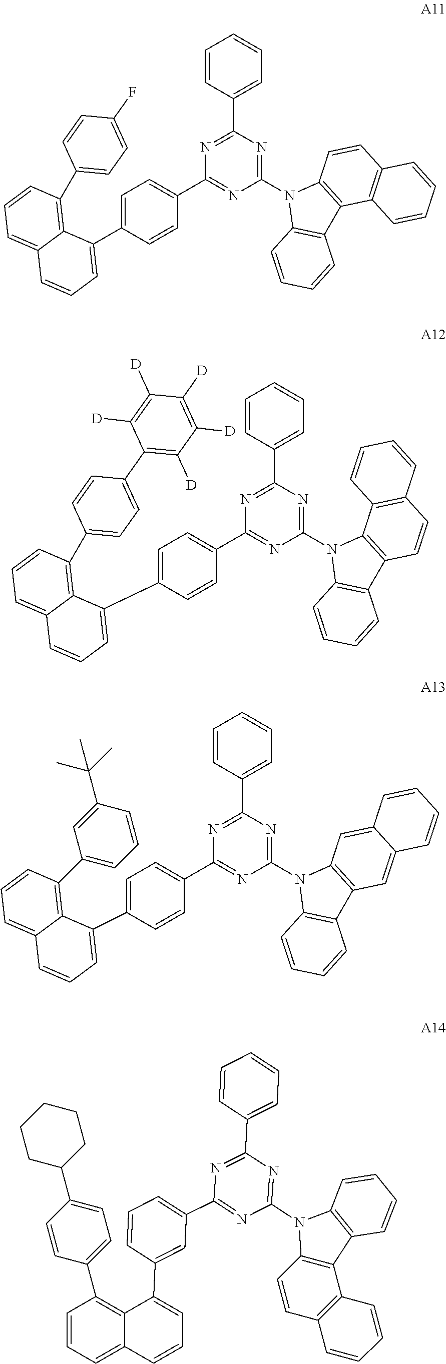 [SMILES: Fc1ccc(-c2cccc3cccc(-c4ccc(-c5nc(-c6ccccc6)nc(-n6c7ccccc7c7c8ccccc8ccc76)n5)cc4)c23)cc1 ; [2H]c1c([2H])c([2H])c(-c2ccc(-c3cccc4cccc(-c5ccc(-c6nc(-c7ccccc7)nc(-n7c8ccccc8c8ccc9ccccc9c87)n6)cc5)c34)cc2)c([2H])c1[2H] ; CC(C)(C)c1cccc(-c2cccc3cccc(-c4ccc(-c5nc(-c6ccccc6)nc(-n6c7ccccc7c7cc8ccccc8cc76)n5)cc4)c23)c1 ; c1ccc(-c2nc(-c3cccc(-c4cccc5cccc(-c6ccc(C7CCCCC7)cc6)c45)c3)nc(-n3c4ccccc4c4c5ccccc5ccc43)n2)cc1]

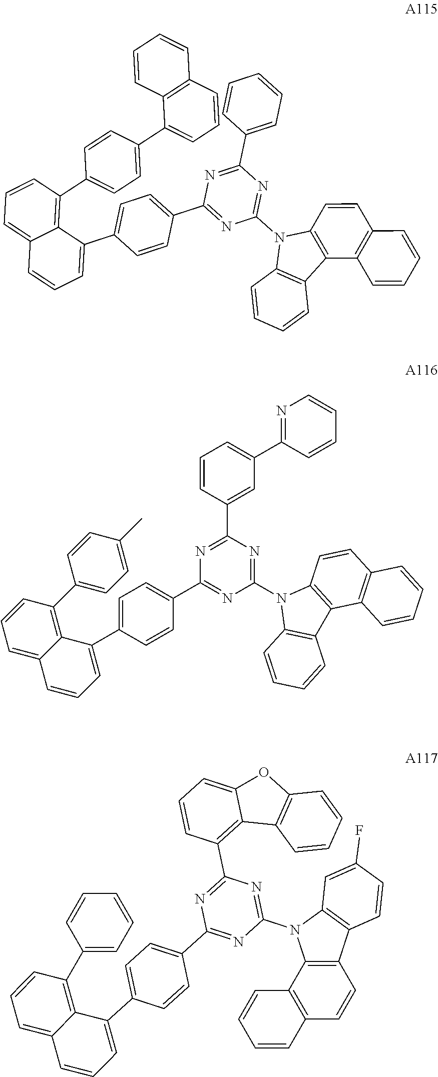 [SMILES: c1ccc(-c2nc(-c3ccc(-c4cccc5cccc(-c6ccc(-c7cccc8ccccc78)cc6)c45)cc3)nc(-n3c4ccccc4c4c5ccccc5ccc43)n2)cc1 ; Cc1ccc(-c2cccc3cccc(-c4ccc(-c5nc(-c6cccc(-c7ccccn7)c6)nc(-n6c7ccccc7c7c8ccccc8ccc76)n5)cc4)c23)cc1 ; Fc1ccc2c3ccc4ccccc4c3n(-c3nc(-c4ccc(-c5cccc6cccc(-c7ccccc7)c56)cc4)nc(-c4cccc5oc6ccccc6c45)n3)c2c1]

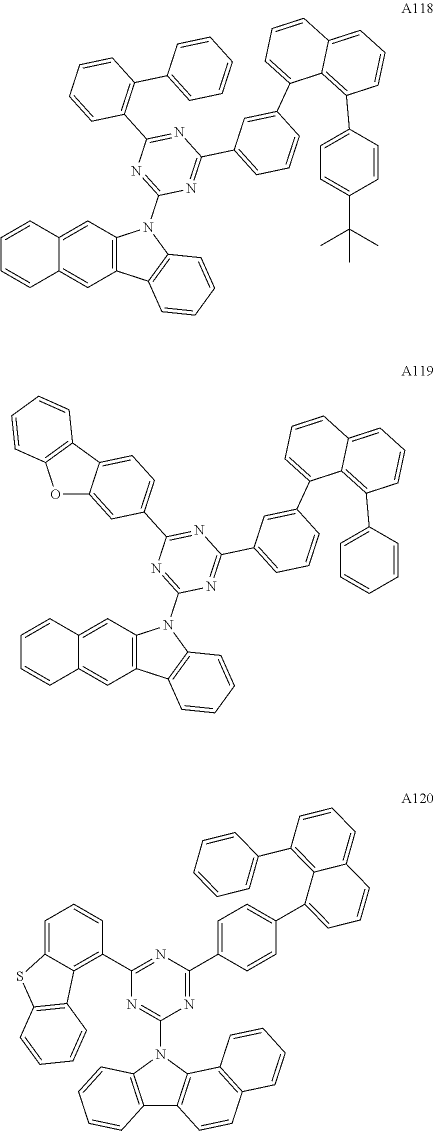 [SMILES: CC(C)(C)c1ccc(-c2cccc3cccc(-c4cccc(-c5nc(-c6ccccc6-c6ccccc6)nc(-n6c7ccccc7c7cc8ccccc8cc76)n5)c4)c23)cc1 ; c1ccc(-c2cccc3cccc(-c4cccc(-c5nc(-c6ccc7c(c6)oc6ccccc67)nc(-n6c7ccccc7c7cc8ccccc8cc76)n5)c4)c23)cc1 ; c1ccc(-c2cccc3cccc(-c4ccc(-c5nc(-c6cccc7sc8ccccc8c67)nc(-n6c7ccccc7c7ccc8ccccc8c76)n5)cc4)c23)cc1]

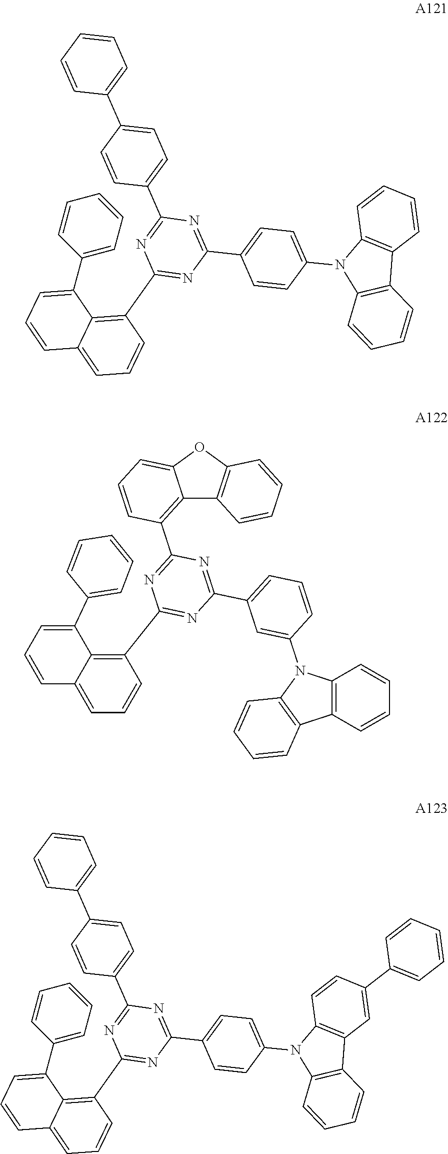 [SMILES: c1ccc(-c2ccc(-c3nc(-c4ccc(-n5c6ccccc6c6ccccc65)cc4)nc(-c4cccc5cccc(-c6ccccc6)c45)n3)cc2)cc1 ; c1ccc(-c2cccc3cccc(-c4nc(-c5cccc(-n6c7ccccc7c7ccccc76)c5)nc(-c5cccc6oc7ccccc7c56)n4)c23)cc1 ; c1ccc(-c2ccc(-c3nc(-c4ccc(-n5c6ccccc6c6cc(-c7ccccc7)ccc65)cc4)nc(-c4cccc5cccc(-c6ccccc6)c45)n3)cc2)cc1]

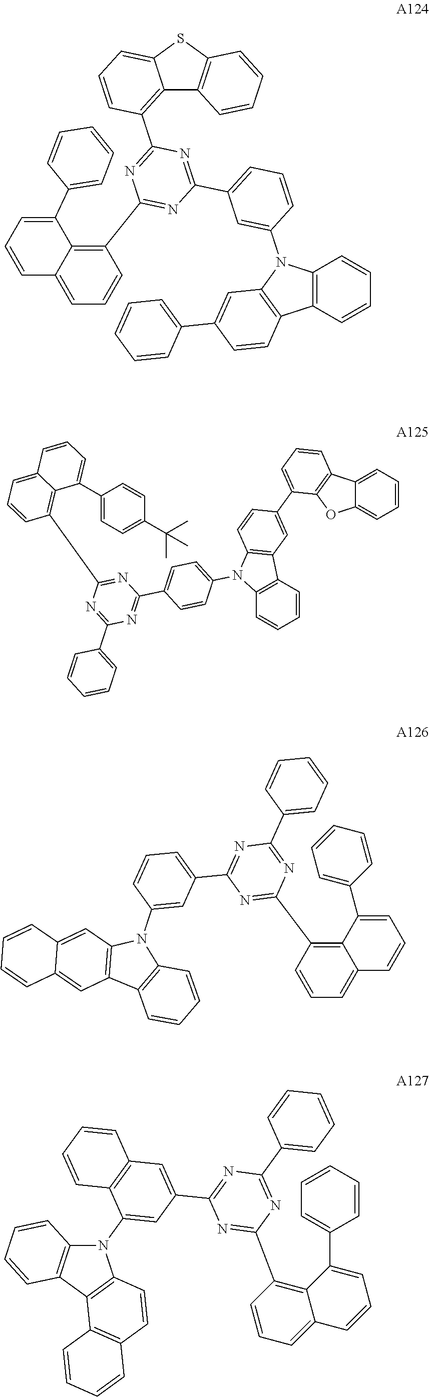 [SMILES: c1ccc(-c2ccc3c4ccccc4n(-c4cccc(-c5nc(-c6cccc7cccc(-c8ccccc8)c67)nc(-c6cccc7sc8ccccc8c67)n5)c4)c3c2)cc1 ; CC(C)(C)c1ccc(-c2cccc3cccc(-c4nc(-c5ccccc5)nc(-c5ccc(-n6c7ccccc7c7cc(-c8cccc9c8oc8ccccc89)ccc76)cc5)n4)c23)cc1 ; c1ccc(-c2nc(-c3cccc(-n4c5ccccc5c5cc6ccccc6cc54)c3)nc(-c3cccc4cccc(-c5ccccc5)c34)n2)cc1 ; c1ccc(-c2nc(-c3cc(-n4c5ccccc5c5c6ccccc6ccc54)c4ccccc4c3)nc(-c3cccc4cccc(-c5ccccc5)c34)n2)cc1]

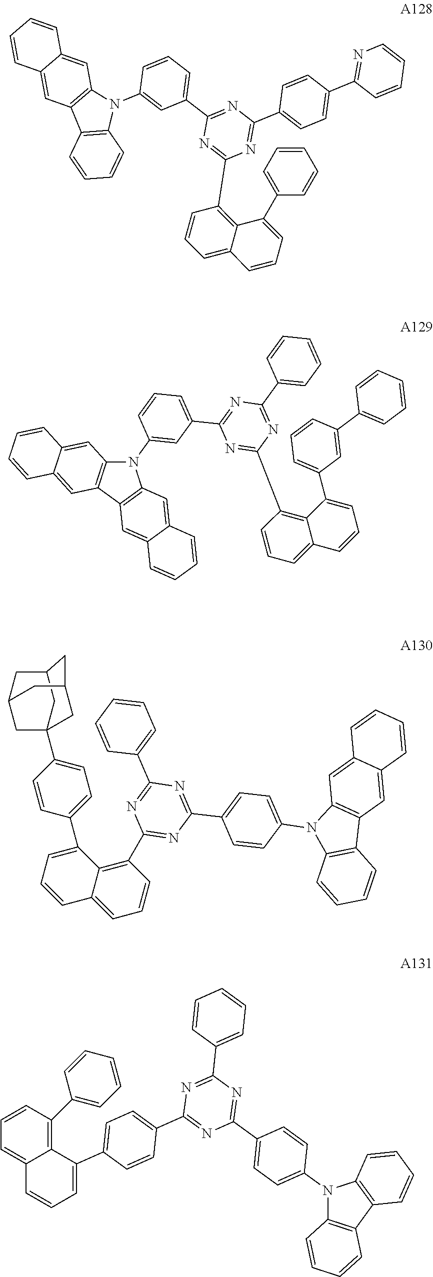 [SMILES: c1ccc(-c2cccc3cccc(-c4nc(-c5ccc(-c6ccccn6)cc5)nc(-c5cccc(-n6c7ccccc7c7cc8ccccc8cc76)c5)n4)c23)cc1 ; c1ccc(-c2cccc(-c3cccc4cccc(-c5nc(-c6ccccc6)nc(-c6cccc(-n7c8cc9ccccc9cc8c8cc9ccccc9cc87)c6)n5)c34)c2)cc1 ; c1ccc(-c2nc(-c3ccc(-n4c5ccccc5c5cc6ccccc6cc54)cc3)nc(-c3cccc4cccc(-c5ccc(C67CC8CC(CC(C8)C6)C7)cc5)c34)n2)cc1 ; c1ccc(-c2nc(-c3ccc(-c4cccc5cccc(-c6ccccc6)c45)cc3)nc(-c3ccc(-n4c5ccccc5c5ccccc54)cc3)n2)cc1]

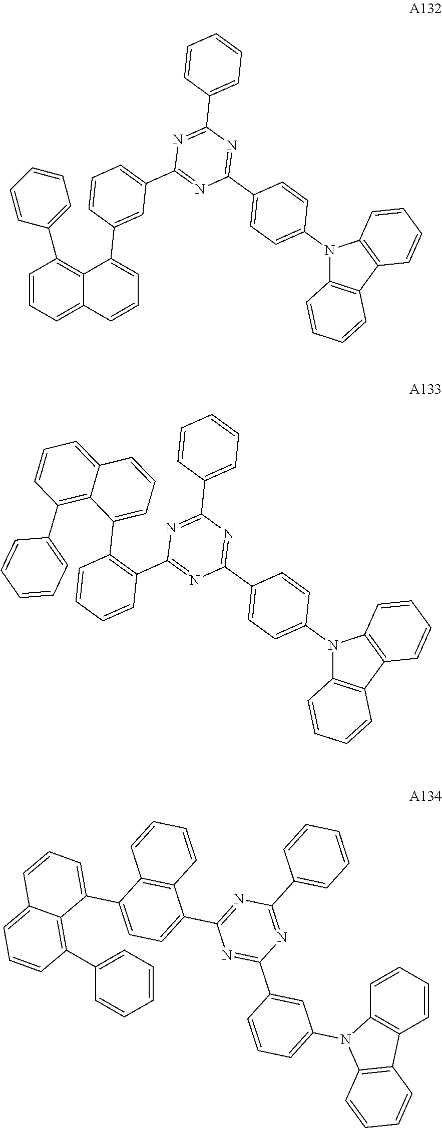 [SMILES: c1ccc(-c2nc(-c3ccc(-n4c5ccccc5c5ccccc54)cc3)nc(-c3cccc(-c4cccc5cccc(-c6ccccc6)c45)c3)n2)cc1 ; c1ccc(-c2nc(-c3ccc(-n4c5ccccc5c5ccccc54)cc3)nc(-c3ccccc3-c3cccc4cccc(-c5ccccc5)c34)n2)cc1 ; c1ccc(-c2nc(-c3cccc(-n4c5ccccc5c5ccccc54)c3)nc(-c3ccc(-c4cccc5cccc(-c6ccccc6)c45)c4ccccc34)n2)cc1]

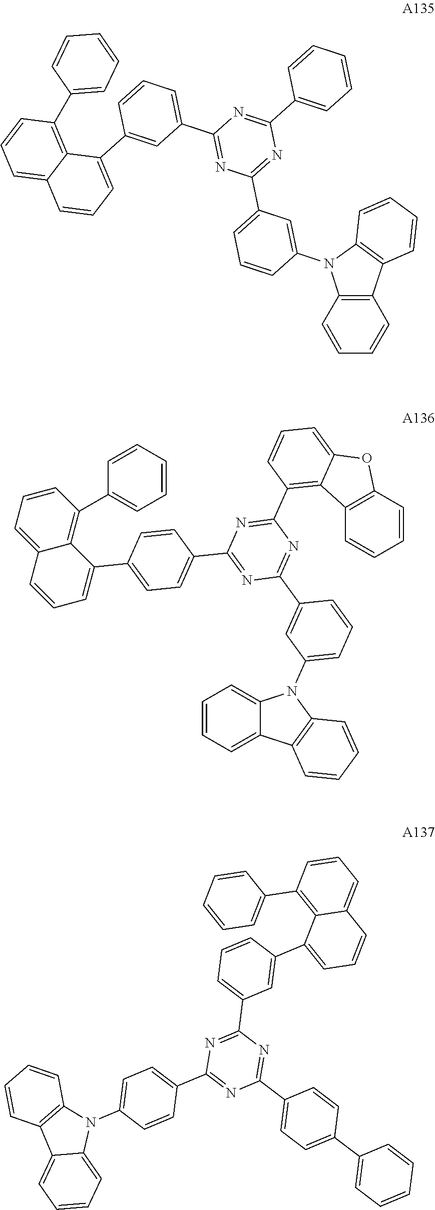 [SMILES: c1ccc(-c2nc(-c3cccc(-c4cccc5cccc(-c6ccccc6)c45)c3)nc(-c3cccc(-n4c5ccccc5c5ccccc54)c3)n2)cc1 ; c1ccc(-c2cccc3cccc(-c4ccc(-c5nc(-c6cccc(-n7c8ccccc8c8ccccc87)c6)nc(-c6cccc7oc8ccccc8c67)n5)cc4)c23)cc1 ; c1ccc(-c2ccc(-c3nc(-c4ccc(-n5c6ccccc6c6ccccc65)cc4)nc(-c4cccc(-c5cccc6cccc(-c7ccccc7)c56)c4)n3)cc2)cc1]

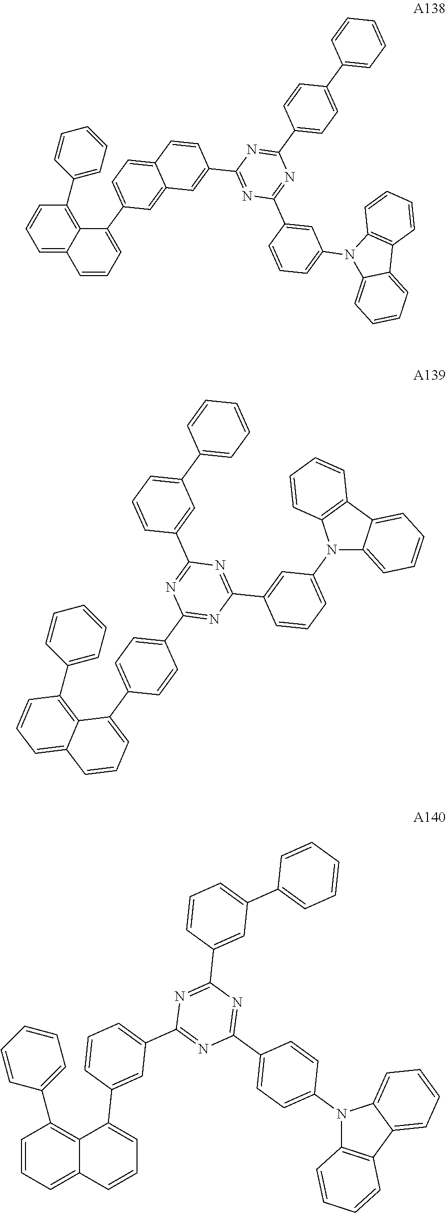 [SMILES: c1ccc(-c2ccc(-c3nc(-c4cccc(-n5c6ccccc6c6ccccc65)c4)nc(-c4ccc5ccc(-c6cccc7cccc(-c8ccccc8)c67)cc5c4)n3)cc2)cc1 ; c1ccc(-c2cccc(-c3nc(-c4ccc(-c5cccc6cccc(-c7ccccc7)c56)cc4)nc(-c4cccc(-n5c6ccccc6c6ccccc65)c4)n3)c2)cc1 ; c1ccc(-c2cccc(-c3nc(-c4ccc(-n5c6ccccc6c6ccccc65)cc4)nc(-c4cccc(-c5cccc6cccc(-c7ccccc7)c56)c4)n3)c2)cc1]

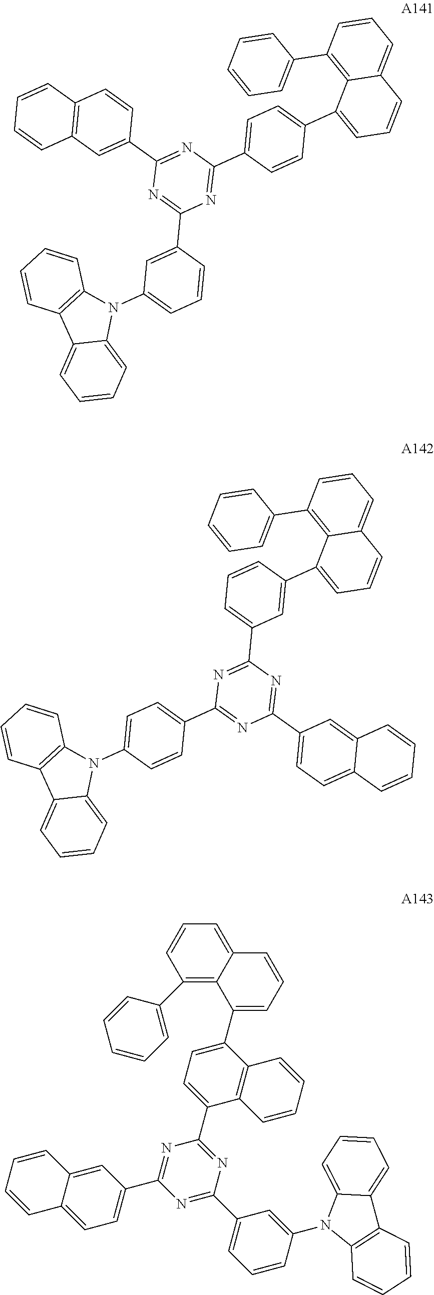 [SMILES: c1ccc(-c2cccc3cccc(-c4ccc(-c5nc(-c6cccc(-n7c8ccccc8c8ccccc87)c6)nc(-c6ccc7ccccc7c6)n5)cc4)c23)cc1 ; c1ccc(-c2cccc3cccc(-c4cccc(-c5nc(-c6ccc(-n7c8ccccc8c8ccccc87)cc6)nc(-c6ccc7ccccc7c6)n5)c4)c23)cc1 ; c1ccc(-c2cccc3cccc(-c4ccc(-c5nc(-c6cccc(-n7c8ccccc8c8ccccc87)c6)nc(-c6ccc7ccccc7c6)n5)c5ccccc45)c23)cc1]

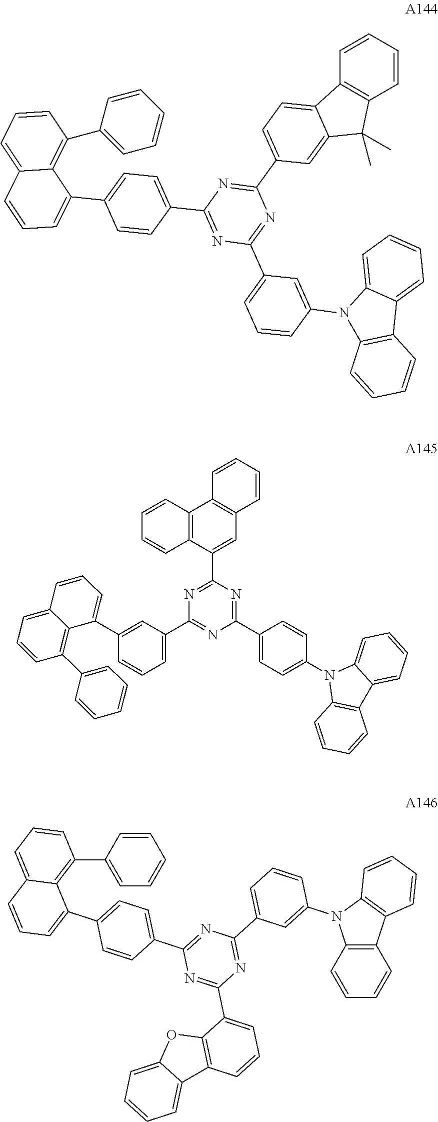 [SMILES: CC1(C)c2ccccc2-c2ccc(-c3nc(-c4ccc(-c5cccc6cccc(-c7ccccc7)c56)cc4)nc(-c4cccc(-n5c6ccccc6c6ccccc65)c4)n3)cc21 ; c1ccc(-c2cccc3cccc(-c4cccc(-c5nc(-c6ccc(-n7c8ccccc8c8ccccc87)cc6)nc(-c6cc7ccccc7c7ccccc67)n5)c4)c23)cc1 ; c1ccc(-c2cccc3cccc(-c4ccc(-c5nc(-c6cccc(-n7c8ccccc8c8ccccc87)c6)nc(-c6cccc7c6oc6ccccc67)n5)cc4)c23)cc1]

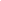

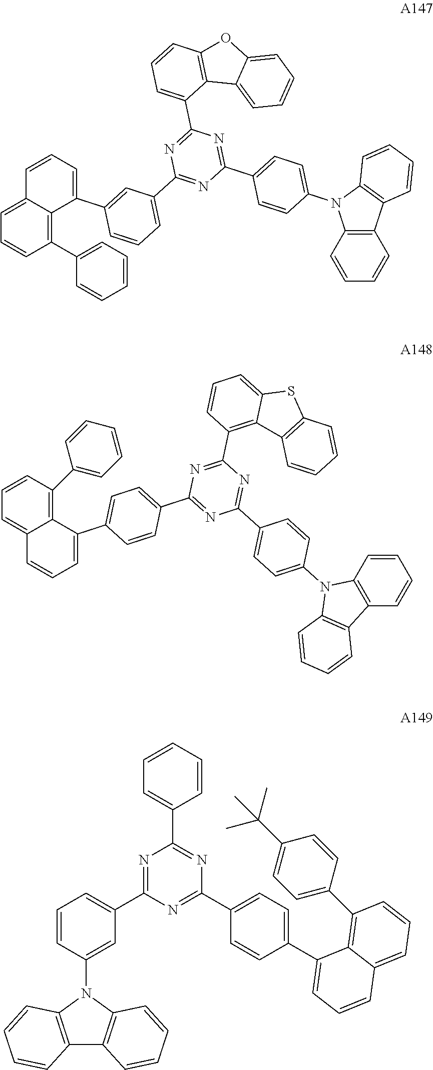 [SMILES: c1ccc(-c2cccc3cccc(-c4cccc(-c5nc(-c6ccc(-n7c8ccccc8c8ccccc87)cc6)nc(-c6cccc7oc8ccccc8c67)n5)c4)c23)cc1 ; c1ccc(-c2cccc3cccc(-c4ccc(-c5nc(-c6ccc(-n7c8ccccc8c8ccccc87)cc6)nc(-c6cccc7sc8ccccc8c67)n5)cc4)c23)cc1 ; CC(C)(C)c1ccc(-c2cccc3cccc(-c4ccc(-c5nc(-c6ccccc6)nc(-c6cccc(-n7c8ccccc8c8ccccc87)c6)n5)cc4)c23)cc1]

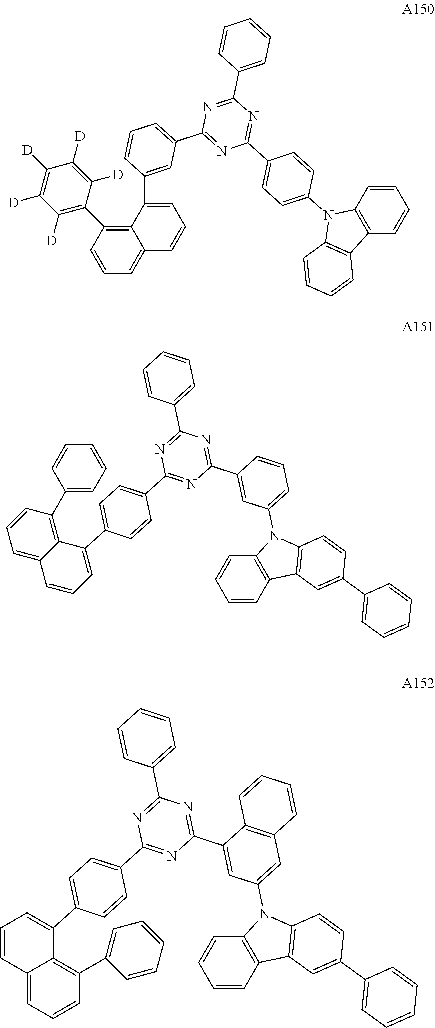 [SMILES: [2H]c1c([2H])c([2H])c(-c2cccc3cccc(-c4cccc(-c5nc(-c6ccccc6)nc(-c6ccc(-n7c8ccccc8c8ccccc87)cc6)n5)c4)c23)c([2H])c1[2H] ; c1ccc(-c2ccc3c(c2)c2ccccc2n3-c2cccc(-c3nc(-c4ccccc4)nc(-c4ccc(-c5cccc6cccc(-c7ccccc7)c56)cc4)n3)c2)cc1 ; c1ccc(-c2ccc3c(c2)c2ccccc2n3-c2cc(-c3nc(-c4ccccc4)nc(-c4ccc(-c5cccc6cccc(-c7ccccc7)c56)cc4)n3)c3ccccc3c2)cc1]

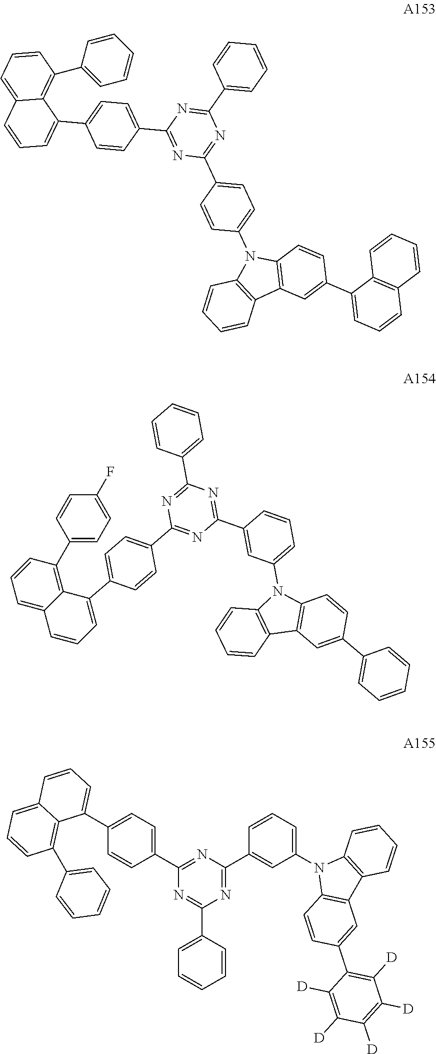 [SMILES: c1ccc(-c2nc(-c3ccc(-c4cccc5cccc(-c6ccccc6)c45)cc3)nc(-c3ccc(-n4c5ccccc5c5cc(-c6cccc7ccccc67)ccc54)cc3)n2)cc1 ; Fc1ccc(-c2cccc3cccc(-c4ccc(-c5nc(-c6ccccc6)nc(-c6cccc(-n7c8ccccc8c8cc(-c9ccccc9)ccc87)c6)n5)cc4)c23)cc1 ; [2H]c1c([2H])c([2H])c(-c2ccc3c(c2)c2ccccc2n3-c2cccc(-c3nc(-c4ccccc4)nc(-c4ccc(-c5cccc6cccc(-c7ccccc7)c56)cc4)n3)c2)c([2H])c1[2H]]

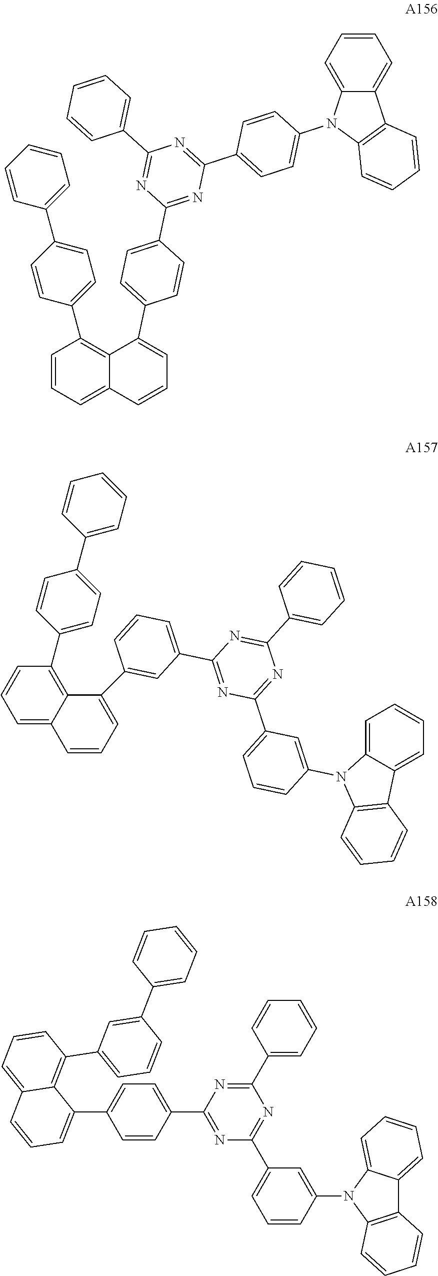 [SMILES: c1ccc(-c2ccc(-c3cccc4cccc(-c5ccc(-c6nc(-c7ccccc7)nc(-c7ccc(-n8c9ccccc9c9ccccc98)cc7)n6)cc5)c34)cc2)cc1 ; c1ccc(-c2ccc(-c3cccc4cccc(-c5cccc(-c6nc(-c7ccccc7)nc(-c7cccc(-n8c9ccccc9c9ccccc98)c7)n6)c5)c34)cc2)cc1 ; c1ccc(-c2cccc(-c3cccc4cccc(-c5ccc(-c6nc(-c7ccccc7)nc(-c7cccc(-n8c9ccccc9c9ccccc98)c7)n6)cc5)c34)c2)cc1]

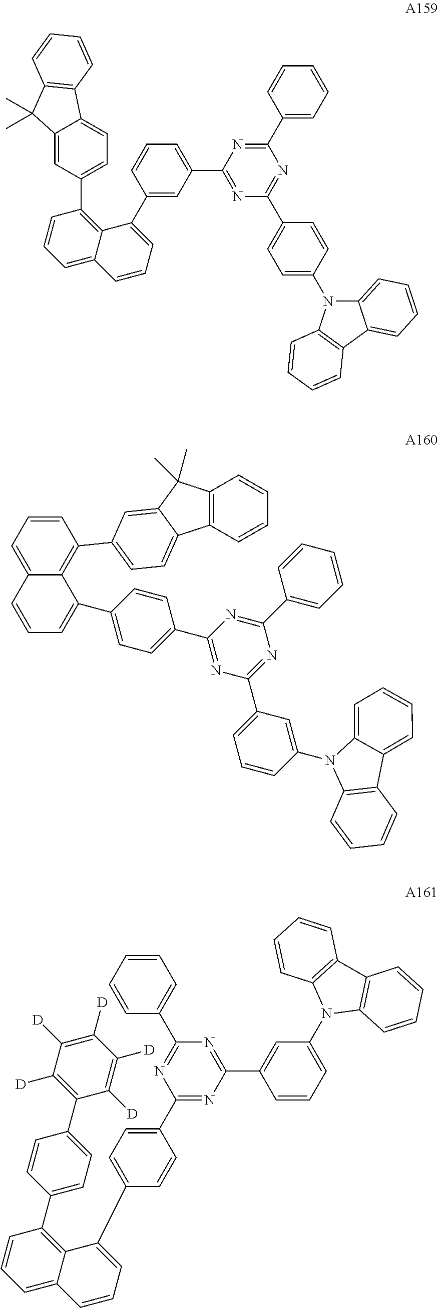 [SMILES: CC1(C)c2ccccc2-c2ccc(-c3cccc4cccc(-c5cccc(-c6nc(-c7ccccc7)nc(-c7ccc(-n8c9ccccc9c9ccccc98)cc7)n6)c5)c34)cc21 ; CC1(C)c2ccccc2-c2ccc(-c3cccc4cccc(-c5ccc(-c6nc(-c7ccccc7)nc(-c7cccc(-n8c9ccccc9c9ccccc98)c7)n6)cc5)c34)cc21 ; [2H]c1c([2H])c([2H])c(-c2ccc(-c3cccc4cccc(-c5ccc(-c6nc(-c7ccccc7)nc(-c7cccc(-n8c9ccccc9c9ccccc98)c7)n6)cc5)c34)cc2)c([2H])c1[2H]]

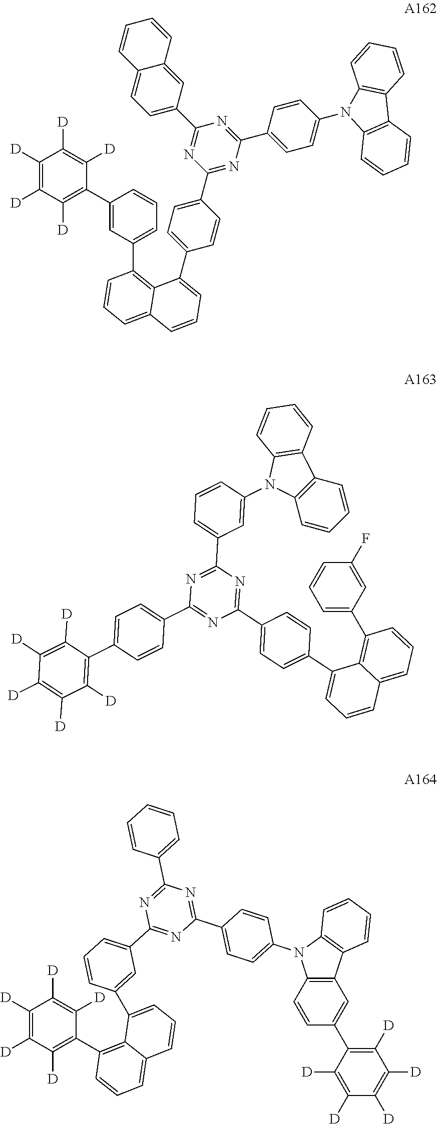 [SMILES: [2H]c1c([2H])c([2H])c(-c2cccc(-c3cccc4cccc(-c5ccc(-c6nc(-c7ccc(-n8c9ccccc9c9ccccc98)cc7)nc(-c7ccc8ccccc8c7)n6)cc5)c34)c2)c([2H])c1[2H] ; [2H]c1c([2H])c([2H])c(-c2ccc(-c3nc(-c4ccc(-c5cccc6cccc(-c7cccc(F)c7)c56)cc4)nc(-c4cccc(-n5c6ccccc6c6ccccc65)c4)n3)cc2)c([2H])c1[2H] ; [2H]c1c([2H])c([2H])c(-c2ccc3c(c2)c2ccccc2n3-c2ccc(-c3nc(-c4ccccc4)nc(-c4cccc(-c5cccc6cccc(-c7c([2H])c([2H])c([2H])c([2H])c7[2H])c56)c4)n3)cc2)c([2H])c1[2H]]

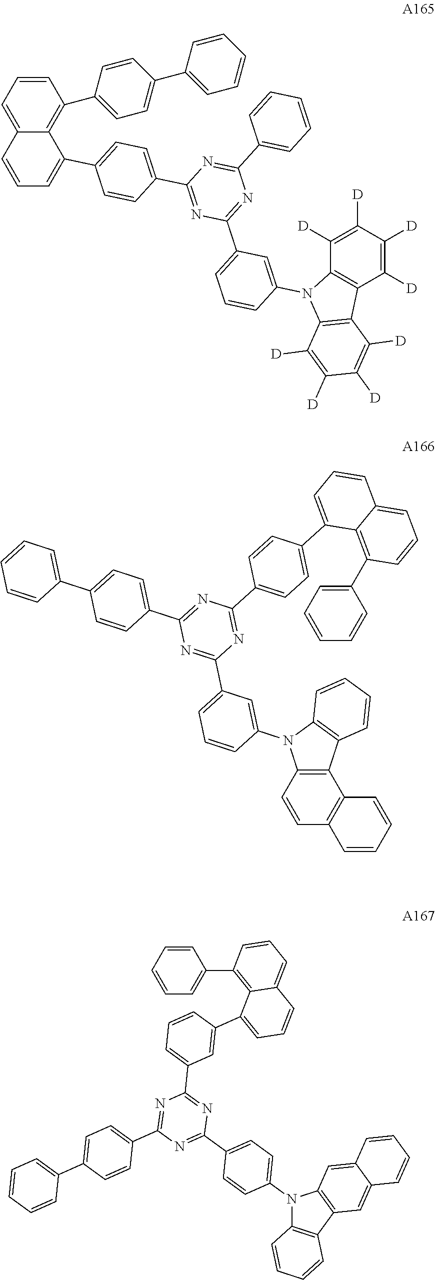 [SMILES: [2H]c1c([2H])c([2H])c2c(c1[2H])c1c([2H])c([2H])c([2H])c([2H])c1n2-c1cccc(-c2nc(-c3ccccc3)nc(-c3ccc(-c4cccc5cccc(-c6ccc(-c7ccccc7)cc6)c45)cc3)n2)c1 ; c1ccc(-c2ccc(-c3nc(-c4ccc(-c5cccc6cccc(-c7ccccc7)c56)cc4)nc(-c4cccc(-n5c6ccccc6c6c7ccccc7ccc65)c4)n3)cc2)cc1 ; c1ccc(-c2ccc(-c3nc(-c4ccc(-n5c6ccccc6c6cc7ccccc7cc65)cc4)nc(-c4cccc(-c5cccc6cccc(-c7ccccc7)c56)c4)n3)cc2)cc1]

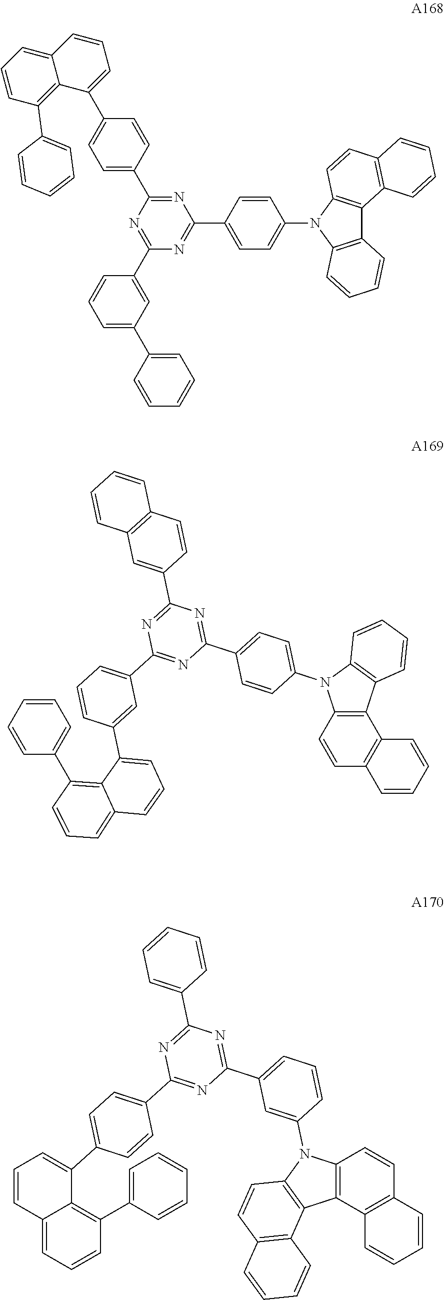 [SMILES: c1ccc(-c2cccc(-c3nc(-c4ccc(-c5cccc6cccc(-c7ccccc7)c56)cc4)nc(-c4ccc(-n5c6ccccc6c6c7ccccc7ccc65)cc4)n3)c2)cc1 ; c1ccc(-c2cccc3cccc(-c4cccc(-c5nc(-c6ccc(-n7c8ccccc8c8c9ccccc9ccc87)cc6)nc(-c6ccc7ccccc7c6)n5)c4)c23)cc1 ; c1ccc(-c2nc(-c3ccc(-c4cccc5cccc(-c6ccccc6)c45)cc3)nc(-c3cccc(-n4c5ccc6ccccc6c5c5c6ccccc6ccc54)c3)n2)cc1]

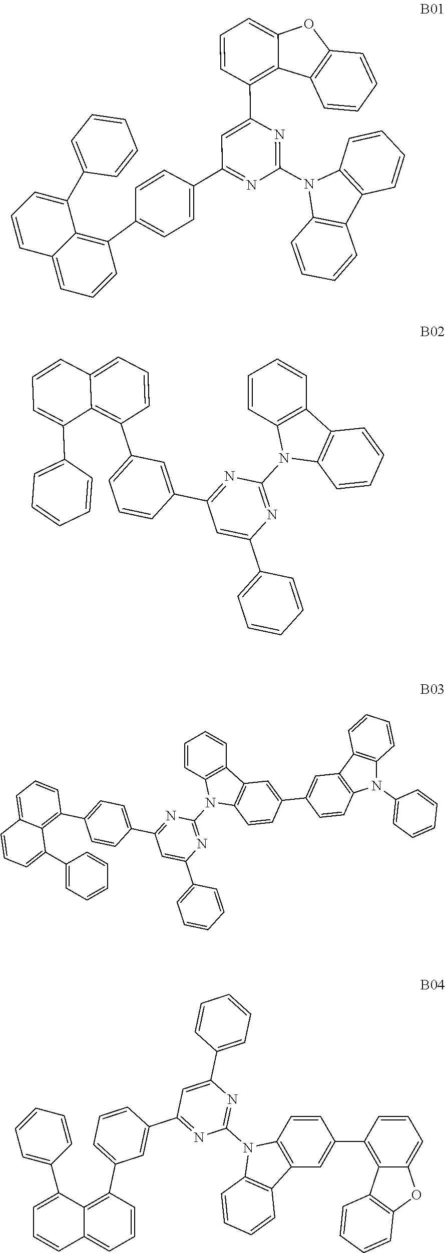 [SMILES: c1ccc(-c2cccc3cccc(-c4ccc(-c5cc(-c6cccc7oc8ccccc8c67)nc(-n6c7ccccc7c7ccccc76)n5)cc4)c23)cc1 ; c1ccc(-c2cc(-c3cccc(-c4cccc5cccc(-c6ccccc6)c45)c3)nc(-n3c4ccccc4c4ccccc43)n2)cc1 ; c1ccc(-c2cc(-c3ccc(-c4cccc5cccc(-c6ccccc6)c45)cc3)nc(-n3c4ccccc4c4cc(-c5ccc6c(c5)c5ccccc5n6-c5ccccc5)ccc43)n2)cc1 ; c1ccc(-c2cc(-c3cccc(-c4cccc5cccc(-c6ccccc6)c45)c3)nc(-n3c4ccccc4c4cc(-c5cccc6oc7ccccc7c56)ccc43)n2)cc1]

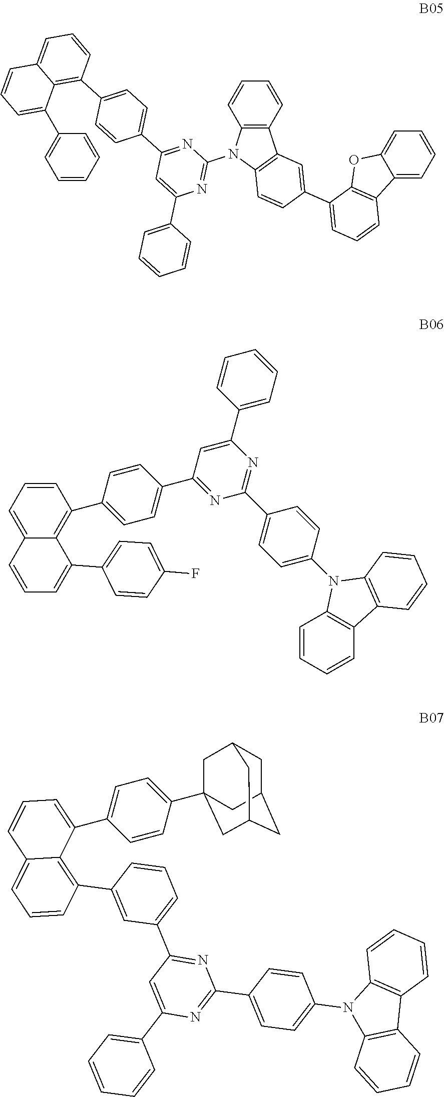 [SMILES: c1ccc(-c2cc(-c3ccc(-c4cccc5cccc(-c6ccccc6)c45)cc3)nc(-n3c4ccccc4c4cc(-c5cccc6c5oc5ccccc56)ccc43)n2)cc1 ; Fc1ccc(-c2cccc3cccc(-c4ccc(-c5cc(-c6ccccc6)nc(-c6ccc(-n7c8ccccc8c8ccccc87)cc6)n5)cc4)c23)cc1 ; c1ccc(-c2cc(-c3cccc(-c4cccc5cccc(-c6ccc(C78CC9CC(CC(C9)C7)C8)cc6)c45)c3)nc(-c3ccc(-n4c5ccccc5c5ccccc54)cc3)n2)cc1]

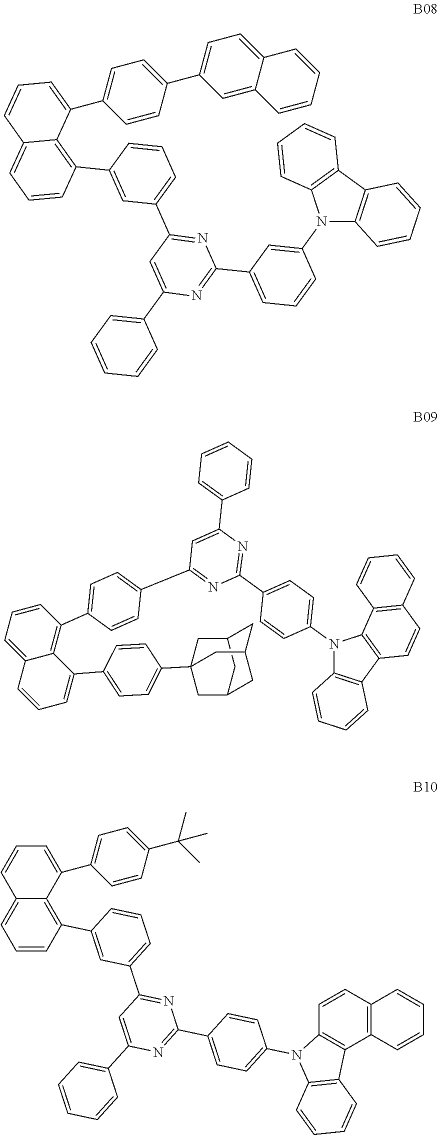 [SMILES: c1ccc(-c2cc(-c3cccc(-c4cccc5cccc(-c6ccc(-c7ccc8ccccc8c7)cc6)c45)c3)nc(-c3cccc(-n4c5ccccc5c5ccccc54)c3)n2)cc1 ; c1ccc(-c2cc(-c3ccc(-c4cccc5cccc(-c6ccc(C78CC9CC(CC(C9)C7)C8)cc6)c45)cc3)nc(-c3ccc(-n4c5ccccc5c5ccc6ccccc6c54)cc3)n2)cc1 ; CC(C)(C)c1ccc(-c2cccc3cccc(-c4cccc(-c5cc(-c6ccccc6)nc(-c6ccc(-n7c8ccccc8c8c9ccccc9ccc87)cc6)n5)c4)c23)cc1]

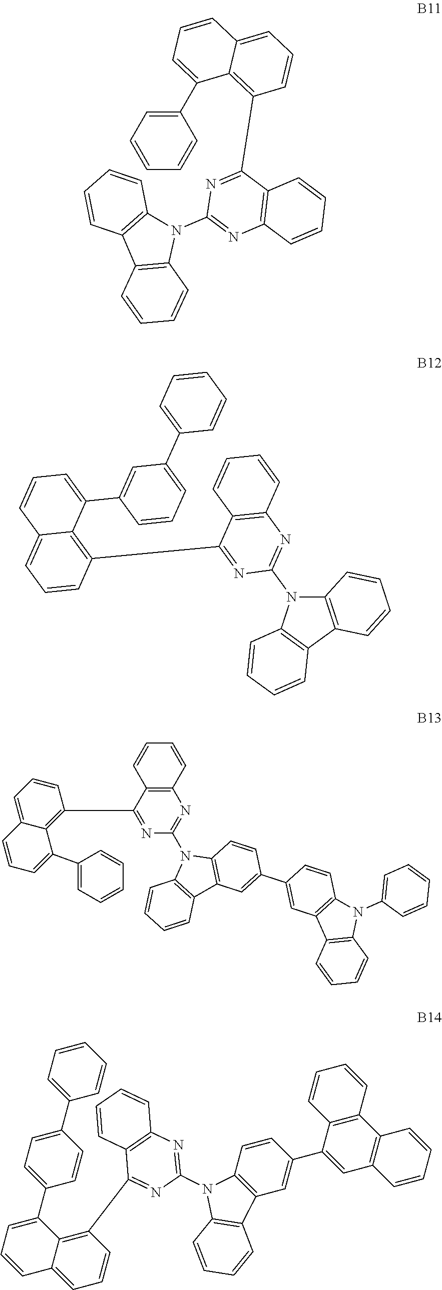 [SMILES: c1ccc(-c2cccc3cccc(-c4nc(-n5c6ccccc6c6ccccc65)nc5ccccc45)c23)cc1 ; c1ccc(-c2cccc(-c3cccc4cccc(-c5nc(-n6c7ccccc7c7ccccc76)nc6ccccc56)c34)c2)cc1 ; c1ccc(-c2cccc3cccc(-c4nc(-n5c6ccccc6c6cc(-c7ccc8c(c7)c7ccccc7n8-c7ccccc7)ccc65)nc5ccccc45)c23)cc1 ; c1ccc(-c2ccc(-c3cccc4cccc(-c5nc(-n6c7ccccc7c7cc(-c8cc9ccccc9c9ccccc89)ccc76)nc6ccccc56)c34)cc2)cc1]

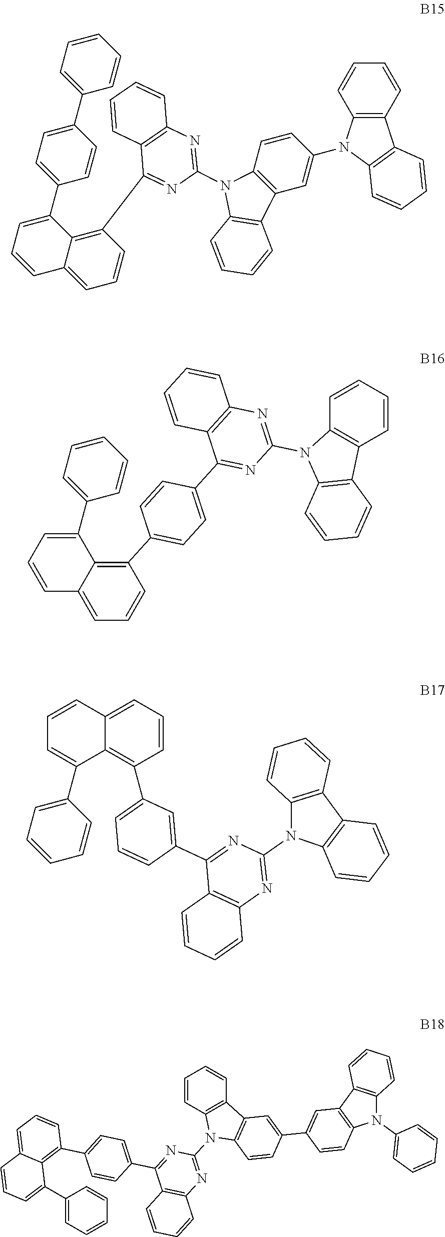 [SMILES: c1ccc(-c2ccc(-c3cccc4cccc(-c5nc(-n6c7ccccc7c7cc(-n8c9ccccc9c9ccccc98)ccc76)nc6ccccc56)c34)cc2)cc1 ; c1ccc(-c2cccc3cccc(-c4ccc(-c5nc(-n6c7ccccc7c7ccccc76)nc6ccccc56)cc4)c23)cc1 ; c1ccc(-c2cccc3cccc(-c4cccc(-c5nc(-n6c7ccccc7c7ccccc76)nc6ccccc56)c4)c23)cc1 ; c1ccc(-c2cccc3cccc(-c4ccc(-c5nc(-n6c7ccccc7c7cc(-c8ccc9c(c8)c8ccccc8n9-c8ccccc8)ccc76)nc6ccccc56)cc4)c23)cc1]

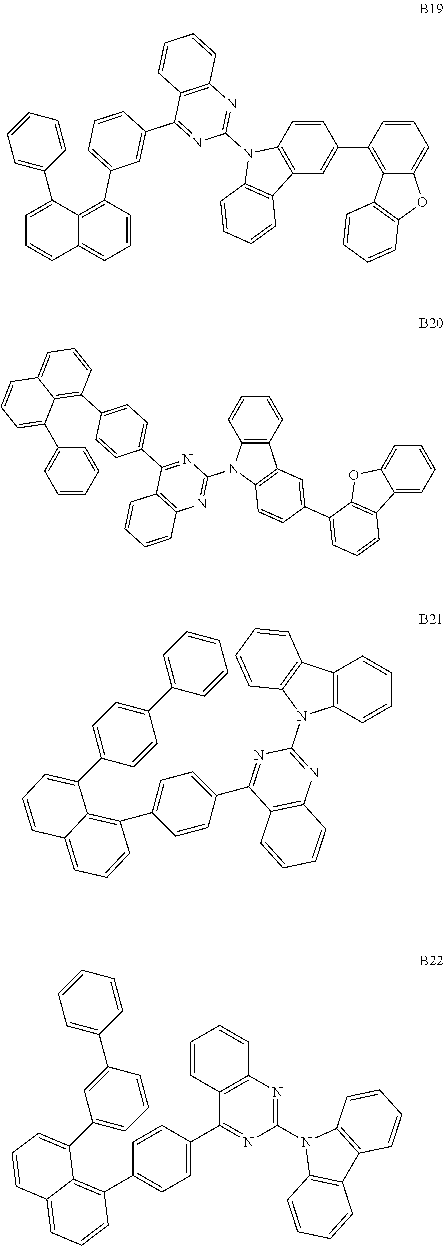 [SMILES: c1ccc(-c2cccc3cccc(-c4cccc(-c5nc(-n6c7ccccc7c7cc(-c8cccc9oc%10ccccc%10c89)ccc76)nc6ccccc56)c4)c23)cc1 ; c1ccc(-c2cccc3cccc(-c4ccc(-c5nc(-n6c7ccccc7c7cc(-c8cccc9c8oc8ccccc89)ccc76)nc6ccccc56)cc4)c23)cc1 ; c1ccc(-c2ccc(-c3cccc4cccc(-c5ccc(-c6nc(-n7c8ccccc8c8ccccc87)nc7ccccc67)cc5)c34)cc2)cc1 ; c1ccc(-c2cccc(-c3cccc4cccc(-c5ccc(-c6nc(-n7c8ccccc8c8ccccc87)nc7ccccc67)cc5)c34)c2)cc1]

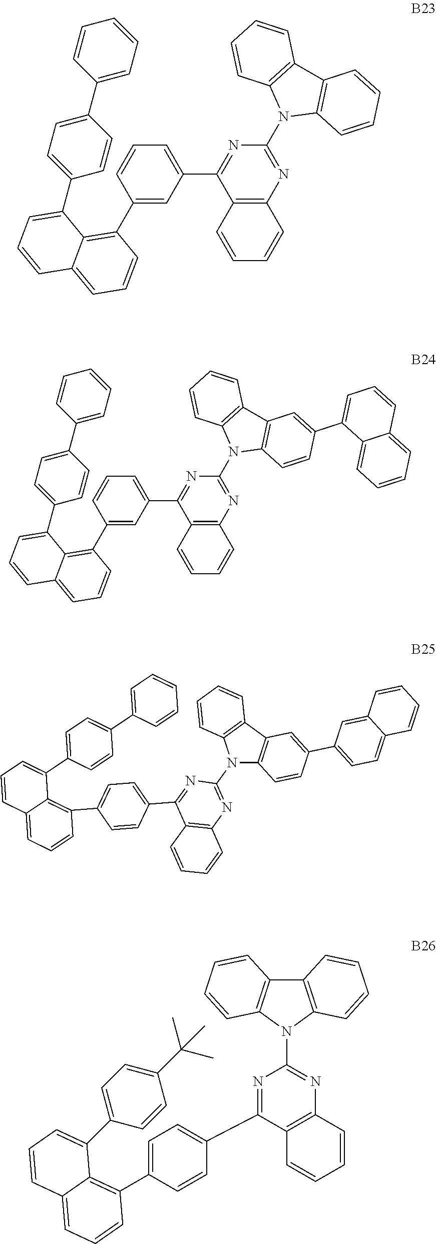 [SMILES: c1ccc(-c2ccc(-c3cccc4cccc(-c5cccc(-c6nc(-n7c8ccccc8c8ccccc87)nc7ccccc67)c5)c34)cc2)cc1 ; c1ccc(-c2ccc(-c3cccc4cccc(-c5cccc(-c6nc(-n7c8ccccc8c8cc(-c9cccc%10ccccc9%10)ccc87)nc7ccccc67)c5)c34)cc2)cc1 ; c1ccc(-c2ccc(-c3cccc4cccc(-c5ccc(-c6nc(-n7c8ccccc8c8cc(-c9ccc%10ccccc%10c9)ccc87)nc7ccccc67)cc5)c34)cc2)cc1 ; CC(C)(C)c1ccc(-c2cccc3cccc(-c4ccc(-c5nc(-n6c7ccccc7c7ccccc76)nc6ccccc56)cc4)c23)cc1]

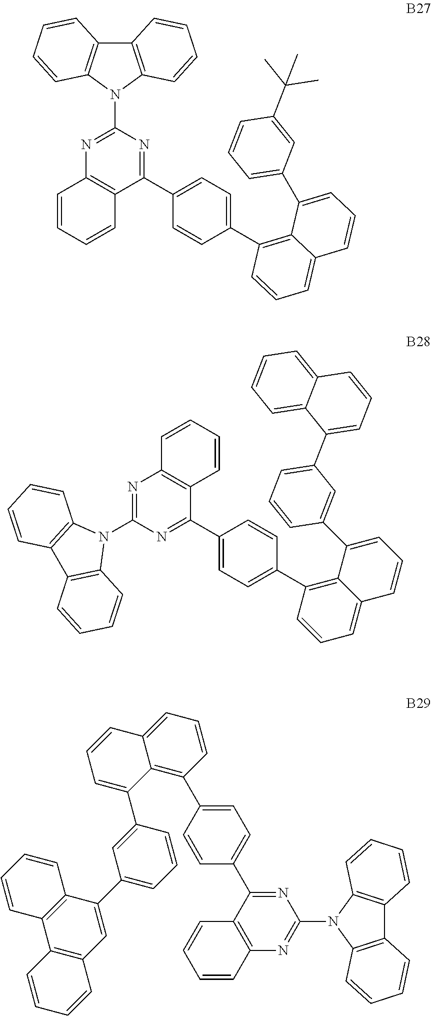 [SMILES: CC(C)(C)c1cccc(-c2cccc3cccc(-c4ccc(-c5nc(-n6c7ccccc7c7ccccc76)nc6ccccc56)cc4)c23)c1 ; c1cc(-c2cccc3ccccc23)cc(-c2cccc3cccc(-c4ccc(-c5nc(-n6c7ccccc7c7ccccc76)nc6ccccc56)cc4)c23)c1 ; c1cc(-c2cc3ccccc3c3ccccc23)cc(-c2cccc3cccc(-c4ccc(-c5nc(-n6c7ccccc7c7ccccc76)nc6ccccc56)cc4)c23)c1]

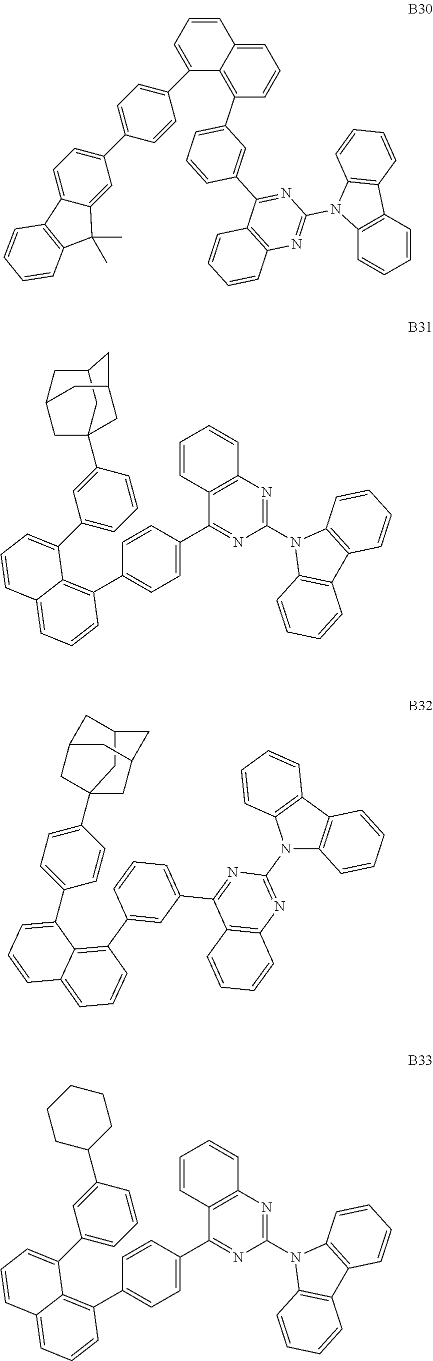 [SMILES: CC1(C)c2ccccc2-c2ccc(-c3ccc(-c4cccc5cccc(-c6cccc(-c7nc(-n8c9ccccc9c9ccccc98)nc8ccccc78)c6)c45)cc3)cc21 ; c1cc(-c2cccc3cccc(-c4ccc(-c5nc(-n6c7ccccc7c7ccccc76)nc6ccccc56)cc4)c23)cc(C23CC4CC(CC(C4)C2)C3)c1 ; c1cc(-c2nc(-n3c4ccccc4c4ccccc43)nc3ccccc23)cc(-c2cccc3cccc(-c4ccc(C56CC7CC(CC(C7)C5)C6)cc4)c23)c1 ; c1cc(-c2cccc3cccc(-c4ccc(-c5nc(-n6c7ccccc7c7ccccc76)nc6ccccc56)cc4)c23)cc(C2CCCCC2)c1]

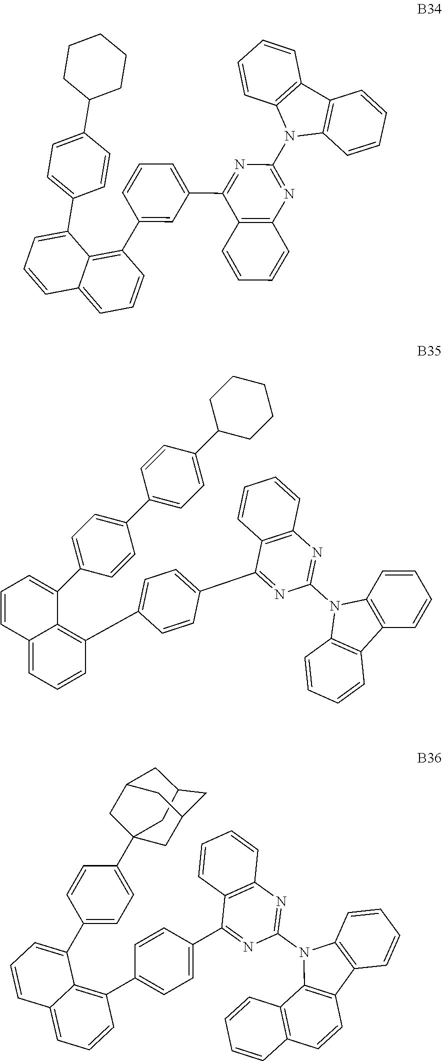 [SMILES: c1cc(-c2nc(-n3c4ccccc4c4ccccc43)nc3ccccc23)cc(-c2cccc3cccc(-c4ccc(C5CCCCC5)cc4)c23)c1 ; c1cc(-c2ccc(-c3ccc(C4CCCCC4)cc3)cc2)c2c(-c3ccc(-c4nc(-n5c6ccccc6c6ccccc65)nc5ccccc45)cc3)cccc2c1 ; c1cc(-c2ccc(-c3nc(-n4c5ccccc5c5ccc6ccccc6c54)nc4ccccc34)cc2)c2c(-c3ccc(C45CC6CC(CC(C6)C4)C5)cc3)cccc2c1]

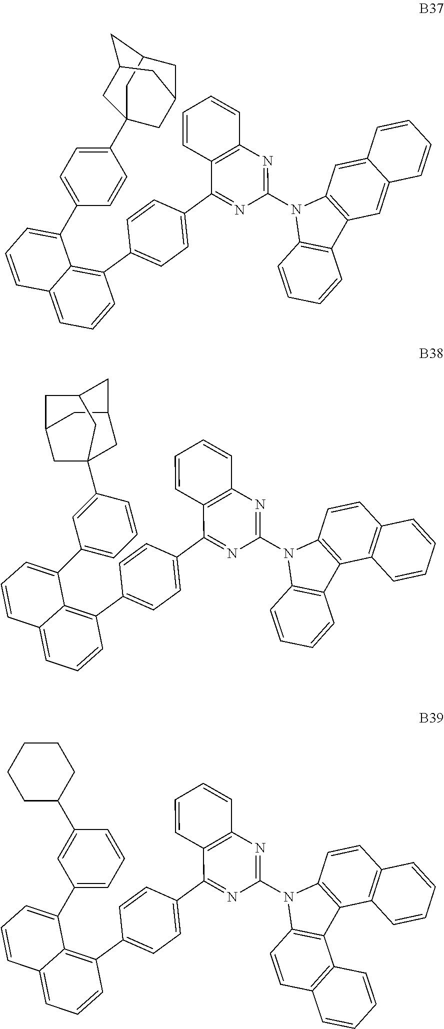 [SMILES: c1ccc2cc3c(cc2c1)c1ccccc1n3-c1nc(-c2ccc(-c3cccc4cccc(-c5ccc(C67CC8CC(CC(C8)C6)C7)cc5)c34)cc2)c2ccccc2n1 ; c1cc(-c2cccc3cccc(-c4ccc(-c5nc(-n6c7ccccc7c7c8ccccc8ccc76)nc6ccccc56)cc4)c23)cc(C23CC4CC(CC(C4)C2)C3)c1 ; c1cc(-c2cccc3cccc(-c4ccc(-c5nc(-n6c7ccc8ccccc8c7c7c8ccccc8ccc76)nc6ccccc56)cc4)c23)cc(C2CCCCC2)c1]

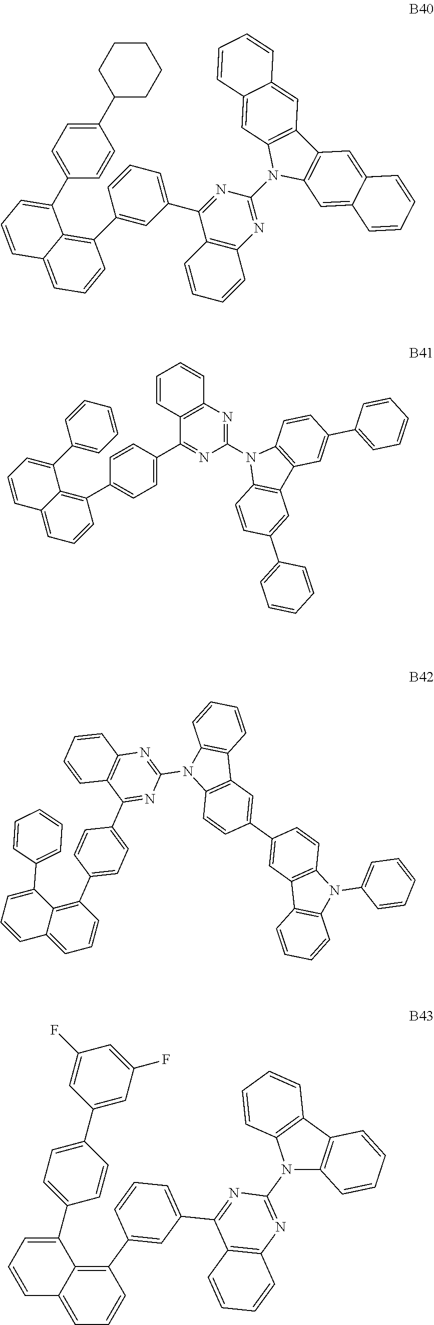 [SMILES: c1cc(-c2nc(-n3c4cc5ccccc5cc4c4cc5ccccc5cc43)nc3ccccc23)cc(-c2cccc3cccc(-c4ccc(C5CCCCC5)cc4)c23)c1 ; c1ccc(-c2ccc3c(c2)c2cc(-c4ccccc4)ccc2n3-c2nc(-c3ccc(-c4cccc5cccc(-c6ccccc6)c45)cc3)c3ccccc3n2)cc1 ; c1ccc(-c2cccc3cccc(-c4ccc(-c5nc(-n6c7ccccc7c7cc(-c8ccc9c(c8)c8ccccc8n9-c8ccccc8)ccc76)nc6ccccc56)cc4)c23)cc1 ; Fc1cc(F)cc(-c2ccc(-c3cccc4cccc(-c5cccc(-c6nc(-n7c8ccccc8c8ccccc87)nc7ccccc67)c5)c34)cc2)c1]

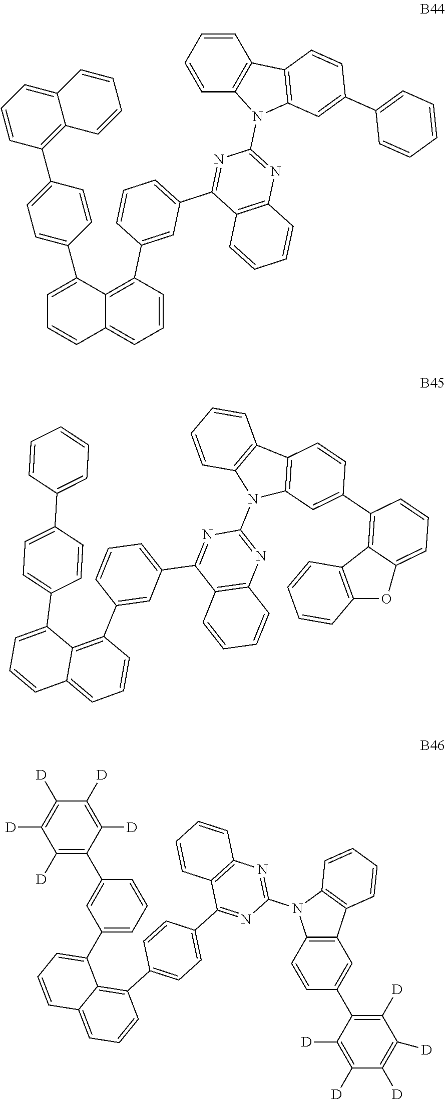 [SMILES: c1ccc(-c2ccc3c4ccccc4n(-c4nc(-c5cccc(-c6cccc7cccc(-c8ccc(-c9cccc%10ccccc9%10)cc8)c67)c5)c5ccccc5n4)c3c2)cc1 ; c1ccc(-c2ccc(-c3cccc4cccc(-c5cccc(-c6nc(-n7c8ccccc8c8ccc(-c9cccc%10oc%11ccccc%11c9%10)cc87)nc7ccccc67)c5)c34)cc2)cc1 ; [2H]c1c([2H])c([2H])c(-c2cccc(-c3cccc4cccc(-c5ccc(-c6nc(-n7c8ccccc8c8cc(-c9c([2H])c([2H])c([2H])c([2H])c9[2H])ccc87)nc7ccccc67)cc5)c34)c2)c([2H])c1[2H]]

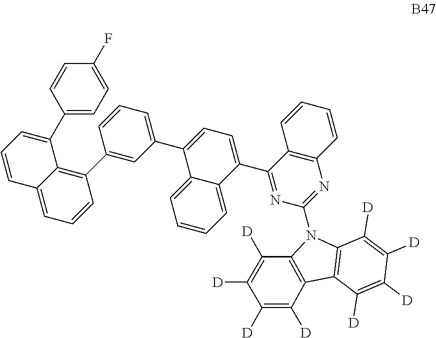 [SMILES: [2H]c1c([2H])c([2H])c2c(c1[2H])c1c([2H])c([2H])c([2H])c([2H])c1n2-c1nc(-c2ccc(-c3cccc(-c4cccc5cccc(-c6ccc(F)cc6)c45)c3)c3ccccc23)c2ccccc2n1]

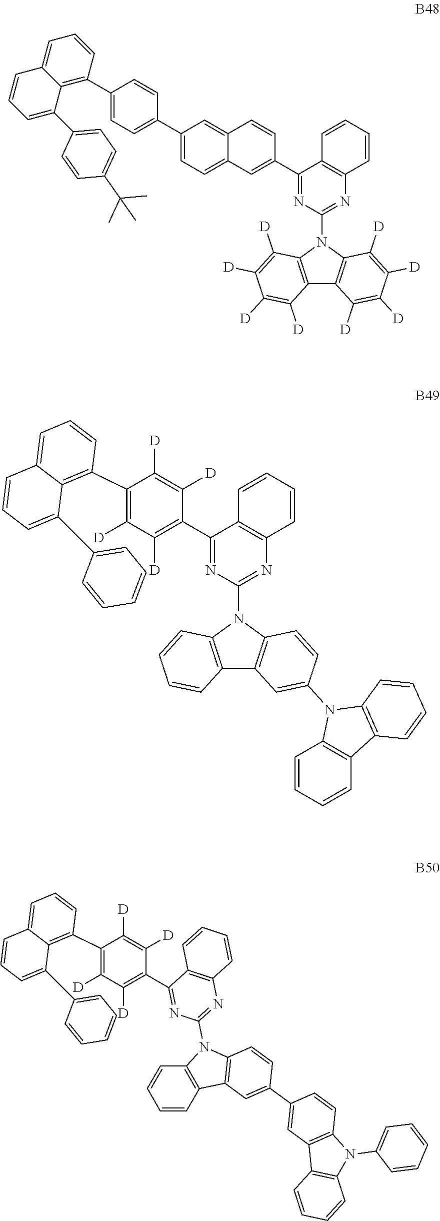 [SMILES: [2H]c1c([2H])c([2H])c2c(c1[2H])c1c([2H])c([2H])c([2H])c([2H])c1n2-c1nc(-c2ccc3cc(-c4ccc(-c5cccc6cccc(-c7ccc(C(C)(C)C)cc7)c56)cc4)ccc3c2)c2ccccc2n1 ; [2H]c1c([2H])c(-c2cccc3cccc(-c4ccccc4)c23)c([2H])c([2H])c1-c1nc(-n2c3ccccc3c3cc(-n4c5ccccc5c5ccccc54)ccc32)nc2ccccc12 ; [2H]c1c([2H])c(-c2cccc3cccc(-c4ccccc4)c23)c([2H])c([2H])c1-c1nc(-n2c3ccccc3c3cc(-c4ccc5c(c4)c4ccccc4n5-c4ccccc4)ccc32)nc2ccccc12]

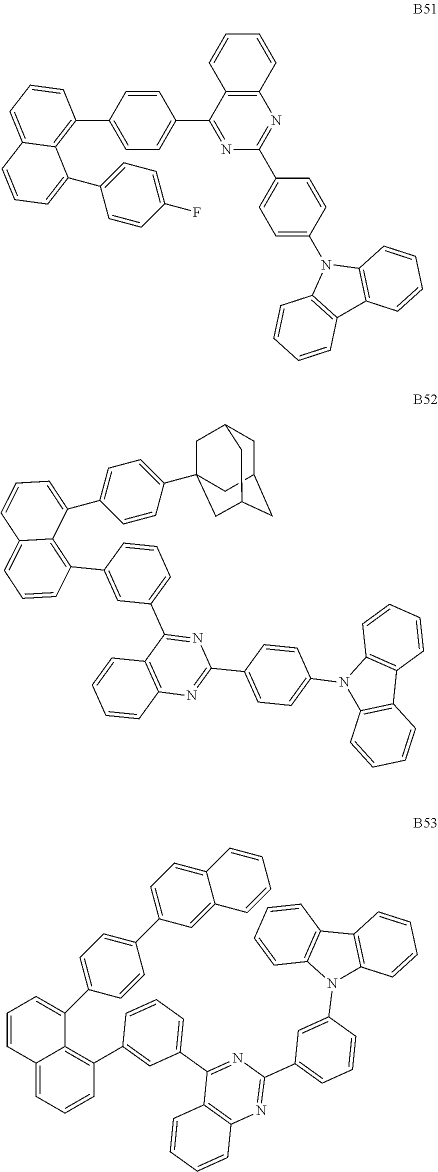 [SMILES: Fc1ccc(-c2cccc3cccc(-c4ccc(-c5nc(-c6ccc(-n7c8ccccc8c8ccccc87)cc6)nc6ccccc56)cc4)c23)cc1 ; c1cc(-c2nc(-c3ccc(-n4c5ccccc5c5ccccc54)cc3)nc3ccccc23)cc(-c2cccc3cccc(-c4ccc(C56CC7CC(CC(C7)C5)C6)cc4)c23)c1 ; c1cc(-c2nc(-c3cccc(-n4c5ccccc5c5ccccc54)c3)nc3ccccc23)cc(-c2cccc3cccc(-c4ccc(-c5ccc6ccccc6c5)cc4)c23)c1]

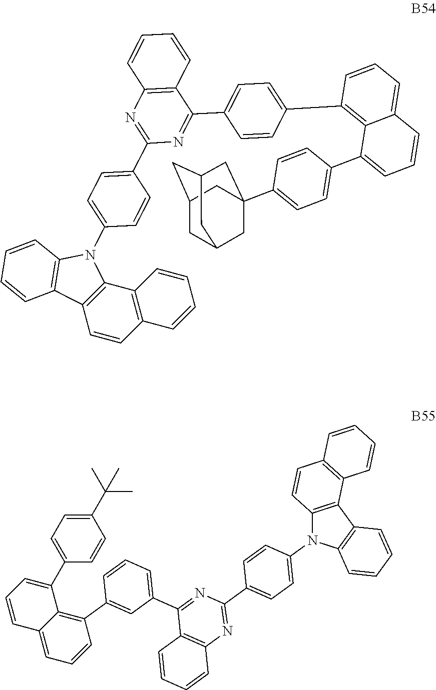 [SMILES: c1cc(-c2ccc(-c3nc(-c4ccc(-n5c6ccccc6c6ccc7ccccc7c65)cc4)nc4ccccc34)cc2)c2c(-c3ccc(C45CC6CC(CC(C6)C4)C5)cc3)cccc2c1 ; CC(C)(C)c1ccc(-c2cccc3cccc(-c4cccc(-c5nc(-c6ccc(-n7c8ccccc8c8c9ccccc9ccc87)cc6)nc6ccccc56)c4)c23)cc1]

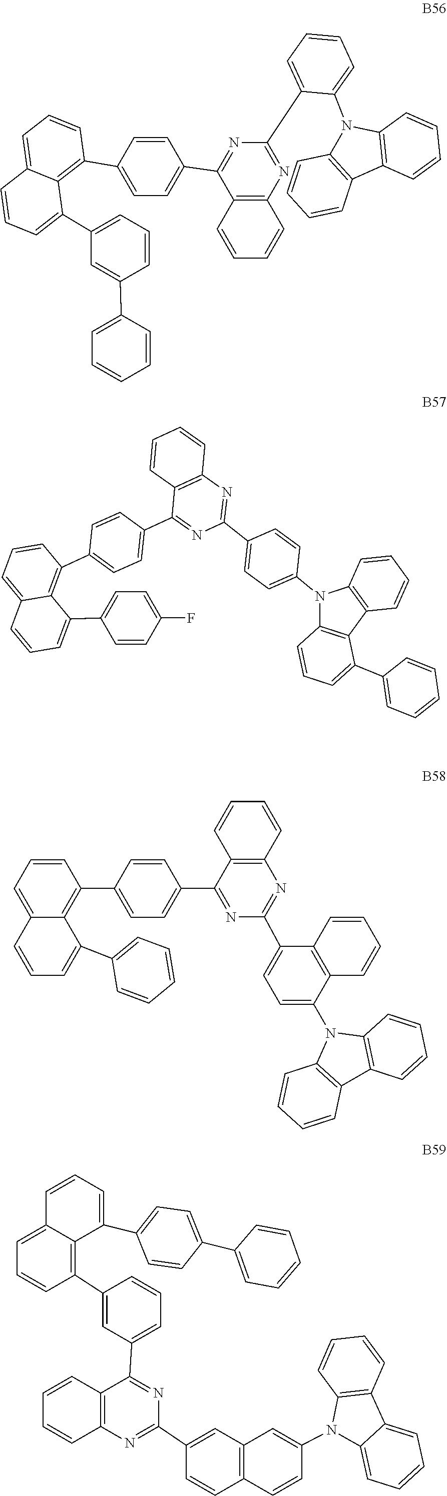 [SMILES: c1ccc(-c2cccc(-c3cccc4cccc(-c5ccc(-c6nc(-c7ccccc7-n7c8ccccc8c8ccccc87)nc7ccccc67)cc5)c34)c2)cc1 ; Fc1ccc(-c2cccc3cccc(-c4ccc(-c5nc(-c6ccc(-n7c8ccccc8c8c(-c9ccccc9)cccc87)cc6)nc6ccccc56)cc4)c23)cc1 ; c1ccc(-c2cccc3cccc(-c4ccc(-c5nc(-c6ccc(-n7c8ccccc8c8ccccc87)c7ccccc67)nc6ccccc56)cc4)c23)cc1 ; c1ccc(-c2ccc(-c3cccc4cccc(-c5cccc(-c6nc(-c7ccc8ccc(-n9c%10ccccc%10c%10ccccc%109)cc8c7)nc7ccccc67)c5)c34)cc2)cc1]

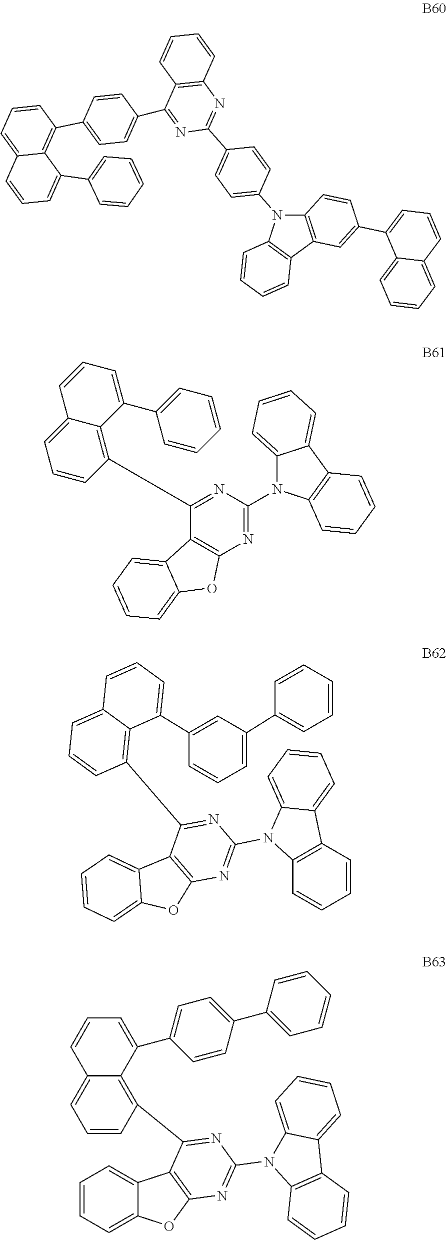 [SMILES: c1ccc(-c2cccc3cccc(-c4ccc(-c5nc(-c6ccc(-n7c8ccccc8c8cc(-c9cccc%10ccccc9%10)ccc87)cc6)nc6ccccc56)cc4)c23)cc1 ; c1ccc(-c2cccc3cccc(-c4nc(-n5c6ccccc6c6ccccc65)nc5oc6ccccc6c45)c23)cc1 ; c1ccc(-c2cccc(-c3cccc4cccc(-c5nc(-n6c7ccccc7c7ccccc76)nc6oc7ccccc7c56)c34)c2)cc1 ; c1ccc(-c2ccc(-c3cccc4cccc(-c5nc(-n6c7ccccc7c7ccccc76)nc6oc7ccccc7c56)c34)cc2)cc1]

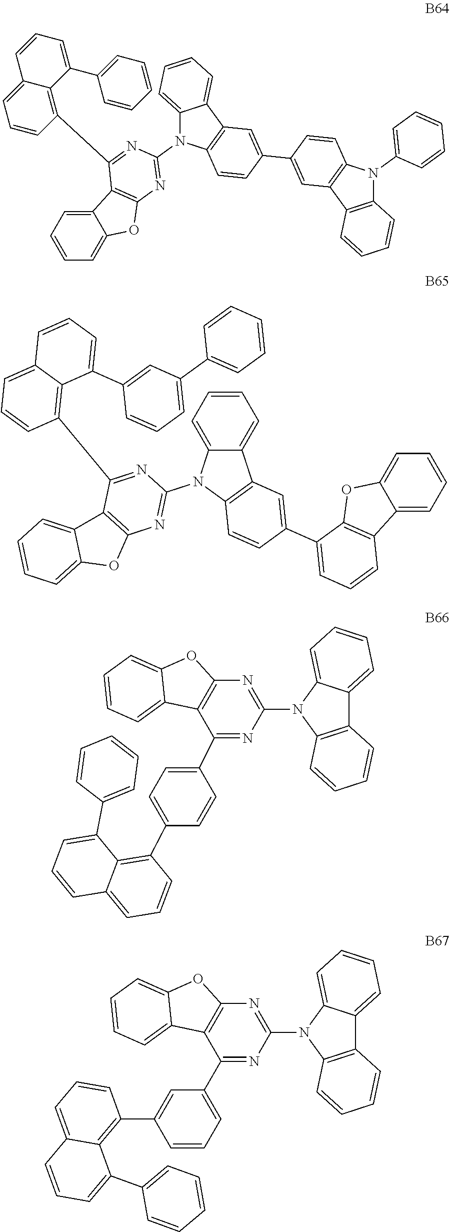

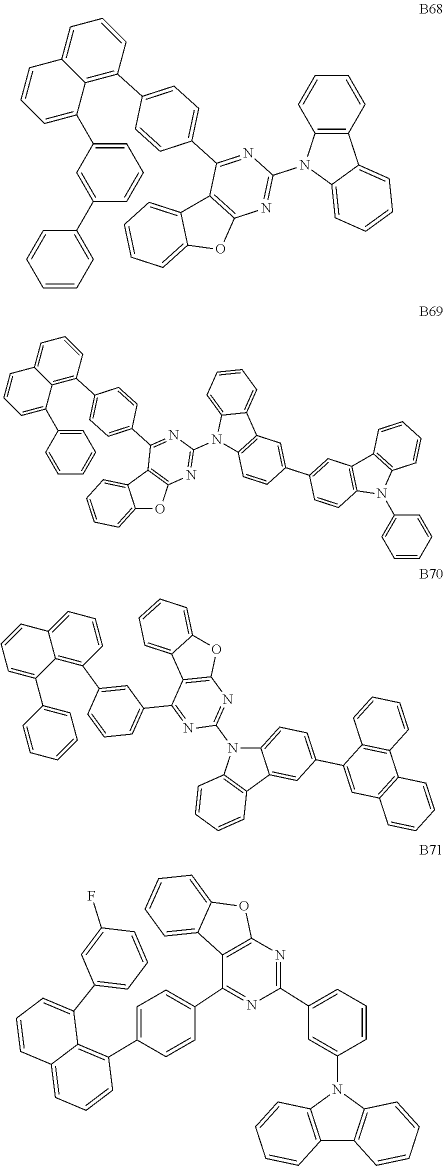 [SMILES: c1ccc(-c2cccc(-c3cccc4cccc(-c5ccc(-c6nc(-n7c8ccccc8c8ccccc87)nc7oc8ccccc8c67)cc5)c34)c2)cc1 ; c1ccc(-c2cccc3cccc(-c4ccc(-c5nc(-n6c7ccccc7c7cc(-c8ccc9c(c8)c8ccccc8n9-c8ccccc8)ccc76)nc6oc7ccccc7c56)cc4)c23)cc1 ; c1ccc(-c2cccc3cccc(-c4cccc(-c5nc(-n6c7ccccc7c7cc(-c8cc9ccccc9c9ccccc89)ccc76)nc6oc7ccccc7c56)c4)c23)cc1 ; Fc1cccc(-c2cccc3cccc(-c4ccc(-c5nc(-c6cccc(-n7c8ccccc8c8ccccc87)c6)nc6oc7ccccc7c56)cc4)c23)c1]

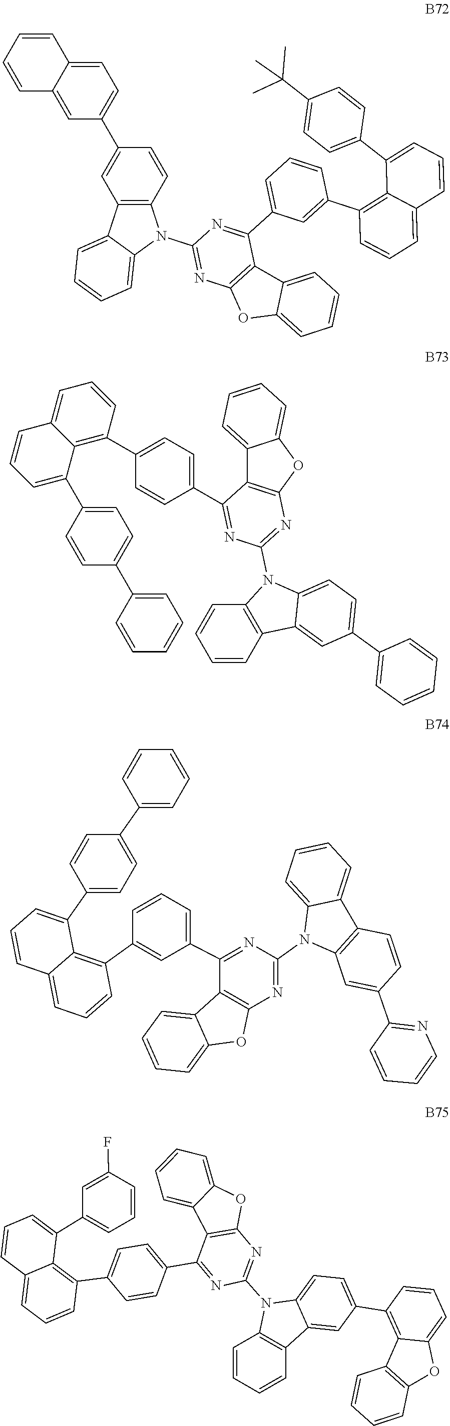 [SMILES: CC(C)(C)c1ccc(-c2cccc3cccc(-c4cccc(-c5nc(-n6c7ccccc7c7cc(-c8ccc9ccccc9c8)ccc76)nc6oc7ccccc7c56)c4)c23)cc1 ; c1ccc(-c2ccc(-c3cccc4cccc(-c5ccc(-c6nc(-n7c8ccccc8c8cc(-c9ccccc9)ccc87)nc7oc8ccccc8c67)cc5)c34)cc2)cc1 ; c1ccc(-c2ccc(-c3cccc4cccc(-c5cccc(-c6nc(-n7c8ccccc8c8ccc(-c9ccccn9)cc87)nc7oc8ccccc8c67)c5)c34)cc2)cc1 ; Fc1cccc(-c2cccc3cccc(-c4ccc(-c5nc(-n6c7ccccc7c7cc(-c8cccc9oc%10ccccc%10c89)ccc76)nc6oc7ccccc7c56)cc4)c23)c1]

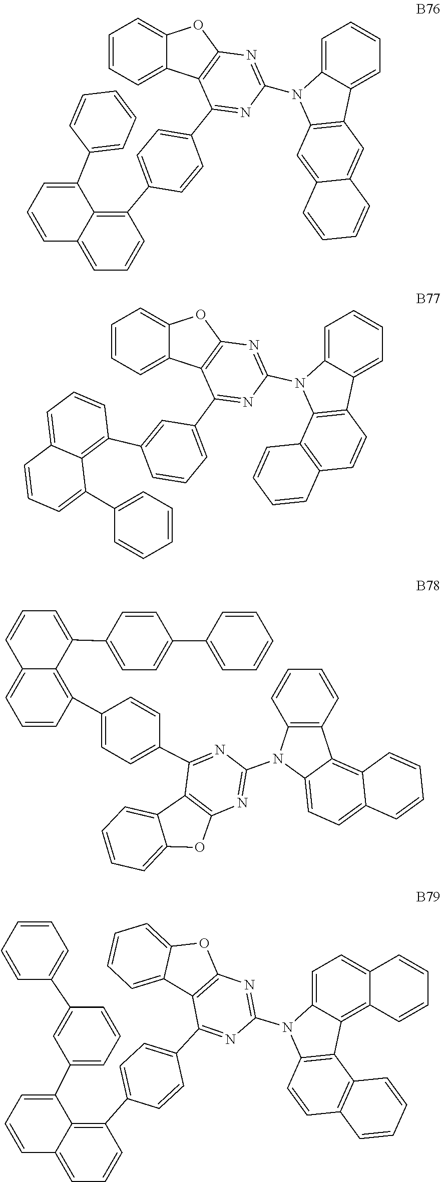

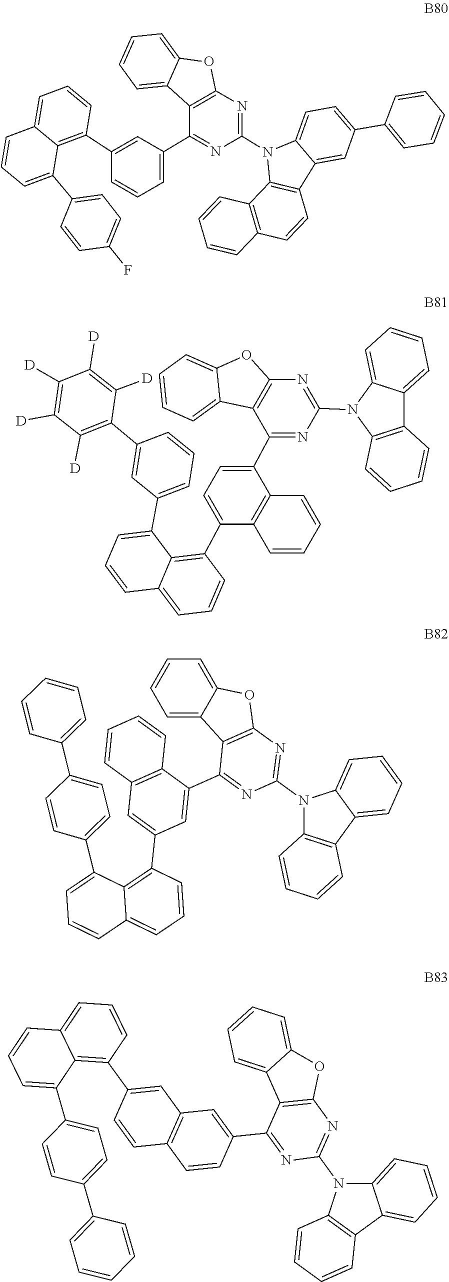

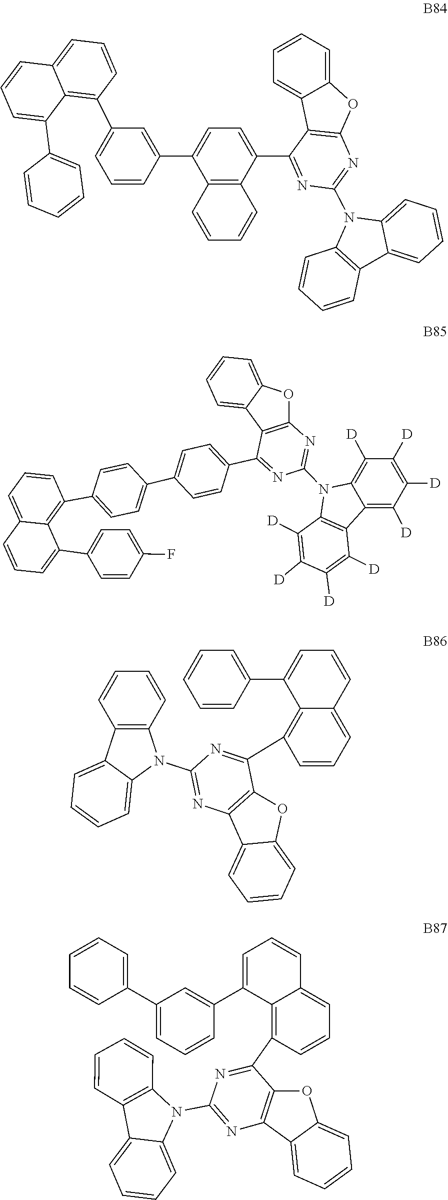

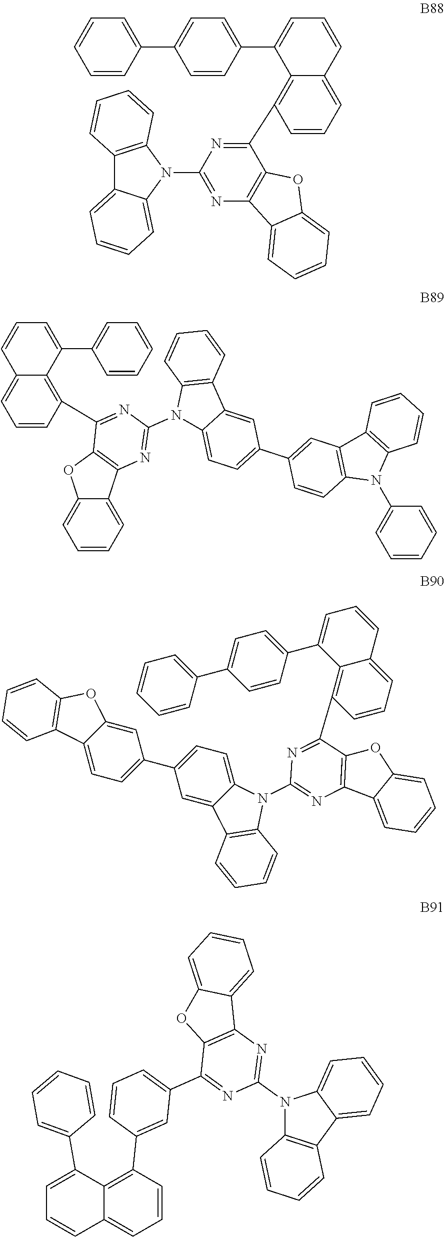 [SMILES: c1ccc(-c2ccc(-c3cccc4cccc(-c5nc(-n6c7ccccc7c7ccccc76)nc6c5oc5ccccc56)c34)cc2)cc1 ; c1ccc(-c2cccc3cccc(-c4nc(-n5c6ccccc6c6cc(-c7ccc8c(c7)c7ccccc7n8-c7ccccc7)ccc65)nc5c4oc4ccccc45)c23)cc1 ; c1ccc(-c2ccc(-c3cccc4cccc(-c5nc(-n6c7ccccc7c7cc(-c8ccc9c(c8)oc8ccccc89)ccc76)nc6c5oc5ccccc56)c34)cc2)cc1 ; c1ccc(-c2cccc3cccc(-c4cccc(-c5nc(-n6c7ccccc7c7ccccc76)nc6c5oc5ccccc56)c4)c23)cc1]

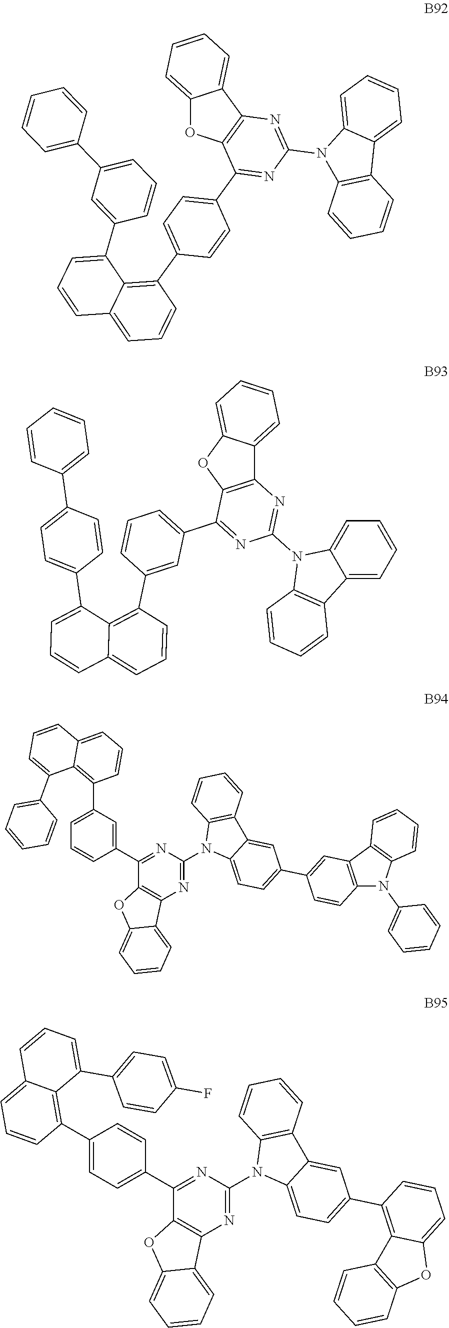 [SMILES: c1ccc(-c2cccc(-c3cccc4cccc(-c5ccc(-c6nc(-n7c8ccccc8c8ccccc87)nc7c6oc6ccccc67)cc5)c34)c2)cc1 ; c1ccc(-c2ccc(-c3cccc4cccc(-c5cccc(-c6nc(-n7c8ccccc8c8ccccc87)nc7c6oc6ccccc67)c5)c34)cc2)cc1 ; c1ccc(-c2cccc3cccc(-c4cccc(-c5nc(-n6c7ccccc7c7cc(-c8ccc9c(c8)c8ccccc8n9-c8ccccc8)ccc76)nc6c5oc5ccccc56)c4)c23)cc1 ; Fc1ccc(-c2cccc3cccc(-c4ccc(-c5nc(-n6c7ccccc7c7cc(-c8cccc9oc%10ccccc%10c89)ccc76)nc6c5oc5ccccc56)cc4)c23)cc1]

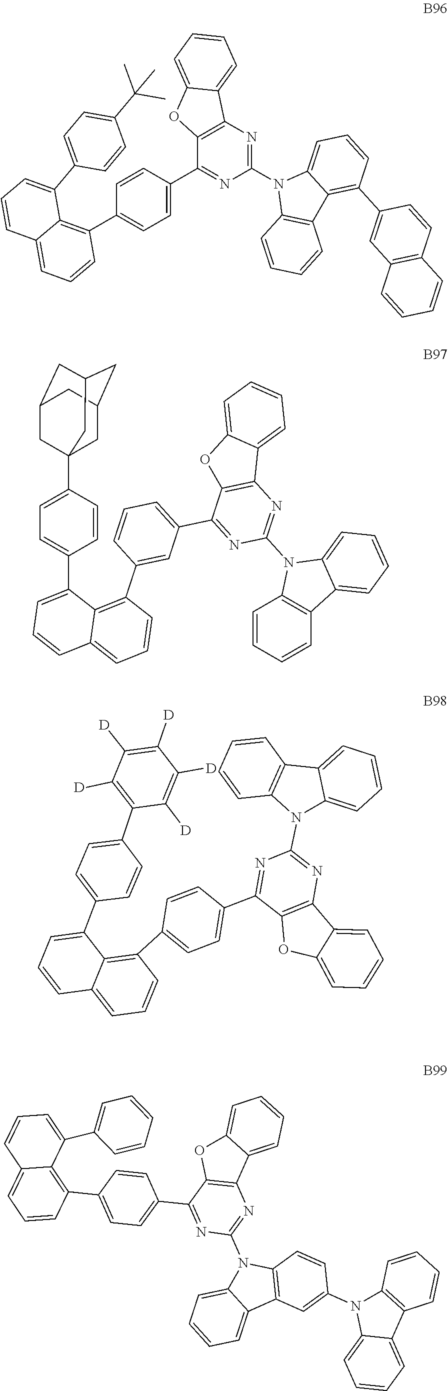 [SMILES: CC(C)(C)c1ccc(-c2cccc3cccc(-c4ccc(-c5nc(-n6c7ccccc7c7c(-c8ccc9ccccc9c8)cccc76)nc6c5oc5ccccc56)cc4)c23)cc1 ; c1cc(-c2nc(-n3c4ccccc4c4ccccc43)nc3c2oc2ccccc23)cc(-c2cccc3cccc(-c4ccc(C56CC7CC(CC(C7)C5)C6)cc4)c23)c1 ; [2H]c1c([2H])c([2H])c(-c2ccc(-c3cccc4cccc(-c5ccc(-c6nc(-n7c8ccccc8c8ccccc87)nc7c6oc6ccccc67)cc5)c34)cc2)c([2H])c1[2H] ; c1ccc(-c2cccc3cccc(-c4ccc(-c5nc(-n6c7ccccc7c7cc(-n8c9ccccc9c9ccccc98)ccc76)nc6c5oc5ccccc56)cc4)c23)cc1]

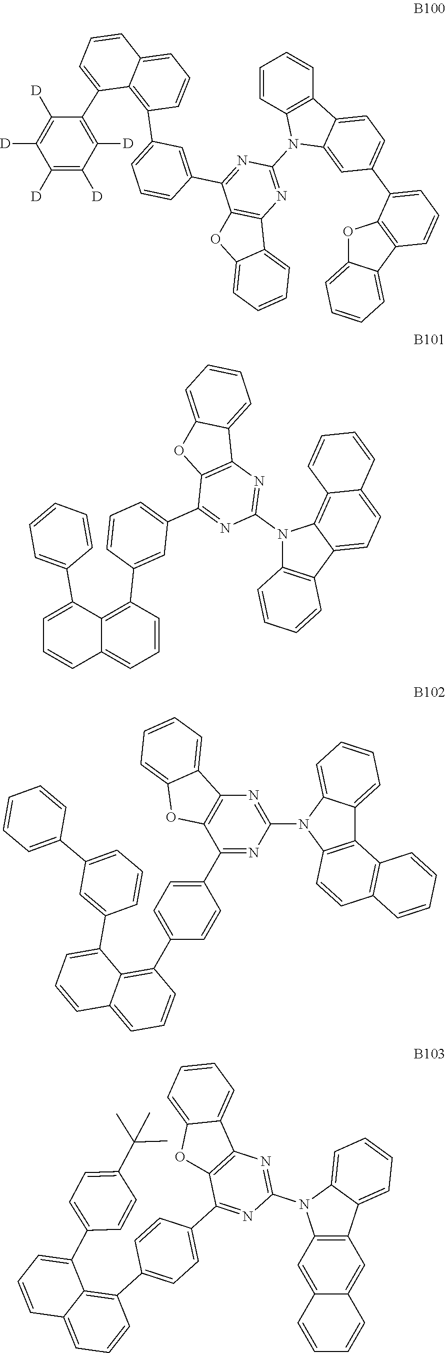

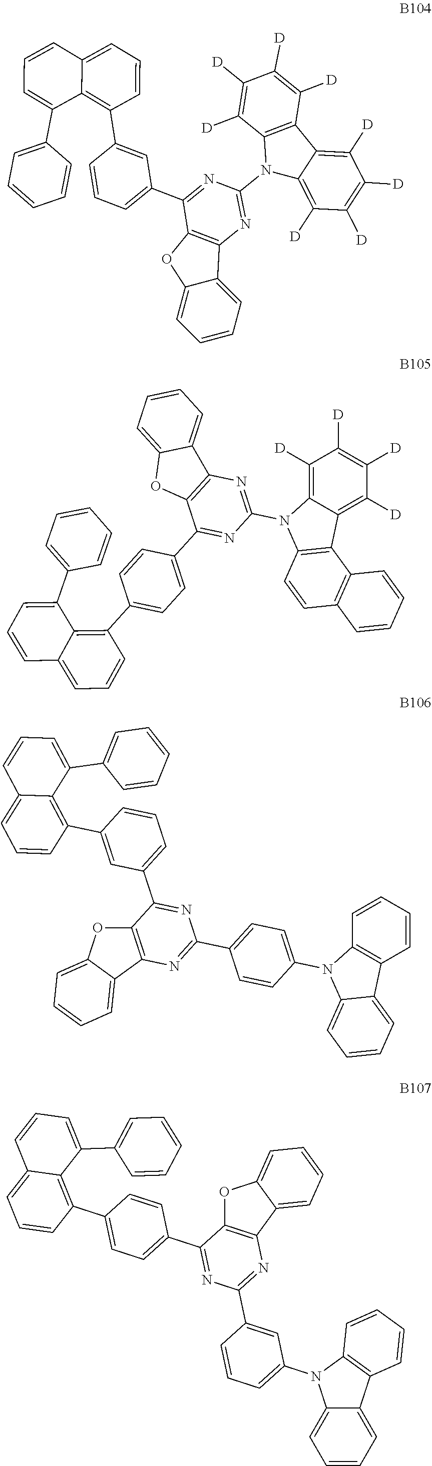

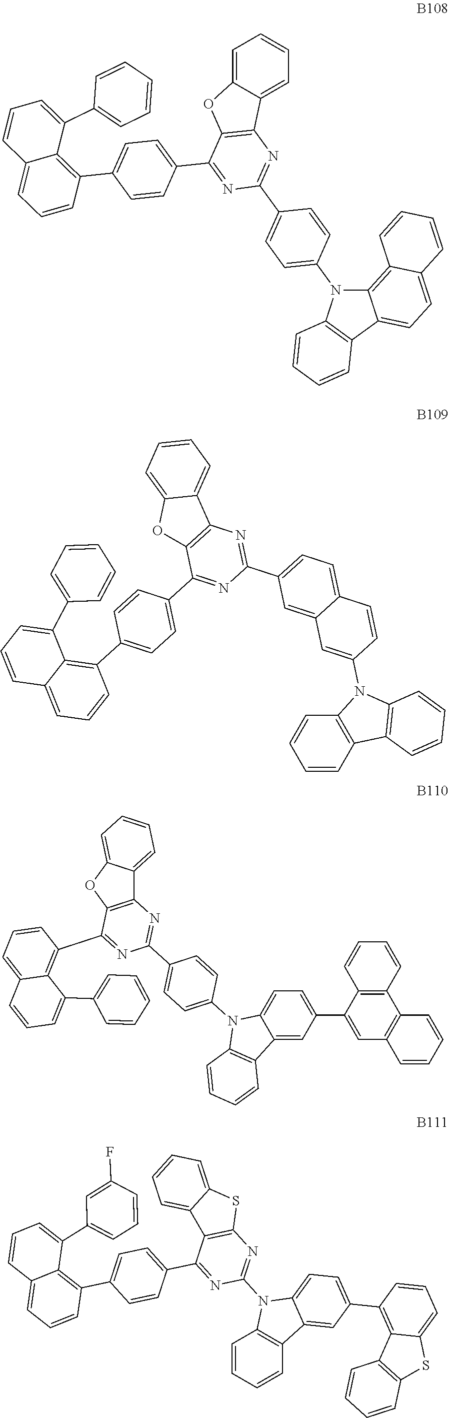 [SMILES: c1ccc(-c2cccc3cccc(-c4ccc(-c5nc(-c6ccc(-n7c8ccccc8c8ccc9ccccc9c87)cc6)nc6c5oc5ccccc56)cc4)c23)cc1 ; c1ccc(-c2cccc3cccc(-c4ccc(-c5nc(-c6ccc7ccc(-n8c9ccccc9c9ccccc98)cc7c6)nc6c5oc5ccccc56)cc4)c23)cc1 ; c1ccc(-c2cccc3cccc(-c4nc(-c5ccc(-n6c7ccccc7c7cc(-c8cc9ccccc9c9ccccc89)ccc76)cc5)nc5c4oc4ccccc45)c23)cc1 ; Fc1cccc(-c2cccc3cccc(-c4ccc(-c5nc(-n6c7ccccc7c7cc(-c8cccc9sc%10ccccc%10c89)ccc76)nc6sc7ccccc7c56)cc4)c23)c1]

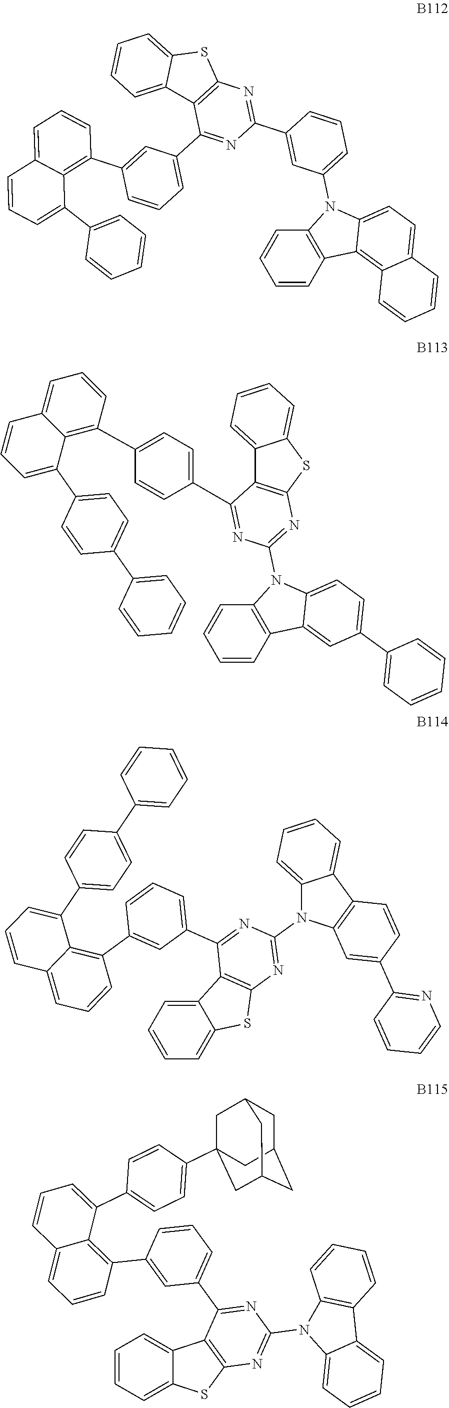 [SMILES: c1ccc(-c2cccc3cccc(-c4cccc(-c5nc(-c6cccc(-n7c8ccccc8c8c9ccccc9ccc87)c6)nc6sc7ccccc7c56)c4)c23)cc1 ; c1ccc(-c2ccc(-c3cccc4cccc(-c5ccc(-c6nc(-n7c8ccccc8c8cc(-c9ccccc9)ccc87)nc7sc8ccccc8c67)cc5)c34)cc2)cc1 ; c1ccc(-c2ccc(-c3cccc4cccc(-c5cccc(-c6nc(-n7c8ccccc8c8ccc(-c9ccccn9)cc87)nc7sc8ccccc8c67)c5)c34)cc2)cc1 ; c1cc(-c2cccc3cccc(-c4ccc(C56CC7CC(CC(C7)C5)C6)cc4)c23)cc(-c2nc(-n3c4ccccc4c4ccccc43)nc3sc4ccccc4c23)c1]

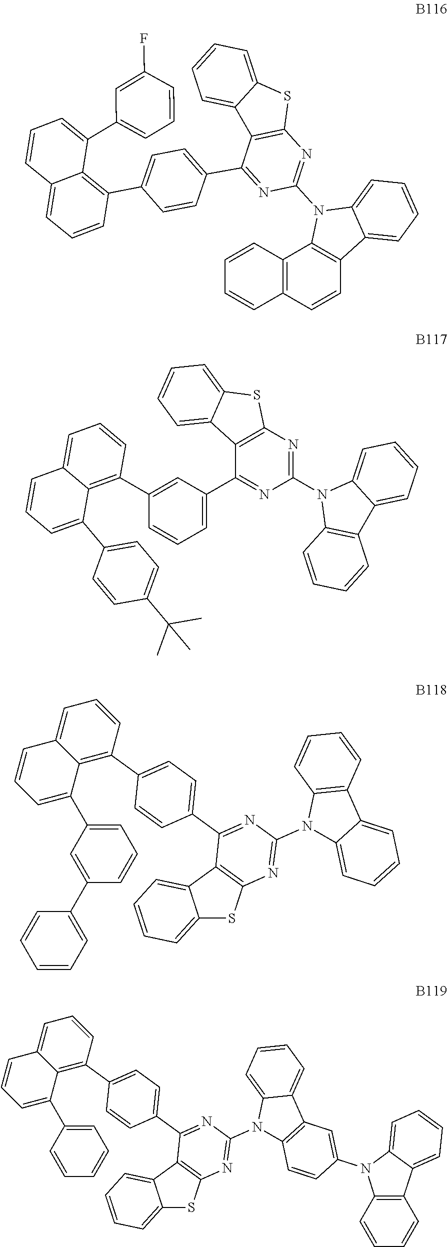 [SMILES: Fc1cccc(-c2cccc3cccc(-c4ccc(-c5nc(-n6c7ccccc7c7ccc8ccccc8c76)nc6sc7ccccc7c56)cc4)c23)c1 ; CC(C)(C)c1ccc(-c2cccc3cccc(-c4cccc(-c5nc(-n6c7ccccc7c7ccccc76)nc6sc7ccccc7c56)c4)c23)cc1 ; c1ccc(-c2cccc(-c3cccc4cccc(-c5ccc(-c6nc(-n7c8ccccc8c8ccccc87)nc7sc8ccccc8c67)cc5)c34)c2)cc1 ; c1ccc(-c2cccc3cccc(-c4ccc(-c5nc(-n6c7ccccc7c7cc(-n8c9ccccc9c9ccccc98)ccc76)nc6sc7ccccc7c56)cc4)c23)cc1]

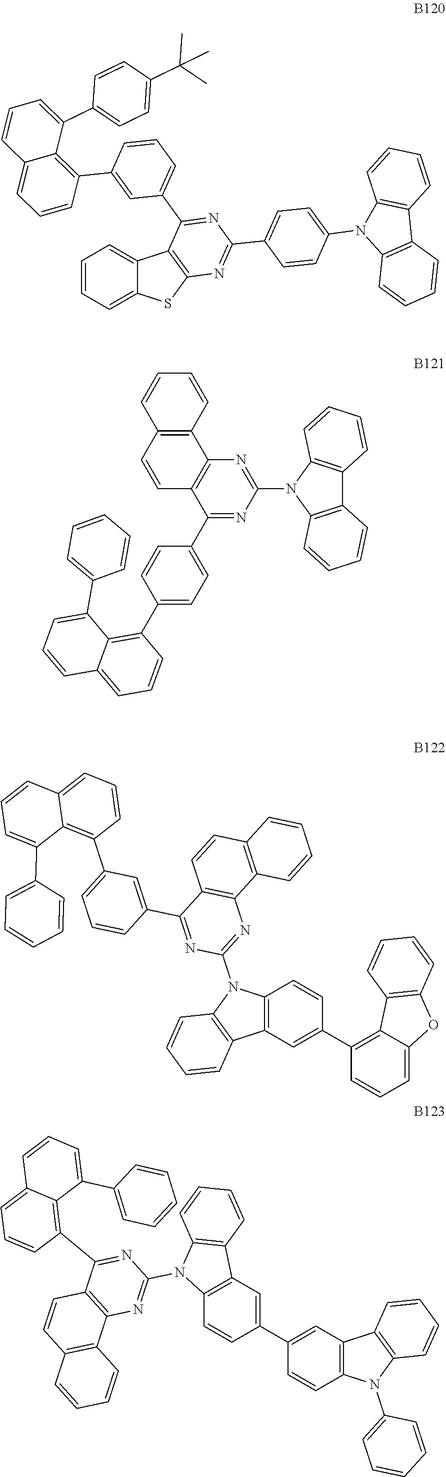 [SMILES: CC(C)(C)c1ccc(-c2cccc3cccc(-c4cccc(-c5nc(-c6ccc(-n7c8ccccc8c8ccccc87)cc6)nc6sc7ccccc7c56)c4)c23)cc1 ; c1ccc(-c2cccc3cccc(-c4ccc(-c5nc(-n6c7ccccc7c7ccccc76)nc6c5ccc5ccccc56)cc4)c23)cc1 ; c1ccc(-c2cccc3cccc(-c4cccc(-c5nc(-n6c7ccccc7c7cc(-c8cccc9oc%10ccccc%10c89)ccc76)nc6c5ccc5ccccc56)c4)c23)cc1 ; c1ccc(-c2cccc3cccc(-c4nc(-n5c6ccccc6c6cc(-c7ccc8c(c7)c7ccccc7n8-c7ccccc7)ccc65)nc5c4ccc4ccccc45)c23)cc1]

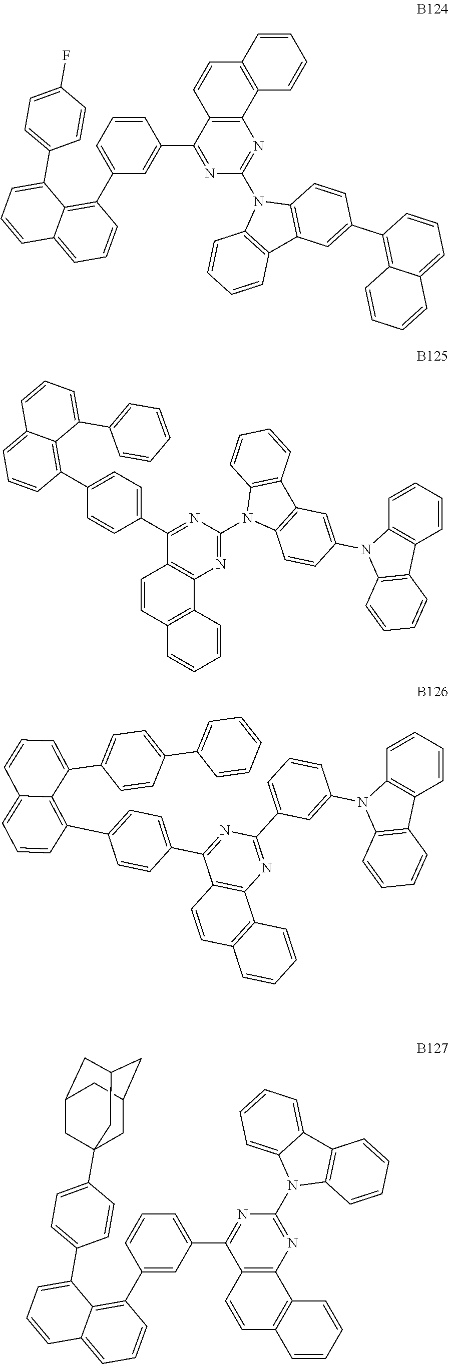 [SMILES: Fc1ccc(-c2cccc3cccc(-c4cccc(-c5nc(-n6c7ccccc7c7cc(-c8cccc9ccccc89)ccc76)nc6c5ccc5ccccc56)c4)c23)cc1 ; c1ccc(-c2cccc3cccc(-c4ccc(-c5nc(-n6c7ccccc7c7cc(-n8c9ccccc9c9ccccc98)ccc76)nc6c5ccc5ccccc56)cc4)c23)cc1 ; c1ccc(-c2ccc(-c3cccc4cccc(-c5ccc(-c6nc(-c7cccc(-n8c9ccccc9c9ccccc98)c7)nc7c6ccc6ccccc67)cc5)c34)cc2)cc1 ; c1cc(-c2nc(-n3c4ccccc4c4ccccc43)nc3c2ccc2ccccc23)cc(-c2cccc3cccc(-c4ccc(C56CC7CC(CC(C7)C5)C6)cc4)c23)c1]

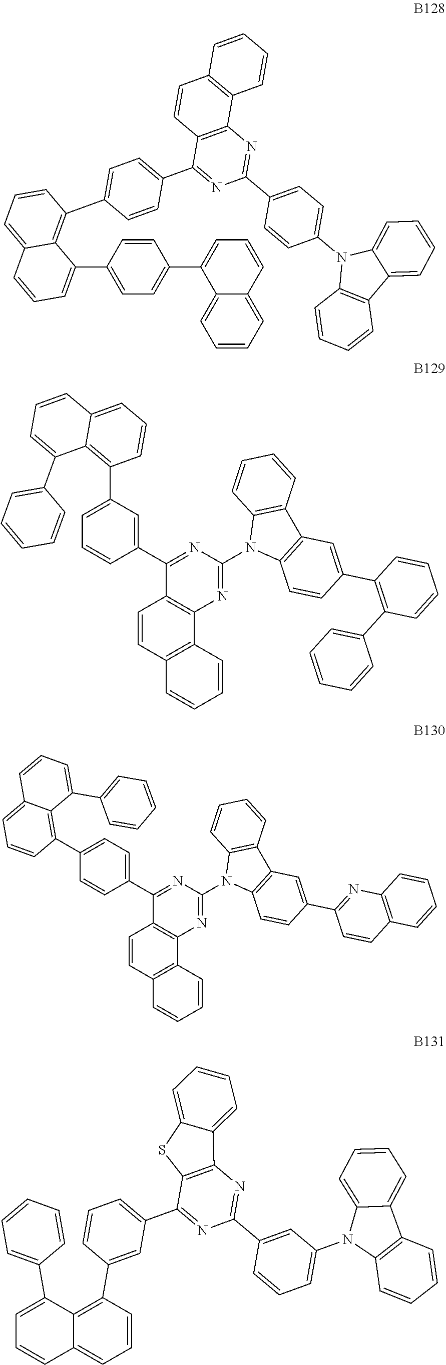 [SMILES: c1ccc2c(-c3ccc(-c4cccc5cccc(-c6ccc(-c7nc(-c8ccc(-n9c%10ccccc%10c%10ccccc%109)cc8)nc8c7ccc7ccccc78)cc6)c45)cc3)cccc2c1 ; c1ccc(-c2ccccc2-c2ccc3c(c2)c2ccccc2n3-c2nc(-c3cccc(-c4cccc5cccc(-c6ccccc6)c45)c3)c3ccc4ccccc4c3n2)cc1 ; c1ccc(-c2cccc3cccc(-c4ccc(-c5nc(-n6c7ccccc7c7cc(-c8ccc9ccccc9n8)ccc76)nc6c5ccc5ccccc56)cc4)c23)cc1 ; c1ccc(-c2cccc3cccc(-c4cccc(-c5nc(-c6cccc(-n7c8ccccc8c8ccccc87)c6)nc6c5sc5ccccc56)c4)c23)cc1]

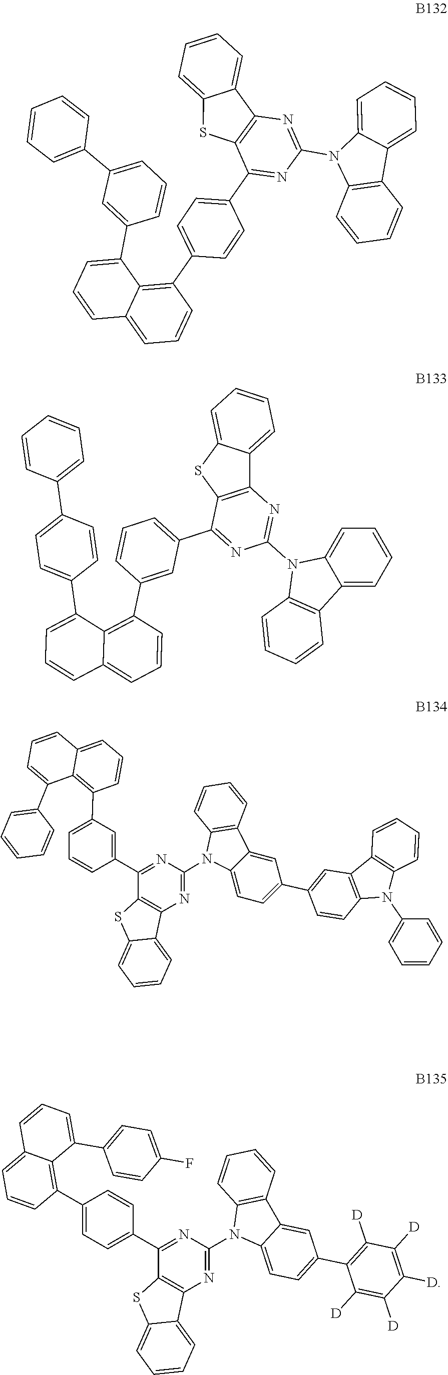

In a second aspect, the present disclosure provides an electronic element, including an anode and a cathode which are oppositely disposed, and a functional layer disposed between the anode and the cathode, where the functional layer includes the organic compound of the present disclosure.

Optionally, the functional layer includes an organic light-emitting layer including the organic compound described in the present disclosure.

Optionally, the electronic element is an organic electroluminescent device.

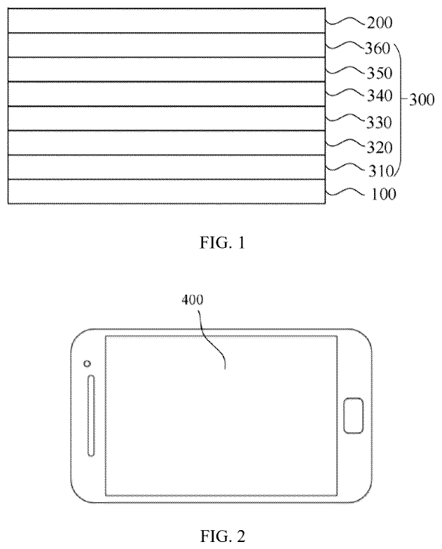

In one embodiment, the electronic element is an organic electroluminescent device. As shown in FIG. 1, the organic electroluminescent device may include an anode 100, a hole transport layer 320, an electron blocking layer 330, an organic light-emitting layer 340, an electron transport layer 350, and a cathode 200 which are sequentially stacked.

In one specific embodiment, the organic electroluminescent device is a red organic electroluminescent device.

Optionally, the anode 100 includes the following anode materials which are optionally materials having a large work function that facilitate hole injection into the functional layer. Specific examples of the anode materials include metals such as nickel, platinum, vanadium, chromium, copper, zinc, and gold, or its alloy; metal oxides such as zinc oxide, indium oxide, indium tin oxide (ITO) and indium zinc oxide (IZO); combined metals and oxides, such as ZnO:Al or SnO₂:Sb; or a conductive polymer such as poly(3-methylthiophene), poly[3,4-(ethylene-1,2-dioxy)thiophene](PEDT), polypyrrole, and polyaniline, but are not limited to this. A transparent electrode including indium tin oxide (ITO) as the anode is preferably included.

Optionally, the hole transport layer 320 includes one or more hole transport materials, and the hole transport materials may be selected from carbazole multimers, carbazole-linked triarylamine compounds, or other types of compounds, which can be selected by those skilled in the art with reference to the prior art. For example, a material of the hole transport layer is selected from the group consisting of the following compounds:

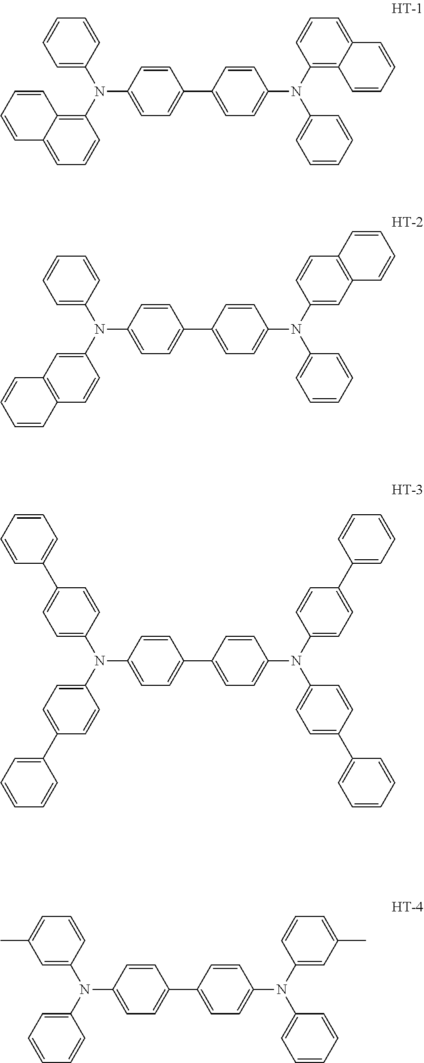

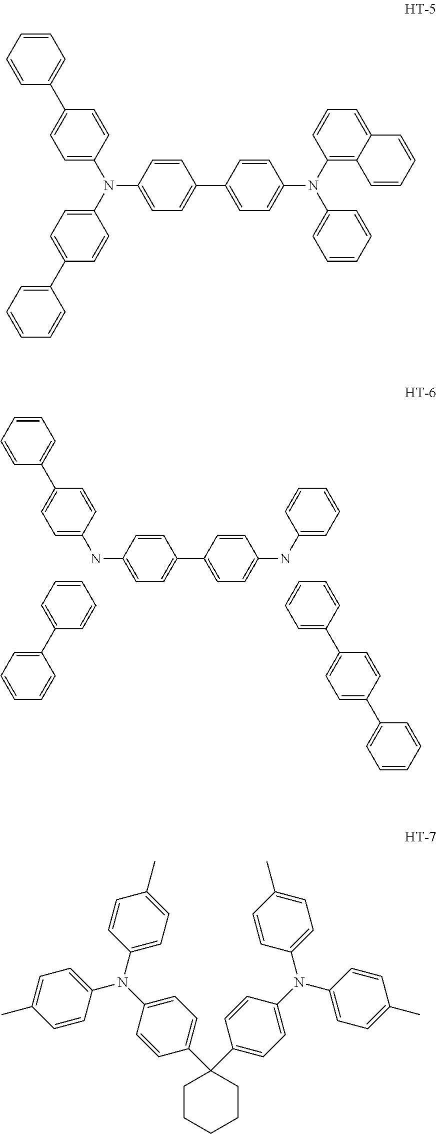

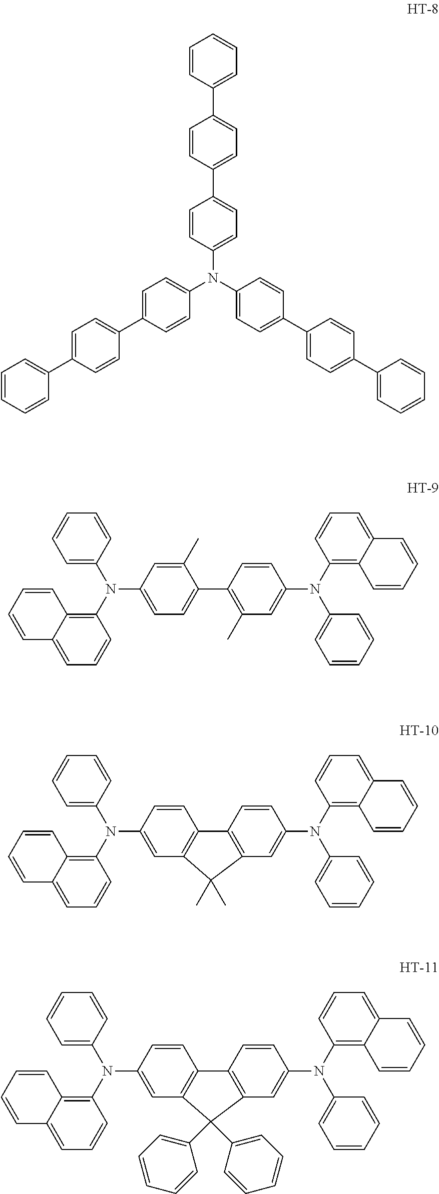 [SMILES: c1ccc(-c2ccc(-c3ccc(N(c4ccc(-c5ccc(-c6ccccc6)cc5)cc4)c4ccc(-c5ccc(-c6ccccc6)cc5)cc4)cc3)cc2)cc1 ; Cc1cc(N(c2ccccc2)c2cccc3ccccc23)ccc1-c1ccc(N(c2ccccc2)c2cccc3ccccc23)cc1C ; CC1(C)c2cc(N(c3ccccc3)c3cccc4ccccc34)ccc2-c2ccc(N(c3ccccc3)c3cccc4ccccc34)cc21 ; c1ccc(N(c2ccc3c(c2)C(c2ccccc2)(c2ccccc2)c2cc(N(c4ccccc4)c4cccc5ccccc45)ccc2-3)c2cccc3ccccc23)cc1]

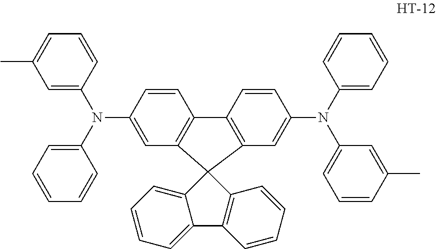

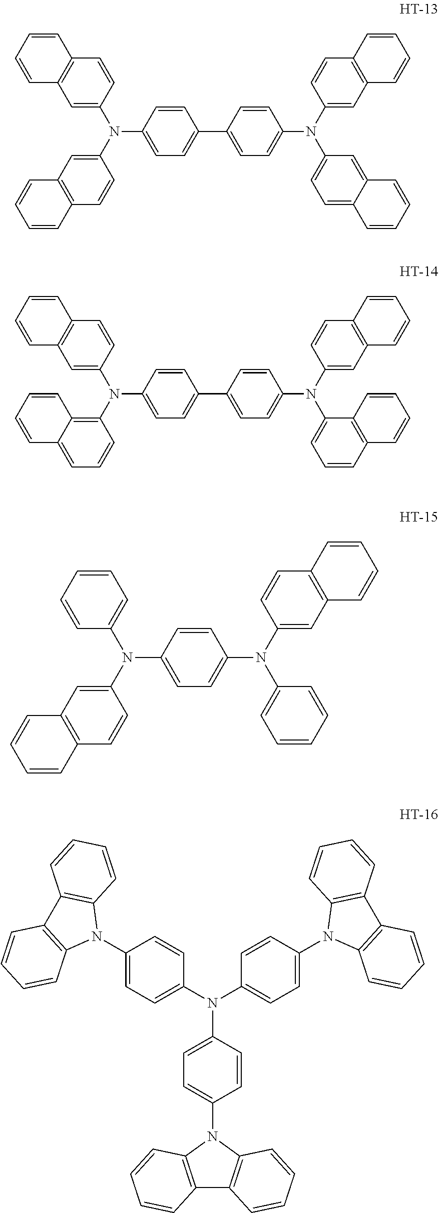 [SMILES: c1ccc2cc(N(c3ccc(-c4ccc(N(c5ccc6ccccc6c5)c5ccc6ccccc6c5)cc4)cc3)c3ccc4ccccc4c3)ccc2c1 ; c1ccc2cc(N(c3ccc(-c4ccc(N(c5ccc6ccccc6c5)c5cccc6ccccc56)cc4)cc3)c3cccc4ccccc34)ccc2c1 ; c1ccc(N(c2ccc(N(c3ccccc3)c3ccc4ccccc4c3)cc2)c2ccc3ccccc3c2)cc1 ; c1ccc2c(c1)c1ccccc1n2-c1ccc(N(c2ccc(-n3c4ccccc4c4ccccc43)cc2)c2ccc(-n3c4ccccc4c4ccccc43)cc2)cc1]

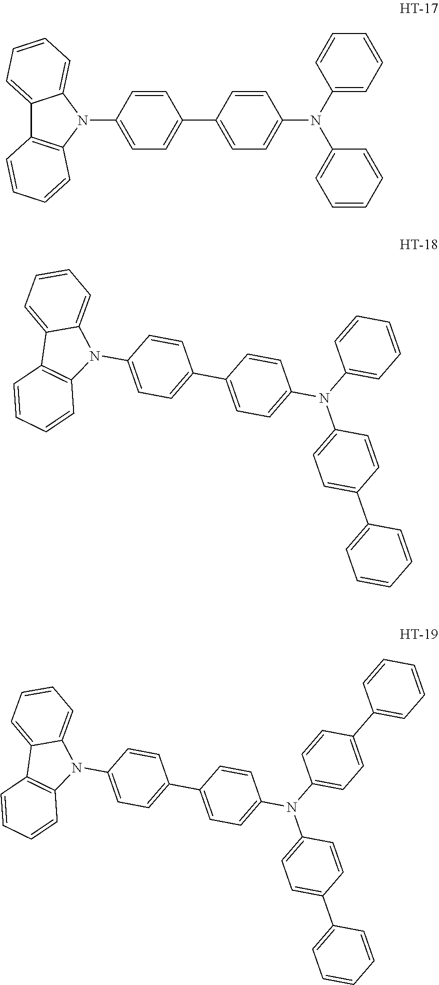 [SMILES: c1ccc(N(c2ccccc2)c2ccc(-c3ccc(-n4c5ccccc5c5ccccc54)cc3)cc2)cc1 ; c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

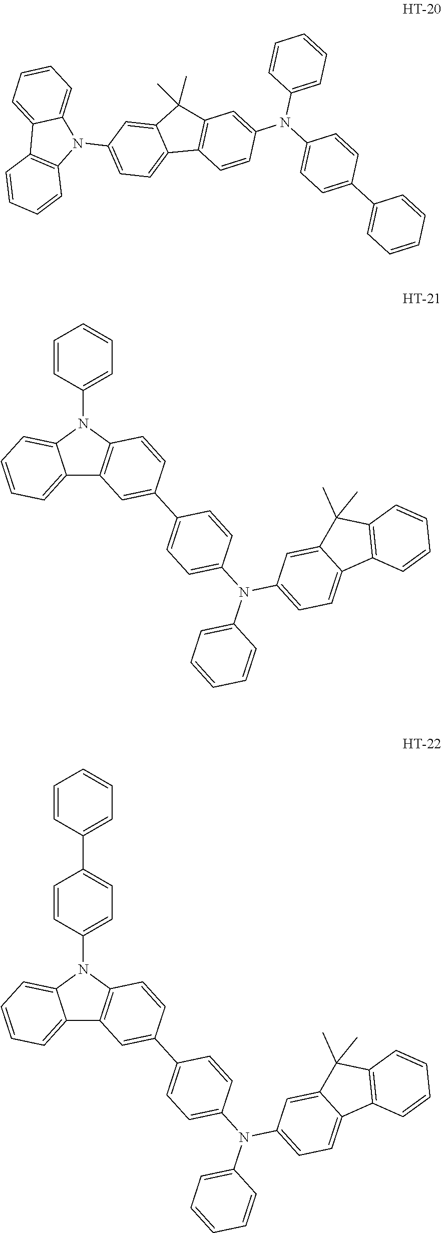 [SMILES: CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

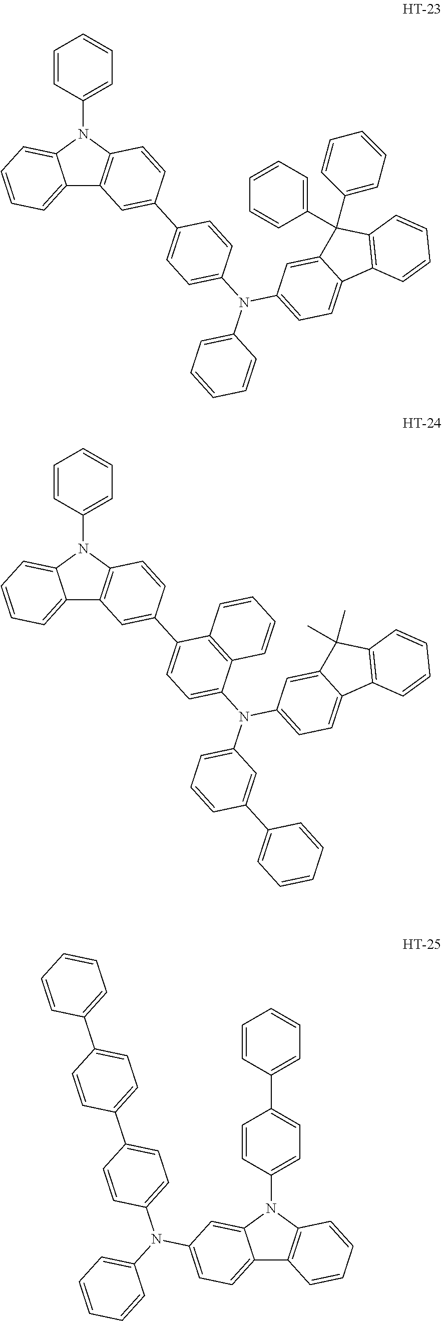 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; c1ccc(-c2ccc(-c3ccc(N(c4ccccc4)c4ccc5c6ccccc6n(-c6ccc(-c7ccccc7)cc6)c5c4)cc3)cc2)cc1]

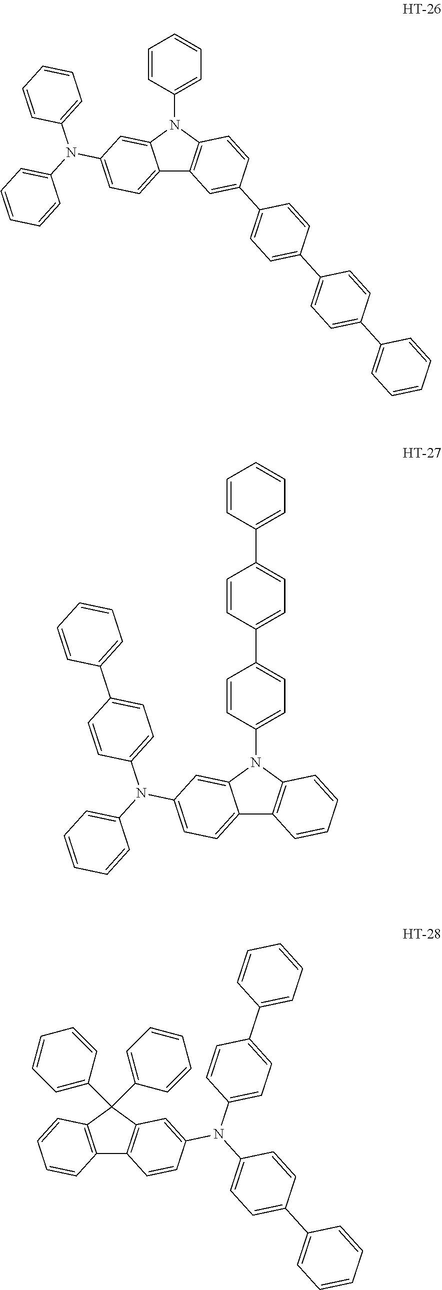 [SMILES: c1ccc(-c2ccc(-c3ccc(-c4ccc5c(c4)c4ccc(N(c6ccccc6)c6ccccc6)cc4n5-c4ccccc4)cc3)cc2)cc1 ; c1ccc(-c2ccc(-c3ccc(-n4c5ccccc5c5ccc(N(c6ccccc6)c6ccc(-c7ccccc7)cc6)cc54)cc3)cc2)cc1 ; c1ccc(-c2ccc(N(c3ccc(-c4ccccc4)cc3)c3ccc4c(c3)C(c3ccccc3)(c3ccccc3)c3ccccc3-4)cc2)cc1]

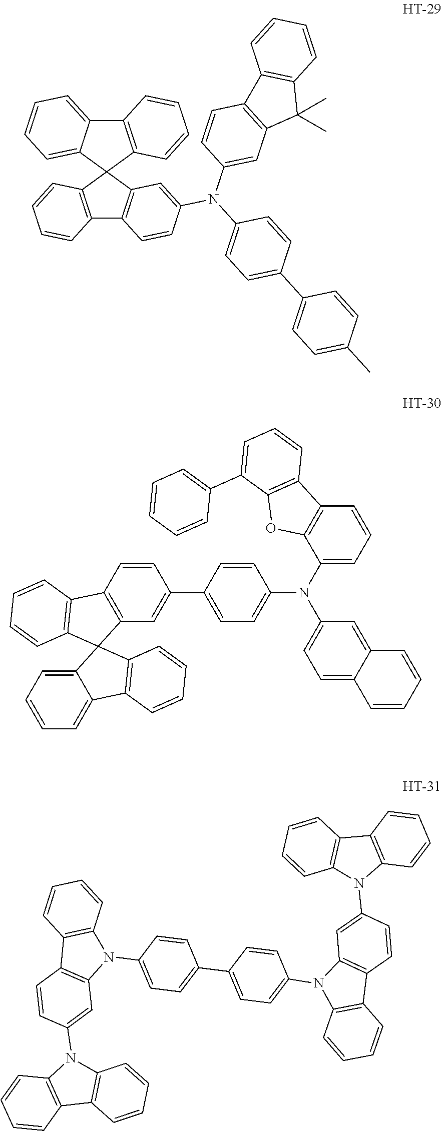 [SMILES: Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1 ; c1ccc2c(c1)c1ccccc1n2-c1ccc2c3ccccc3n(-c3ccc(-c4ccc(-n5c6ccccc6c6ccc(-n7c8ccccc8c8ccccc87)cc65)cc4)cc3)c2c1]

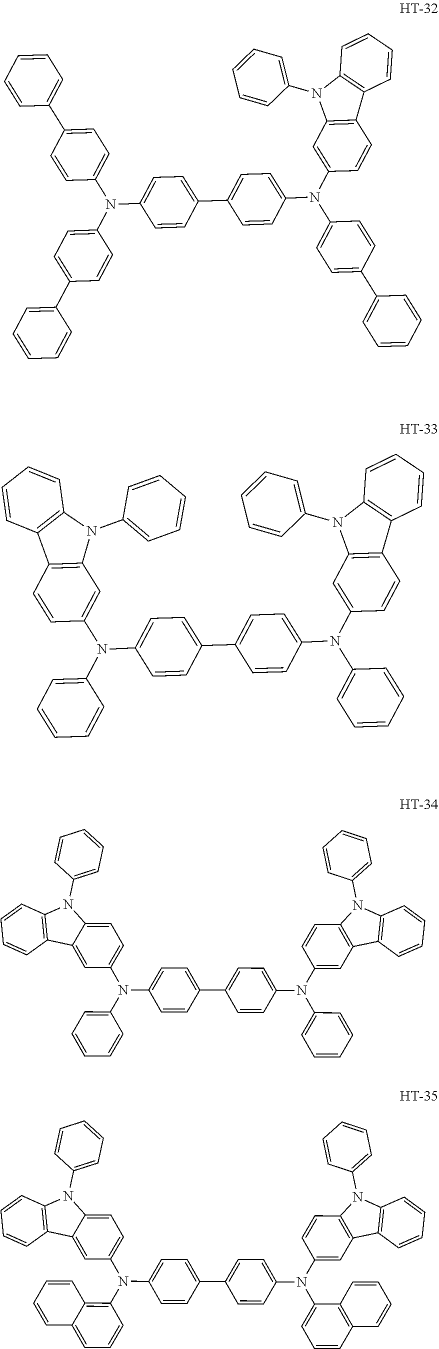 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(N(c2ccc(-c3ccc(N(c4ccccc4)c4ccc5c6ccccc6n(-c6ccccc6)c5c4)cc3)cc2)c2ccc3c4ccccc4n(-c4ccccc4)c3c2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1]

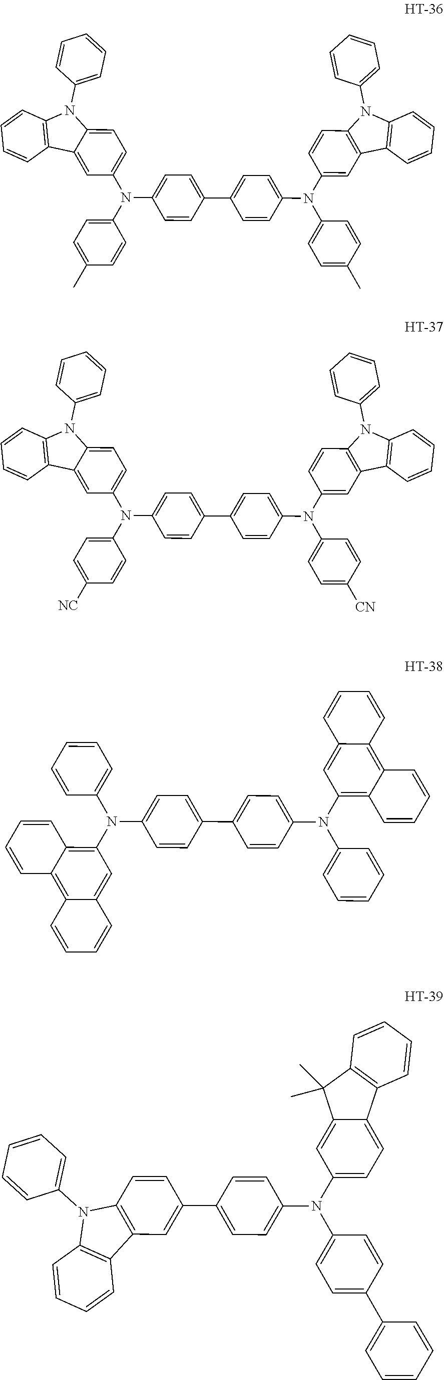 [SMILES: Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1 ; c1ccc(N(c2ccc(-c3ccc(N(c4ccccc4)c4cc5ccccc5c5ccccc45)cc3)cc2)c2cc3ccccc3c3ccccc23)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21]

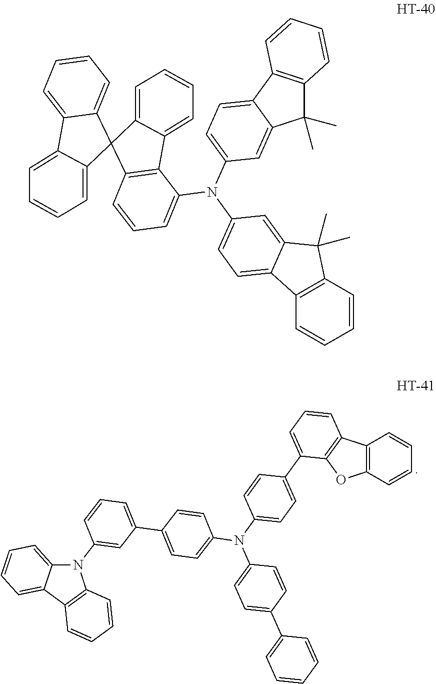 [SMILES: CC1(C)c2ccccc2-c2ccc(N(c3ccc4c(c3)C(C)(C)c3ccccc3-4)c3cccc4c3-c3ccccc3C43c4ccccc4-c4ccccc43)cc21 ; c1ccc(-c2ccc(N(c3ccc(-c4cccc(-n5c6ccccc6c6ccccc65)c4)cc3)c3ccc(-c4cccc5c4oc4ccccc45)cc3)cc2)cc1]

In one specific embodiment, the hole transport layer 320 is made of HT-1.

Optionally, the electron blocking layer 330 includes one or more electron blocking materials, and the electron blocking materials may be selected from carbazole multimers or other types of compounds, which are not particularly limited in the present disclosure. In one specific embodiment, the electron blocking layer 330 is made of TCAC.

Optionally, the organic light-emitting layer 340 may consist of a single light-emitting layer material, and may also include a host material and a doping material. Optionally, the organic light-emitting layer 340 is composed of the host material and the doping material, holes injected into the organic light-emitting layer 340 and electrons injected into the organic light-emitting layer 340 may be recombined in the organic light-emitting layer 340 to form excitons, the excitons transfer energy to the host material, and the host material transfers energy to the doping material, thus enabling the doping material to emit light.

The host material of the organic light-emitting layer 340 may be a metal chelated compound, a bisstyryl derivative, an aromatic amine derivative, a dibenzofuran derivative, or other types of materials, which are not particularly limited in the present disclosure. The host material may be a single host material or a mixed host material.

In one embodiment of the present disclosure, the host material of the organic light-emitting layer 340 is the organic compound of the present disclosure.

The doping material of the organic light-emitting layer 340 may be selected with reference to the prior art, and for example, may be selected from an iridium (III) organometallic complex, a platinum (II) organometallic complex, a ruthenium (II) complex, and the like. Specific examples of the doping material include, but are not limited to,

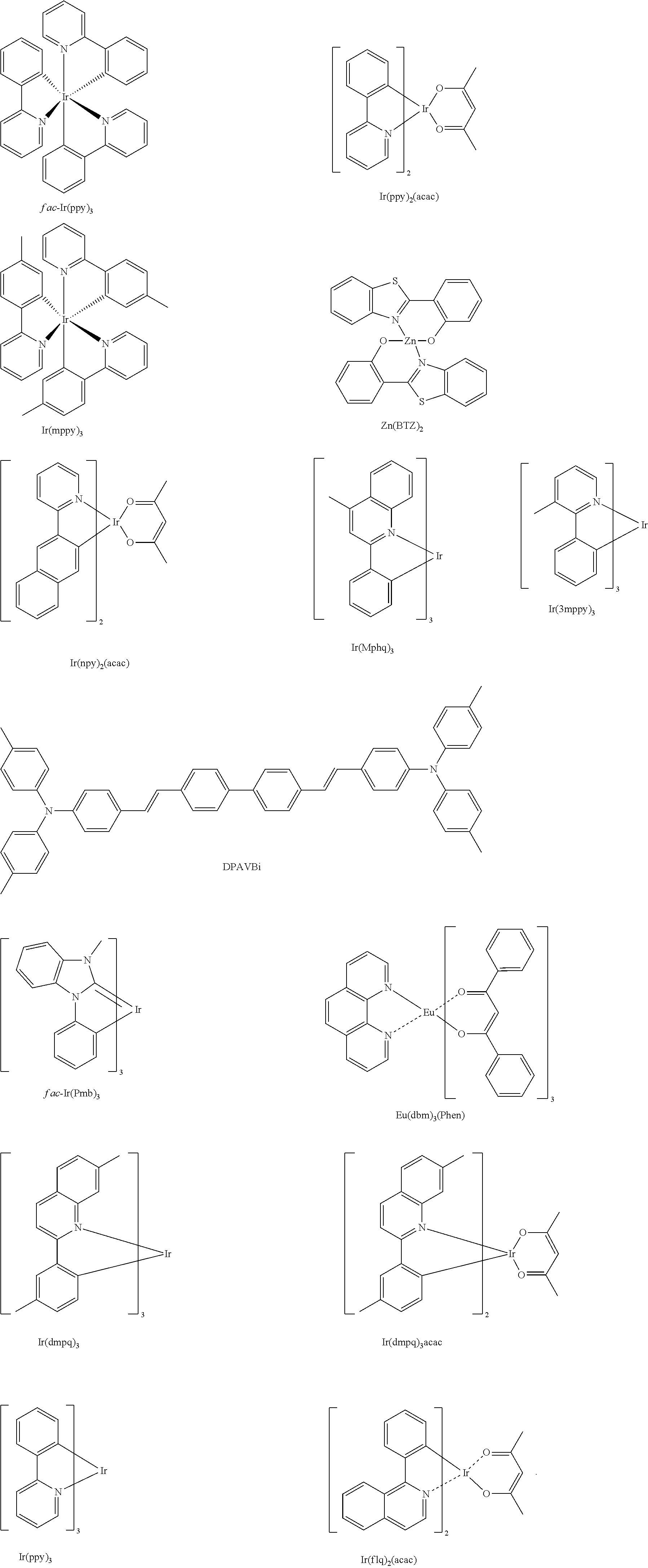

In one embodiment of the present disclosure, the doping material of the organic light-emitting layer 340 is Ir(flq)₂(acac).

Optionally, the electron transport layer 350 may have a single-layer structure or a multi-layer structure, and may include one or more electron transport materials, and the electron transport materials may generally include a metal complex or a nitrogen-containing heterocyclic derivative, where the metal complex material may be selected from, for example, LiQ, Alq₃, Bepq₂, etc.; the nitrogen-containing heterocyclic derivative may be an aromatic ring having a nitrogen-containing six-membered ring or five-membered ring skeleton, a condensed aromatic ring compound having a nitrogen-containing six-membered ring or five-membered ring skeleton, or the like, and specific examples include, but are not limited to, 1,10-phenanthroline compounds such as ET-01, Bphen, NBphen, DBimiBphen, and BimiBphen, or an anthracene compound, a triazine compound, or a pyrimidine compound containing azaaryl with the structure shown below. In one embodiment of the present disclosure, the electron transport layer 350 consists of ET-01 and LiQ.

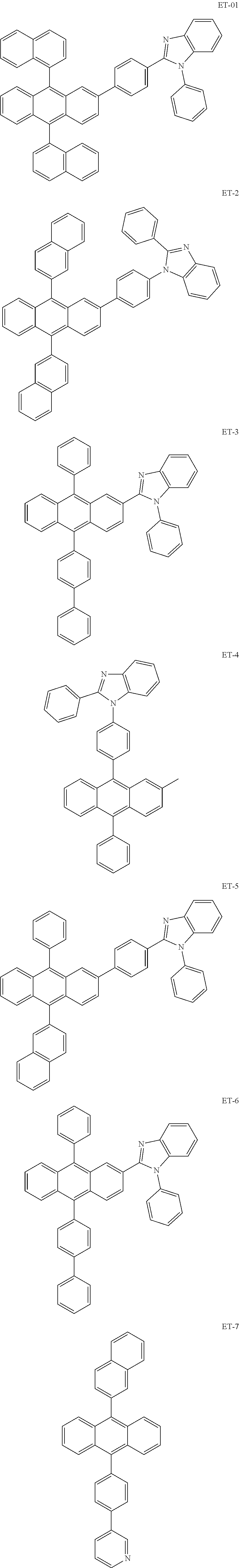

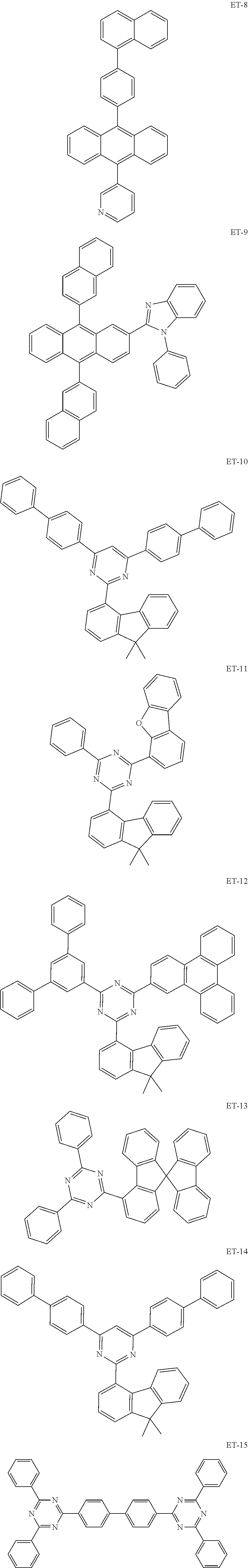

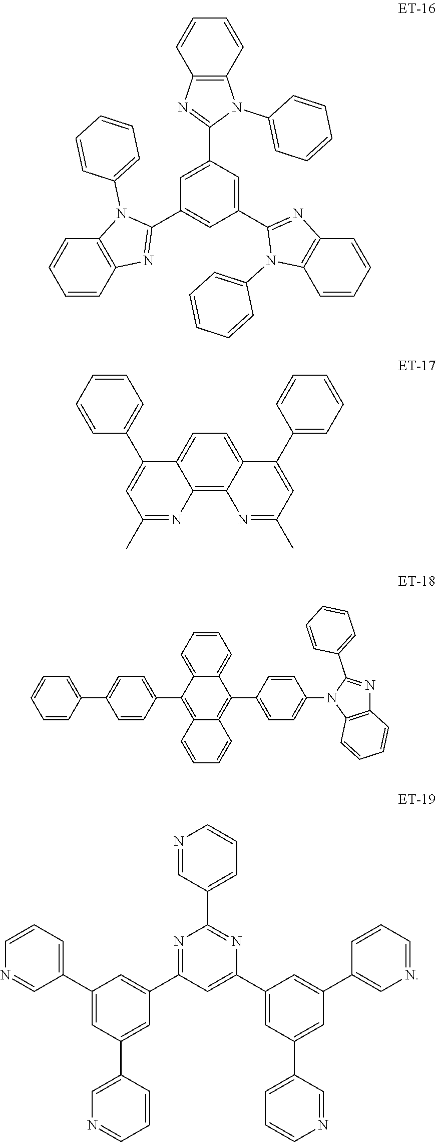 [SMILES: c1ccc(-n2c(-c3cc(-c4nc5ccccc5n4-c4ccccc4)cc(-c4nc5ccccc5n4-c4ccccc4)c3)nc3ccccc32)cc1 ; Cc1cc(-c2ccccc2)c2ccc3c(-c4ccccc4)cc(C)nc3c2n1 ; c1ccc(-c2ccc(-c3c4ccccc4c(-c4ccc(-n5c(-c6ccccc6)nc6ccccc65)cc4)c4ccccc34)cc2)cc1 ; c1cncc(-c2cc(-c3cccnc3)cc(-c3cc(-c4cc(-c5cccnc5)cc(-c5cccnc5)c4)nc(-c4cccnc4)n3)c2)c1]

In the present disclosure, the cathode 200 includes a cathode material which is a material having a small work function that facilitates electron injection into the functional layer. Specific examples of the cathode material include: metals such as magnesium, calcium, sodium, potassium, titanium, indium, yttrium, lithium, gadolinium, aluminum, silver, tin and lead or its alloy; or multilayer materials such as LiF/Al, Liq/Al, LiO₂/Al, LiF/Ca, LiF/Al and BaF₂/Ca. A metal electrode including magnesium and silver is preferably included as the cathode.

Optionally, as shown in FIG. 1, a hole injection layer 310 may also be disposed between the anode 100 and the hole transport layer 320 to enhance the ability to inject holes into the hole transport layer 320. The hole injection layer 310 may be made of a benzidine derivative, a starburst arylamine compound, a phthalocyanine derivative, or other materials, which is not particularly limited in the present disclosure. For example, the hole injection layer 310 contains a compound selected from the group consisting of the following compounds.

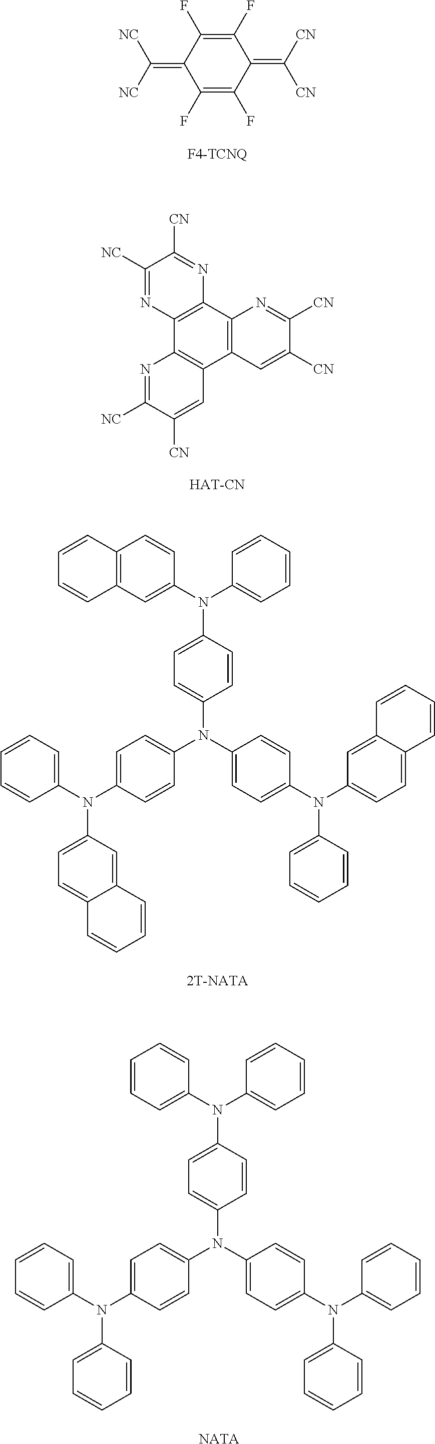 [SMILES: N#CC(C#N)=c1c(F)c(F)c(=C(C#N)C#N)c(F)c1F ; N#Cc1cc2c3cc(C#N)c(C#N)nc3c3nc(C#N)c(C#N)nc3c2nc1C#N ; c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccccc2)c2ccc(N(c3ccc(N(c4ccccc4)c4ccccc4)cc3)c3ccc(N(c4ccccc4)c4ccccc4)cc3)cc2)cc1 ; "NATA"]

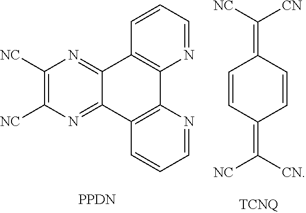

In one specific embodiment of the present disclosure, the hole injection layer 310 is made of F4-TCNQ.

Optionally, as shown in FIG. 1, an electron injection layer 360 is further disposed between the cathode 200 and the electron transport layer 350 to enhance the ability to inject electrons into the electron transport layer 350. The electron injection layer 360 may include an inorganic material such as an alkali metal sulfide or an alkali metal halide, or may include a complex of an alkali metal and an organic substance. In one specific embodiment of the present disclosure, the electron injection layer 360 is made of LiQ.

In a third aspect, the present disclosure provides an electronic apparatus, including the electronic element in the second aspect of the present disclosure.

According to one embodiment, as shown in FIG. 2, the electronic apparatus is a first electronic apparatus 400 including the organic electroluminescent device described above. The first electronic apparatus 400 may be, for example, a display device, a lighting device, an optical communication device, or other types of electronic devices, and may include, for example, but not limited to, a computer screen, a mobile phone screen, a television, electronic paper, an emergency light, a light module, and the like.

A synthesis method for the organic compound of the present disclosure is specifically described below with reference to synthesis examples, but the present disclosure is not limited thus in any way.

Compounds of which synthetic methods not mentioned in the present disclosure are commercially available raw material products.

### Synthetic Example

### 1. Synthesis of IM Cz-x

### Synthesis of IM Cz-01

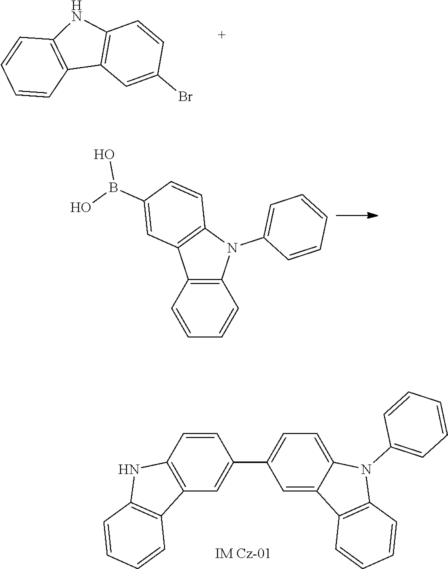 [SMILES: Brc1ccc2[nH]c3ccccc3c2c1 ; OB(O)c1ccc2c(c1)c1ccccc1n2-c1ccccc1 ; c1ccc(-n2c3ccccc3c3cc(-c4ccc5[nH]c6ccccc6c5c4)ccc32)cc1]

3-Bromocarbazole (10.00 g, 40.63 mmol), 9-phenylcarbazole-3-boronic acid (12.25 g, 42.66 mmol), tetrakis(triphenylphosphine)palladium (0.94 g, 0.81 mmol), tetrabutylammonium bromide (2.62 g, 8.13 mmol), potassium carbonate (12.35 g, 89.39 mmol), toluene (100 mL), ethanol (40 mL) and water (20 mL) were added into a reaction flask, heated to reflux under the protection of nitrogen, and stirred for 5 hours. After the reaction solution was cooled to room temperature, the reaction solution was extracted with dichloromethane and water, an organic layer was dried over anhydrous magnesium sulfate and filtered, and after filtration, a filtrate was allowed to pass through a short silica gel column, distillation was performed under reduced pressure to remove a solvent, and a crude product was purified by recrystallization using dichloromethane/petroleum ether (1:4) to obtain IM Cz-01 (13.03 g, yield: 78.5%).

IM Cz-x was synthesized by the same method as that for IM Cz-01 except that Raw material 1 was used instead of 3-bromocarbazole and Raw material 2 was used instead of 9-phenylcarbazole-3-boronic acid, where the main raw materials used, the intermediates synthesized and their yields are shown in Table 1.

TABLE 1

| Raw material 1 | Raw material 2 | IM Cz-x | Yield/% |
|---|---|---|---|
| 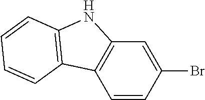 [SMILES: Brc1ccc2c(c1)[nH]c1ccccc12] | 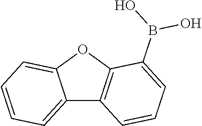 [SMILES: OB(O)c1cccc2c1oc1ccccc12] | 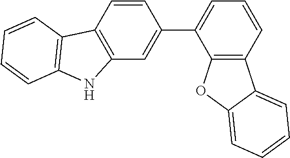 [SMILES: c1ccc2c(c1)[nH]c1cc(-c3cccc4c3oc3ccccc34)ccc12] | 72.4 |
| | | IM Cz-02 | |
| 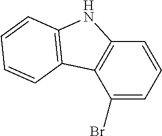 [SMILES: Brc1cccc2[nH]c3ccccc3c12] | 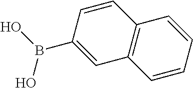 [SMILES: OB(O)c1ccc2ccccc2c1] | 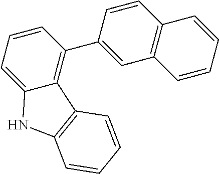 [SMILES: c1ccc2cc(-c3cccc4[nH]c5ccccc5c34)ccc2c1] | 65.4 |
| | | IM Cz-04 | |
| 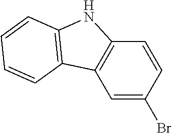 [SMILES: Brc1ccc2[nH]c3ccccc3c2c1] | 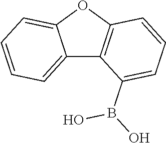 [SMILES: OB(O)c1cccc2oc3ccccc3c12] | 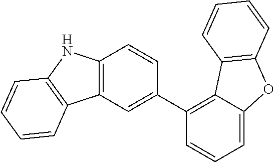 [SMILES: c1ccc2c(c1)[nH]c1ccc(-c3cccc4oc5ccccc5c34)cc12] | 76.3 |
| | | IM Cz-03 | |
| | 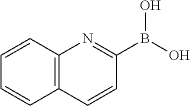 [SMILES: OB(O)c1ccc2ccccc2n1] | 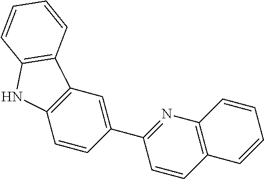 [SMILES: c1ccc2nc(-c3ccc4[nH]c5ccccc5c4c3)ccc2c1] | 69.6 |
| | | IM Cz-05 | |
| | 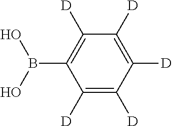 [SMILES: [2H]c1c([2H])c([2H])c(B(O)O)c([2H])c1[2H]] | 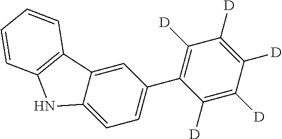 [SMILES: [2H]c1c([2H])c([2H])c(-c2ccc3[nH]c4ccccc4c3c2)c([2H])c1[2H]] | 72.8 |
| | | IM Cz-06 | |

### 2. Synthesis of IM BN-x

### Synthesis of IM BN-1

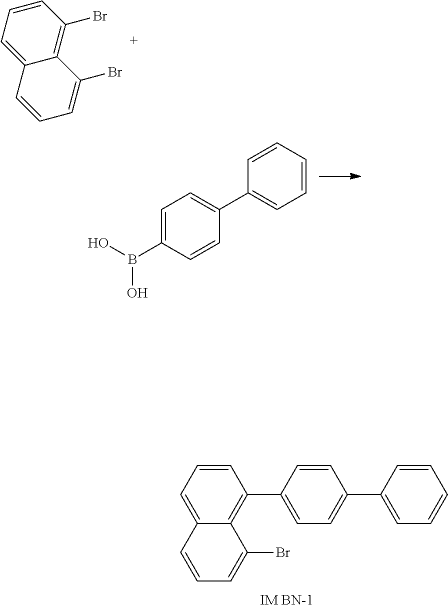 [SMILES: Brc1cccc2cccc(Br)c12 ; OB(O)c1ccc(-c2ccccc2)cc1 ; Brc1cccc2cccc(-c3ccc(-c4ccccc4)cc3)c12]

1,8-Dibromonaphthalene (10.00 g, 34.97 mmol), 4-biphenylboronic acid (6.93 g, 34.97 mmol), tetrakis(triphenylphosphine)palladium (0.81 g, 0.70 mmol), tetrabutylammonium bromide (2.25 g, 6.99 mmol), potassium carbonate (10.63 g, 76.93 mmol), toluene (100 mL), ethanol (40 mL) and water (20 mL) were added into a reaction flask, heated to reflux under the protection of nitrogen, and stirred for 5 hours. After the reaction solution was cooled to room temperature, the reaction solution was extracted with dichloromethane and water, and an organic layer was dried over anhydrous magnesium sulfate and filtered; after filtration, a filtrate was allowed to pass through a short silica gel column, and distillation was performed under reduced pressure to remove a solvent, and the obtained crude product was purified by silica gel chromatography using ethyl acetate/n-heptane (1:5) as a mobile phase, and a solution obtained after passing through the column is distilled under reduced pressure to remove a solvent to obtain IM BN-1 (7.65 g, yield: 60.9%).

IM BN-x was synthesized by the same method as that for IM BN-1 except that Raw material 3 was used instead of 4-biphenylboronic acid, where the main raw materials used, the intermediates synthesized and their yields are shown in Table 2.

TABLE 2

| Raw material 3 | IM BN-X | Yield/% |
|---|---|---|
| 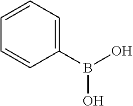 | 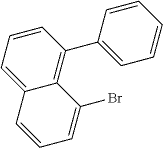 [SMILES: Brc1cccc2cccc(-c3ccccc3)c12] | 64.3 |
| | IM BN-2 | |
| 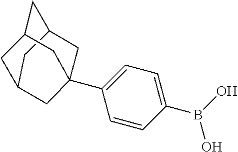 [SMILES: OB(O)c1ccc(C23CC4CC(CC(C4)C2)C3)cc1] | 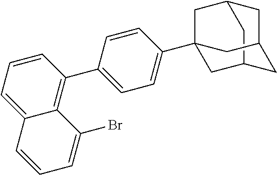 [SMILES: Brc1cccc2cccc(-c3ccc(C45CC6CC(CC(C6)C4)C5)cc3)c12] | 58.2 |
| | IM BN-3 | |
| 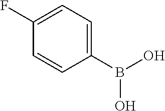 | 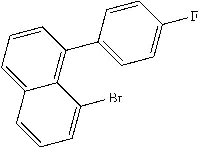 [SMILES: Fc1ccc(-c2cccc3cccc(Br)c23)cc1] | 61.7 |
| | IM BN-4 | |
| 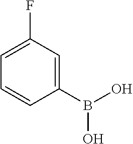 | 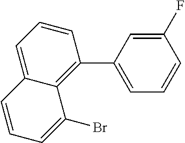 [SMILES: Fc1cccc(-c2cccc3cccc(Br)c23)c1] | 59.9 |
| | IM BN-5 | |
| 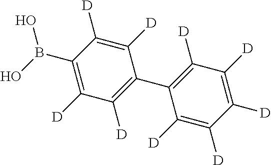 [SMILES: [2H]c1c([2H])c([2H])c(-c2c([2H])c([2H])c(B(O)O)c([2H])c2[2H])c([2H])c1[2H]] | 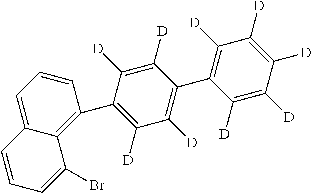 [SMILES: [2H]c1c([2H])c([2H])c(-c2c([2H])c([2H])c(-c3cccc4cccc(Br)c34)c([2H])c2[2H])c([2H])c1[2H]] | 52.8 |
| | IM BN-6 | |
| 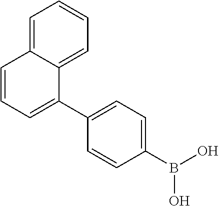 [SMILES: OB(O)c1ccc(-c2cccc3ccccc23)cc1] | 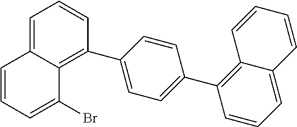 [SMILES: Brc1cccc2cccc(-c3ccc(-c4cccc5ccccc45)cc3)c12] | 55.0 |
| | IM BN-7 | |
| 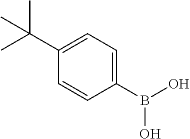 | 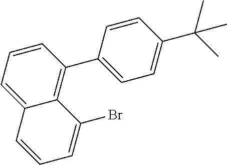 [SMILES: CC(C)(C)c1ccc(-c2cccc3cccc(Br)c23)cc1] | 59.3 |
| | IM BN-8 | |
| 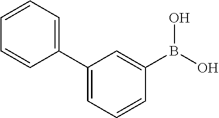 [SMILES: OB(O)c1cccc(-c2ccccc2)c1] | 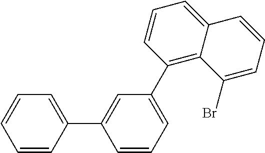 [SMILES: Brc1cccc2cccc(-c3cccc(-c4ccccc4)c3)c12] | 61.1 |
| | IM BN-9 | |
| 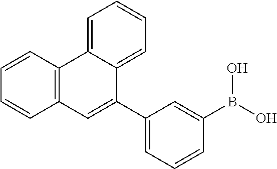 [SMILES: OB(O)c1cccc(-c2cc3ccccc3c3ccccc23)c1] | 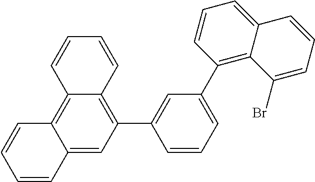 [SMILES: Brc1cccc2cccc(-c3cccc(-c4cc5ccccc5c5ccccc45)c3)c12] | 52.4 |
| | IM BN-10 | |

### 3. Synthesis of IM Nx

### Synthesis of IM N1

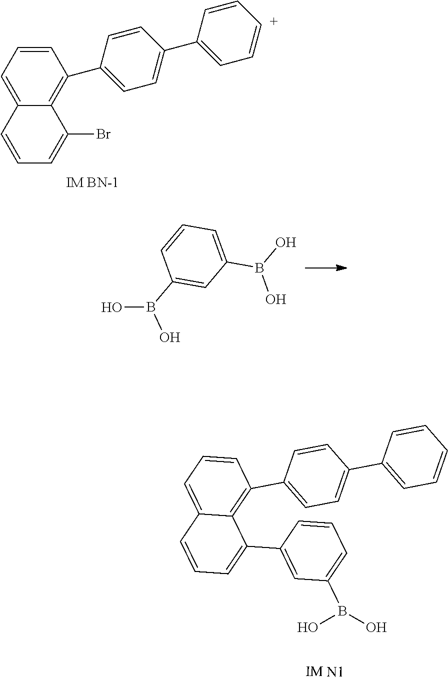 [SMILES: Brc1cccc2cccc(-c3ccc(C4=CC=[C+]C=C4)cc3)c12 ; OB(O)c1cccc(B(O)O)c1 ; OB(O)c1cccc(-c2cccc3cccc(-c4ccc(-c5ccccc5)cc4)c23)c1]

IM BN-1 (4.00 g, 11.13 mmol), 1,3-benzenediboronic acid (2.20 g, 11.13 mmol), tetrakis(triphenylphosphine)palladium (0.26 g, 0.22 mmol), tetrabutylammonium bromide (0.72 g, 2.23 mmol), potassium carbonate (3.38 g, 24.49 mmol), toluene (40 mL), ethanol (10 mL) and water (5 mL) were added into a reaction flask, heated to reflux under the protection of nitrogen, and stirred for 5 hours. After the reaction solution was cooled to room temperature, the reaction solution was extracted with dichloromethane and water, an organic layer was dried over anhydrous magnesium sulfate and filtered, and after filtration, a filtrate was filtered through a short silica gel column, and distillation was performed under reduced pressure to remove a solvent, and the obtained crude product was purified by silica gel chromatography using ethyl acetate/n-heptane (1:5) as a mobile phase, and a solution obtained after passing through the column is distilled under reduced pressure to remove a solvent to obtain IM N1 (2.87 g, yield: 64.50%).

IM Nx was synthesized by the same method as that for IM N1 except that Raw material 4 was used instead of 1,3-benzenediboronic acid, respectively, where the main raw materials used, the intermediates synthesized and their yields are shown in Table 3.

TABLE 3

| IM BN-X | Raw material 4 | IM Nx | Yield/% |
|---|---|---|---|
| 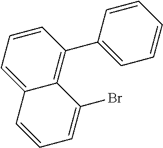 [SMILES: Brc1cccc2cccc(-c3ccccc3)c12] | 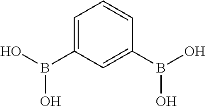 [SMILES: OB(O)c1cccc(B(O)O)c1] | 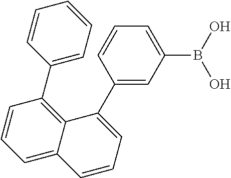 [SMILES: OB(O)c1cccc(-c2cccc3cccc(-c4ccccc4)c23)c1] | 68.3 |
| IM BN-2 | | IM N2 | |
| | 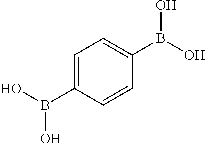 [SMILES: OB(O)c1ccc(B(O)O)cc1] | 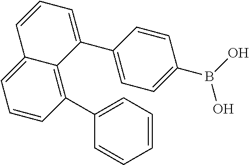 [SMILES: OB(O)c1ccc(-c2cccc3cccc(-c4ccccc4)c23)cc1] | 72.2 |
| | | IM N3 | |
| | 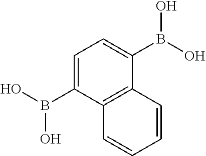 [SMILES: OB(O)c1ccc(B(O)O)c2ccccc12] | 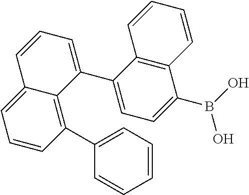 [SMILES: OB(O)c1ccc(-c2cccc3cccc(-c4ccccc4)c23)c2ccccc12] | 53.8 |
| | | IM N4 | |
| 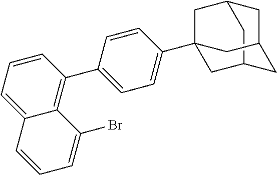 [SMILES: Brc1cccc2cccc(-c3ccc(C45CC6CC(CC(C6)C4)C5)cc3)c12] | 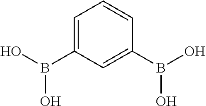 [SMILES: OB(O)c1cccc(B(O)O)c1] | 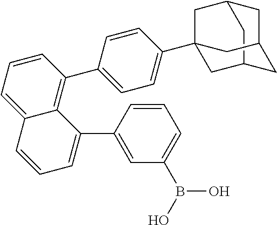 [SMILES: OB(O)c1cccc(-c2cccc3cccc(-c4ccc(C56CC7CC(CC(C7)C5)C6)cc4)c23)c1] | 60.5 |
| IM BN-3 | | IM N5 | |
| | 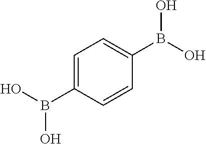 [SMILES: OB(O)c1ccc(B(O)O)cc1] | 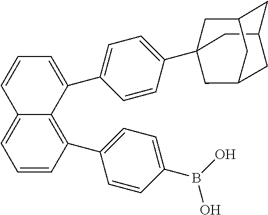 [SMILES: OB(O)c1ccc(-c2cccc3cccc(-c4ccc(C56CC7CC(CC(C7)C5)C6)cc4)c23)cc1] | 61.4 |
| | | IM N6 | |
| 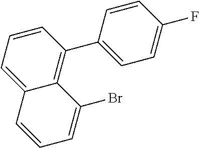 [SMILES: Fc1ccc(-c2cccc3cccc(Br)c23)cc1] | 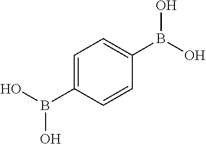 [SMILES: OB(O)c1ccc(B(O)O)cc1] | 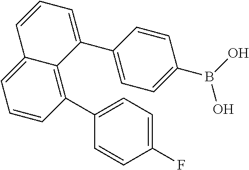 [SMILES: OB(O)c1ccc(-c2cccc3cccc(-c4ccc(F)cc4)c23)cc1] | 65.3 |
| IM BN-4 | | IM N7 | |
| | 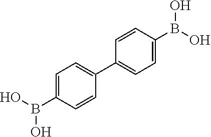 [SMILES: OB(O)c1ccc(-c2ccc(B(O)O)cc2)cc1] | 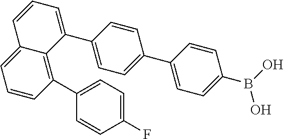 [SMILES: OB(O)c1ccc(-c2ccc(-c3cccc4cccc(-c5ccc(F)cc5)c34)cc2)cc1] | 59.5 |
| | | IM N8 | |
| 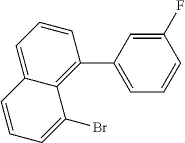 [SMILES: Fc1cccc(-c2cccc3cccc(Br)c23)c1] | 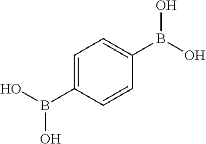 [SMILES: OB(O)c1ccc(B(O)O)cc1] | 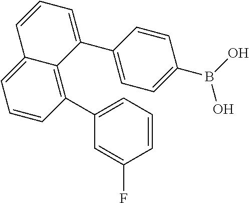 [SMILES: OB(O)c1ccc(-c2cccc3cccc(-c4cccc(F)c4)c23)cc1] | 63.6 |
| IM BN-5 | | IM N9 | |
| 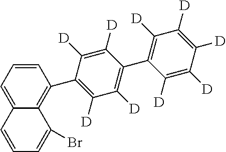 [SMILES: [2H]c1c([2H])c([2H])c(-c2c([2H])c([2H])c(-c3cccc4cccc(Br)c34)c([2H])c2[2H])c([2H])c1[2H]] | 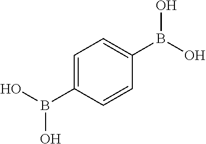 [SMILES: OB(O)c1ccc(B(O)O)cc1] | 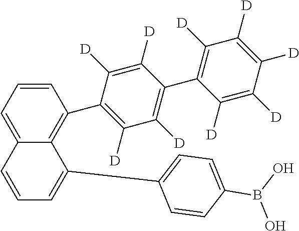 [SMILES: [2H]c1c([2H])c([2H])c(-c2c([2H])c([2H])c(-c3cccc4cccc(-c5ccc(B(O)O)cc5)c34)c([2H])c2[2H])c([2H])c1[2H]] | 62.6 |
| IM BN-6 | | IM N10 | |
| 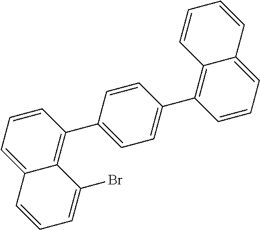 [SMILES: Brc1cccc2cccc(-c3ccc(-c4cccc5ccccc45)cc3)c12] | 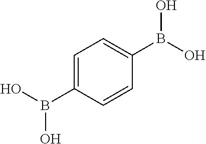 [SMILES: OB(O)c1ccc(B(O)O)cc1] | 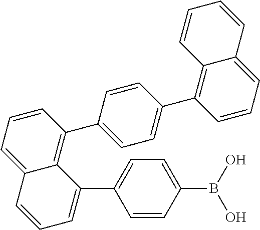 [SMILES: OB(O)c1ccc(-c2cccc3cccc(-c4ccc(-c5cccc6ccccc56)cc4)c23)cc1] | 58.2 |
| IM BN-7 | | IM N11 | |
| 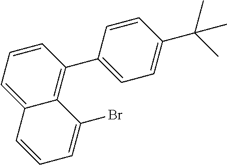 [SMILES: CC(C)(C)c1ccc(-c2cccc3cccc(Br)c23)cc1] | 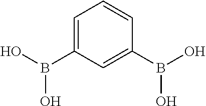 [SMILES: OB(O)c1cccc(B(O)O)c1] | 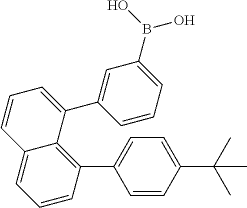 [SMILES: CC(C)(C)c1ccc(-c2cccc3cccc(-c4cccc(B(O)O)c4)c23)cc1] | 63.9 |
| IM BN-8 | | IM N12 | |
| | 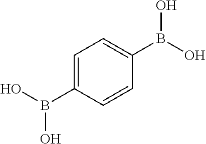 [SMILES: OB(O)c1ccc(B(O)O)cc1] | 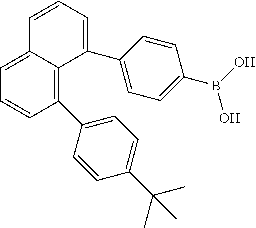 [SMILES: CC(C)(C)c1ccc(-c2cccc3cccc(-c4ccc(B(O)O)cc4)c23)cc1] | 62.4 |
| | | IM N13 | |
| 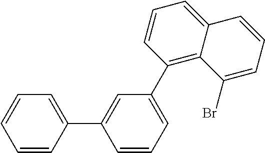 [SMILES: Brc1cccc2cccc(-c3cccc(-c4ccccc4)c3)c12] | 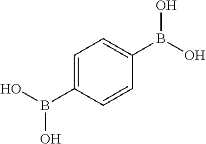 [SMILES: OB(O)c1ccc(B(O)O)cc1] | 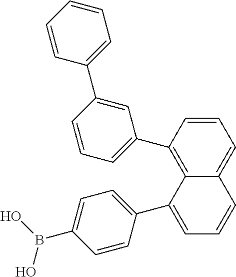 [SMILES: OB(O)c1ccc(-c2cccc3cccc(-c4cccc(-c5ccccc5)c4)c23)cc1] | 60.4 |
| IM NB-9 | | IM N14 | |
| 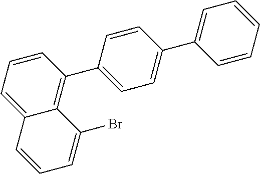 [SMILES: Brc1cccc2cccc(-c3ccc(-c4ccccc4)cc3)c12] | 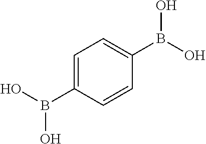 [SMILES: OB(O)c1ccc(B(O)O)cc1] | 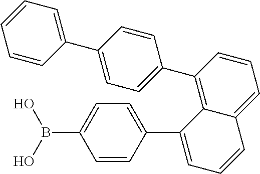 [SMILES: OB(O)c1ccc(-c2cccc3cccc(-c4ccc(-c5ccccc5)cc4)c23)cc1] | 58.8 |
| IM BN-1 | | IM N15 | |
| 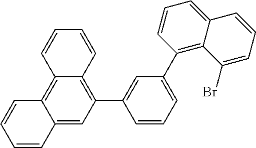 [SMILES: Brc1cccc2cccc(-c3cccc(-c4cc5ccccc5c5ccccc45)c3)c12] | 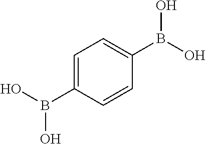 [SMILES: OB(O)c1ccc(B(O)O)cc1] | 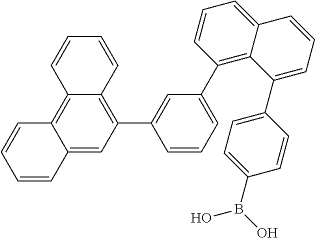 [SMILES: OB(O)c1ccc(-c2cccc3cccc(-c4cccc(-c5cc6ccccc6c6ccccc56)c4)c23)cc1] | 55.2 |
| IM BN-10 | | IM N16 | |

### 4. Synthesis of IM Tr-Cz-x

### Synthesis of IM Tr-Cz-01

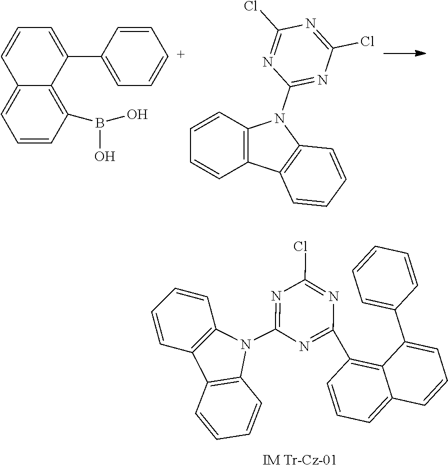

8-Phenyl-1-naphthaleneboronic acid (4.80 g, 19.35 mmol), 9-(4,6-dichloro-[1,3,5]triazin-2-yl)-carbazole (6.09 g, 19.34 mmol), palladium acetate (0.22 g, 0.97 mmol), 2-dicyclohexylphosphine-2,4,6-triisopropylbiphenyl (0.46 g, 0.97 mmol), potassium carbonate (5.88 g, 42.56 mmol), toluene (50 mL), ethanol (20 mL) and water (10 mL) were added into a reaction flask, heated to reflux under the protection of nitrogen, and stirred for 5 hours. After the reaction solution was cooled to room temperature, the reaction solution was extracted with dichloromethane and water, an organic layer was dried over anhydrous magnesium sulfate and filtered, and after filtration, a filtrate was filtered through a short silica gel column, and distillation was performed under reduced pressure to remove a solvent, and a crude product was recrystallized by using an ethyl acetate/petroleum ether (1:3) system to obtain IM Tr-Cz-01 (6.75 g, yield: 72.2%).

IM-Tr-Cz-x shown in Table 4 was synthesized by the same method as that for IM Tr-Cz-01 except that 8-phenyl-1-naphthaleneboronic acid was replaced with IM Nx, where the main raw materials used, the intermediates synthesized and their yields are shown in Table 4.

TABLE 4

| IM Nx | IM-Tr-Cz-x | Yield/% |
|---|---|---|
| 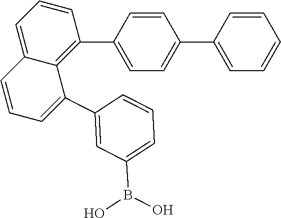 [SMILES: OB(O)c1cccc(-c2cccc3cccc(-c4ccc(-c5ccccc5)cc4)c23)c1] | 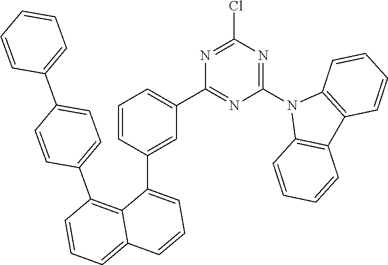 [SMILES: Clc1nc(-c2cccc(-c3cccc4cccc(-c5ccc(-c6ccccc6)cc5)c34)c2)nc(-n2c3ccccc3c3ccccc32)n1] | 73.8 |
| IM N1 | IM Tr-Cz-02 | |
| 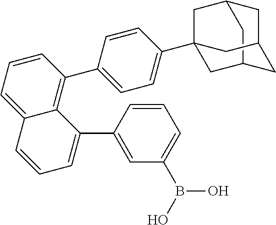 [SMILES: OB(O)c1cccc(-c2cccc3cccc(-c4ccc(C56CC7CC(CC(C7)C5)C6)cc4)c23)c1] | 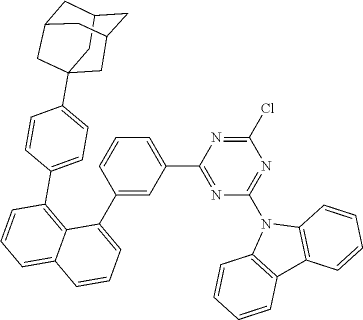 [SMILES: Clc1nc(-c2cccc(-c3cccc4cccc(-c5ccc(C67CC8CC(CC(C8)C6)C7)cc5)c34)c2)nc(-n2c3ccccc3c3ccccc32)n1] | 70.4 |
| IM N5 | IM-Tr-Cz-03 | |
| 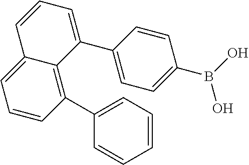 [SMILES: OB(O)c1ccc(-c2cccc3cccc(-c4ccccc4)c23)cc1] | 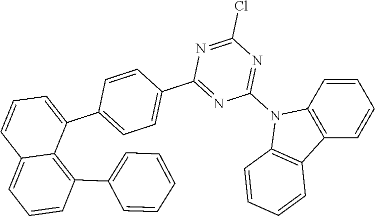 [SMILES: Clc1nc(-c2ccc(-c3cccc4cccc(-c5ccccc5)c34)cc2)nc(-n2c3ccccc3c3ccccc32)n1] | 71.5 |
| IM N3 | IM-Tr-Cz-04 | |
| 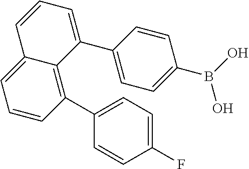 [SMILES: OB(O)c1ccc(-c2cccc3cccc(-c4ccc(F)cc4)c23)cc1] | 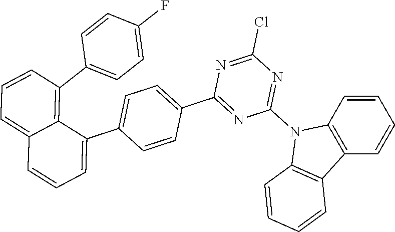 [SMILES: Fc1ccc(-c2cccc3cccc(-c4ccc(-c5nc(Cl)nc(-n6c7ccccc7c7ccccc76)n5)cc4)c23)cc1] | 69.5 |
| IM N7 | IM-Tr-Cz-05 | |
| 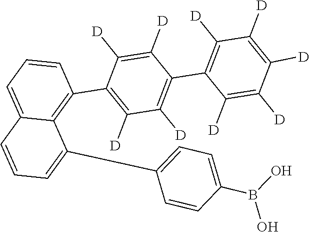 [SMILES: [2H]c1c([2H])c([2H])c(-c2c([2H])c([2H])c(-c3cccc4cccc(-c5ccc(B(O)O)cc5)c34)c([2H])c2[2H])c([2H])c1[2H]] | 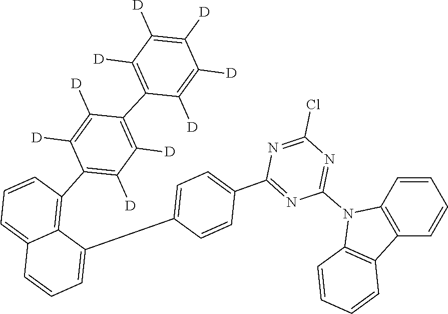 [SMILES: [2H]c1c([2H])c([2H])c(-c2c([2H])c([2H])c(-c3cccc4cccc(-c5ccc(-c6nc(Cl)nc(-n7c8ccccc8c8ccccc87)n6)cc5)c34)c([2H])c2[2H])c([2H])c1[2H]] | 63.4 |
| IM N10 | IM-Tr-Cz-06 | |

### 5. Synthesis of IM Tr-x

### Synthesis of IM Tr-01

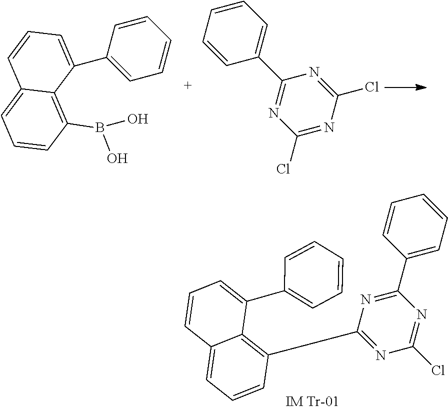

8-Phenyl-1-naphthaleneboronic acid (5.50 g, 22.17 mmol), 2,4-dichloro-6-phenyl-1,3,5-triazine (5.01 g, 22.17 mmol), palladium acetate (0.25 g, 1.11 mmol), 2-dicyclohexylphosphine-2,4,6-triisopropylbiphenyl (0.53 g, 1.11 mmol), potassium carbonate (6.74 g, 48.77 mmol) and toluene (55 mL), ethanol (20 mL) and water (10 mL) were added into a reaction flask, heated to reflux under the protection of nitrogen, and stirred for 4 hours. After the reaction solution was cooled to room temperature, the reaction solution was extracted with dichloromethane and water, an organic layer was dried over anhydrous magnesium sulfate and filtered, and after filtration, a filtrate was filtered through a short silica gel column, and distillation was performed under reduced pressure to remove a solvent, and a crude product was recrystallized by using ethyl acetate/petroleum ether (1:3) to obtain IM Tr-01 (6.32 g, yield: 72.4%).

IM Tr-x shown in Table 5 was synthesized by the same method as that for IM Tr-01 except that 8-phenyl-1-naphthaleneboronic acid was replaced with IM Nx and Raw material 5 was used instead of 2,4-dichloro-6-phenyl-1,3,5-triazine, where the main starting material used, the intermediates synthesized and their yields are shown in Table 5.

TABLE 5

| IM Nx | Raw material 5 | IM Tr-x | Yield/% |
|---|---|---|---|
| 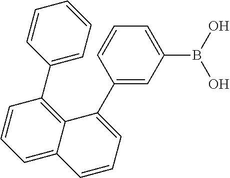 [SMILES: OB(O)c1cccc(-c2cccc3cccc(-c4ccccc4)c23)c1] | 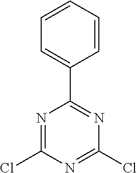 [SMILES: Clc1nc(Cl)nc(-c2ccccc2)n1] | 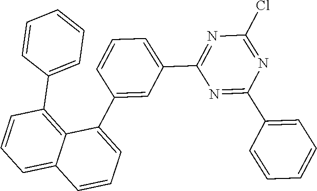 [SMILES: Clc1nc(-c2ccccc2)nc(-c2cccc(-c3cccc4cccc(-c5ccccc5)c34)c2)n1] | 69.7 |
| IM N2 | | IM Tr-2 | |
| | 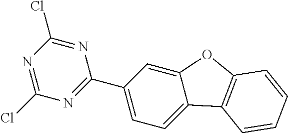 [SMILES: Clc1nc(Cl)nc(-c2ccc3c(c2)oc2ccccc23)n1] | 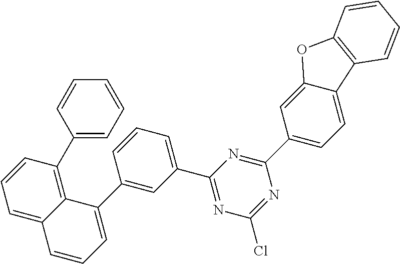 [SMILES: Clc1nc(-c2cccc(-c3cccc4cccc(-c5ccccc5)c34)c2)nc(-c2ccc3c(c2)oc2ccccc23)n1] | 62.8 |
| | | IM Tr-3 | |
| | 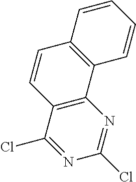 [SMILES: Clc1nc(Cl)c2ccc3ccccc3c2n1] | 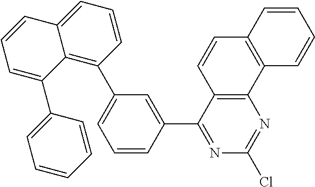 [SMILES: Clc1nc(-c2cccc(-c3cccc4cccc(-c5ccccc5)c34)c2)c2ccc3ccccc3c2n1] | 51.5 |
| | | IM Tr-4 | |
| 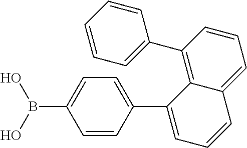 [SMILES: OB(O)c1ccc(-c2cccc3cccc(-c4ccccc4)c23)cc1] | 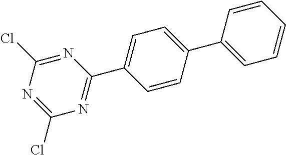 [SMILES: Clc1nc(Cl)nc(-c2ccc(-c3ccccc3)cc2)n1] | 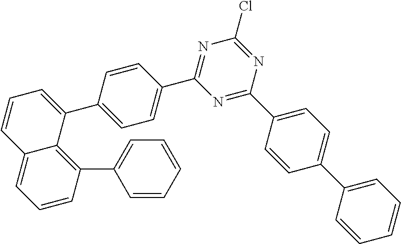 [SMILES: Clc1nc(-c2ccc(-c3ccccc3)cc2)nc(-c2ccc(-c3cccc4cccc(-c5ccccc5)c34)cc2)n1] | 70.4 |
| IM N3 | | IM Tr-5 | |
| | 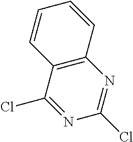 [SMILES: Clc1nc(Cl)c2ccccc2n1] | 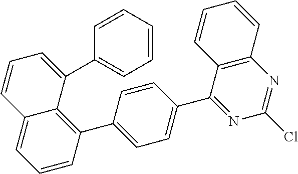 [SMILES: Clc1nc(-c2ccc(-c3cccc4cccc(-c5ccccc5)c34)cc2)c2ccccc2n1] | 51.2 |
| | | IM Tr-6 | |
| | 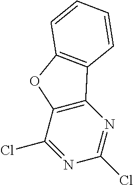 [SMILES: Clc1nc(Cl)c2oc3ccccc3c2n1] | 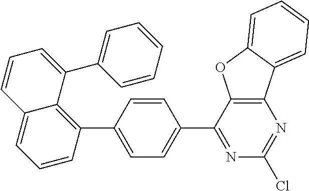 [SMILES: Clc1nc(-c2ccc(-c3cccc4cccc(-c5ccccc5)c34)cc2)c2oc3ccccc3c2n1] | 54.8 |
| | | IM Tr-7 | |
| | 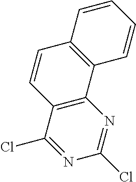 [SMILES: Clc1nc(Cl)c2ccc3ccccc3c2n1] | 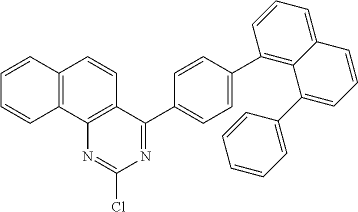 [SMILES: Clc1nc(-c2ccc(-c3cccc4cccc(-c5ccccc5)c34)cc2)c2ccc3ccccc3c2n1] | 50.3 |
| | | IM Tr-8 | |
| | 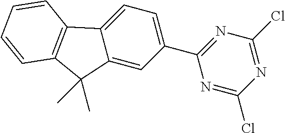 [SMILES: CC1(C)c2ccccc2-c2ccc(-c3nc(Cl)nc(Cl)n3)cc21] | 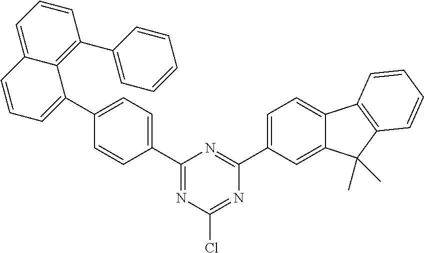 [SMILES: CC1(C)c2ccccc2-c2ccc(-c3nc(Cl)nc(-c4ccc(-c5cccc6cccc(-c7ccccc7)c56)cc4)n3)cc21] | 65.2 |
| | | IM Tr-9 | |
| | 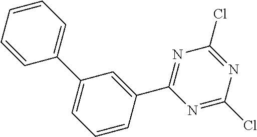 [SMILES: Clc1nc(Cl)nc(-c2cccc(-c3ccccc3)c2)n1] | 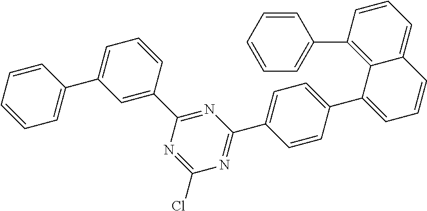 [SMILES: Clc1nc(-c2ccc(-c3cccc4cccc(-c5ccccc5)c34)cc2)nc(-c2cccc(-c3ccccc3)c2)n1] | 67.0 |
| | | IM Tr-10 | |
| 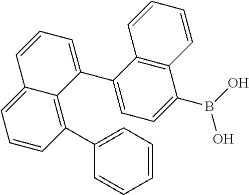 [SMILES: OB(O)c1ccc(-c2cccc3cccc(-c4ccccc4)c23)c2ccccc12] | 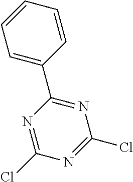 [SMILES: Clc1nc(Cl)nc(-c2ccccc2)n1] | 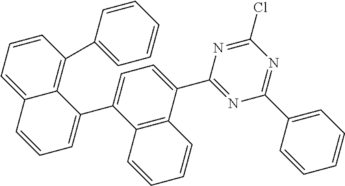 [SMILES: Clc1nc(-c2ccccc2)nc(-c2ccc(-c3cccc4cccc(-c5ccccc5)c34)c3ccccc23)n1] | 61.4 |
| IM N4 | | IM Tr-11 | |
| 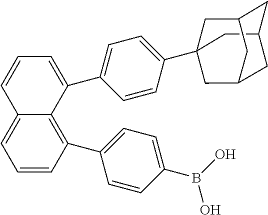 [SMILES: OB(O)c1ccc(-c2cccc3cccc(-c4ccc(C56CC7CC(CC(C7)C5)C6)cc4)c23)cc1] | 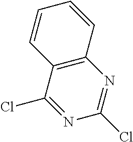 [SMILES: Clc1nc(Cl)c2ccccc2n1] | 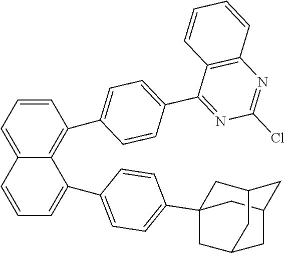 [SMILES: Clc1nc(-c2ccc(-c3cccc4cccc(-c5ccc(C67CC8CC(CC(C8)C6)C7)cc5)c34)cc2)c2ccccc2n1] | 52.6 |
| IM N6 | | IM Tr-12 | |
| 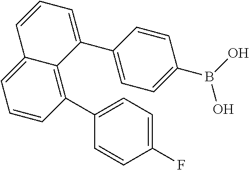 [SMILES: OB(O)c1ccc(-c2cccc3cccc(-c4ccc(F)cc4)c23)cc1] | 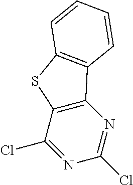 [SMILES: Clc1nc(Cl)c2sc3ccccc3c2n1] | 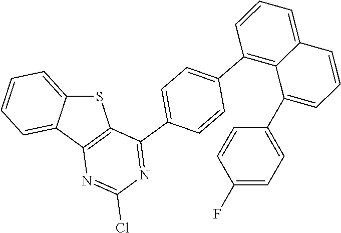 [SMILES: Fc1ccc(-c2cccc3cccc(-c4ccc(-c5nc(Cl)nc6c5sc5ccccc56)cc4)c23)cc1] | 52.1 |
| IM N7 | | IM Tr-13 | |
| | 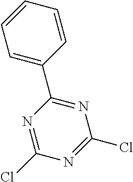 [SMILES: Clc1nc(Cl)nc(-c2ccccc2)n1] | 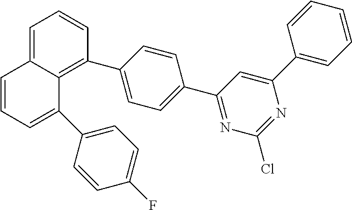 [SMILES: Fc1ccc(-c2cccc3cccc(-c4ccc(-c5cc(-c6ccccc6)nc(Cl)n5)cc4)c23)cc1] | 61.7 |
| | | IM Tr-14 | |
| 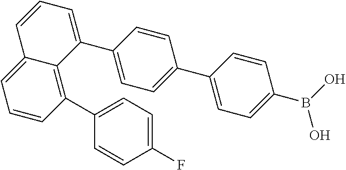 [SMILES: OB(O)c1ccc(-c2ccc(-c3cccc4cccc(-c5ccc(F)cc5)c34)cc2)cc1] | 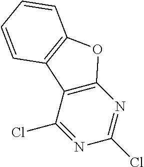 [SMILES: Clc1nc(Cl)c2c(n1)oc1ccccc12] | 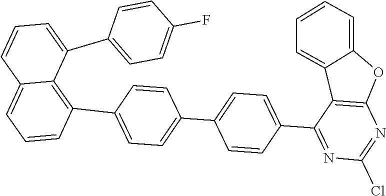 [SMILES: Fc1ccc(-c2cccc3cccc(-c4ccc(-c5ccc(-c6nc(Cl)nc7oc8ccccc8c67)cc5)cc4)c23)cc1] | 51.3 |
| IM N8 | | IM Tr-15 | |
| 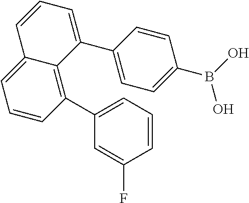 [SMILES: OB(O)c1ccc(-c2cccc3cccc(-c4cccc(F)c4)c23)cc1] | 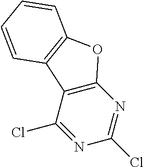 [SMILES: Clc1nc(Cl)c2c(n1)oc1ccccc12] | 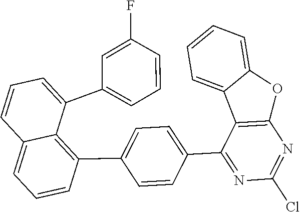 [SMILES: Fc1cccc(-c2cccc3cccc(-c4ccc(-c5nc(Cl)nc6oc7ccccc7c56)cc4)c23)c1] | 50.4 |
| IM N9 | | IM Tr-16 | |
| 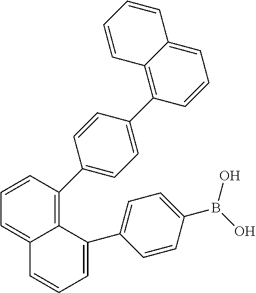 [SMILES: OB(O)c1ccc(-c2cccc3cccc(-c4ccc(-c5cccc6ccccc56)cc4)c23)cc1] | 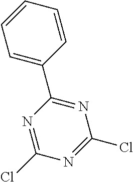 [SMILES: Clc1nc(Cl)nc(-c2ccccc2)n1] | 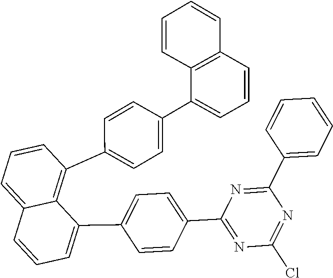 [SMILES: Clc1nc(-c2ccccc2)nc(-c2ccc(-c3cccc4cccc(-c5ccc(-c6cccc7ccccc67)cc5)c34)cc2)n1] | 66.0 |
| IM N11 | | IM Tr-17 | |
| 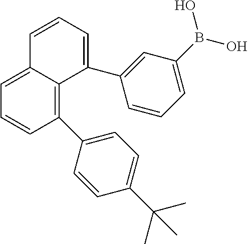 [SMILES: CC(C)(C)c1ccc(-c2cccc3cccc(-c4cccc(B(O)O)c4)c23)cc1] | 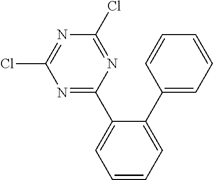 [SMILES: Clc1nc(Cl)nc(-c2ccccc2-c2ccccc2)n1] | 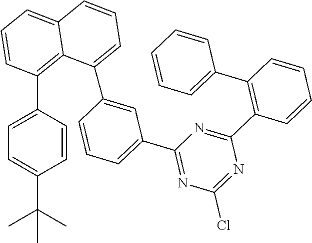 [SMILES: CC(C)(C)c1ccc(-c2cccc3cccc(-c4cccc(-c5nc(Cl)nc(-c6ccccc6-c6ccccc6)n5)c4)c23)cc1] | 61.9 |
| IM N12 | | IM Tr-18 | |
| 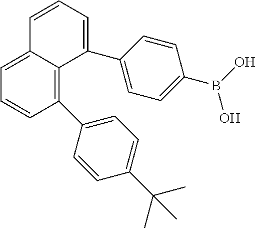 [SMILES: CC(C)(C)c1ccc(-c2cccc3cccc(-c4ccc(B(O)O)cc4)c23)cc1] | 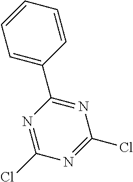 [SMILES: Clc1nc(Cl)nc(-c2ccccc2)n1] | 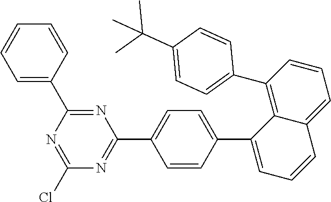 [SMILES: CC(C)(C)c1ccc(-c2cccc3cccc(-c4ccc(-c5nc(Cl)nc(-c6ccccc6)n5)cc4)c23)cc1] | 66.3 |
| IM N13 | | IM Tr-19 | |
| | 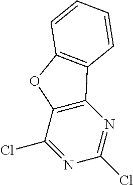 [SMILES: Clc1nc(Cl)c2oc3ccccc3c2n1] | 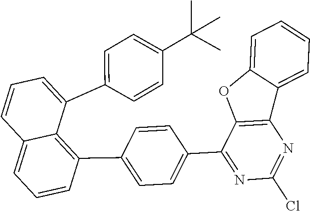 [SMILES: CC(C)(C)c1ccc(-c2cccc3cccc(-c4ccc(-c5nc(Cl)nc6c5oc5ccccc56)cc4)c23)cc1] | 53.6 |
| | | IM Tr-20 | |
| 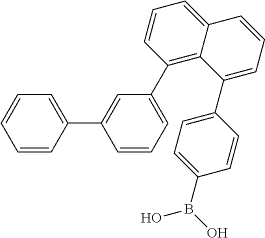 [SMILES: OB(O)c1ccc(-c2cccc3cccc(-c4cccc(-c5ccccc5)c4)c23)cc1] | 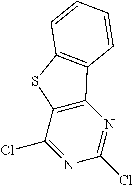 [SMILES: Clc1nc(Cl)c2sc3ccccc3c2n1] | 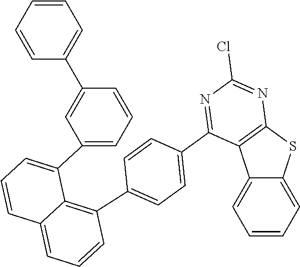 [SMILES: Clc1nc(-c2ccc(-c3cccc4cccc(-c5cccc(-c6ccccc6)c5)c34)cc2)c2c(n1)sc1ccccc12] | 49.4 |
| IM N14 | | IM Tr-21 | |
| 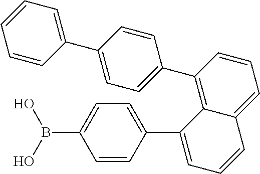 [SMILES: OB(O)c1ccc(-c2cccc3cccc(-c4ccc(-c5ccccc5)cc4)c23)cc1] | 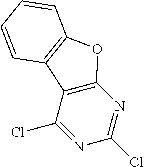 [SMILES: Clc1nc(Cl)c2c(n1)oc1ccccc12] | 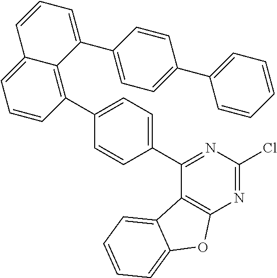 [SMILES: Clc1nc(-c2ccc(-c3cccc4cccc(-c5ccc(-c6ccccc6)cc5)c34)cc2)c2c(n1)oc1ccccc12] | 52.0 |
| IM N15 | | IM Tr-22 | |
| 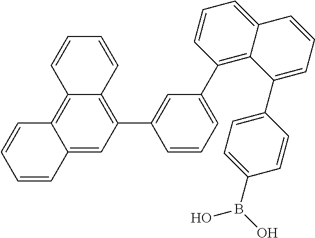 [SMILES: OB(O)c1ccc(-c2cccc3cccc(-c4cccc(-c5cc6ccccc6c6ccccc56)c4)c23)cc1] | 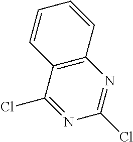 [SMILES: Clc1nc(Cl)c2ccccc2n1] | 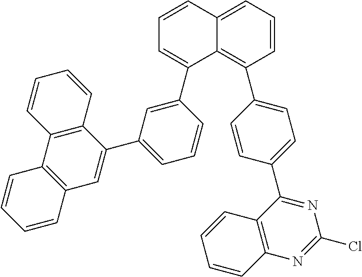 [SMILES: Clc1nc(-c2ccc(-c3cccc4cccc(-c5cccc(-c6cc7ccccc7c7ccccc67)c5)c34)cc2)c2ccccc2n1] | 58.8 |
| IM N16 | | IM Tr-23 | |

### 6. Synthesis of Compounds

### (1) Synthesis of a Compound A04

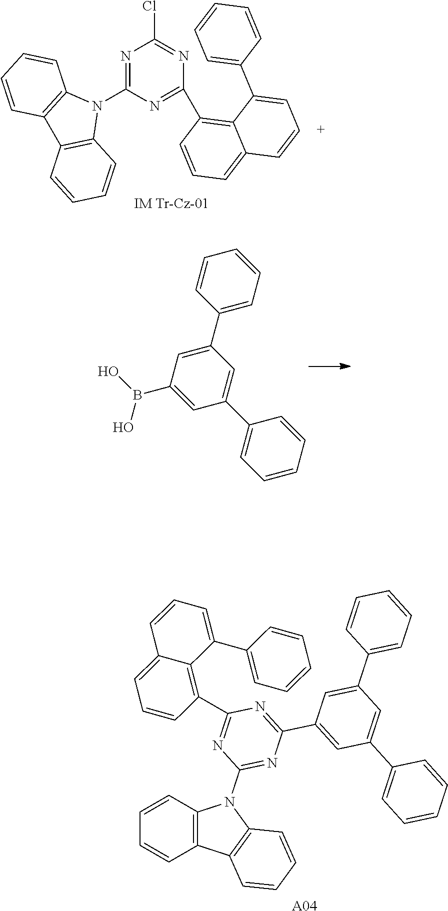

IM Tr-Cz-01 (6.00 g, 12.42 mmol), (3,5-diphenylphenyl)boronic acid (3.58 g, 13.05 mmol), palladium acetate (0.14 g, 0.62 mmol), 2-dicyclohexylphosphine-2,4,6-triisopropylbiphenyl (0.29 g, 0.62 mmol), potassium carbonate (3.78 g, 27.33 mmol), toluene (60 mL), ethanol (25 mL) and water (15 mL) were added into a reaction flask, heated to reflux under the protection of nitrogen, and stirred for 5 hours. After the reaction solution was cooled to room temperature, the reaction solution was extracted with dichloromethane and water, an organic layer was dried over anhydrous magnesium sulfate and filtered, and after filtration, a filtrate was filtered through a short silica gel column, and distillation was performed under reduced pressure to remove a solvent, and a crude product was recrystallized by using a toluene/n-heptane (1:3) system to obtain a Compound A04 (4.92 g, yield: 58.5%), mass spectrum (m/z)=677.3 [M+H]⁺.

Compounds shown in Table 6 were synthesized by the same method as that for the compound A04 except that IM-Tr-Cz-x was used instead of IM Tr-Cz-01 and Raw material 6 was used instead of (3,5-diphenylphenyl)boronic acid, where the main raw material used, the synthesized compounds and their yields and mass spectra are shown in Table 6.

TABLE 6

| | | | | Mass |
|---|---|---|---|---|
| | | | | spectrum |
| | Raw | | Yield/ | (m/z)/ |
| IM-Tr-Cz-x | material 6 | Compound | % | [M + H]⁺ |
| 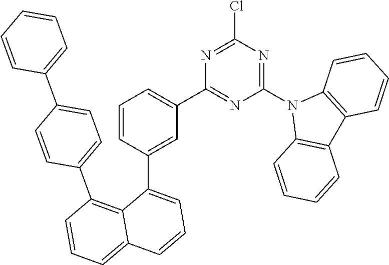 [SMILES: Clc1nc(-c2cccc(-c3cccc4cccc(-c5ccc(-c6ccccc6)cc5)c34)c2)nc(-n2c3ccccc3c3ccccc32)n1] | 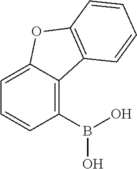 [SMILES: OB(O)c1cccc2oc3ccccc3c12] | 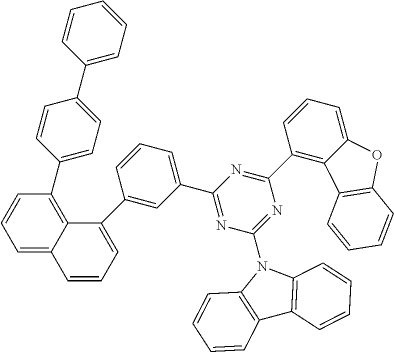 [SMILES: c1ccc(-c2ccc(-c3cccc4cccc(-c5cccc(-c6nc(-c7cccc8oc9ccccc9c78)nc(-n7c8ccccc8c8ccccc87)n6)c5)c34)cc2)cc1] | 55.8 | 767.3 |
| IM Tr-Cz-02 | | A22 | | |
| 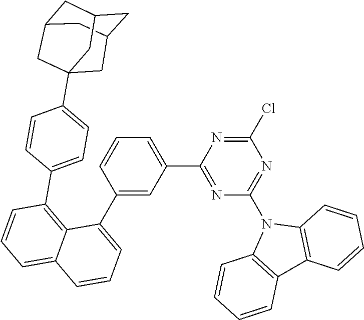 [SMILES: Clc1nc(-c2cccc(-c3cccc4cccc(-c5ccc(C67CC8CC(CC(C8)C6)C7)cc5)c34)c2)nc(-n2c3ccccc3c3ccccc32)n1] | 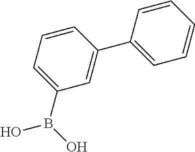 [SMILES: OB(O)c1cccc(-c2ccccc2)c1] | 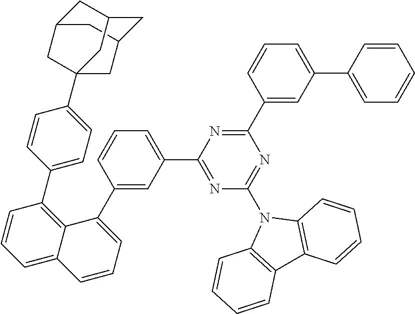 [SMILES: c1ccc(-c2cccc(-c3nc(-c4cccc(-c5cccc6cccc(-c7ccc(C89CC%10CC(CC(C%10)C8)C9)cc7)c56)c4)nc(-n4c5ccccc5c5ccccc54)n3)c2)cc1] | 50.4 | 811.4 |
| IM-Tr-Cz-03 | | A35 | | |
| 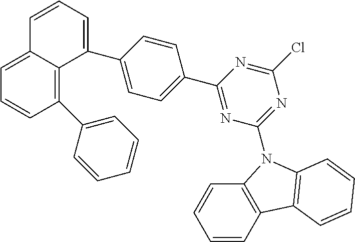 [SMILES: Clc1nc(-c2ccc(-c3cccc4cccc(-c5ccccc5)c34)cc2)nc(-n2c3ccccc3c3ccccc32)n1] | 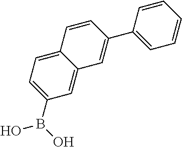 [SMILES: OB(O)c1ccc2ccc(-c3ccccc3)cc2c1] | 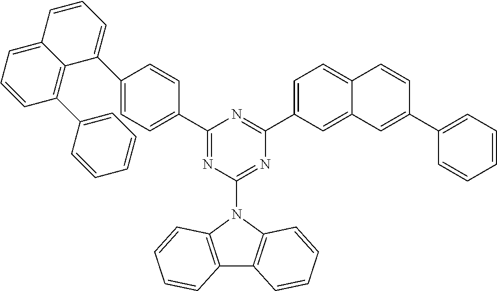 [SMILES: c1ccc(-c2ccc3ccc(-c4nc(-c5ccc(-c6cccc7cccc(-c8ccccc8)c67)cc5)nc(-n5c6ccccc6c6ccccc65)n4)cc3c2)cc1] | 42.9 | 727.3 |
| IM-Tr-Cz-04 | | A43 | | |
| 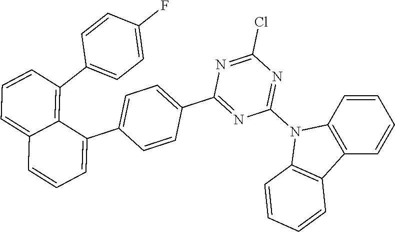 [SMILES: Fc1ccc(-c2cccc3cccc(-c4ccc(-c5nc(Cl)nc(-n6c7ccccc7c7ccccc76)n5)cc4)c23)cc1] | 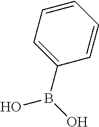 | 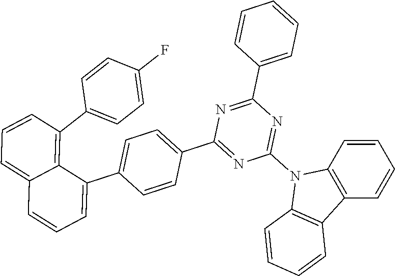 [SMILES: Fc1ccc(-c2cccc3cccc(-c4ccc(-c5nc(-c6ccccc6)nc(-n6c7ccccc7c7ccccc76)n5)cc4)c23)cc1] | 55.1 | 619.2 |
| IM-Tr-Cz-05 | | A64 | | |
| 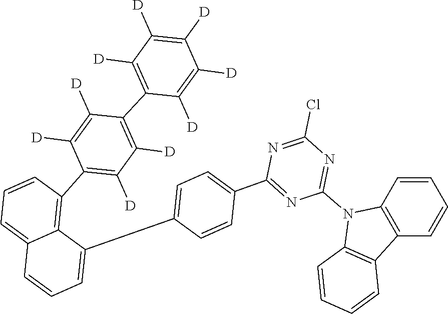 [SMILES: [2H]c1c([2H])c([2H])c(-c2c([2H])c([2H])c(-c3cccc4cccc(-c5ccc(-c6nc(Cl)nc(-n7c8ccccc8c8ccccc87)n6)cc5)c34)c([2H])c2[2H])c([2H])c1[2H]] | 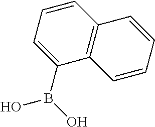 [SMILES: OB(O)c1cccc2ccccc12] | 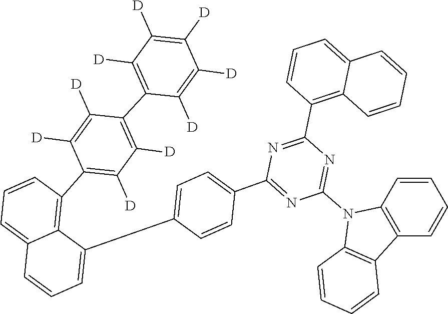 [SMILES: [2H]c1c([2H])c([2H])c(-c2c([2H])c([2H])c(-c3cccc4cccc(-c5ccc(-c6nc(-c7cccc8ccccc78)nc(-n7c8ccccc8c8ccccc87)n6)cc5)c34)c([2H])c2[2H])c([2H])c1[2H]] | 37.2 | 736.3 |
| IM-Tr-Cz-06 | | A76 | | |

### (2) Synthesis of a Compound A07

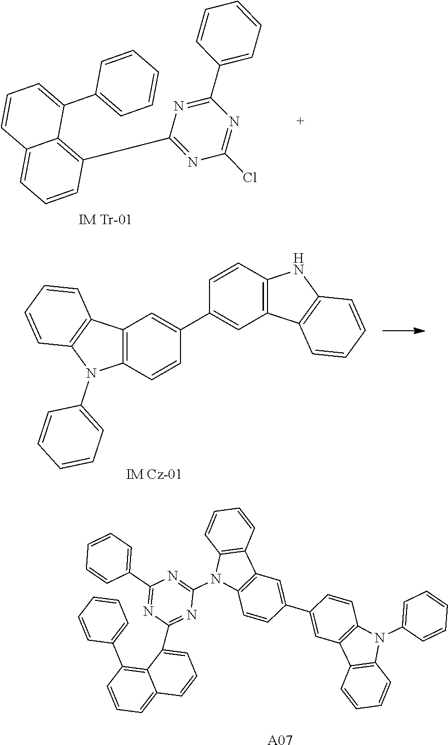 [SMILES: Clc1nc(-c2ccccc2)nc(-c2cccc3cccc(-c4ccccc4)c23)n1 ; "+" ; c1ccc(-n2c3ccccc3c3cc(-c4ccc5[nH]c6ccccc6c5c4)ccc32)cc1 ; c1ccc(-c2nc(-c3cccc4cccc(-c5ccccc5)c34)nc(-n3c4ccccc4c4cc(-c5ccc6c(c5)c5ccccc5n6-c5ccccc5)ccc43)n2)cc1 ; "A07"]

Under N₂ protection, IM Tr-01 (5.06 g, 12.85 mmol), IM Cz-01 (5.00 g, 12.24 mmol), sodium hydride (0.44 g, 18.36 mmol) and dry DMIF (50 mL) were sequentially added into a three-necked flask, and stirred at room temperature for 6 hours; the reaction was quenched by adding 50 mL of deionized water, then the reaction solution was extracted with toluene, and washed with water to be neutral, liquid separation, drying, and filtering were performed, and a filtrate was allowed to pass through a short silica gel column, and concentration was performed under reduced pressure until a solid was precipitated, distillation was stopped, and the distilled material was allowed to naturally stand, and cooled to room temperature, and the precipitated crystal was recrystallized by using toluene/n-hexane and dried to obtain a Compound A07 (4.93 g, yield: 52.60%), mass spectrum (m/z)=766.3 [M+H]⁺.

Compounds in Table 7 were prepared in the same synthesis method as that for the compound A07 except that IIM Tr-x was used instead of IM Tr-01 and Raw material 7 was used instead of IM Cz-01, where the main raw materials used, the compounds synthesized and their yields and mass spectra are shown in Table 7.

TABLE 7

| | | | | Mass |
|---|---|---|---|---|
| | | | | spectrum |
| | | | Yield/ | (m/z)/ |
| IM Tr-x | Raw material 7 | Compound | % | [M + H]⁺ |
| 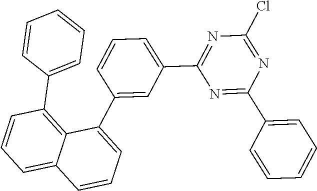 [SMILES: Clc1nc(-c2ccccc2)nc(-c2cccc(-c3cccc4cccc(-c5ccccc5)c34)c2)n1] | 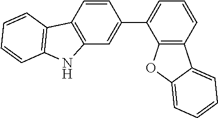 [SMILES: c1ccc2c(c1)[nH]c1cc(-c3cccc4c3oc3ccccc34)ccc12] | 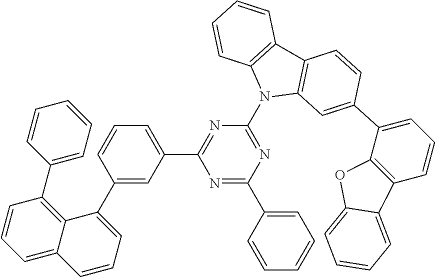 [SMILES: c1ccc(-c2nc(-c3cccc(-c4cccc5cccc(-c6ccccc6)c45)c3)nc(-n3c4ccccc4c4ccc(-c5cccc6c5oc5ccccc56)cc43)n2)cc1] | 39.7 | 767.3 |
| IM Tr-2 | IM-Cz-02 | A57 | | |
| 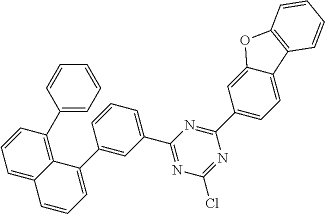 [SMILES: Clc1nc(-c2cccc(-c3cccc4cccc(-c5ccccc5)c34)c2)nc(-c2ccc3c(c2)oc2ccccc23)n1] | 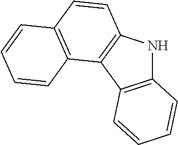 [SMILES: c1ccc2c(c1)ccc1[nH]c3ccccc3c12] | 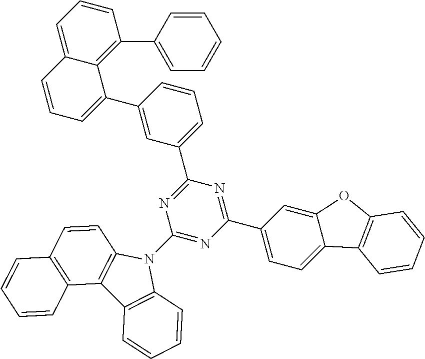 [SMILES: c1ccc(-c2cccc3cccc(-c4cccc(-c5nc(-c6ccc7c(c6)oc6ccccc67)nc(-n6c7ccccc7c7c8ccccc8ccc76)n5)c4)c23)cc1] | 42.8 | 741.3 |
| IM Tr-3 | | A104 | | |
| 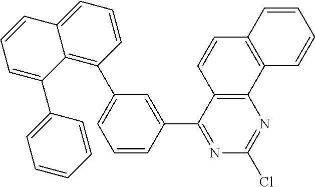 [SMILES: Clc1nc(-c2cccc(-c3cccc4cccc(-c5ccccc5)c34)c2)c2ccc3ccccc3c2n1] | 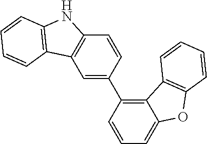 [SMILES: c1ccc2c(c1)[nH]c1ccc(-c3cccc4oc5ccccc5c34)cc12] | 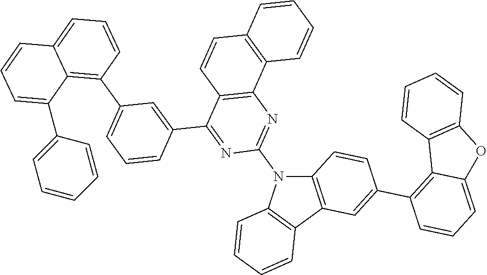 [SMILES: c1ccc(-c2cccc3cccc(-c4cccc(-c5nc(-n6c7ccccc7c7cc(-c8cccc9oc%10ccccc%10c89)ccc76)nc6c5ccc5ccccc56)c4)c23)cc1] | 41.5 | 790.3 |
| IM Tr-4 | IM-Cz-03 | B122 | | |
| 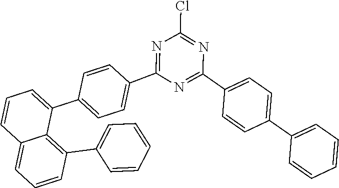 [SMILES: Clc1nc(-c2ccc(-c3ccccc3)cc2)nc(-c2ccc(-c3cccc4cccc(-c5ccccc5)c34)cc2)n1] | 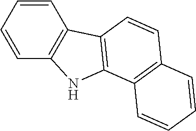 [SMILES: c1ccc2c(c1)ccc1c3ccccc3[nH]c21] | 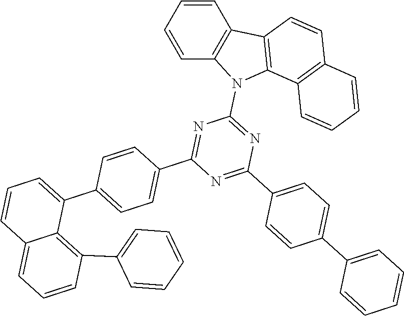 [SMILES: c1ccc(-c2ccc(-c3nc(-c4ccc(-c5cccc6cccc(-c7ccccc7)c56)cc4)nc(-n4c5ccccc5c5ccc6ccccc6c54)n3)cc2)cc1] | 42.4 | 727.3 |
| IM Tr-5 | | A87 | | |
| 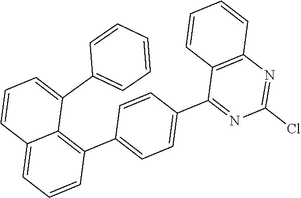 [SMILES: Clc1nc(-c2ccc(-c3cccc4cccc(-c5ccccc5)c34)cc2)c2ccccc2n1] | 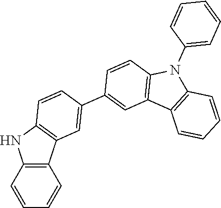 [SMILES: c1ccc(-n2c3ccccc3c3cc(-c4ccc5[nH]c6ccccc6c5c4)ccc32)cc1] | 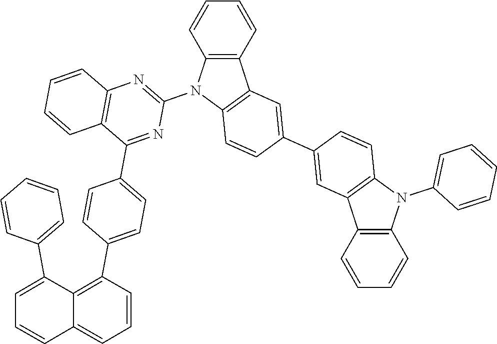 [SMILES: c1ccc(-c2cccc3cccc(-c4ccc(-c5nc(-n6c7ccccc7c7cc(-c8ccc9c(c8)c8ccccc8n9-c8ccccc8)ccc76)nc6ccccc56)cc4)c23)cc1] | 51.2 | 815.3 |
| IM Tr-6 | IM Cz-01 | B42 | | |
| 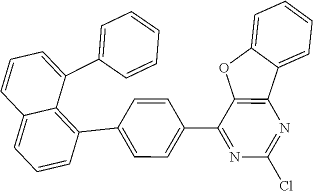 [SMILES: Clc1nc(-c2ccc(-c3cccc4cccc(-c5ccccc5)c34)cc2)c2oc3ccccc3c2n1] | 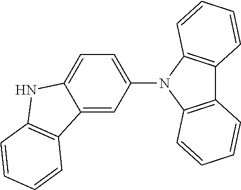 [SMILES: c1ccc2c(c1)[nH]c1ccc(-n3c4ccccc4c4ccccc43)cc12] | 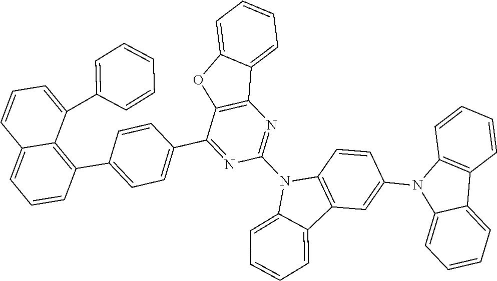 [SMILES: c1ccc(-c2cccc3cccc(-c4ccc(-c5nc(-n6c7ccccc7c7cc(-n8c9ccccc9c9ccccc98)ccc76)nc6c5oc5ccccc56)cc4)c23)cc1] | 44.8 | 779.3 |
| IM Tr-7 | | B99 | | |
| 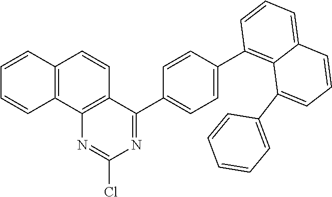 [SMILES: Clc1nc(-c2ccc(-c3cccc4cccc(-c5ccccc5)c34)cc2)c2ccc3ccccc3c2n1] | 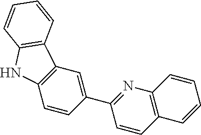 [SMILES: c1ccc2nc(-c3ccc4[nH]c5ccccc5c4c3)ccc2c1] | 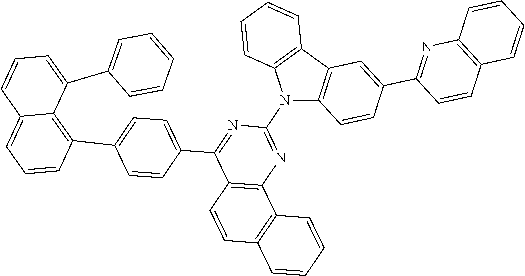 [SMILES: c1ccc(-c2cccc3cccc(-c4ccc(-c5nc(-n6c7ccccc7c7cc(-c8ccc9ccccc9n8)ccc76)nc6c5ccc5ccccc56)cc4)c23)cc1] | 40.3 | 751.3 |
| IM Tr-8 | IM-Cz-05 | B130 | | |
| 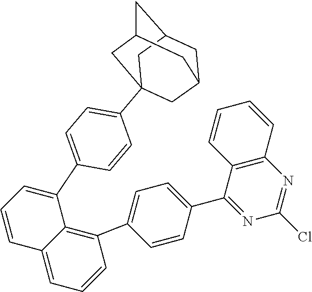 [SMILES: Clc1nc(-c2ccc(-c3cccc4cccc(-c5ccc(C67CC8CC(CC(C8)C6)C7)cc5)c34)cc2)c2ccccc2n1] | 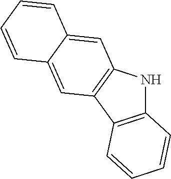 [SMILES: c1ccc2cc3c(cc2c1)[nH]c1ccccc13] | 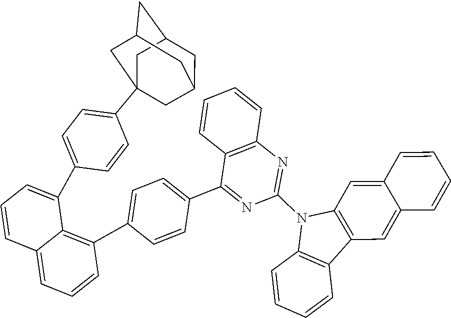 [SMILES: c1ccc2cc3c(cc2c1)c1ccccc1n3-c1nc(-c2ccc(-c3cccc4cccc(-c5ccc(C67CC8CC(CC(C8)C6)C7)cc5)c34)cc2)c2ccccc2n1] | 46.3 | 758.4 |
| IM Tr-12 | | B37 | | |
| 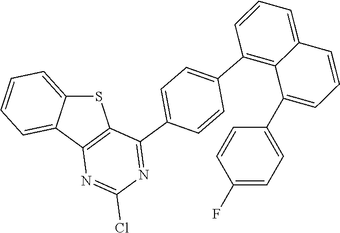 [SMILES: Fc1ccc(-c2cccc3cccc(-c4ccc(-c5nc(Cl)nc6c5sc5ccccc56)cc4)c23)cc1] | 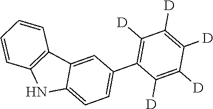 [SMILES: [2H]c1c([2H])c([2H])c(-c2ccc3[nH]c4ccccc4c3c2)c([2H])c1[2H]] | 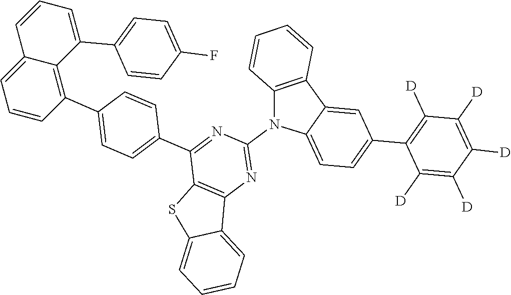 [SMILES: [2H]c1c([2H])c([2H])c(-c2ccc3c(c2)c2ccccc2n3-c2nc(-c3ccc(-c4cccc5cccc(-c6ccc(F)cc6)c45)cc3)c3sc4ccccc4c3n2)c([2H])c1[2H]] | 42.1 | 729.3 |
| IM Tr-13 | IM-Cz-06 | B135 | | |
| 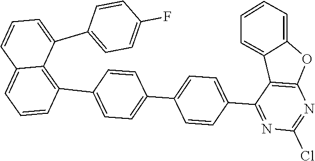 [SMILES: Fc1ccc(-c2cccc3cccc(-c4ccc(-c5ccc(-c6nc(Cl)nc7oc8ccccc8c67)cc5)cc4)c23)cc1] | 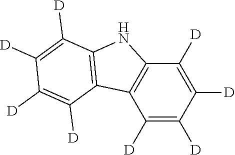 [SMILES: [2H]c1c([2H])c([2H])c2c([nH]c3c([2H])c([2H])c([2H])c([2H])c32)c1[2H]] | 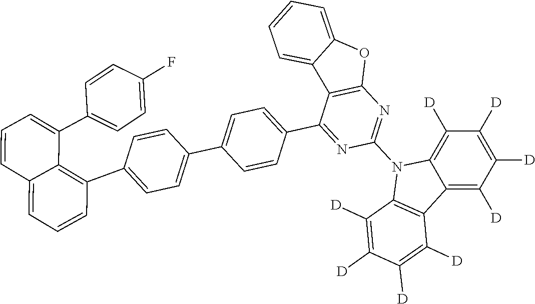 [SMILES: [2H]c1c([2H])c([2H])c2c(c1[2H])c1c([2H])c([2H])c([2H])c([2H])c1n2-c1nc(-c2ccc(-c3ccc(-c4cccc5cccc(-c6ccc(F)cc6)c45)cc3)cc2)c2c(n1)oc1ccccc12] | 41.3 | 716.3 |
| IM Tr-15 | | B85 | | |
| 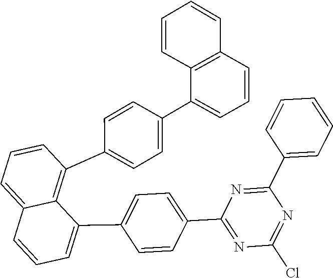 [SMILES: Clc1nc(-c2ccccc2)nc(-c2ccc(-c3cccc4cccc(-c5ccc(-c6cccc7ccccc67)cc5)c34)cc2)n1] | 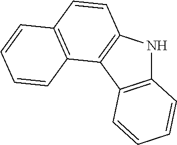 [SMILES: c1ccc2c(c1)ccc1[nH]c3ccccc3c12] | 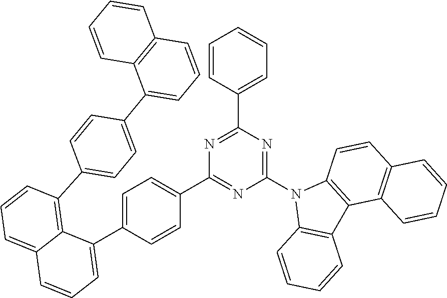 [SMILES: c1ccc(-c2nc(-c3ccc(-c4cccc5cccc(-c6ccc(-c7cccc8ccccc78)cc6)c45)cc3)nc(-n3c4ccccc4c4c5ccccc5ccc43)n2)cc1] | 46.0 | 777.3 |
| IM Tr-17 | | A99 | | |
| 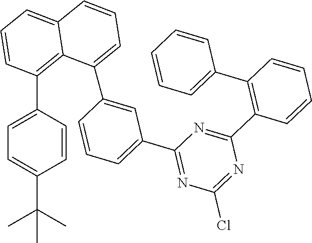 [SMILES: CC(C)(C)c1ccc(-c2cccc3cccc(-c4cccc(-c5nc(Cl)nc(-c6ccccc6-c6ccccc6)n5)c4)c23)cc1] | 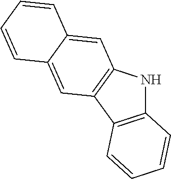 [SMILES: c1ccc2cc3c(cc2c1)[nH]c1ccccc13] | 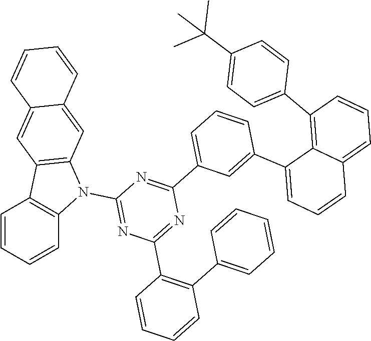 [SMILES: CC(C)(C)c1ccc(-c2cccc3cccc(-c4cccc(-c5nc(-c6ccccc6-c6ccccc6)nc(-n6c7ccccc7c7cc8ccccc8cc76)n5)c4)c23)cc1] | 41.9 | 783.3 |
| IM Tr-18 | | A118 | | |
| 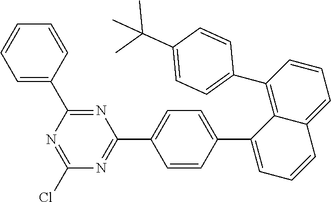 [SMILES: CC(C)(C)c1ccc(-c2cccc3cccc(-c4ccc(-c5nc(Cl)nc(-c6ccccc6)n5)cc4)c23)cc1] | 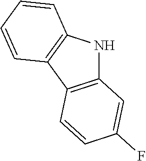 [SMILES: Fc1ccc2c(c1)[nH]c1ccccc12] | 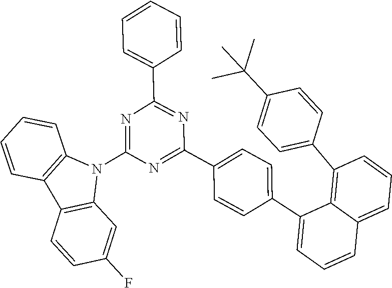 [SMILES: CC(C)(C)c1ccc(-c2cccc3cccc(-c4ccc(-c5nc(-c6ccccc6)nc(-n6c7ccccc7c7ccc(F)cc76)n5)cc4)c23)cc1] | 56.3 | 675.3 |
| IM Tr-19 | | A49 | | |
| 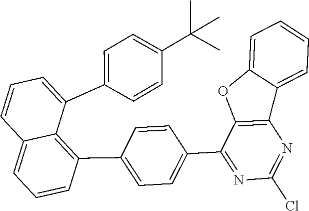 [SMILES: CC(C)(C)c1ccc(-c2cccc3cccc(-c4ccc(-c5nc(Cl)nc6c5oc5ccccc56)cc4)c23)cc1] | 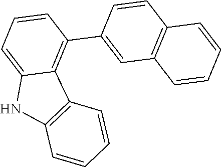 [SMILES: c1ccc2cc(-c3cccc4[nH]c5ccccc5c34)ccc2c1] | 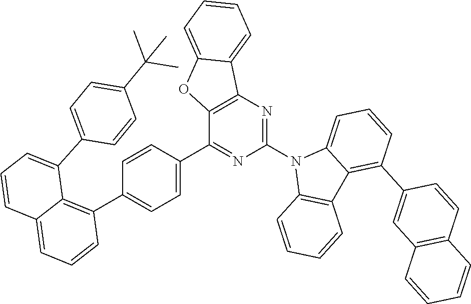 [SMILES: CC(C)(C)c1ccc(-c2cccc3cccc(-c4ccc(-c5nc(-n6c7ccccc7c7c(-c8ccc9ccccc9c8)cccc76)nc6c5oc5ccccc56)cc4)c23)cc1] | 43.6 | 796.3 |
| IM Tr-20 | IM Cz-04 | B96 | | |
| 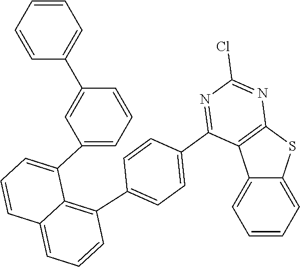 [SMILES: Clc1nc(-c2ccc(-c3cccc4cccc(-c5cccc(-c6ccccc6)c5)c34)cc2)c2c(n1)sc1ccccc12] | 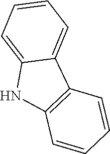 [SMILES: c1ccc2c(c1)[nH]c1ccccc12] | 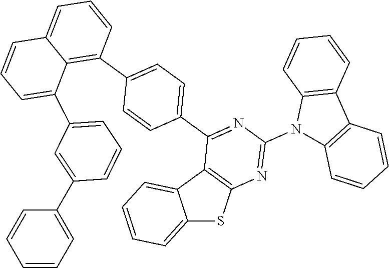 [SMILES: c1ccc(-c2cccc(-c3cccc4cccc(-c5ccc(-c6nc(-n7c8ccccc8c8ccccc87)nc7sc8ccccc8c67)cc5)c34)c2)cc1] | 39.4 | 706.2 |
| IM Tr-21 | | B118 | | |
| 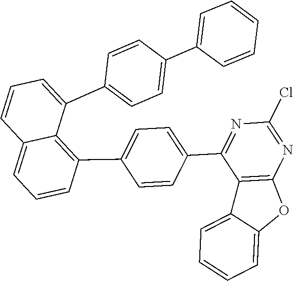 [SMILES: Clc1nc(-c2ccc(-c3cccc4cccc(-c5ccc(-c6ccccc6)cc5)c34)cc2)c2c(n1)oc1ccccc12] | 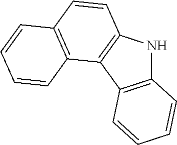 [SMILES: c1ccc2c(c1)ccc1[nH]c3ccccc3c12] | 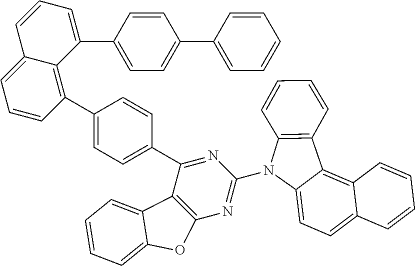 [SMILES: c1ccc(-c2ccc(-c3cccc4cccc(-c5ccc(-c6nc(-n7c8ccccc8c8c9ccccc9ccc87)nc7oc8ccccc8c67)cc5)c34)cc2)cc1] | 42.0 | 740.3 |
| IM Tr-22 | | B78 | | |
| 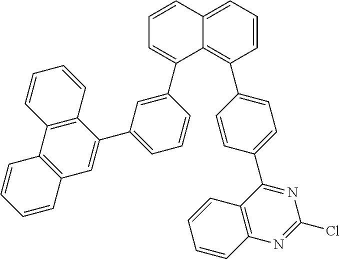 [SMILES: Clc1nc(-c2ccc(-c3cccc4cccc(-c5cccc(-c6cc7ccccc7c7ccccc67)c5)c34)cc2)c2ccccc2n1] | 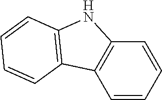 [SMILES: c1ccc2c(c1)[nH]c1ccccc12] | 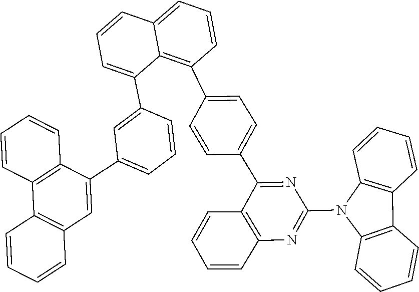 [SMILES: c1cc(-c2cc3ccccc3c3ccccc23)cc(-c2cccc3cccc(-c4ccc(-c5nc(-n6c7ccccc7c7ccccc76)nc6ccccc56)cc4)c23)c1] | 38.8 | 750.3 |
| IM Tr-23 | | B29 | | |

### (3) Synthesis of a Compound A134

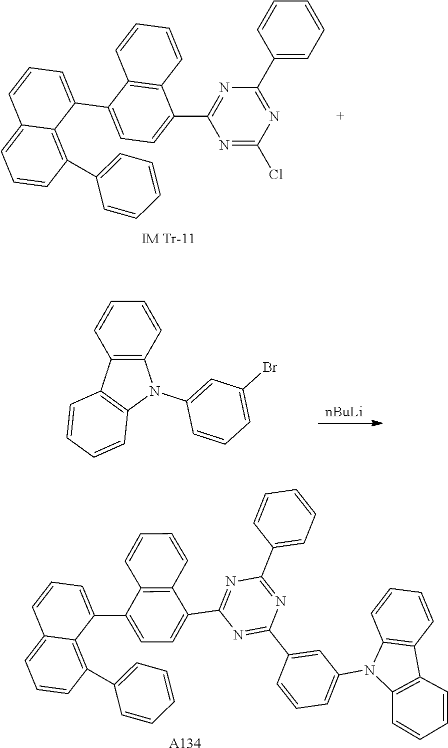

Under N₂ protection, 9-(3-bromophenyl)-9H-carbazole (3.20 g, 9.93 mmol) and dry THF (35 mL) were added into a three-necked flask, stirred to be uniformly dissolved, and cooled to −78° C. with a liquid nitrogen/ethanol bath, followed by slow dropwise addition of a 2 M hexane solution of n-butyllithium (6 mL, 12.00 mmol). After completion of dropwise addition, stirring was performed for 1 hour while heat preservation, then IM Tr-11 (5.16 g, 9.93 mmol) was added, and stirring was performed for 30 min while heat preservation, then the temperature was slowly raised to room temperature, the reaction was quenched by addition of dilute hydrochloric acid, and a pH was adjusted to 5 to 6. The reaction solution was extracted with dichloromethane, an organic phase was washed with water to be neutral, liquid separation, drying, and filtering were performed, and distillation was performed under reduced pressure to remove a solvent, and the obtained crude product was recrystallized by using toluene/petroleum ether to obtain A134 (3.90 g, yield: 54.0%) as a white crystal, mass spectrum (m/z)=727.3 [M+H]⁺.

Compounds shown in Table 8 were synthesized by the same method as that for the compound A134 except that IM Tr-x was used instead of IM Tr-11 and Raw material 8 was used instead of 9-(3-bromophenyl)-9H-carbazole, where the main raw materials used, the compounds synthesized and their yields and mass spectra are shown in Table 8.

TABLE 8

| | | | | Mass |
|---|---|---|---|---|
| | | | | spectrum |
| | | | Yield/ | (m/z)/ |
| IM Tr-x | Raw material 8 | Compound | % | [M + H]⁺ |
| 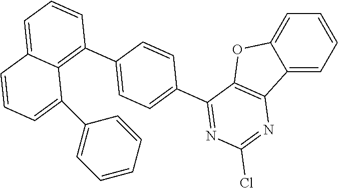 [SMILES: Clc1nc(-c2ccc(-c3cccc4cccc(-c5ccccc5)c34)cc2)c2oc3ccccc3c2n1] | 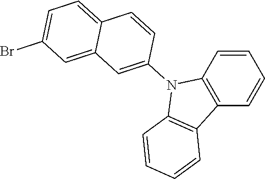 [SMILES: Brc1ccc2ccc(-n3c4ccccc4c4ccccc43)cc2c1] | 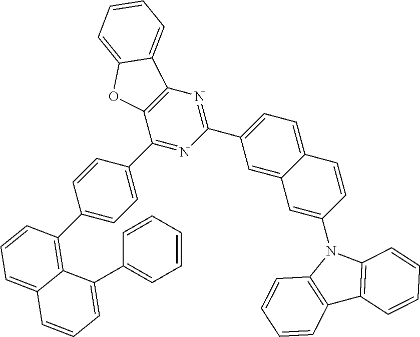 [SMILES: c1ccc(-c2cccc3cccc(-c4ccc(-c5nc(-c6ccc7ccc(-n8c9ccccc9c9ccccc98)cc7c6)nc6c5oc5ccccc56)cc4)c23)cc1] | 40.9 | 740.3 |
| IM Tr-7 | | B109 | | |
| 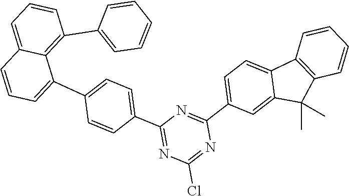 [SMILES: CC1(C)c2ccccc2-c2ccc(-c3nc(Cl)nc(-c4ccc(-c5cccc6cccc(-c7ccccc7)c56)cc4)n3)cc21] | 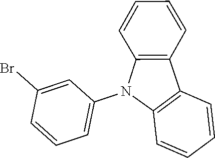 [SMILES: Brc1cccc(-n2c3ccccc3c3ccccc32)c1] | 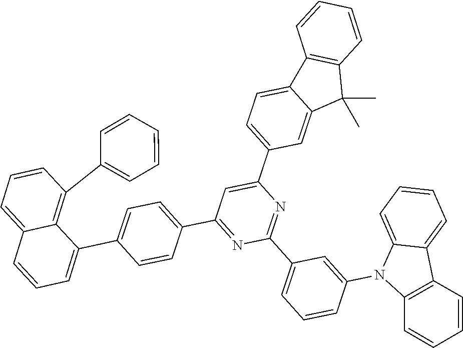 [SMILES: CC1(C)c2ccccc2-c2ccc(-c3cc(-c4ccc(-c5cccc6cccc(-c7ccccc7)c56)cc4)nc(-c4cccc(-n5c6ccccc6c6ccccc65)c4)n3)cc21] | 55.2 | 793.3 |
| IM Tr-9 | | A144 | | |
| 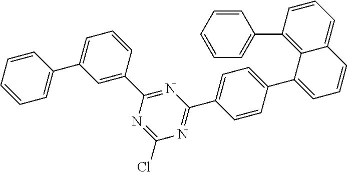 [SMILES: Clc1nc(-c2ccc(-c3cccc4cccc(-c5ccccc5)c34)cc2)nc(-c2cccc(-c3ccccc3)c2)n1] | 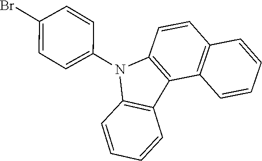 [SMILES: Brc1ccc(-n2c3ccccc3c3c4ccccc4ccc32)cc1] | 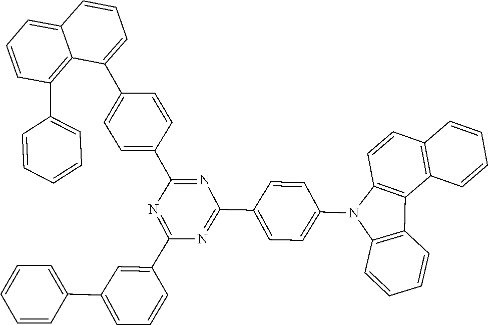 [SMILES: c1ccc(-c2cccc(-c3nc(-c4ccc(-c5cccc6cccc(-c7ccccc7)c56)cc4)nc(-c4ccc(-n5c6ccccc6c6c7ccccc7ccc65)cc4)n3)c2)cc1] | 49.7 | 803.3 |
| IM Tr-10 | | A168 | | |
| 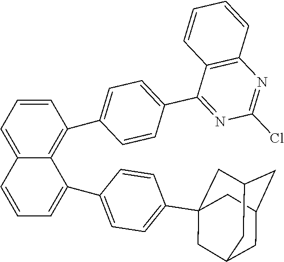 [SMILES: Clc1nc(-c2ccc(-c3cccc4cccc(-c5ccc(C67CC8CC(CC(C8)C6)C7)cc5)c34)cc2)c2ccccc2n1] | 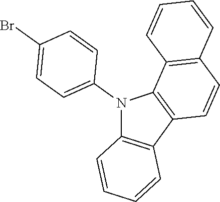 [SMILES: Brc1ccc(-n2c3ccccc3c3ccc4ccccc4c32)cc1] | 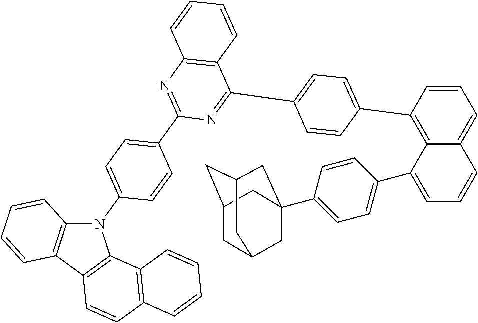 [SMILES: c1cc(-c2ccc(-c3nc(-c4ccc(-n5c6ccccc6c6ccc7ccccc7c65)cc4)nc4ccccc34)cc2)c2c(-c3ccc(C45CC6CC(CC(C6)C4)C5)cc3)cccc2c1] | 45.8 | 834.4 |
| IM Tr-12 | | B54 | | |
| 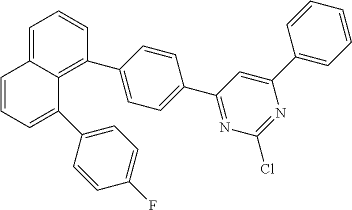 [SMILES: Fc1ccc(-c2cccc3cccc(-c4ccc(-c5cc(-c6ccccc6)nc(Cl)n5)cc4)c23)cc1] | 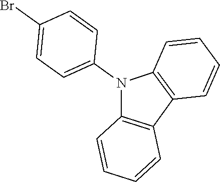 [SMILES: Brc1ccc(-n2c3ccccc3c3ccccc32)cc1] | 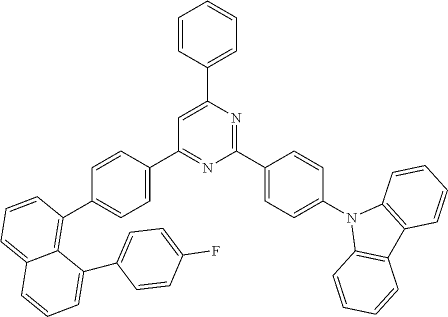 [SMILES: Fc1ccc(-c2cccc3cccc(-c4ccc(-c5cc(-c6ccccc6)nc(-c6ccc(-n7c8ccccc8c8ccccc87)cc6)n5)cc4)c23)cc1] | 52.6 | 694.3 |
| IM Tr-14 | | B06 | | |
| 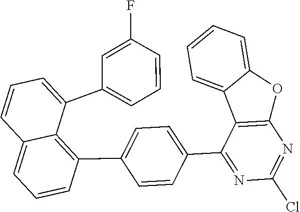 [SMILES: Fc1cccc(-c2cccc3cccc(-c4ccc(-c5nc(Cl)nc6oc7ccccc7c56)cc4)c23)c1] | 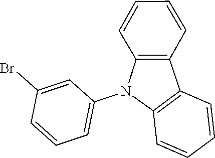 [SMILES: Brc1cccc(-n2c3ccccc3c3ccccc32)c1] | 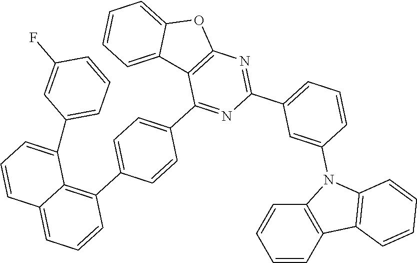 [SMILES: Fc1cccc(-c2cccc3cccc(-c4ccc(-c5nc(-c6cccc(-n7c8ccccc8c8ccccc87)c6)nc6oc7ccccc7c56)cc4)c23)c1] | 42.7 | 708.2 |
| IM Tr-16 | | B71 | | |

NMR data of some compounds are shown below:

NMR data of a compound A07: ¹H-NM/IR (CDCl₃, 300 MHz): δ (ppm) 8.68 (d, 1H), 8.57-8.53 (m, 2H), 8.35 (s, 1H), 8.24 (d, 1H), 8.20 (d, 1H), 8.13 (s, 1H), 8.10-8.07 (m, 2H), 7.98 (d, 1H), 7.95 (d, 1H), 7.83-7.75 (m, 4H), 7.64-7.53 (m, 9H), 7.51-7.47 (m, 4H), 7.44 (t, 1H), 7.37-7.31 (m, 3H), 7.26-7.22 (m, 2H), 7.13 (d, 1H).

NMR data of a compound A22: ¹H-NMR (CDCl₃, 300 MHz): δ (ppm) 8.85 (s, 1H), 8.59 (d, 1H), 8.35-8.27 (m, 2H), 8.20 (d, 2H), 8.12 (d, 2H), 7.88-7.84 (m, 3H), 7.75 (d, 2H), 7.72-7.66 (m, 6H), 7.59 (d, 2H), 7.55-7.47 (m, 8H), 7.42 (t, 1H), 7.27-7.23 (m, 3H), 7.19 (d, 1H).

NMR data of a compound A134: ¹H-NMR (CDCl₃, 300 MHz): δ (ppm) 8.87 (d, 2H), 8.72 (d, 1H), 8.51 (s, 1H), 8.45 (d, 1H), 8.40 (d, 1H), 8.33-8.29 (m, 1H), 8.06 (d, 2H), 7.87-7.71 (m, 5H), 7.68-7.62 (m, 5H), 7.59-7.54 (m, 4H), 7.47-7.41 (m, 5H), 7.34 (t, 2H), 7.25 (t, 2H), 7.19-7.14 (m, 2H).

NMR data of a compound B122: ¹H-NMR (CDCl₃, 300 MHz): δ (ppm) 8.91 (d, 1H), 8.68 (s, 1H), 8.37 (s, 1H), 8.23-8.15 (m, 4H), 8.09 (d, 1H), 8.02-7.09 (m, 2H), 7.87 (d, 1H), 7.81-7.78 (m, 3H), 7.74-7.68 (m, 4H), 7.65-7.54 (m, 6H), 7.52-7.49 (m, 2H), 7.46-7.39 (m, 5H), 7.36-7.33 (m, 1H), 7.26-7.21 (m, 2H), 7.13 (d, 1H).

### Manufacture and Evaluation of Organic Electroluminescent Device:

### Example 1: Manufacture of Red Organic Electroluminescent Device

An ITO/Ag/ITO substrate (manufactured by Corning) having a thickness of 1500 Å was cut to a size of 40 mm (length)×40 mm (width)×0.7 mm (thickness), and prepared into an experimental substrate having an anode and an insulating layer pattern by using a photoetching process, and surface treatment was performed by using UV ozone and O₂:N₂ plasma to improve the work function of the substrate (the anode).

First, F4-TCNQ was vacuum-evaporated on the experimental substrate (the anode) to form a hole injection layer with a thickness of 100 Å, and HT-1 was evaporated on the hole injection layer to form a hole transport layer with a thickness of 1000 Å.

TCAC was vacuum-evaporated on the hole transport layer to form an electron blocking layer with a thickness of 650 Å.

A compound A04 and a compound Ir(flq)₂(acac) were co-evaporated on the electron blocking layer at an evaporation ratio of 98.5%:1.5% to form an organic light-emitting layer with a thickness of 400 Å.

A compound ET-01 and LiQ were co-evaporated on the organic light-emitting layer at an evaporation ratio of 1:1 to form an electron transport layer having a thickness of 300 Å, LiQ was evaporated on the electron transport layer to form an electron injection layer having a thickness of 15 Å, and then magnesium (Mg) and silver (Ag) were vacuum-evaporated on the electron injection layer at an evaporation rate of 1:9 to form a cathode having a thickness of 128 Å.

Finally, CP-01 was evaporated on the cathode to form an organic capping layer having a thickness of 720 Å, thus completing the manufacture of a red organic light-emitting device.

### Examples 2 to 31

An organic electroluminescent device was manufactured by the same method as that in Example 1, except that compounds shown in Table 10 were used instead of Compound A04 as a host material of the light-emitting layer when the organic light-emitting layer was formed.

### Comparative Examples 1 to 5

An organic electroluminescent device was manufactured by the same method as that in Example 1, except that Compounds A, B, C, D, and E were used instead of Compound A04 as a host material of the light-emitting layer when the organic light-emitting layer was formed.

The structures of main materials used in the above Examples and Comparative examples are shown in Table 9 below.

TABLE 9

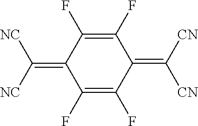 [SMILES: N#CC(C#N)=c1c(F)c(F)c(=C(C#N)C#N)c(F)c1F]

F4-TCQN

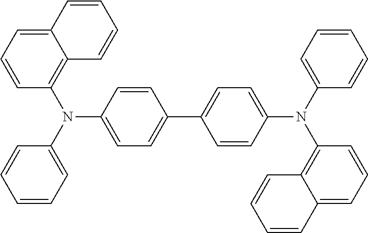 [SMILES: c1ccc(N(c2ccc(-c3ccc(N(c4ccccc4)c4cccc5ccccc45)cc3)cc2)c2cccc3ccccc23)cc1]

HT-1

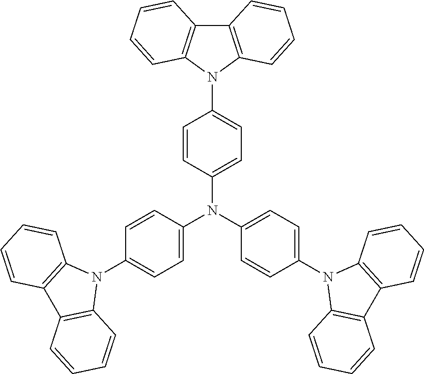 [SMILES: c1ccc2c(c1)c1ccccc1n2-c1ccc(N(c2ccc(-n3c4ccccc4c4ccccc43)cc2)c2ccc(-n3c4ccccc4c4ccccc43)cc2)cc1]

TCAC

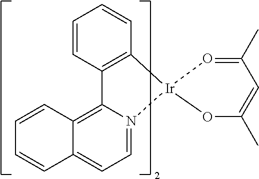

Ir(flq)₂(acac)

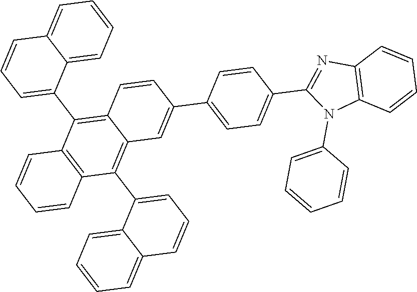 [SMILES: c1ccc(-n2c(-c3ccc(-c4ccc5c(-c6cccc7ccccc67)c6ccccc6c(-c6cccc7ccccc67)c5c4)cc3)nc3ccccc32)cc1]

ET-01

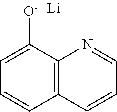 [SMILES: [Li+] ; [O-]c1cccc2cccnc12]

LiQ

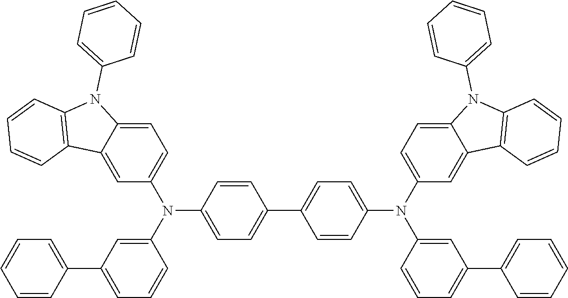 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

CP-01

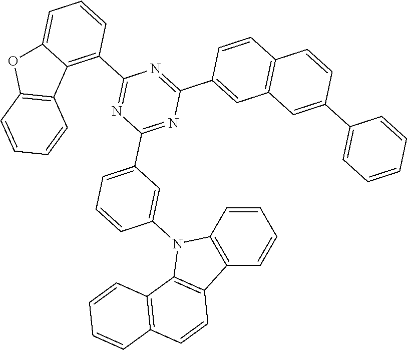 [SMILES: c1ccc(-c2ccc3ccc(-c4nc(-c5cccc(-n6c7ccccc7c7ccc8ccccc8c76)c5)nc(-c5cccc6oc7ccccc7c56)n4)cc3c2)cc1]

Compound A

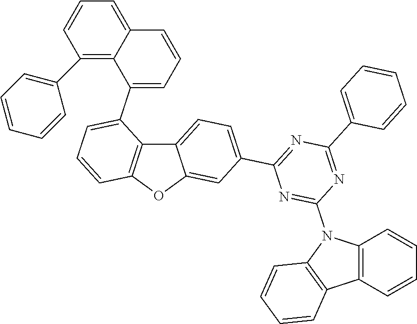 [SMILES: c1ccc(-c2nc(-c3ccc4c(c3)oc3cccc(-c5cccc6cccc(-c7ccccc7)c56)c34)nc(-n3c4ccccc4c4ccccc43)n2)cc1]

Compound B

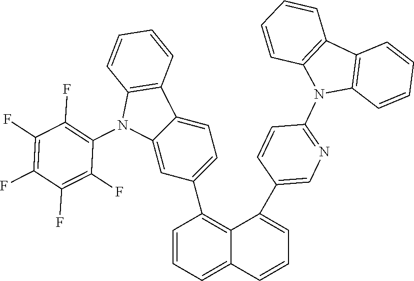 [SMILES: Fc1c(F)c(F)c(-n2c3ccccc3c3ccc(-c4cccc5cccc(-c6ccc(-n7c8ccccc8c8ccccc87)nc6)c45)cc32)c(F)c1F]

Compound C

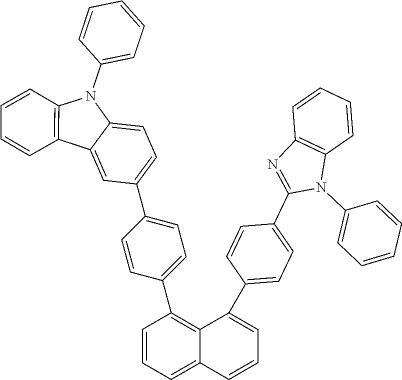 [SMILES: c1ccc(-n2c(-c3ccc(-c4cccc5cccc(-c6ccc(-c7ccc8c(c7)c7ccccc7n8-c7ccccc7)cc6)c45)cc3)nc3ccccc32)cc1]

Compound D

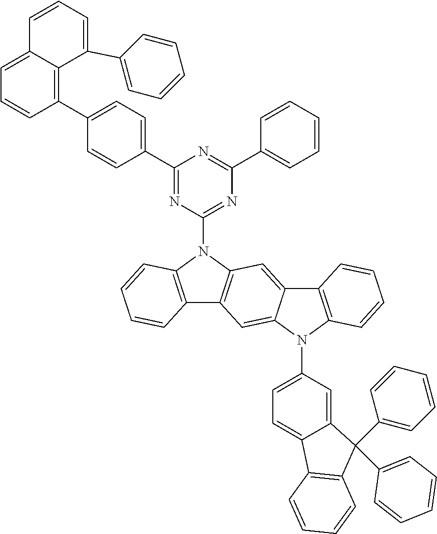 [SMILES: c1ccc(-c2nc(-c3ccc(-c4cccc5cccc(-c6ccccc6)c45)cc3)nc(-n3c4ccccc4c4cc5c(cc43)c3ccccc3n5-c3ccc4c(c3)C(c3ccccc3)(c3ccccc3)c3ccccc3-4)n2)cc1]

Compound E

Performance tests were performed on devices manufactured in Examples and Comparative examples, where IVL (driving voltage and current efficiency) data was tested at a current density of 15 mA/cm² and T95 service life was tested at a current density of 30 mA/cm², and the results are shown in Table 10.

TABLE 10

| | | Driving | Current | Chromaticity | Service |
|---|---|---|---|---|---|
| Example | Host | voltage | efficiency | coordinate | life T95 |
| No. | material | (V) | (Cd/A) | CIEx, CIEy | (h) |
| Example 1 | A04 | 3.81 | 40.51 | 0.680, 0.320 | 370 |
| Example 2 | A07 | 3.85 | 41.84 | 0.680, 0.320 | 371 |
| Example 3 | A22 | 3.84 | 40.83 | 0.680, 0.320 | 380 |
| Example 4 | A35 | 3.88 | 41.65 | 0.680, 0.320 | 374 |
| Example 5 | A43 | 3.82 | 40.68 | 0.680, 0.320 | 377 |
| Example 6 | A49 | 3.78 | 42.11 | 0.680, 0.320 | 373 |
| Example 7 | A57 | 3.83 | 41.21 | 0.680, 0.320 | 374 |
| Example 8 | A64 | 3.88 | 42.42 | 0.680, 0.320 | 379 |
| Example 9 | A76 | 3.84 | 40.77 | 0.680, 0.320 | 375 |
| Example 10 | A87 | 3.88 | 42.65 | 0.680, 0.320 | 377 |
| Example 11 | A99 | 3.87 | 40.29 | 0.680, 0.320 | 379 |
| Example 12 | A104 | 3.85 | 42.18 | 0.680, 0.320 | 378 |
| Example 13 | A118 | 3.81 | 41.56 | 0.680, 0.320 | 371 |
| Example 14 | A134 | 3.79 | 40.64 | 0.680, 0.320 | 372 |
| Example 15 | A144 | 3.85 | 40.17 | 0.680, 0.320 | 380 |
| Example 16 | A168 | 3.89 | 41.54 | 0.680, 0.320 | 376 |
| Example 17 | B06 | 3.75 | 35.63 | 0.680, 0.320 | 341 |
| Example 18 | B29 | 3.78 | 35.67 | 0.680, 0.320 | 339 |
| Example 19 | B37 | 3.77 | 35.39 | 0.680, 0.320 | 345 |
| Example 20 | B42 | 3.71 | 35.37 | 0.680, 0.320 | 342 |
| Example 21 | B54 | 3.72 | 37.74 | 0.680, 0.320 | 340 |
| Example 22 | B71 | 3.76 | 36.72 | 0.680, 0.320 | 348 |
| Example 23 | B78 | 3.73 | 36.11 | 0.680, 0.320 | 336 |
| Example 24 | B85 | 3.71 | 35.91 | 0.680, 0.320 | 346 |
| Example 25 | B96 | 3.74 | 35.45 | 0.680, 0.320 | 347 |
| Example 26 | B99 | 3.72 | 35.55 | 0.680, 0.320 | 332 |
| Example 27 | B109 | 3.79 | 35.87 | 0.680, 0.320 | 337 |
| Example 28 | B118 | 3.82 | 35.49 | 0.680, 0.320 | 344 |
| Example 29 | B122 | 3.78 | 37.43 | 0.680, 0.320 | 335 |
| Example 30 | B130 | 3.76 | 37.36 | 0.680, 0.320 | 343 |
| Example 31 | B135 | 3.77 | 35.48 | 0.680, 0.320 | 334 |
| Comparative | Compound | 3.99 | 30.98 | 0.680, 0.320 | 285 |
| example 1 | A | | | | |
| Comparative | Compound | 4.05 | 31.29 | 0.680, 0.320 | 298 |
| example 2 | B | | | | |
| Comparative | Compound | 3.98 | 30.17 | 0.680, 0.320 | 267 |
| example 3 | C | | | | |
| Comparative | Compound | 3.93 | 29.95 | 0.680, 0.320 | 269 |
| example 4 | D | | | | |
| Comparative | Compound | 4.07 | 27.83 | 0.680, 0.320 | 243 |
| example 5 | E | | | | |

According to the results shown in Table 10, it can be seen that Examples 1 to 31 using the organic compound of the present disclosure as the organic light-emitting layer have the advantages that the current efficiency (Cd/A) is improved by at least 13.04% and the service life is improved by at least 11% compared with Comparative examples 1 to 5 of devices corresponding to known compounds.

The preferred embodiments of the present disclosure are described in detail above, however, the present disclosure is not limited to the specific details in the above embodiments, and various simple variations can be made to the technical solutions of the present disclosure within the scope of the technical idea of the present disclosure, and these simple variations all fall within the protection scope of the present disclosure.

1. An organic compound, having a structure represented by a Formula 1: 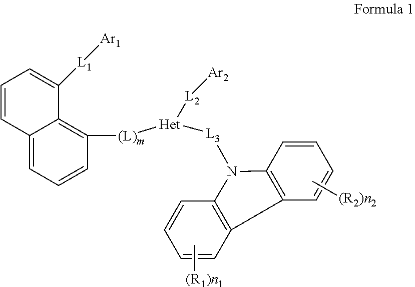
wherein Het is a 6- to 18-membered nitrogen-containing heteroarylene;
Ar₁ is selected from a substituted or unsubstituted aryl with 6 to 40 carbon atoms;
Ar₂ is selected from hydrogen, a substituted or unsubstituted aryl with 6 to 40 carbon atoms, or a substituted or unsubstituted heteroaryl with 3 to 40 carbon atoms;
L, L₁, L₂ and L₃ are the same or different, and are each independently selected from a single bond, or a substituted or unsubstituted arylene with 6 to 30 carbon atoms;
m is selected from 1 or 2;
substituent(s) of L, L₁, L₂, L₃, Ar₁ and Ar₂ are the same or different, and are each independently selected from deuterium, a halogen group, a cyano, an alkyl with 1 to 10 carbon atoms, a trialkylsilyl with 3 to 12 carbon atoms, a haloalkyl with 1 to 10 carbon atoms, a cycloalkyl with 3 to 10 carbon atoms, an aryl with 6 to 20 carbon atoms, or a heteroaryl with 3 to 20 carbon atoms;
R₁ and R₂ are the same or different, and are each independently selected from deuterium, a halogen group, a cyano, an alkyl with 1 to 10 carbon atoms, a trialkylsilyl with 3 to 12 carbon atoms, a cycloalkyl with 3 to 10 carbon atoms, a substituted or unsubstituted aryl with 6 to 20 carbon atoms, or a substituted or unsubstituted heteroaryl with 3 to 20 carbon atoms;
n₁ represents the number of R₁, n₁ is selected from 0, 1, 2, 3 or 4, in the case where n₁ is greater than 1, any two R₁ are the same or different, and optionally, any two adjacent R₁ form an aromatic ring having 6 to 14 carbon atoms;
n₂ represents the number of R₂, n₂ is selected from 0, 1, 2, 3 or 4, in the case where n₂ is greater than 1, any two R₂ are the same or different, and optionally, any two adjacent R₂ form an aromatic ring having 6 to 14 carbon atoms; and
substituent(s) of R₁ and R₂ are the same or different, and are each independently selected from deuterium, a halogen group, a cyano, an alkyl with 1 to 5 carbon atoms, an aryl with 6 to 12 carbon atoms, or a heteroaryl with 3 to 12 carbon atoms.

2. The organic compound according to claim 1, wherein Het in the formula 1 is selected from: 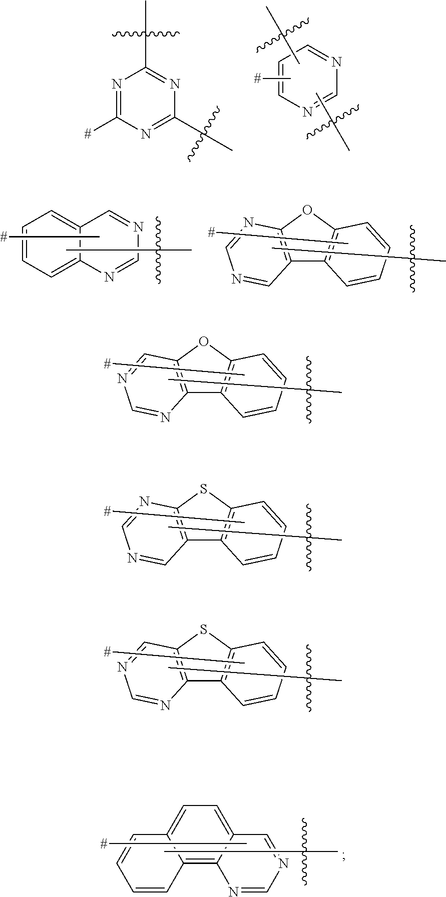 [symbol]
represents a bond connected to L₃, and [symbol] represents a bond connected to L or L₂; and in the case where only one [symbol] is present in the Het group, [symbol] represents a bond connected to L, and at this time, L₂ is a single bond and Ar₂ is hydrogen.

3. The organic compound according to claim 1, wherein Ar₁ is selected from a substituted or unsubstituted aryl with 6 to 20 carbon atoms; and
preferably, substituent(s) of Ar₁ are each independently selected from a deuterium, a fluorine, a cyano, an alkyl with 1 to 5 carbon atoms, a trimethylsilyl, a trifluoromethyl, a cycloalkyl with 5 to 10 carbon atoms, or an aryl with 6 to 12 carbon atoms.

4. The organic compound according to claim 1, wherein Ar₁ is selected from a substituted or unsubstituted phenyl, a substituted or unsubstituted naphthyl, a substituted or unsubstituted biphenyl, a substituted or unsubstituted fluorenyl, a substituted or unsubstituted phenanthryl, or a substituted or unsubstituted terphenyl; and
preferably, substituent(s) of Ar₁ are each independently selected from a deuterium, a fluorine, a cyano, a methyl, an ethyl, an isopropyl, a tert-butyl, a trimethylsilyl, a trifluoromethyl, a cyclopentyl, a cyclohexyl, an adamantyl, a phenyl, a naphthyl or a biphenyl.

5. The organic compound according to claim 1, wherein Ar₁ is selected from:

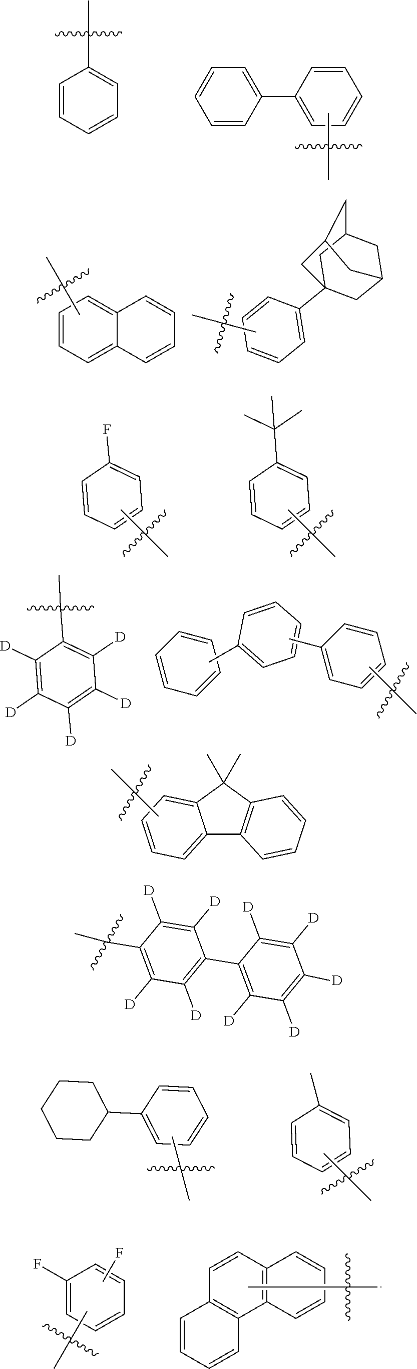

6. The organic compound according to claim 1, wherein Ar₂ is selected from hydrogen, a substituted or unsubstituted aryl with 6 to 25 carbon atoms, or a substituted or unsubstituted heteroaryl with 5 to 18 carbon atoms; and
preferably, substituent(s) of Ar₂ are each independently selected from a deuterium, a fluorine, a cyano, an alkyl with 1 to 5 carbon atoms, a trimethylsilyl, a trifluoromethyl, a cycloalkyl with 5 to 10 carbon atoms, an aryl with 6 to 12 carbon atoms, or a heteroaryl with 5 to 12 carbon atoms.

7. The organic compound according to claim 1, wherein Ar₂ is selected from hydrogen, a substituted or unsubstituted phenyl, a substituted or unsubstituted naphthyl, a substituted or unsubstituted biphenyl, a substituted or unsubstituted fluorenyl, a substituted or unsubstituted phenanthryl, a substituted or unsubstituted terphenyl, a substituted or unsubstituted pyridyl, a substituted or unsubstituted quinolyl, a substituted or unsubstituted dibenzofuranyl, a substituted or unsubstituted dibenzothienyl, or a substituted or unsubstituted carbazolyl; and
preferably, substituent(s) of Ar₂ are each independently selected from a deuterium, a fluorine, a cyano, a methyl, an ethyl, an isopropyl, a tert-butyl, a trimethylsilyl, a trifluoromethyl, a cyclopentyl, a cyclohexyl, an adamantyl, a phenyl, a naphthyl, a biphenyl, a dibenzofuranyl, a dibenzothienyl or a carbazolyl.

8. The organic compound according to claim 1, wherein Ar₂ is selected from hydrogen or the following groups:

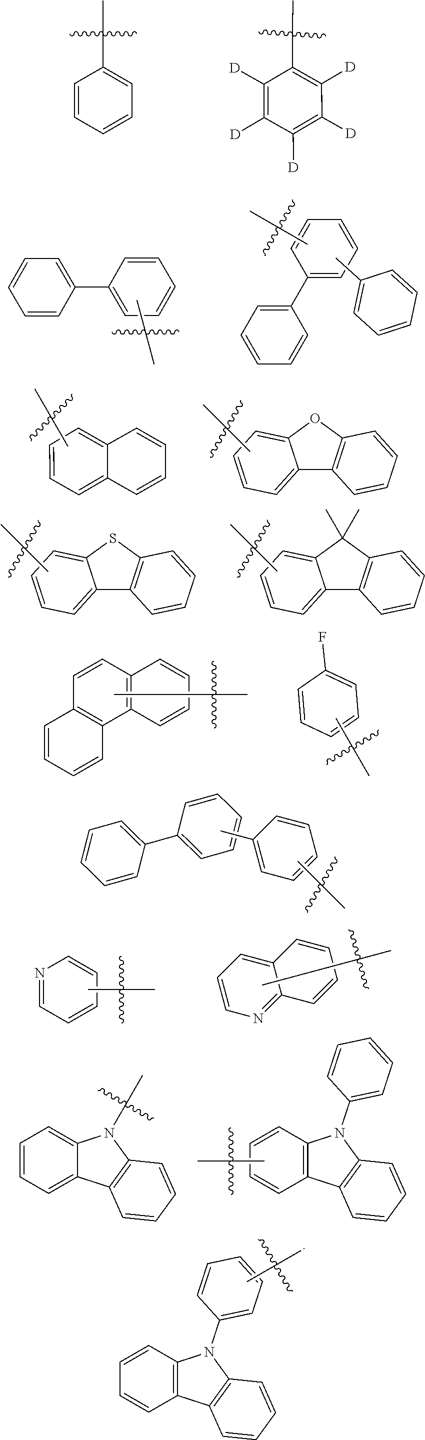

9. The organic compound according to claim 1, wherein L, L₁, L₂, and L₃ are each independently selected from a single bond or a substituted or unsubstituted arylene with 6 to 20 carbon atoms; and
preferably, substituent(s) of L, L₁, L₂, and L₃ are each independently selected from a deuterium, a fluorine, a cyano, an alkyl with 1 to 5 carbon atoms or an aryl with 6 to 12 carbon atoms.

10. The organic compound according to claim 1, wherein L, L₁, L₂, and L₃ are each independently selected from a single bond, a substituted or unsubstituted phenylene, a substituted or unsubstituted naphthylene, or a substituted or unsubstituted biphenylene; and
preferably, substituent(s) of L, L₁, L₂, and L₃ are each independently selected from a deuterium, a fluorine, a cyano, a methyl, an ethyl, an isopropyl, a tert-butyl, a phenyl, a naphthyl or a biphenyl.

11. The organic compound according to claim 1, wherein L₁, L₂, and L₃ are each independently selected from a single bond or the following group: 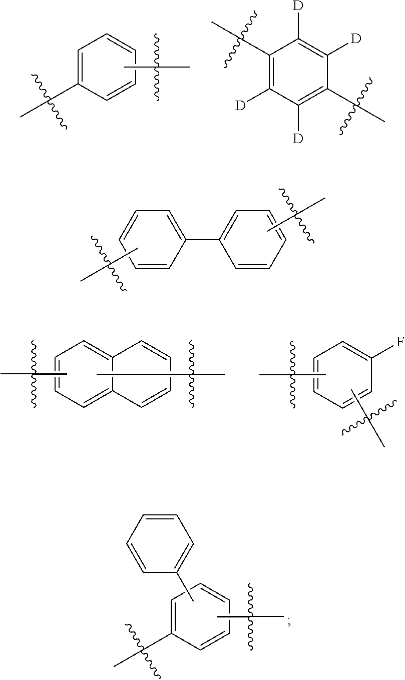
and
optionally, -(L)ₘ- is selected from a single bond or the following group:

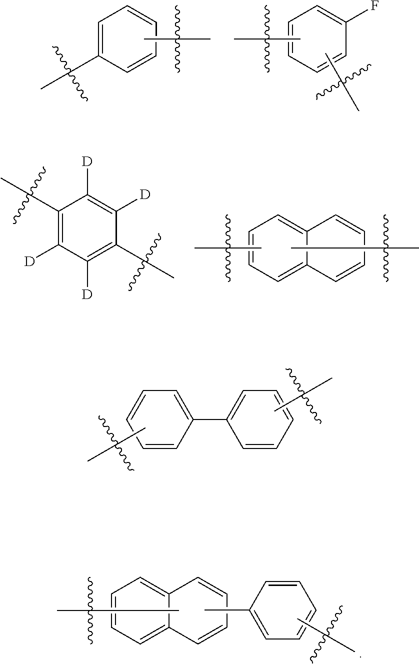

12. The organic compound according to claim 1, wherein R₁ and R₂ are each independently selected from a deuterium, a fluorine, a cyano, a methyl, an ethyl, an isopropyl, a tert-butyl, a trimethylsilyl, a deuterium-substituted phenyl, a phenyl, a naphthyl, a biphenyl, a phenanthryl, a pyridyl, a quinolyl, a 9,9-dimethylfluorenyl, a dibenzofuranyl, a dibenzothienyl, a N-carbazolyl, or a N-phenylcarbazolyl; or any two adjacent R₁ form a benzene ring or a naphthalene ring; or any two adjacent R₂ form a benzene ring or a naphthalene ring.

13. The organic compound according to claim 1, wherein the organic compound is selected from the group consisting of the following compounds:

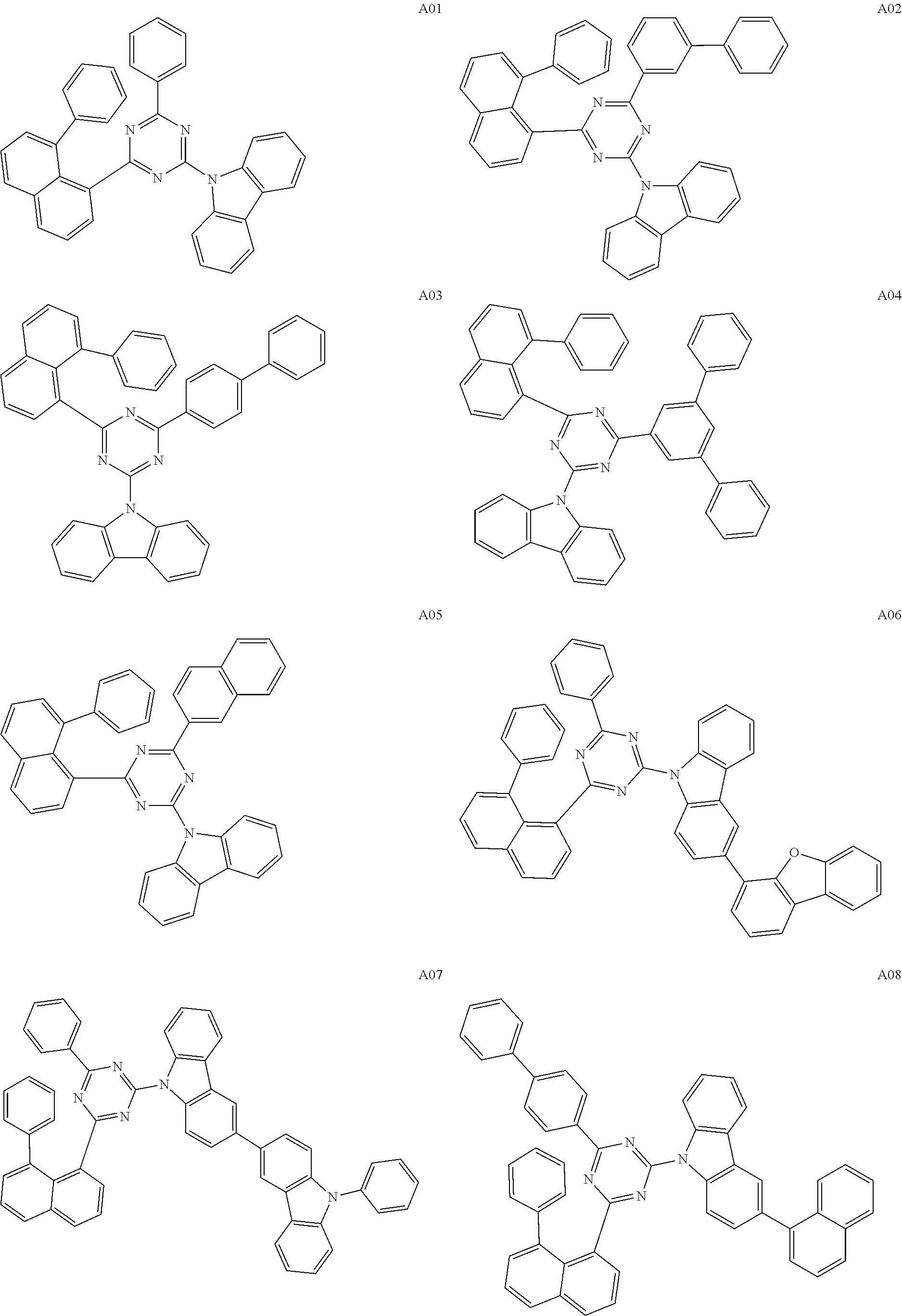

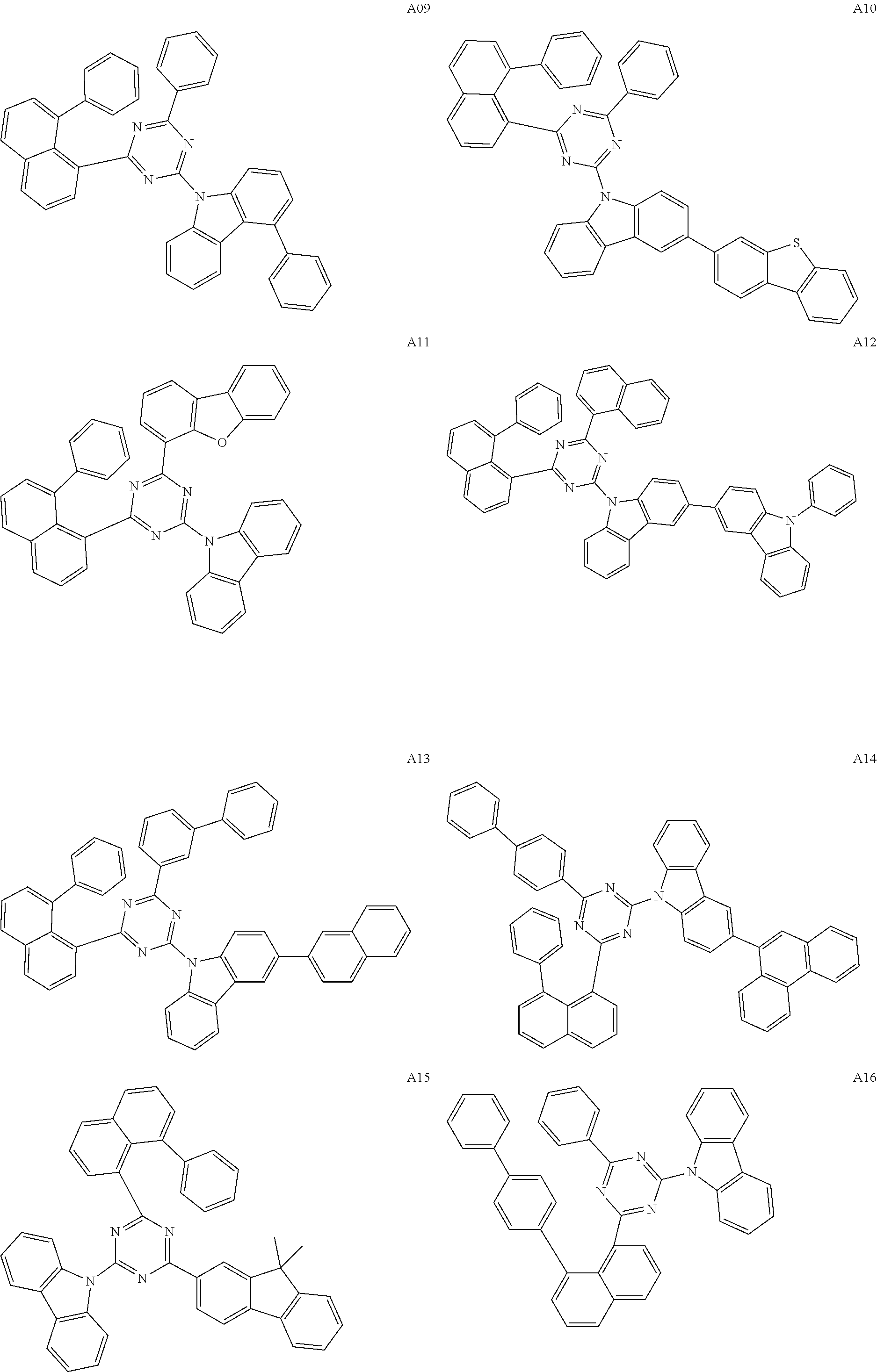

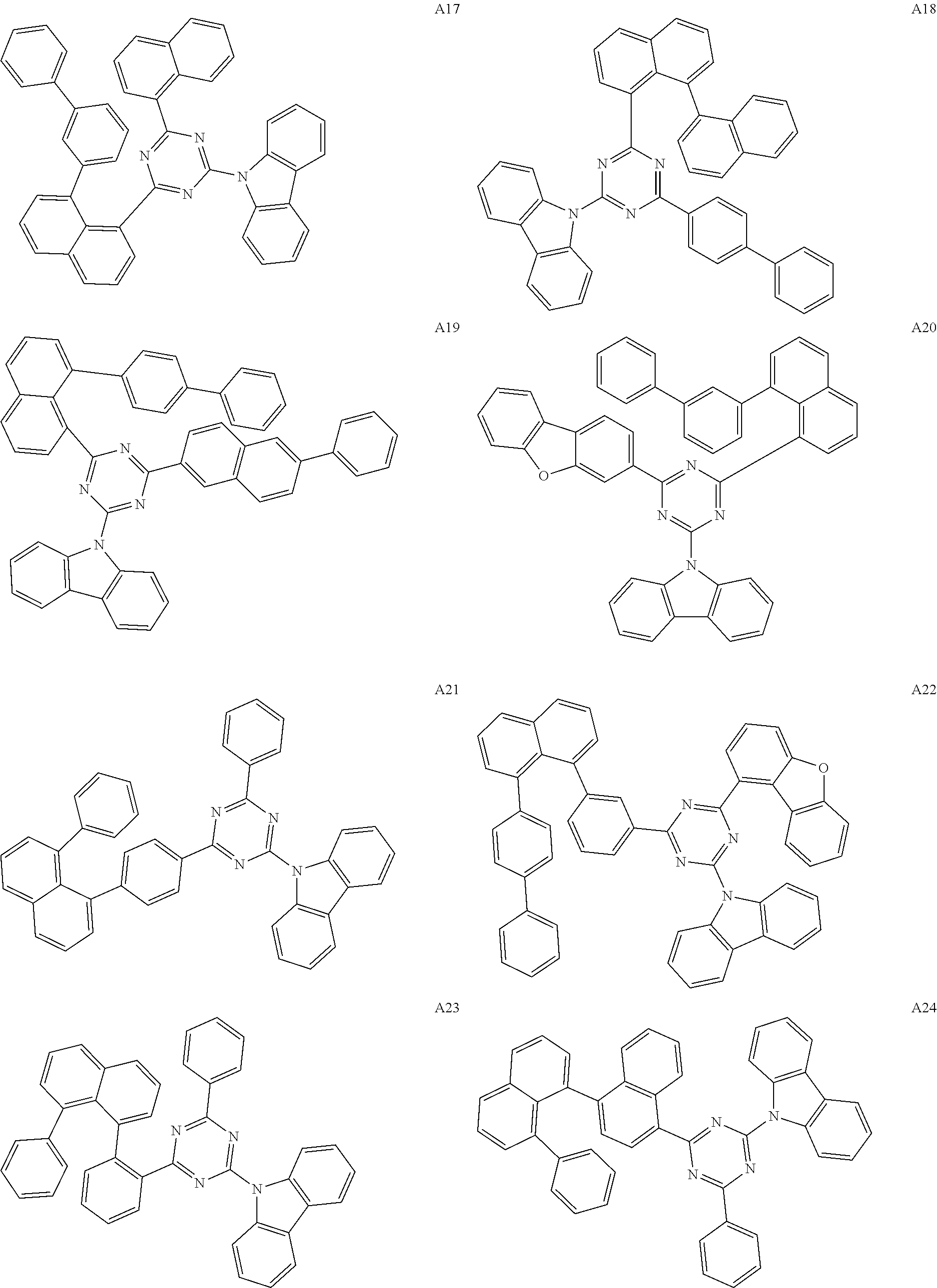

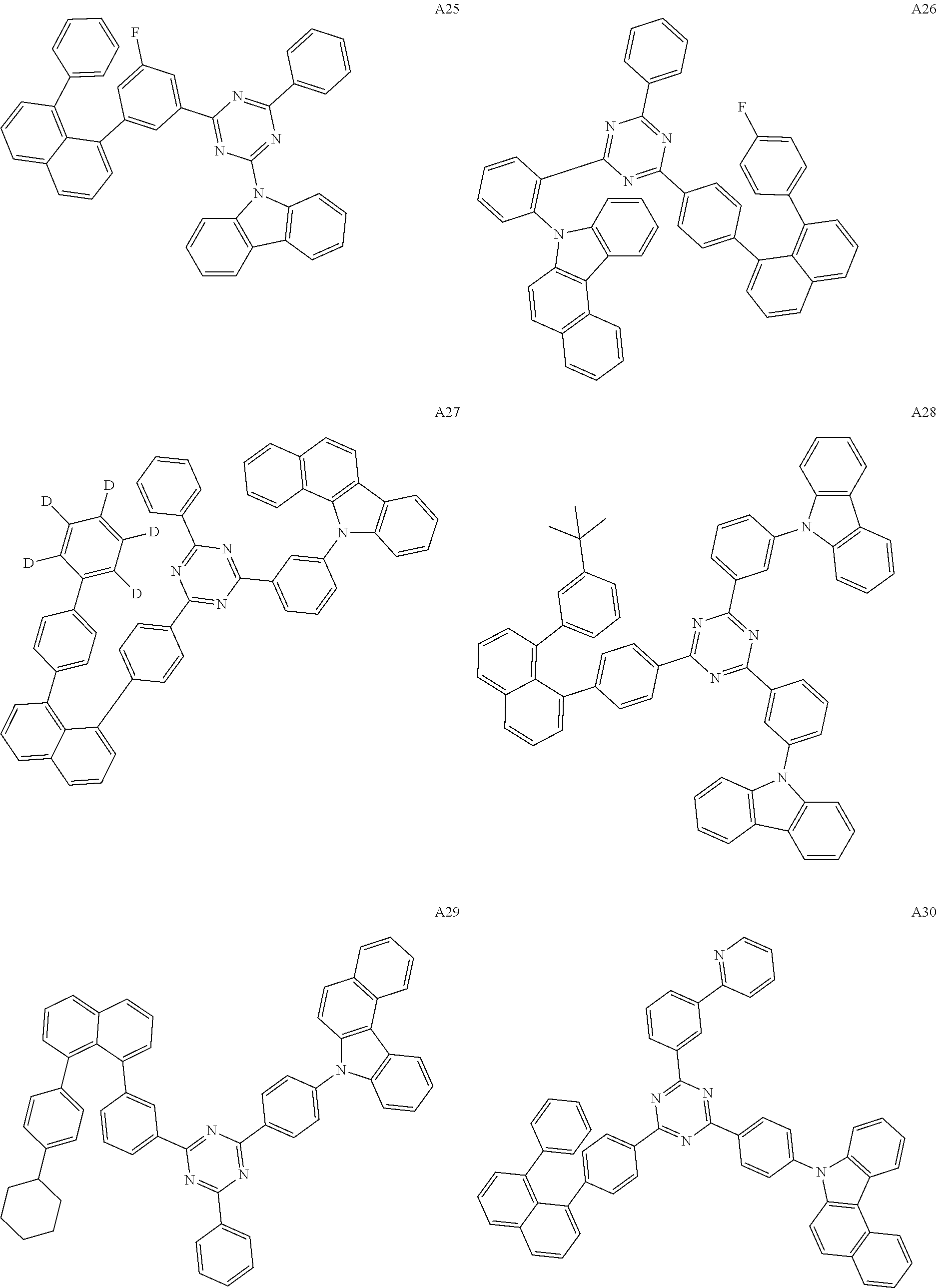

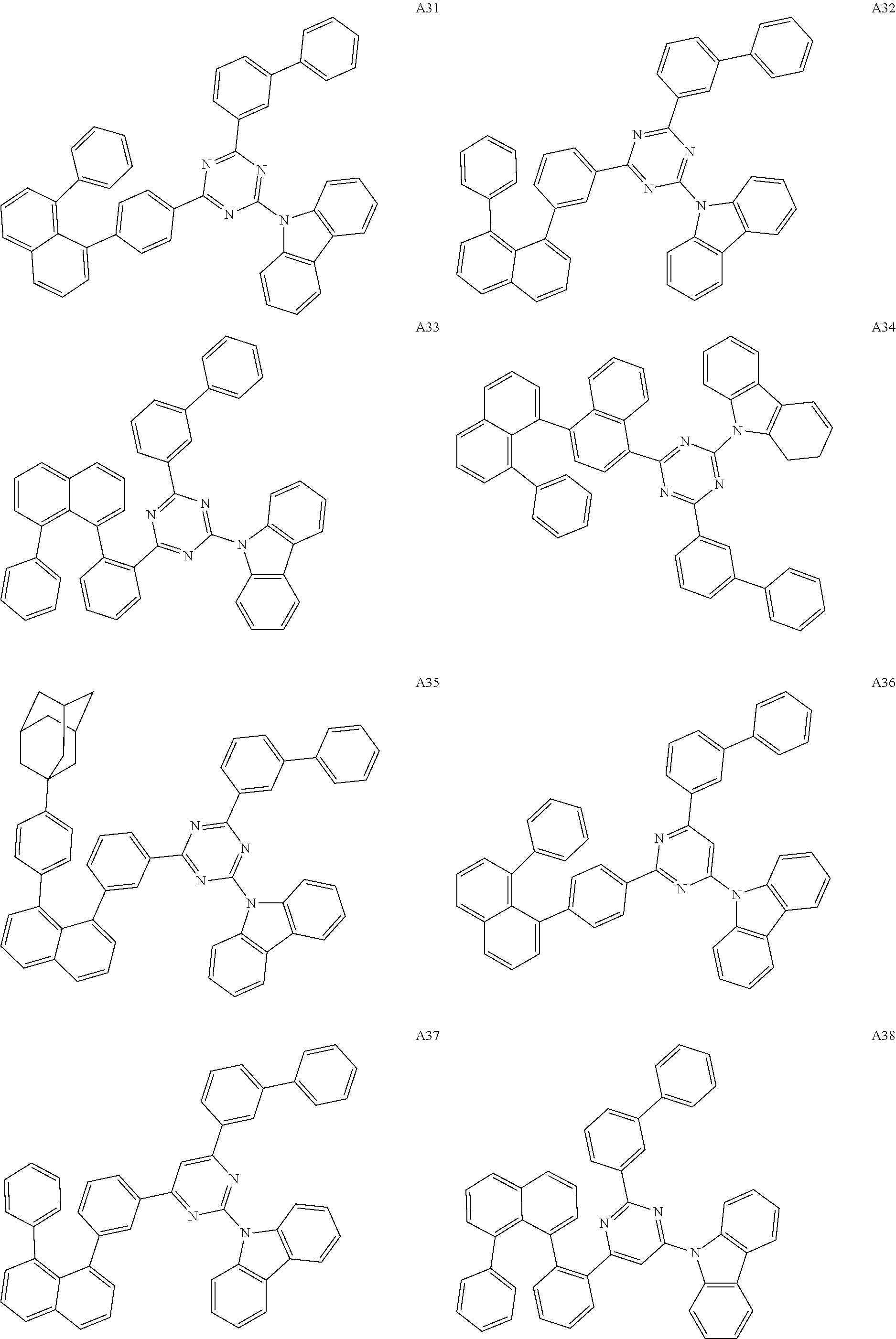

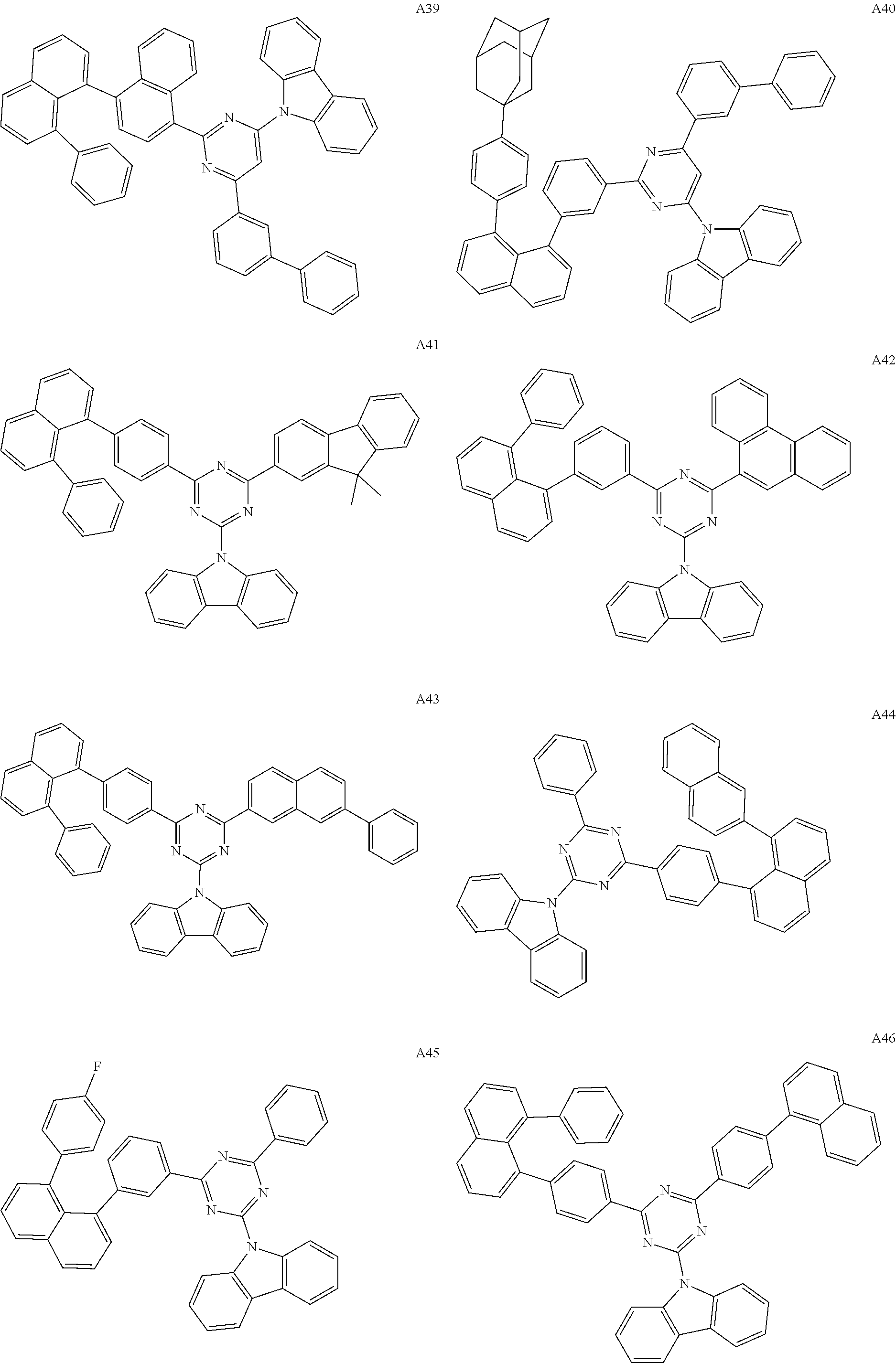

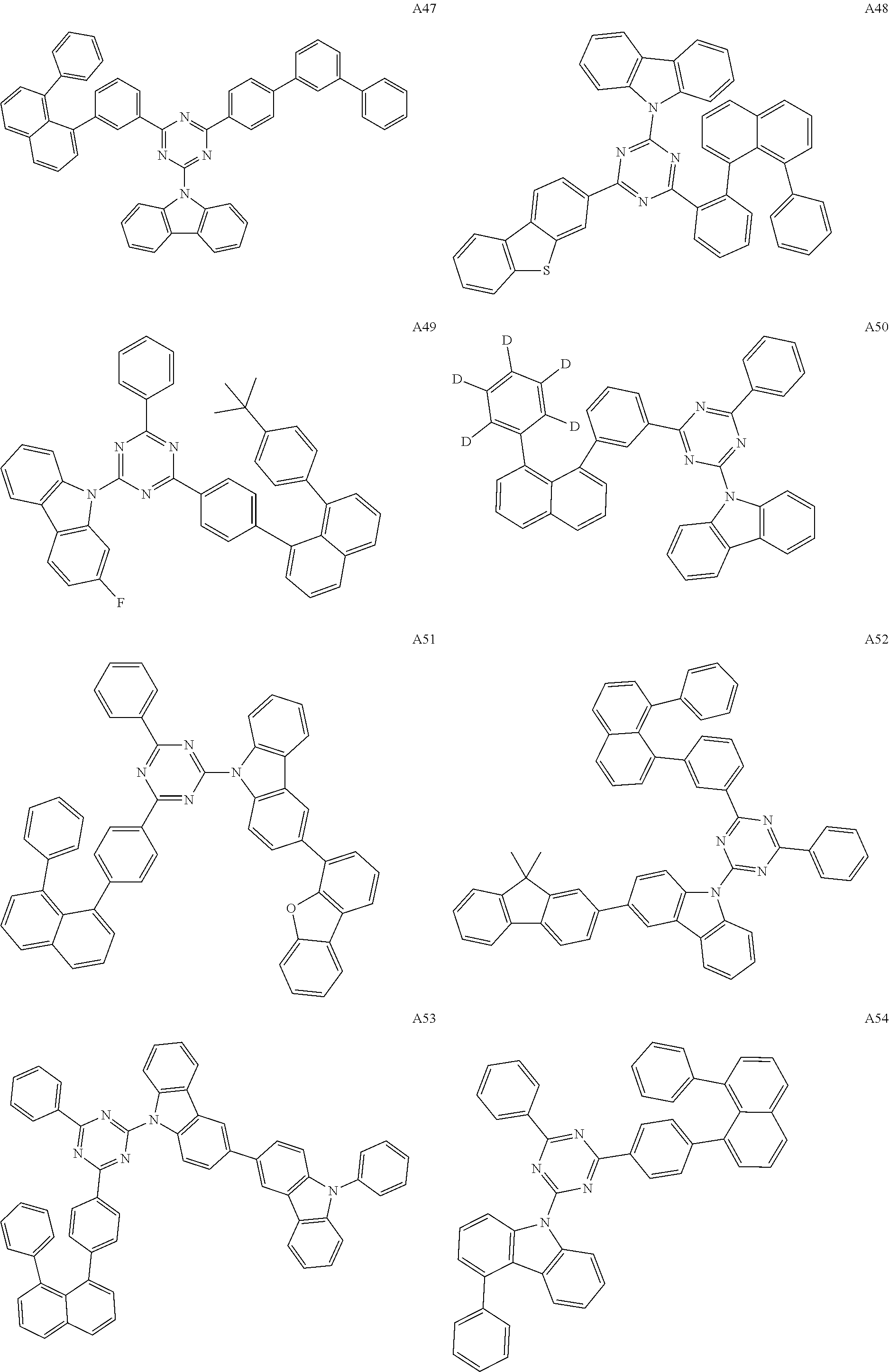

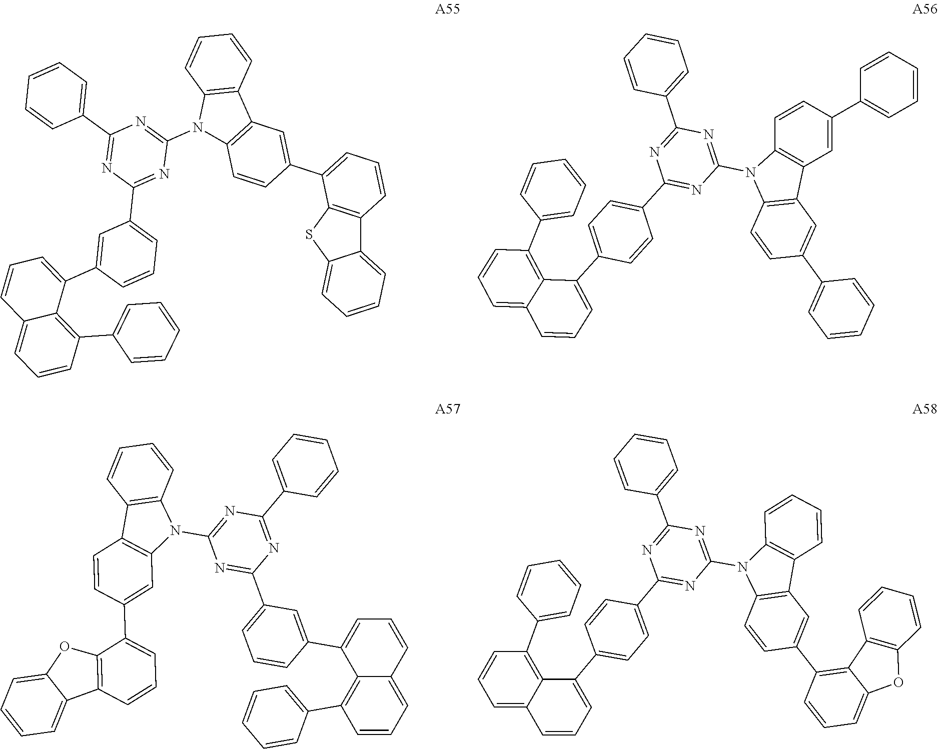 [SMILES: c1ccc(-c2nc(-c3cccc(-c4cccc5cccc(-c6ccccc6)c45)c3)nc(-n3c4ccccc4c4cc(-c5cccc6c5sc5ccccc56)ccc43)n2)cc1 ; c1ccc(-c2ccc3c(c2)c2cc(-c4ccccc4)ccc2n3-c2nc(-c3ccccc3)nc(-c3ccc(-c4cccc5cccc(-c6ccccc6)c45)cc3)n2)cc1 ; c1ccc(-c2nc(-c3cccc(-c4cccc5cccc(-c6ccccc6)c45)c3)nc(-n3c4ccccc4c4ccc(-c5cccc6c5oc5ccccc56)cc43)n2)cc1 ; c1ccc(-c2nc(-c3ccc(-c4cccc5cccc(-c6ccccc6)c45)cc3)nc(-n3c4ccccc4c4cc(-c5cccc6oc7ccccc7c56)ccc43)n2)cc1]

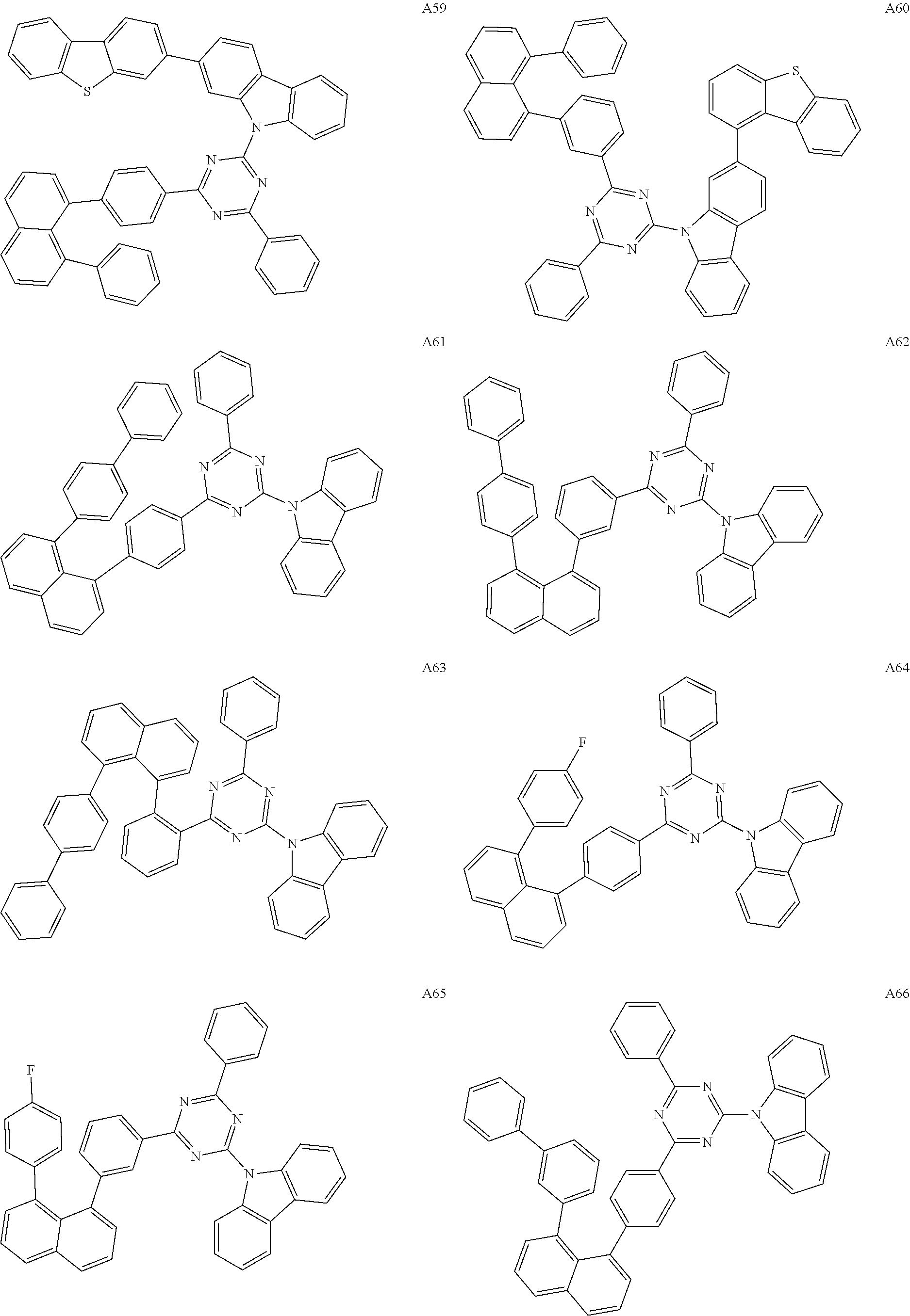

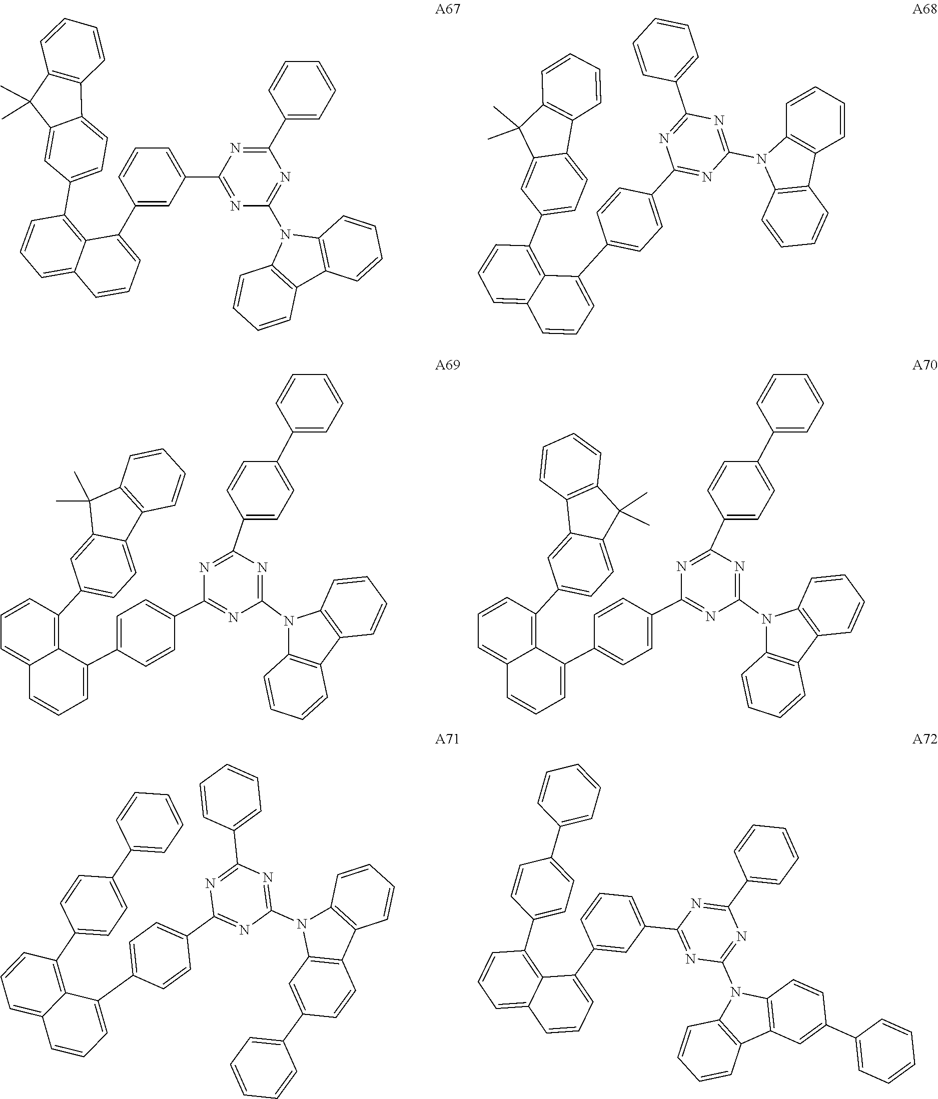

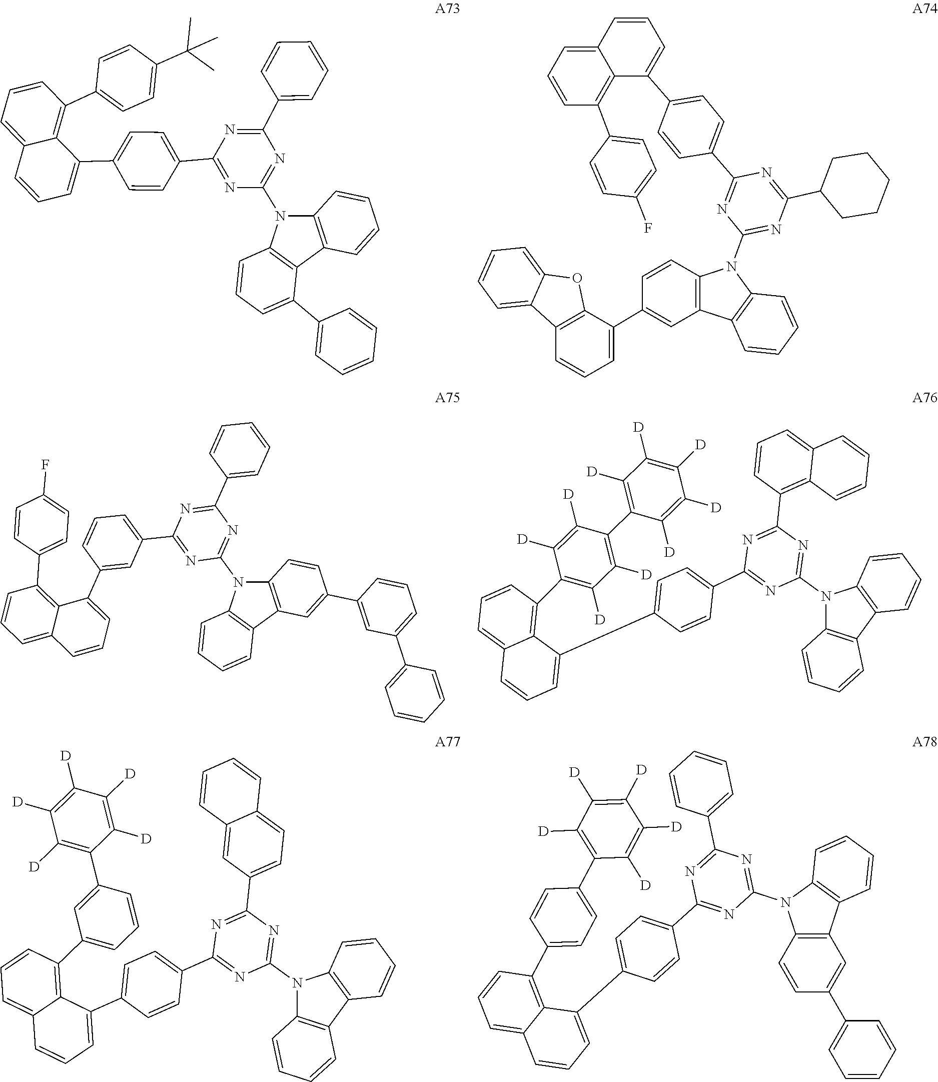

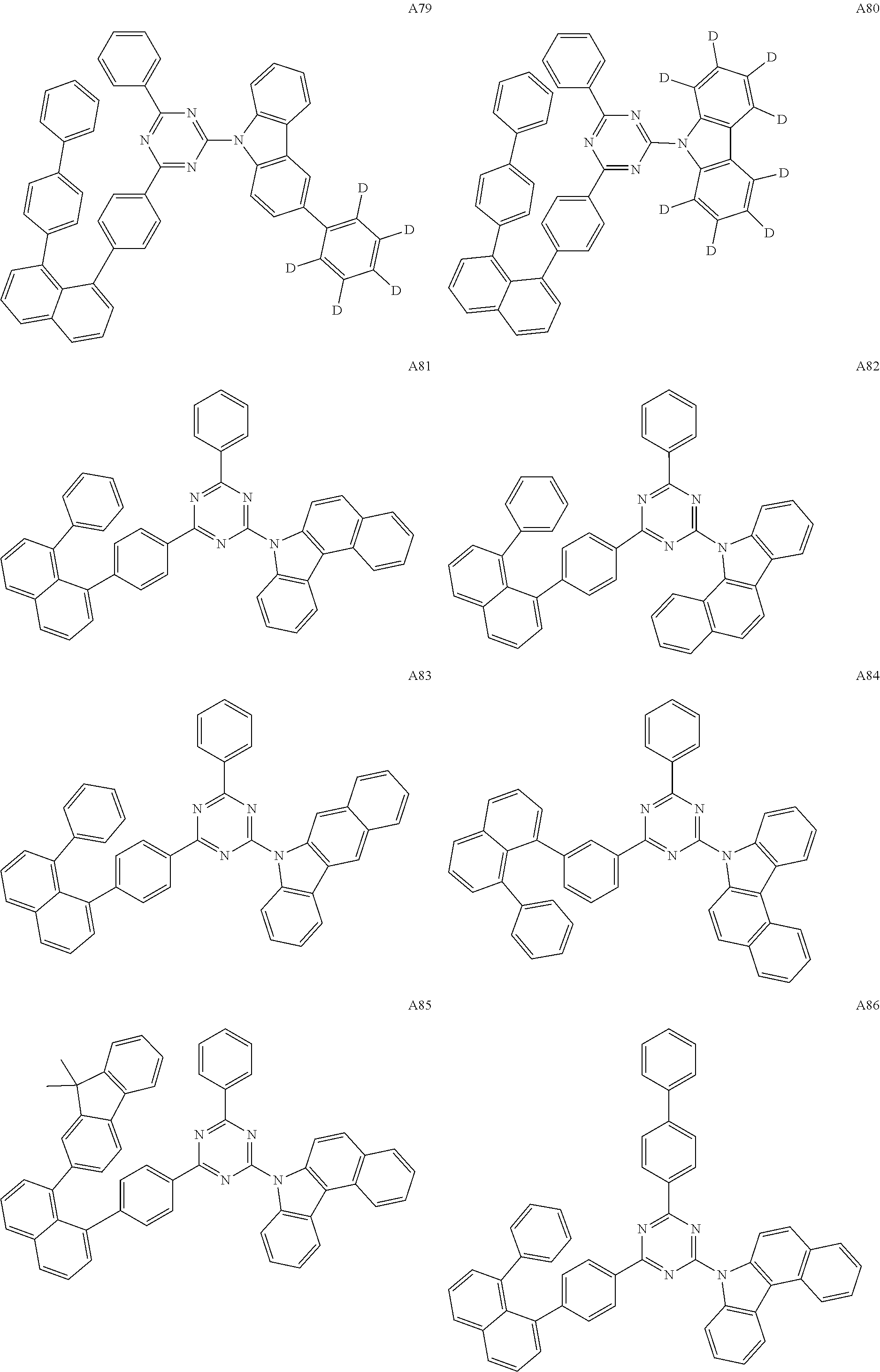

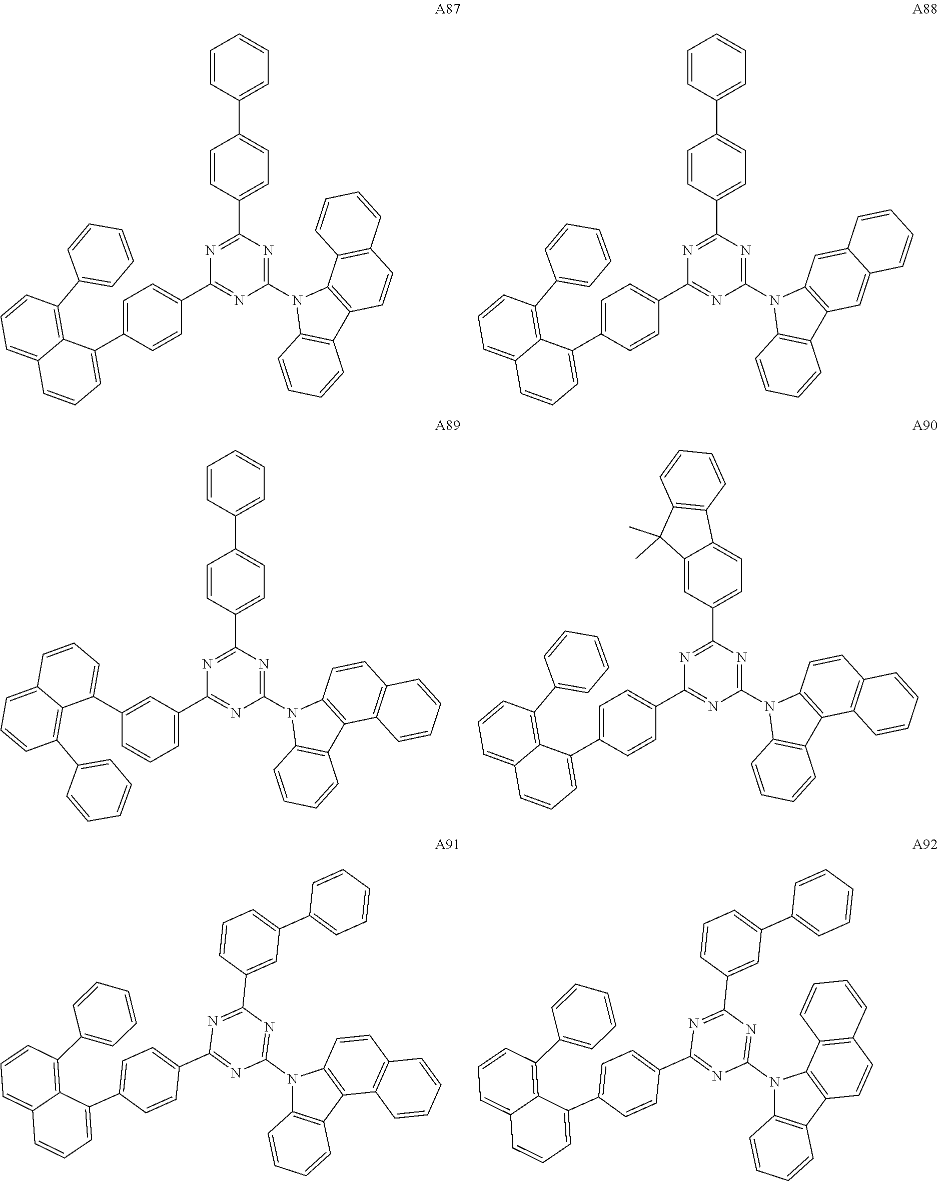

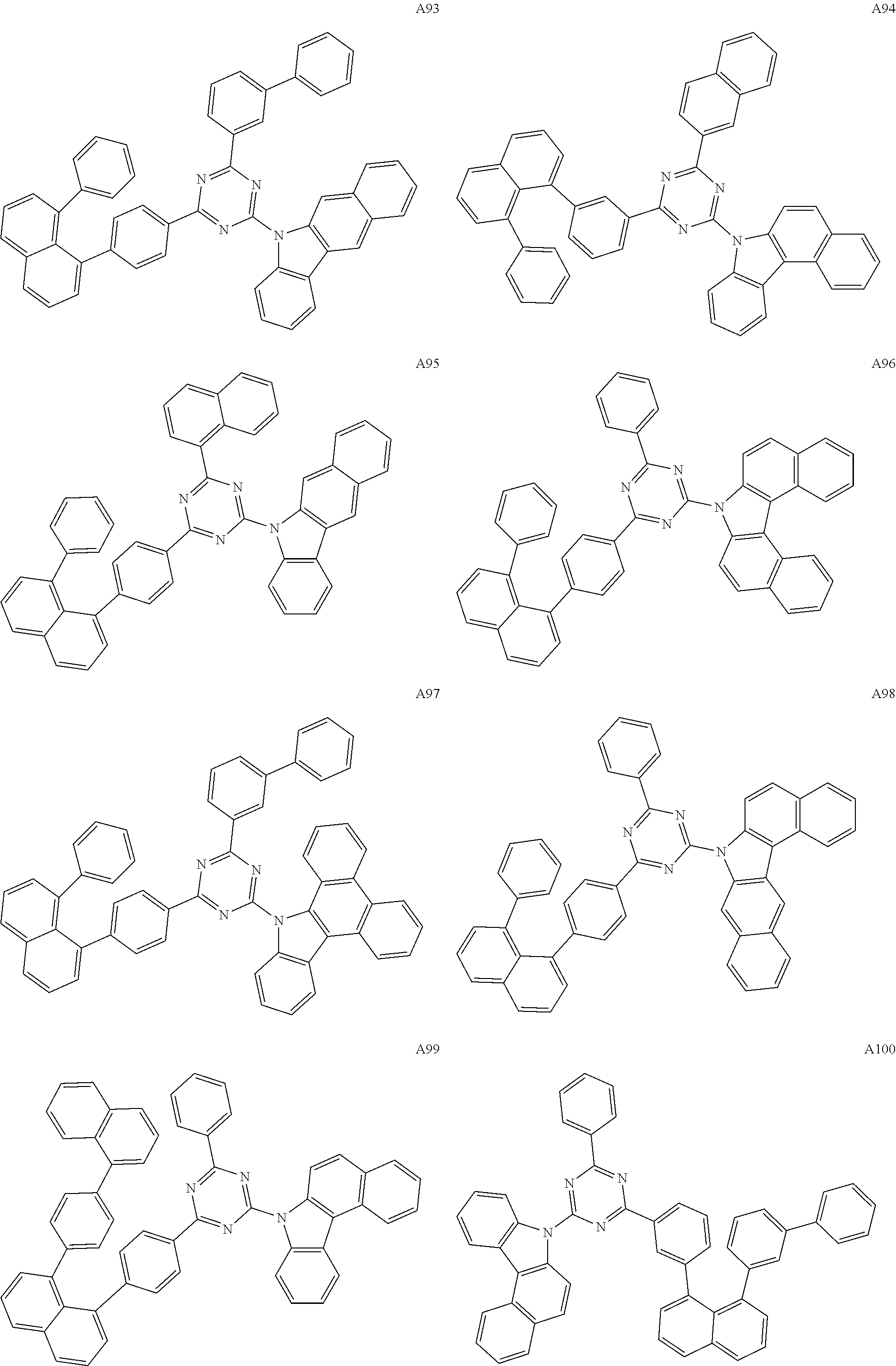

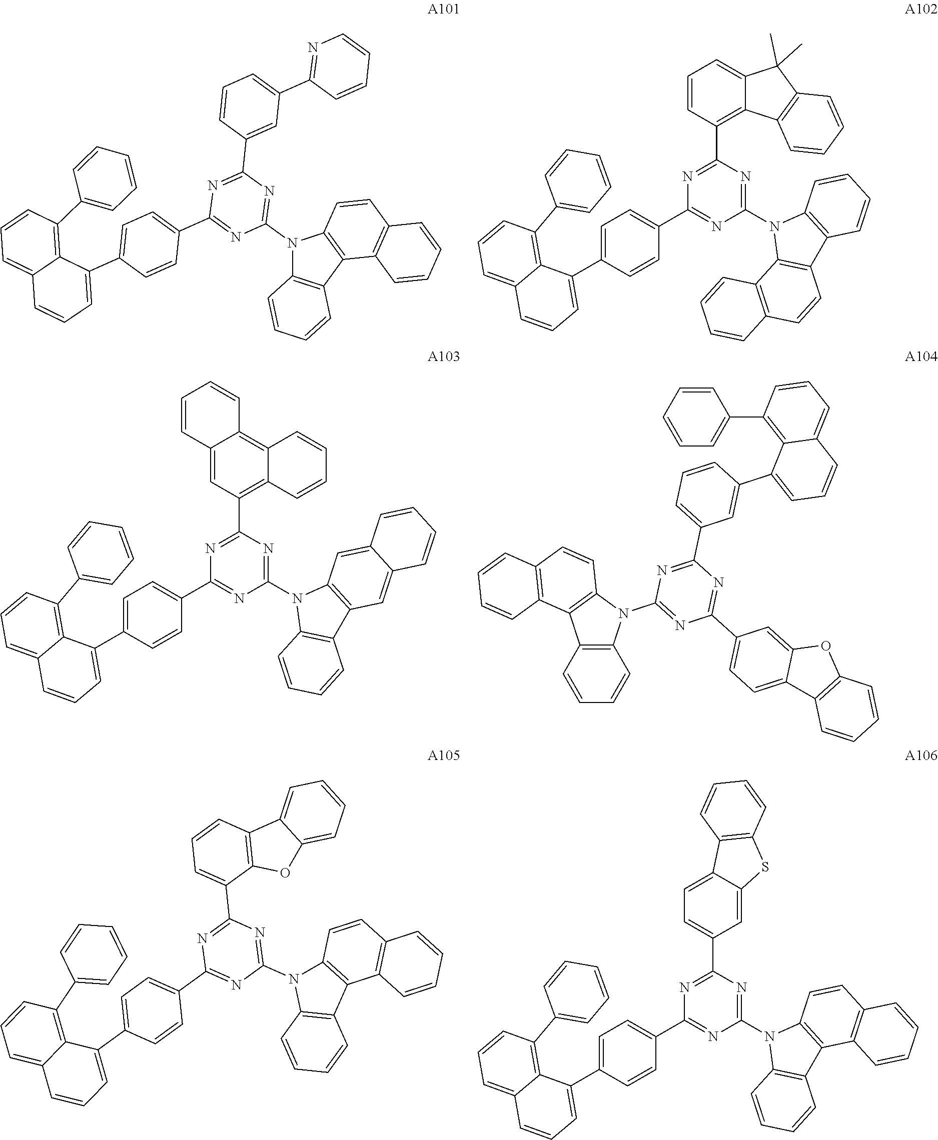

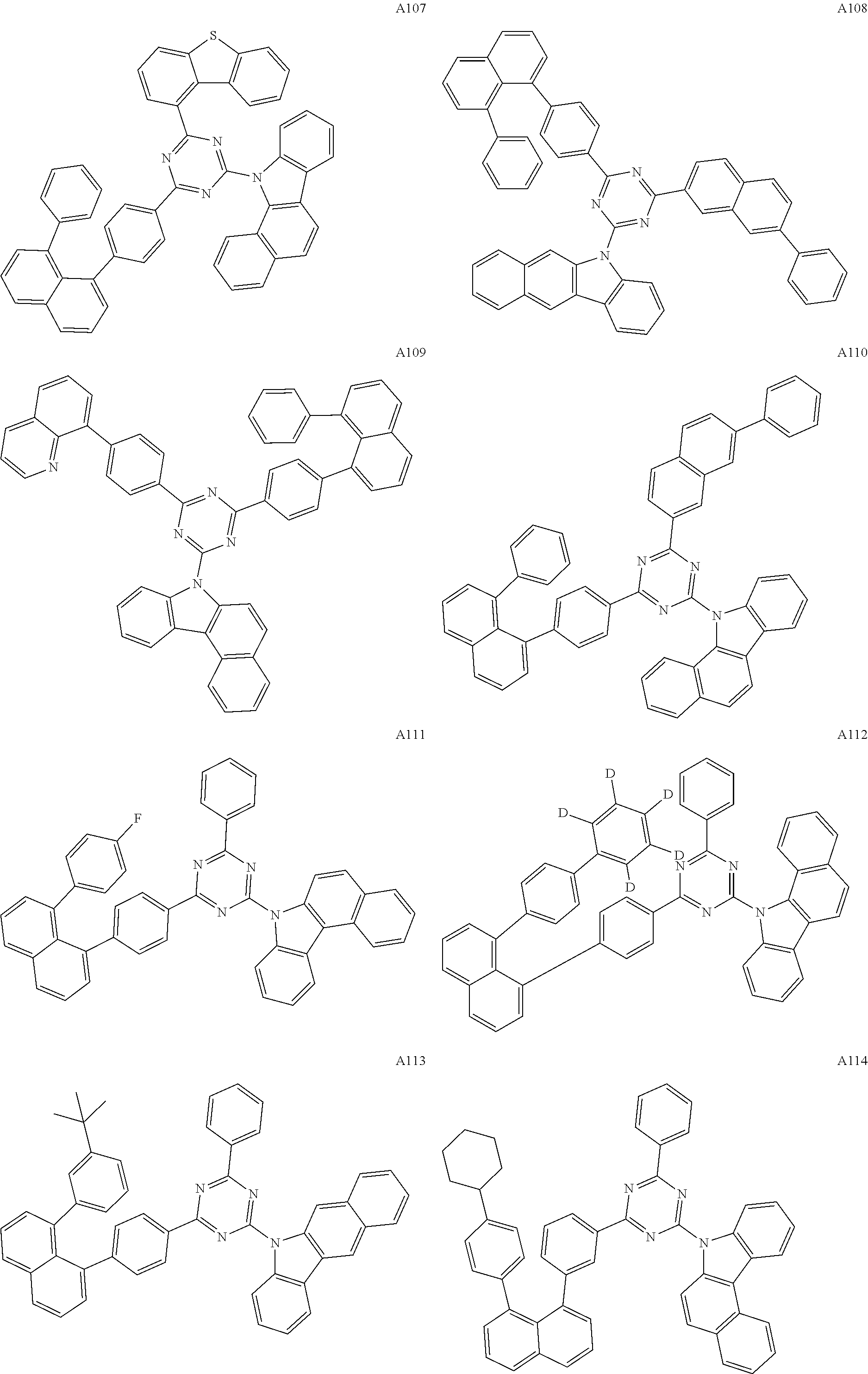

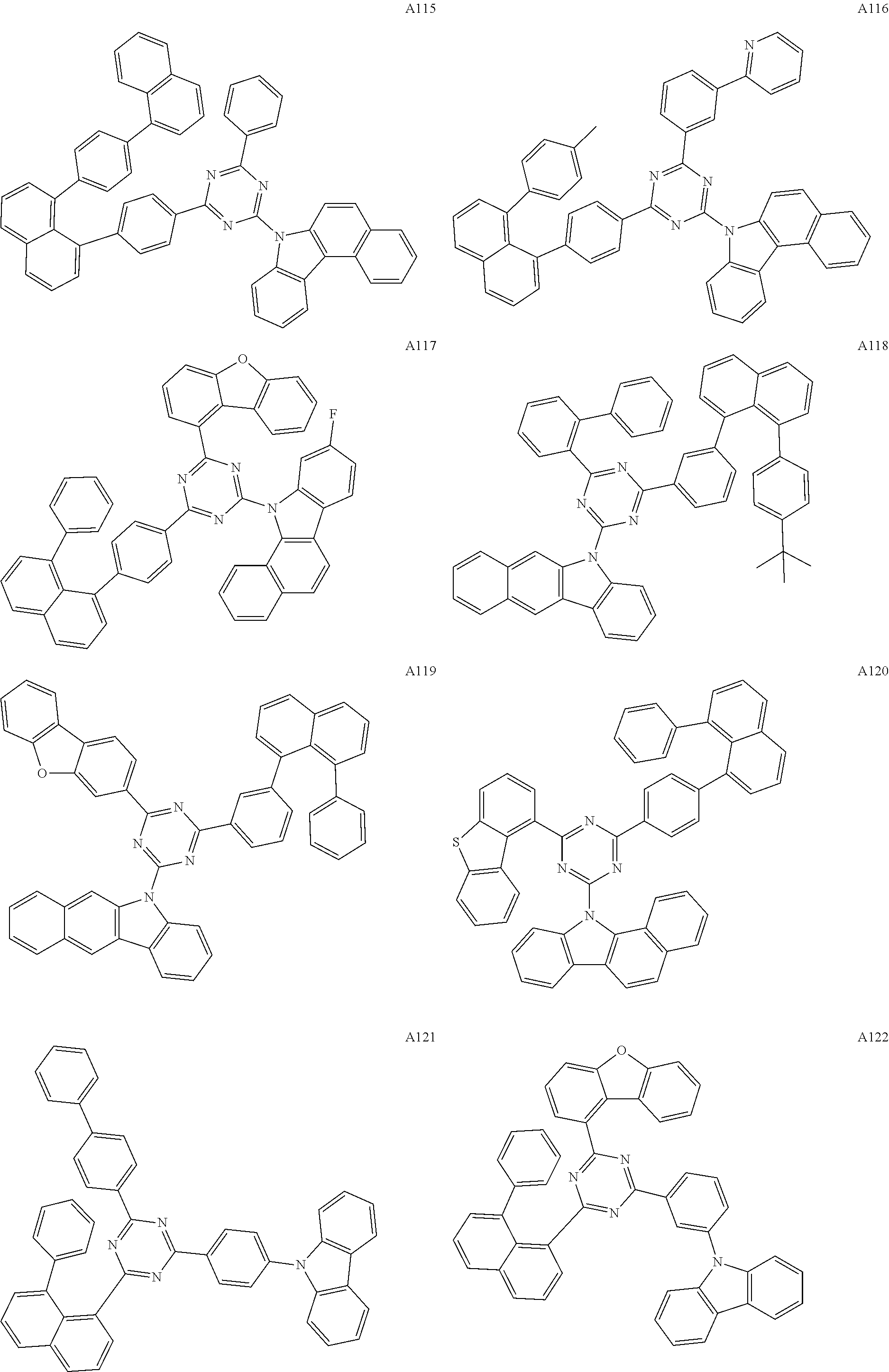

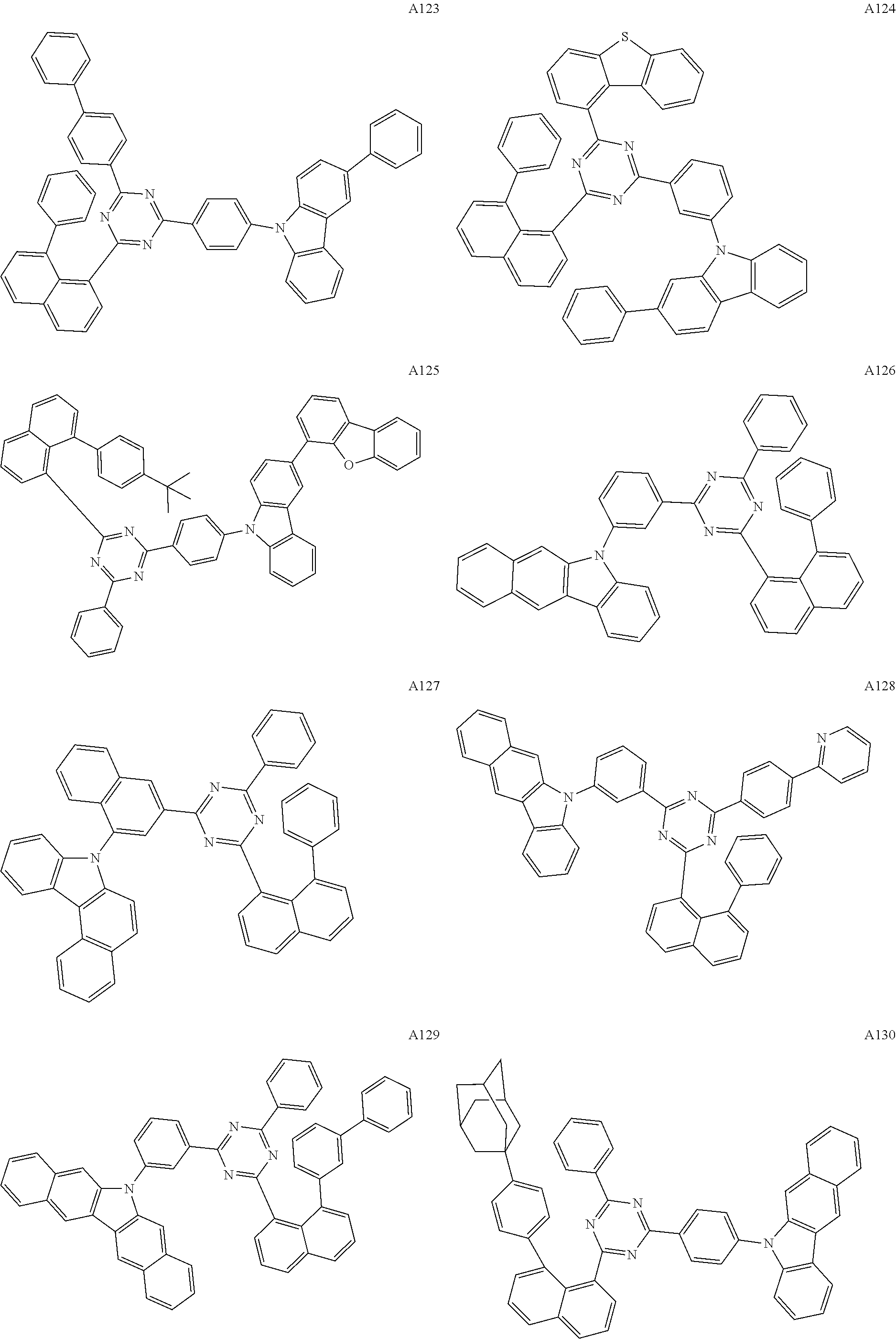

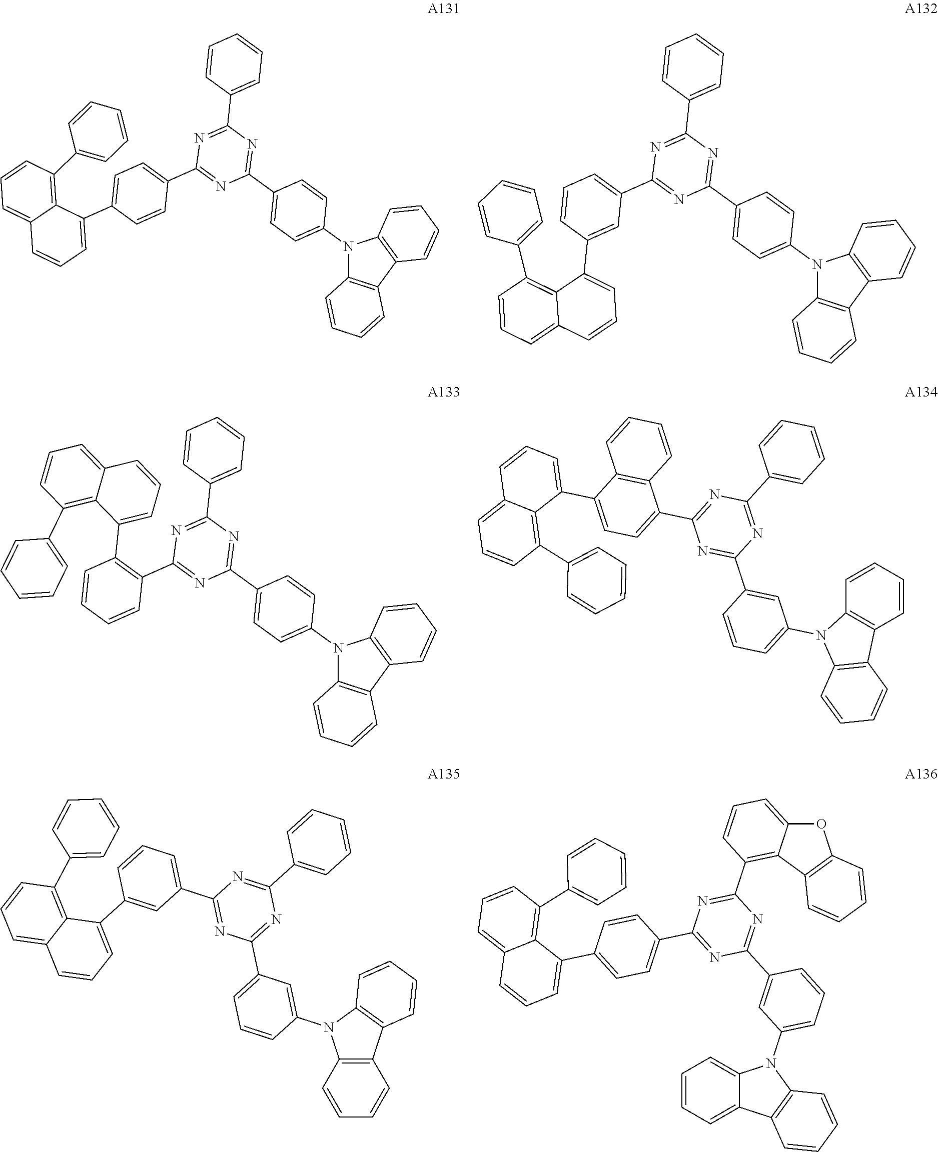

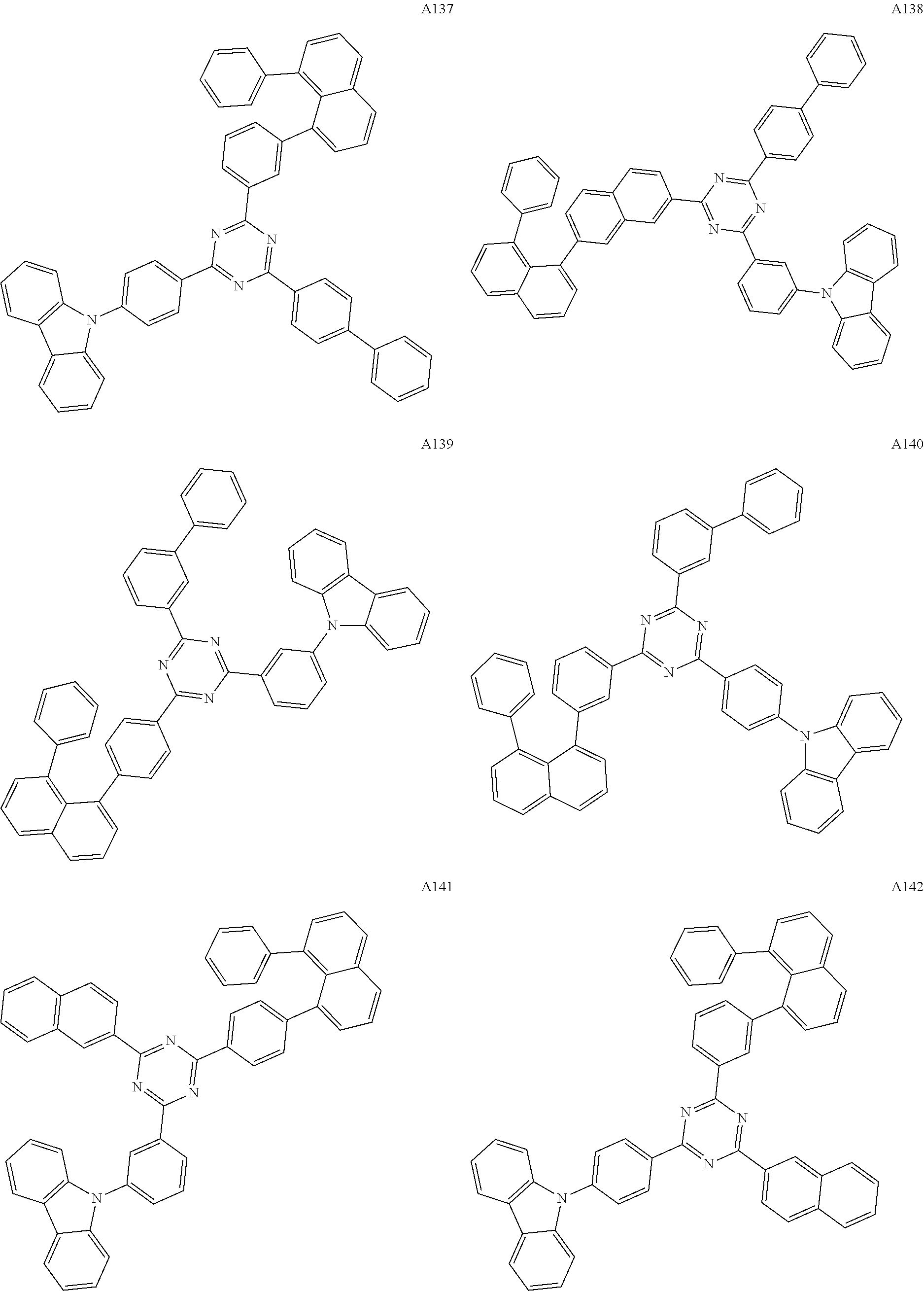

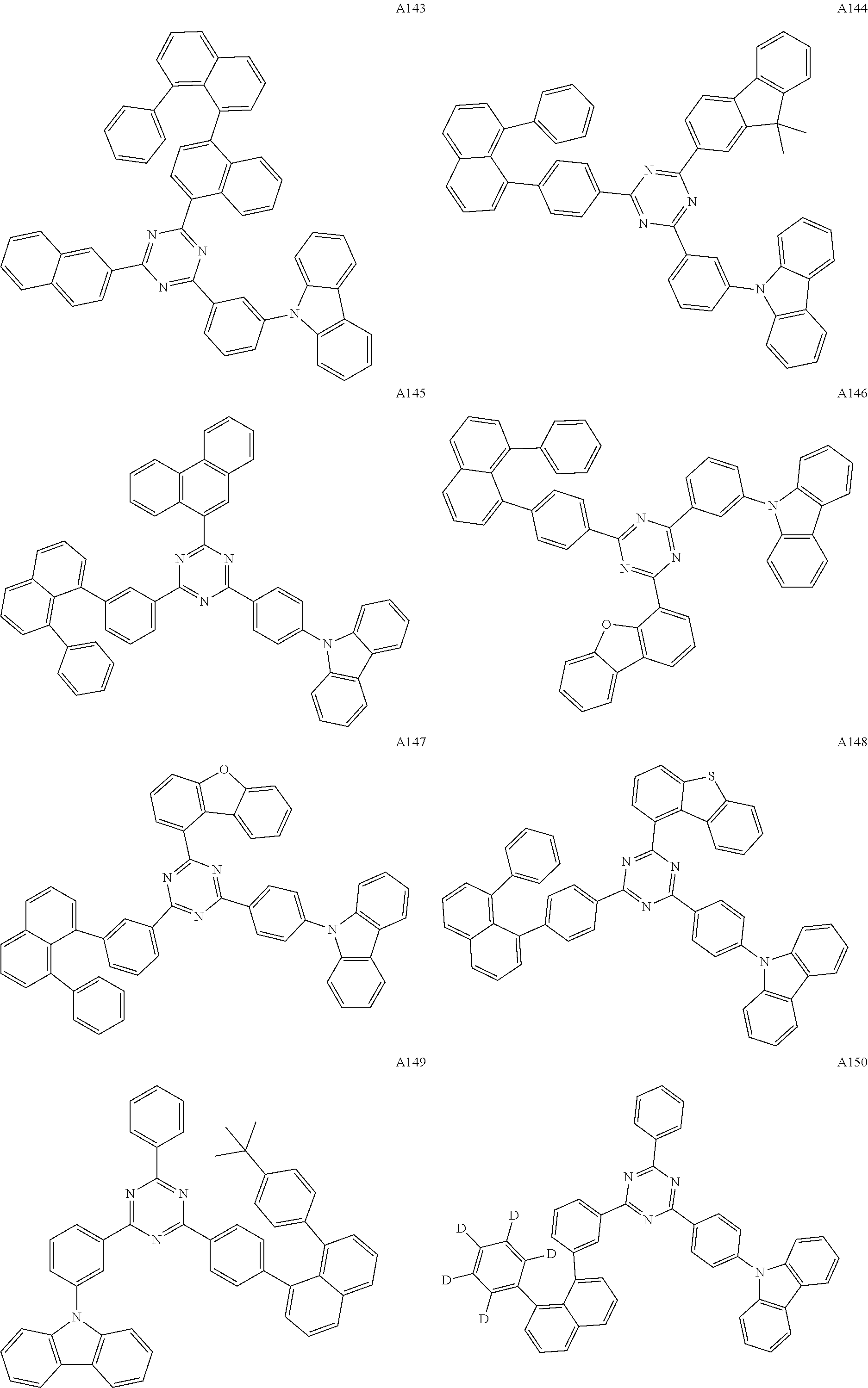

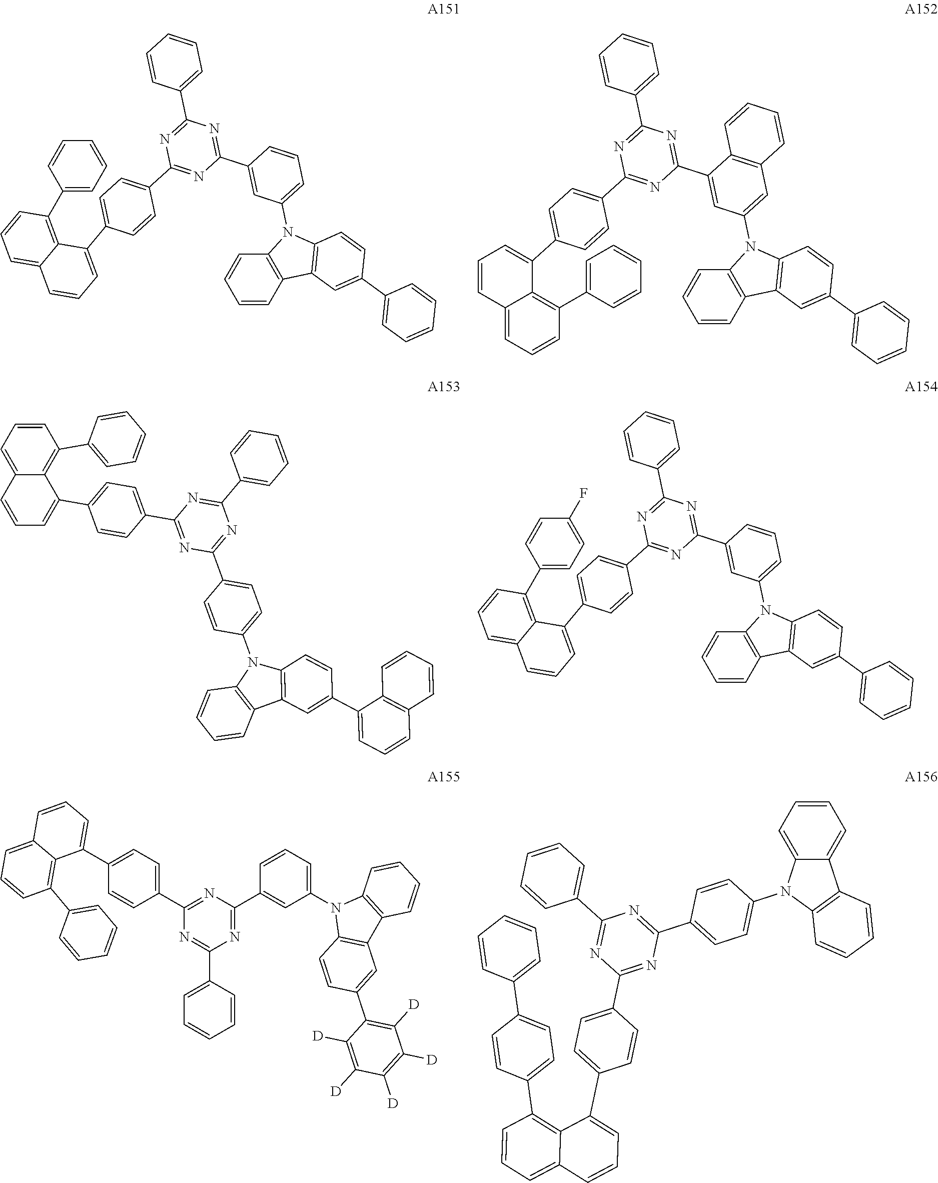

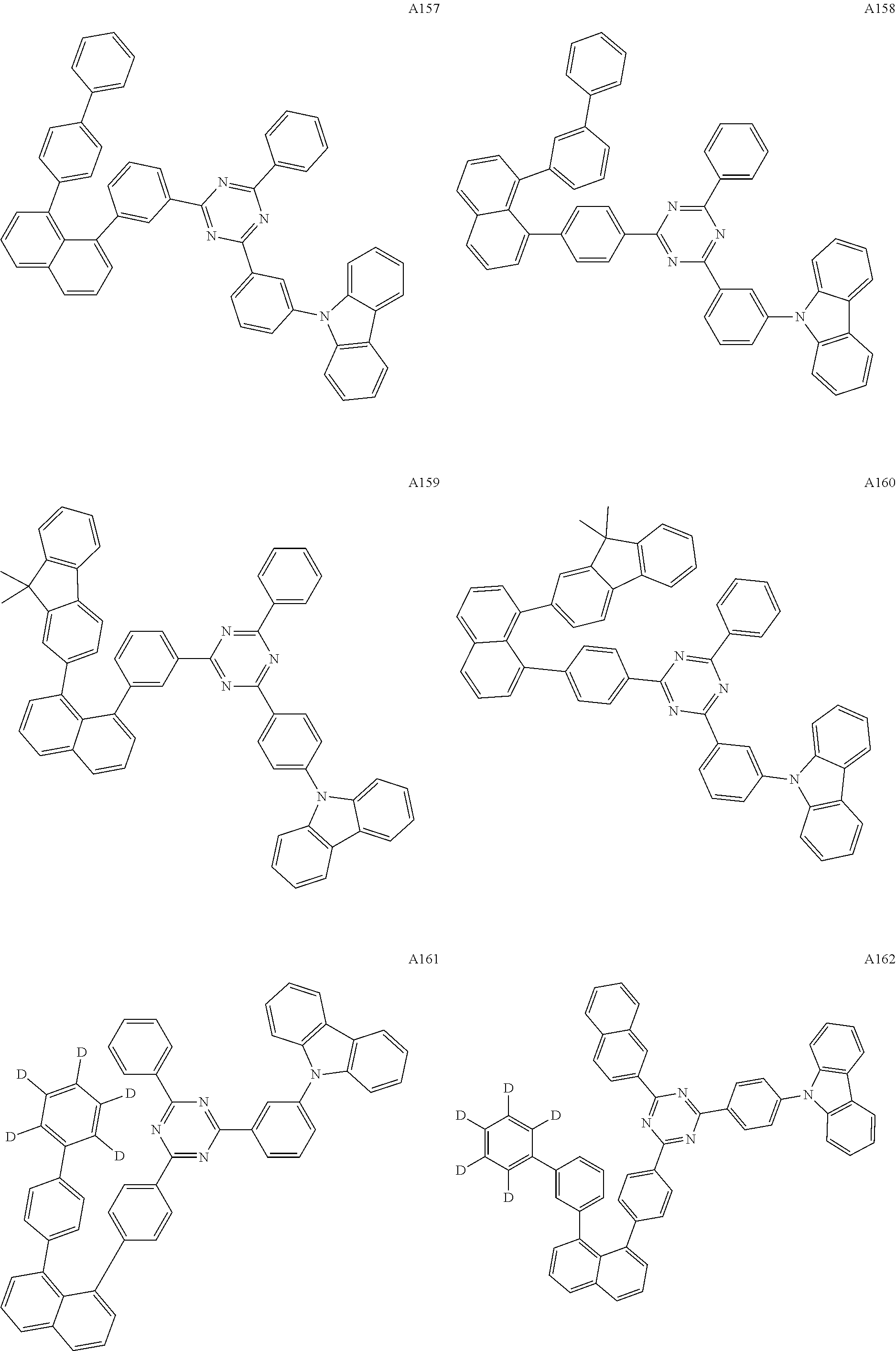

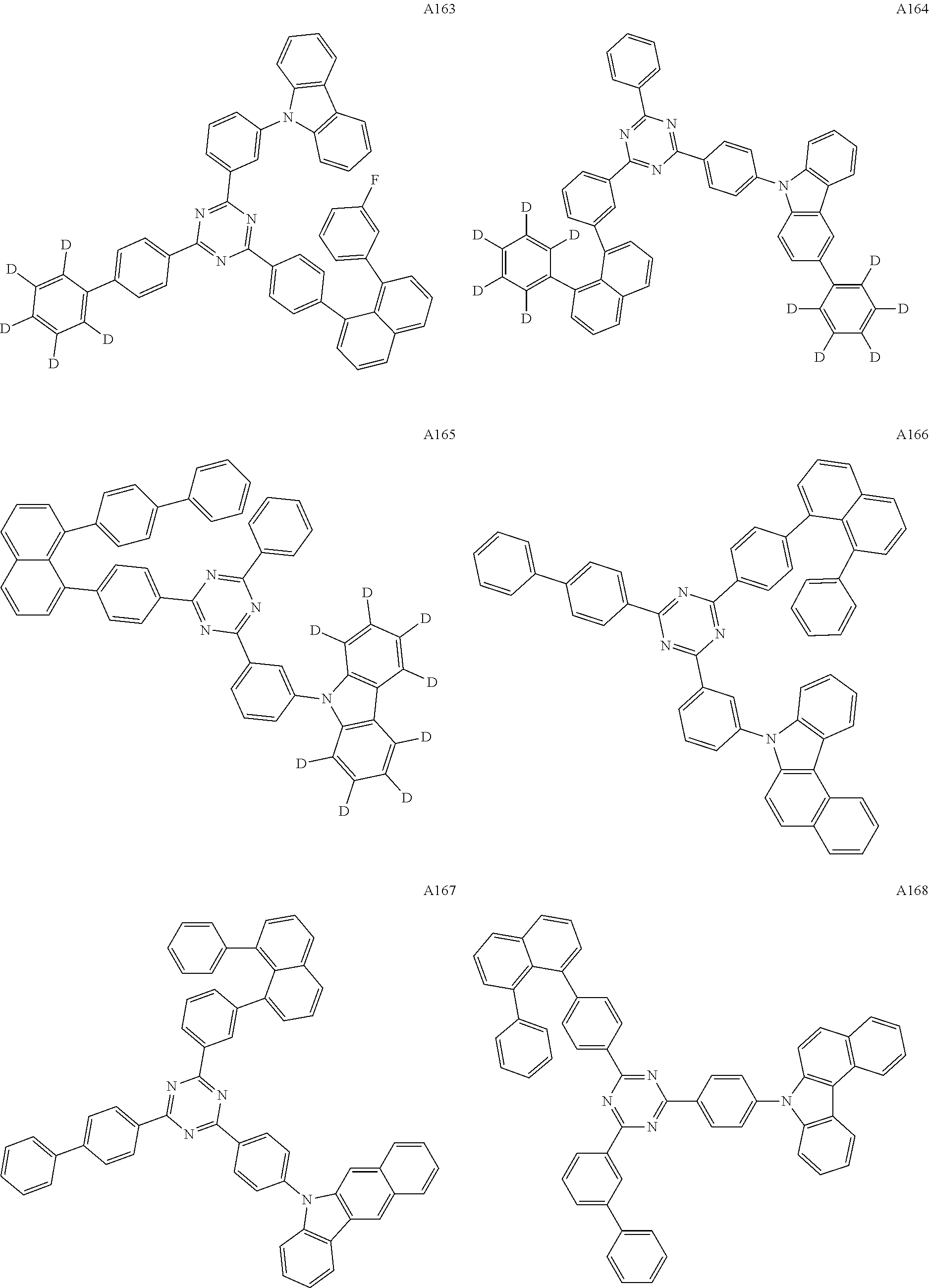

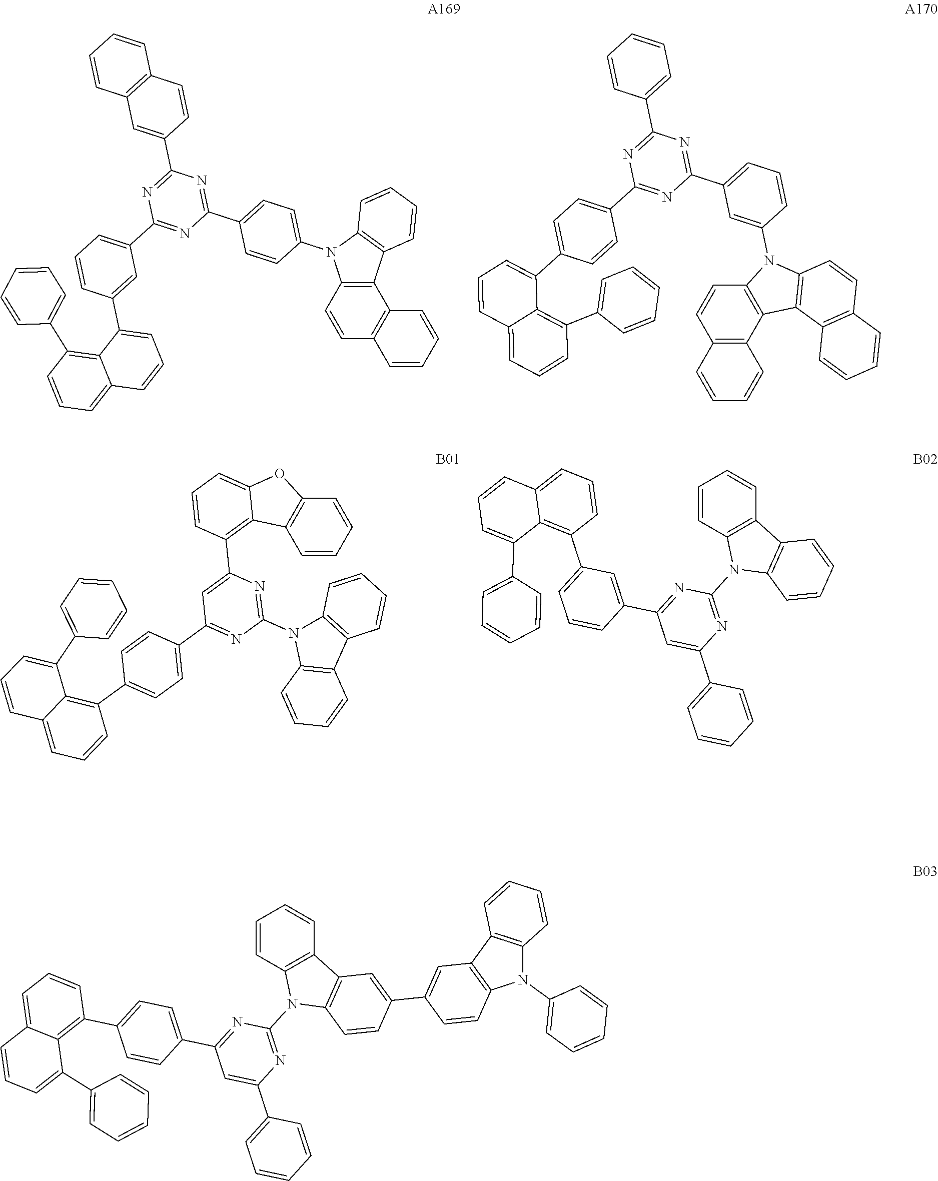

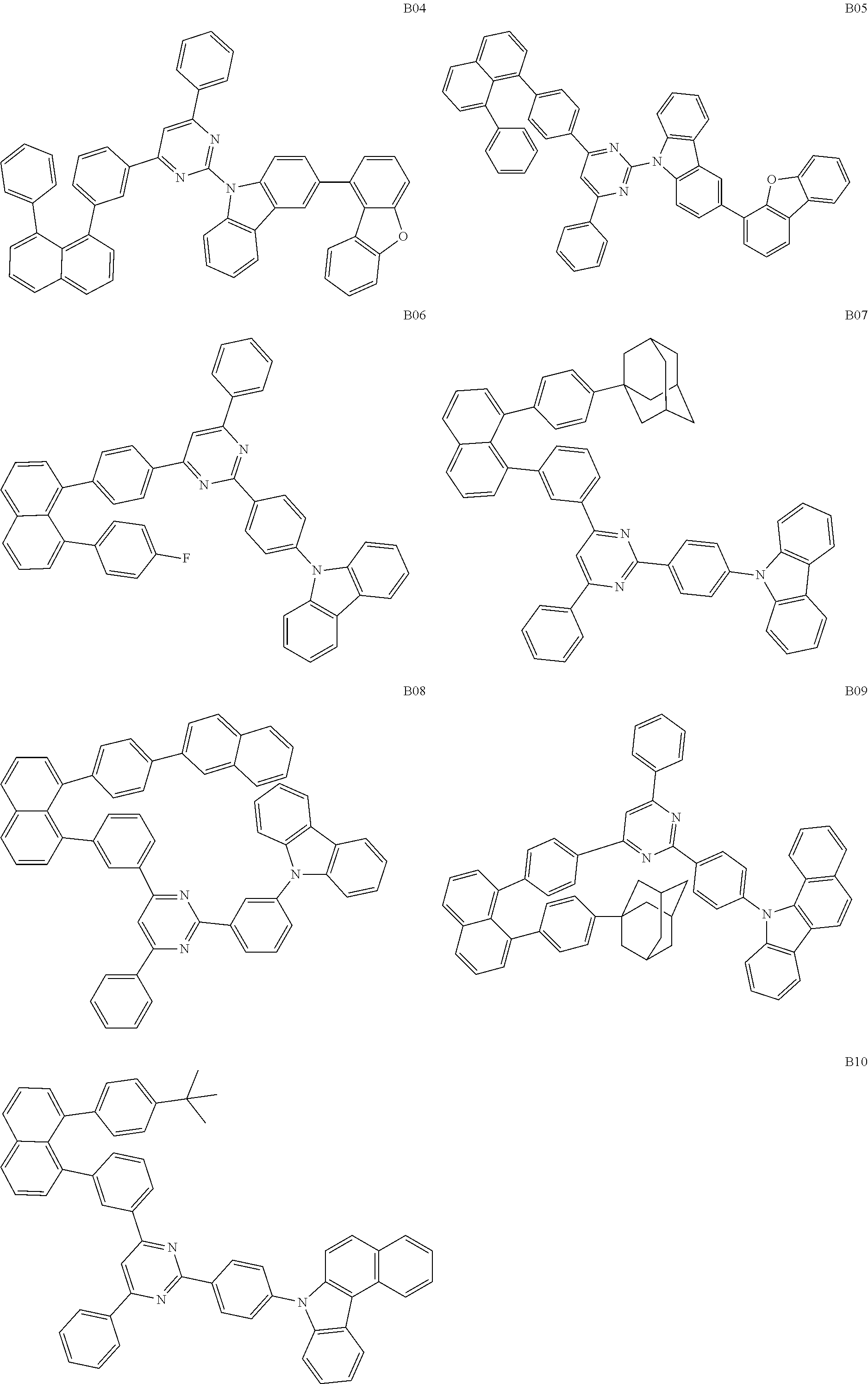

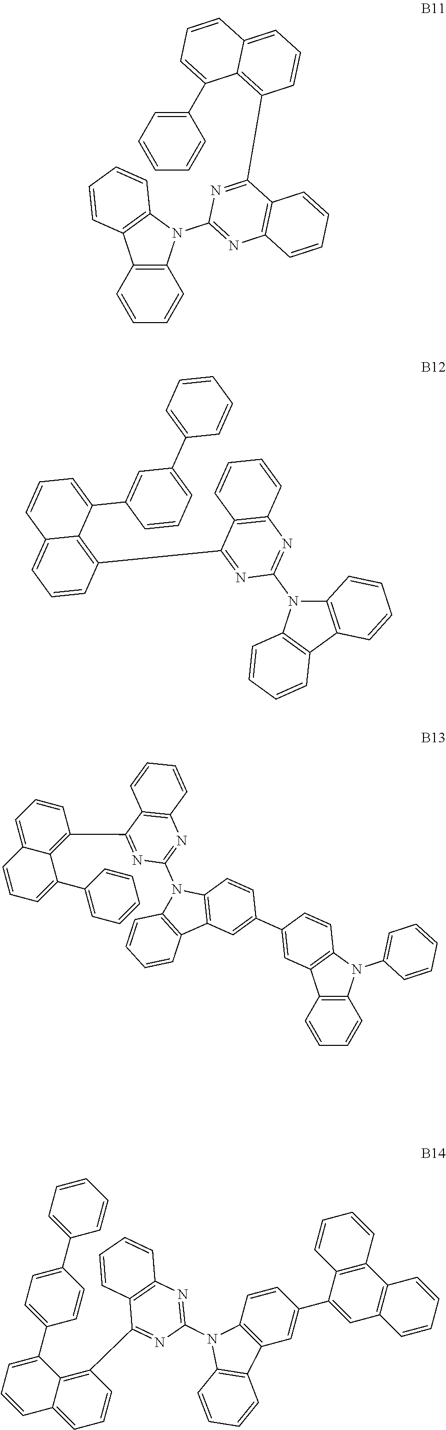 [SMILES: c1ccc(-c2cccc3cccc(-c4nc(-n5c6ccccc6c6ccccc65)nc5ccccc45)c23)cc1 ; c1ccc(-c2cccc(-c3cccc4cccc(-c5nc(-n6c7ccccc7c7ccccc76)nc6ccccc56)c34)c2)cc1 ; c1ccc(-c2cccc3cccc(-c4nc(-n5c6ccccc6c6cc(-c7ccc8c(c7)c7ccccc7n8-c7ccccc7)ccc65)nc5ccccc45)c23)cc1 ; c1ccc(-c2ccc(-c3cccc4cccc(-c5nc(-n6c7ccccc7c7cc(-c8cc9ccccc9c9ccccc89)ccc76)nc6ccccc56)c34)cc2)cc1]

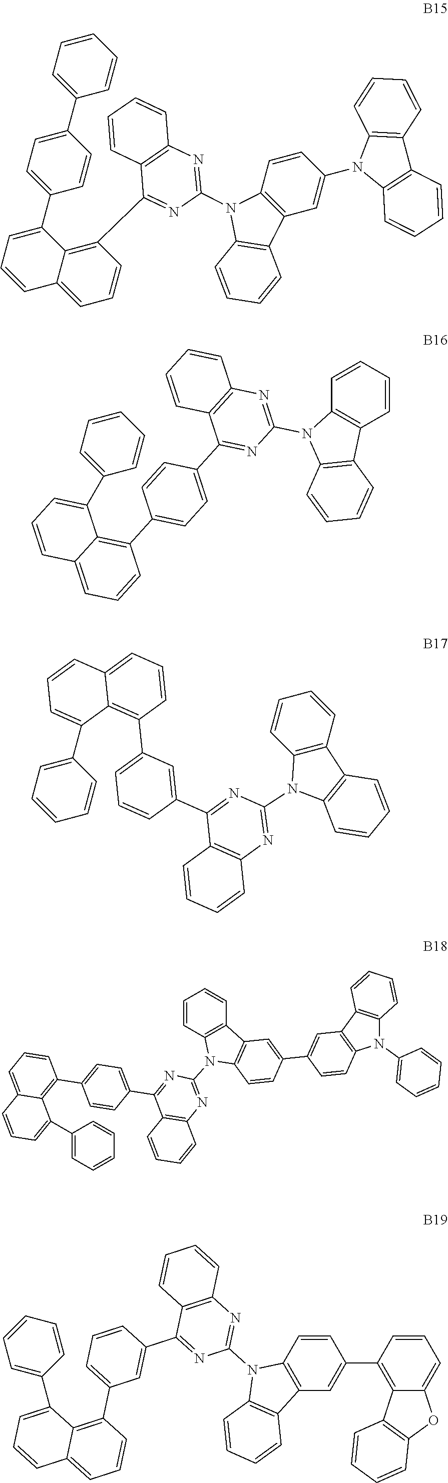 [SMILES: c1ccc(-c2ccc(-c3cccc4cccc(-c5nc(-n6c7ccccc7c7cc(-n8c9ccccc9c9ccccc98)ccc76)nc6ccccc56)c34)cc2)cc1 ; c1ccc(-c2cccc3cccc(-c4ccc(-c5nc(-n6c7ccccc7c7ccccc76)nc6ccccc56)cc4)c23)cc1 ; c1ccc(-c2cccc3cccc(-c4cccc(-c5nc(-n6c7ccccc7c7ccccc76)nc6ccccc56)c4)c23)cc1 ; c1ccc(-c2cccc3cccc(-c4ccc(-c5nc(-n6c7ccccc7c7cc(-c8ccc9c(c8)c8ccccc8n9-c8ccccc8)ccc76)nc6ccccc56)cc4)c23)cc1 ; c1ccc(-c2cccc3cccc(-c4cccc(-c5nc(-n6c7ccccc7c7cc(-c8cccc9oc%10ccccc%10c89)ccc76)nc6ccccc56)c4)c23)cc1]

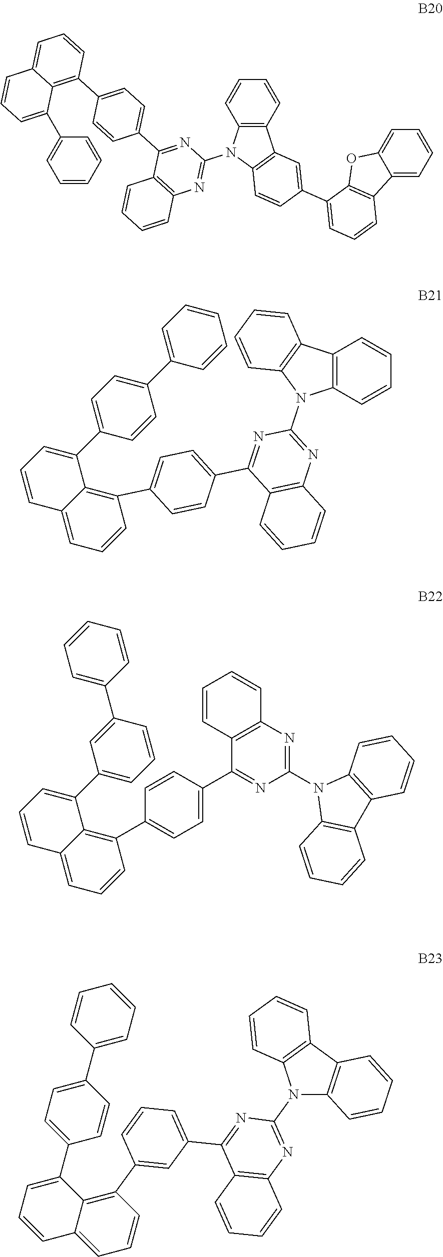 [SMILES: c1ccc(-c2cccc3cccc(-c4ccc(-c5nc(-n6c7ccccc7c7cc(-c8cccc9c8oc8ccccc89)ccc76)nc6ccccc56)cc4)c23)cc1 ; c1ccc(-c2ccc(-c3cccc4cccc(-c5ccc(-c6nc(-n7c8ccccc8c8ccccc87)nc7ccccc67)cc5)c34)cc2)cc1 ; c1ccc(-c2cccc(-c3cccc4cccc(-c5ccc(-c6nc(-n7c8ccccc8c8ccccc87)nc7ccccc67)cc5)c34)c2)cc1 ; c1ccc(-c2ccc(-c3cccc4cccc(-c5cccc(-c6nc(-n7c8ccccc8c8ccccc87)nc7ccccc67)c5)c34)cc2)cc1]

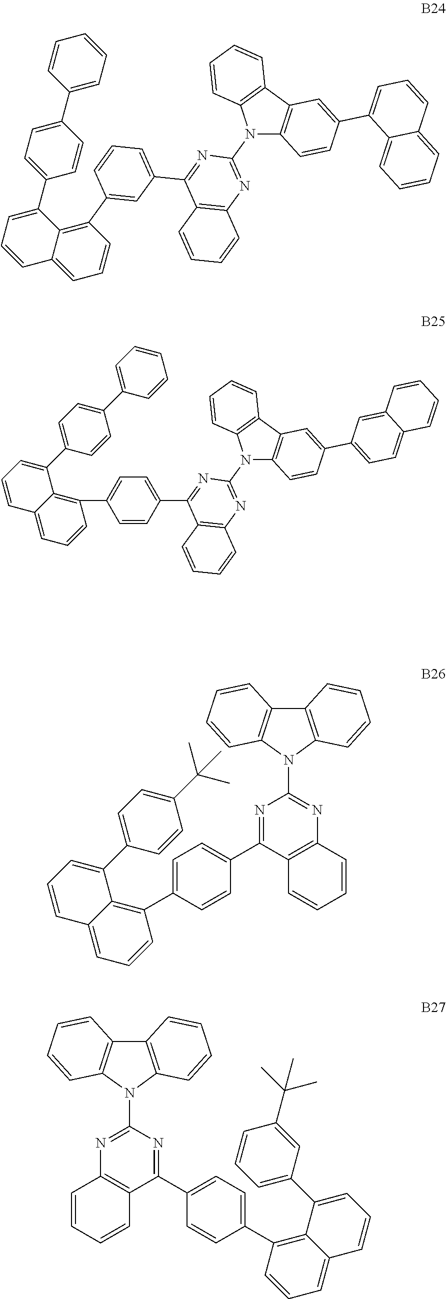 [SMILES: c1ccc(-c2ccc(-c3cccc4cccc(-c5cccc(-c6nc(-n7c8ccccc8c8cc(-c9cccc%10ccccc9%10)ccc87)nc7ccccc67)c5)c34)cc2)cc1 ; c1ccc(-c2ccc(-c3cccc4cccc(-c5ccc(-c6nc(-n7c8ccccc8c8cc(-c9ccc%10ccccc%10c9)ccc87)nc7ccccc67)cc5)c34)cc2)cc1 ; CC(C)(C)c1ccc(-c2cccc3cccc(-c4ccc(-c5nc(-n6c7ccccc7c7ccccc76)nc6ccccc56)cc4)c23)cc1 ; CC(C)(C)c1cccc(-c2cccc3cccc(-c4ccc(-c5nc(-n6c7ccccc7c7ccccc76)nc6ccccc56)cc4)c23)c1]

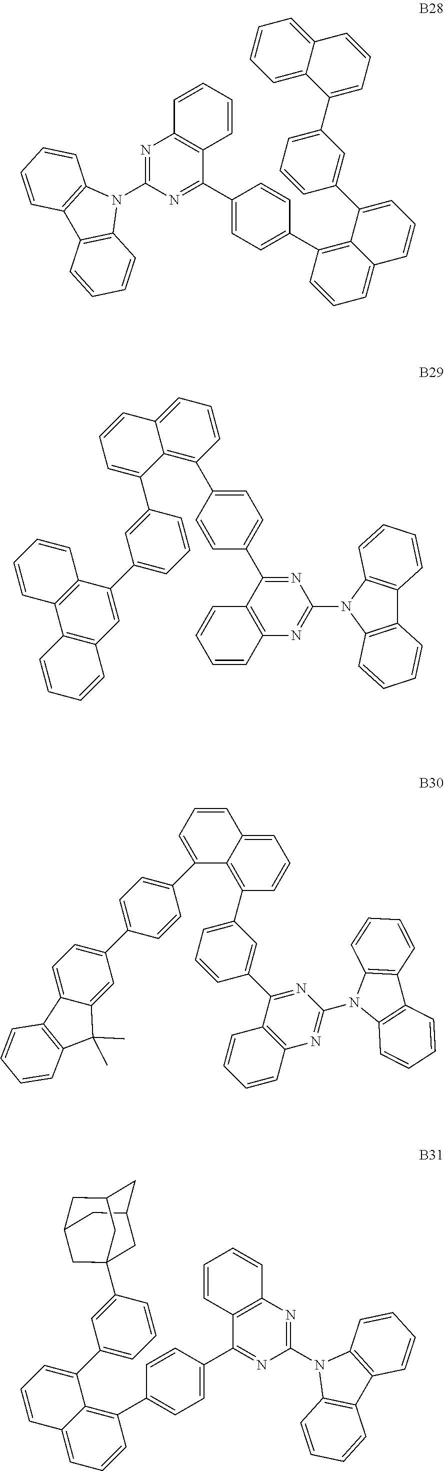 [SMILES: c1cc(-c2cccc3ccccc23)cc(-c2cccc3cccc(-c4ccc(-c5nc(-n6c7ccccc7c7ccccc76)nc6ccccc56)cc4)c23)c1 ; c1cc(-c2cc3ccccc3c3ccccc23)cc(-c2cccc3cccc(-c4ccc(-c5nc(-n6c7ccccc7c7ccccc76)nc6ccccc56)cc4)c23)c1 ; CC1(C)c2ccccc2-c2ccc(-c3ccc(-c4cccc5cccc(-c6cccc(-c7nc(-n8c9ccccc9c9ccccc98)nc8ccccc78)c6)c45)cc3)cc21 ; c1cc(-c2cccc3cccc(-c4ccc(-c5nc(-n6c7ccccc7c7ccccc76)nc6ccccc56)cc4)c23)cc(C23CC4CC(CC(C4)C2)C3)c1]

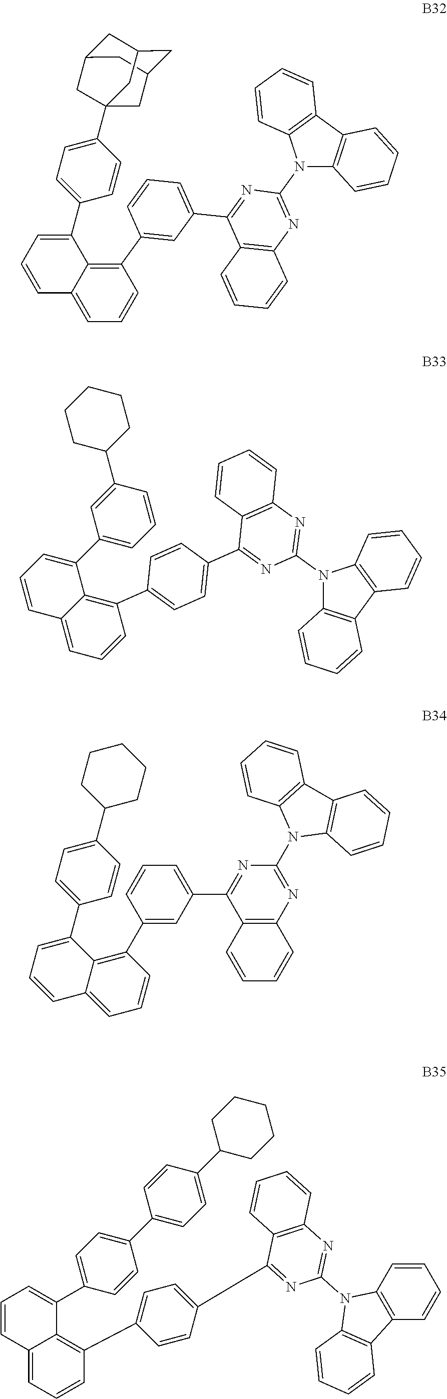 [SMILES: c1cc(-c2nc(-n3c4ccccc4c4ccccc43)nc3ccccc23)cc(-c2cccc3cccc(-c4ccc(C56CC7CC(CC(C7)C5)C6)cc4)c23)c1 ; c1cc(-c2cccc3cccc(-c4ccc(-c5nc(-n6c7ccccc7c7ccccc76)nc6ccccc56)cc4)c23)cc(C2CCCCC2)c1 ; c1cc(-c2nc(-n3c4ccccc4c4ccccc43)nc3ccccc23)cc(-c2cccc3cccc(-c4ccc(C5CCCCC5)cc4)c23)c1 ; c1cc(-c2ccc(-c3ccc(C4CCCCC4)cc3)cc2)c2c(-c3ccc(-c4nc(-n5c6ccccc6c6ccccc65)nc5ccccc45)cc3)cccc2c1]

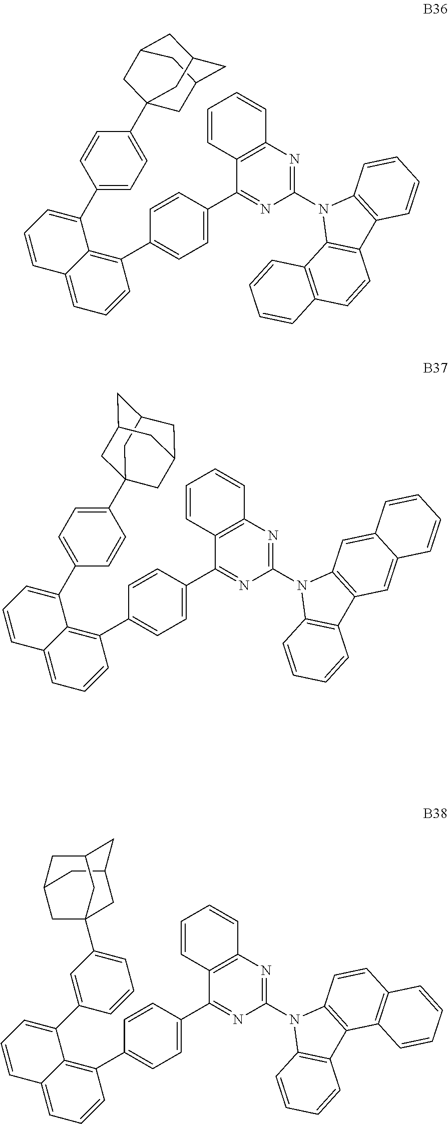 [SMILES: c1cc(-c2ccc(-c3nc(-n4c5ccccc5c5ccc6ccccc6c54)nc4ccccc34)cc2)c2c(-c3ccc(C45CC6CC(CC(C6)C4)C5)cc3)cccc2c1 ; c1ccc2cc3c(cc2c1)c1ccccc1n3-c1nc(-c2ccc(-c3cccc4cccc(-c5ccc(C67CC8CC(CC(C8)C6)C7)cc5)c34)cc2)c2ccccc2n1 ; c1cc(-c2cccc3cccc(-c4ccc(-c5nc(-n6c7ccccc7c7c8ccccc8ccc76)nc6ccccc56)cc4)c23)cc(C23CC4CC(CC(C4)C2)C3)c1]

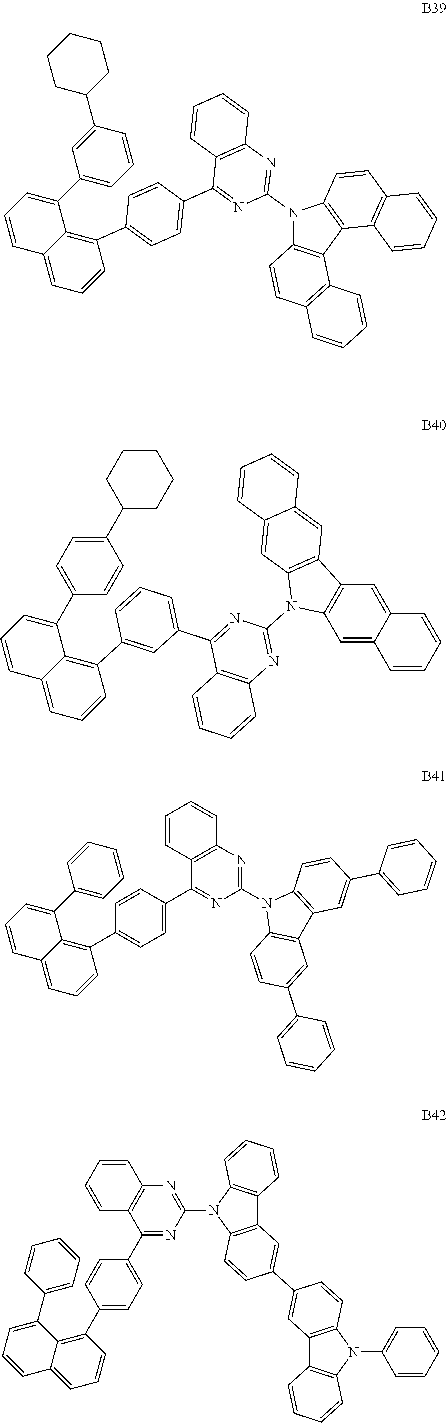 [SMILES: c1cc(-c2cccc3cccc(-c4ccc(-c5nc(-n6c7ccc8ccccc8c7c7c8ccccc8ccc76)nc6ccccc56)cc4)c23)cc(C2CCCCC2)c1 ; c1cc(-c2nc(-n3c4cc5ccccc5cc4c4cc5ccccc5cc43)nc3ccccc23)cc(-c2cccc3cccc(-c4ccc(C5CCCCC5)cc4)c23)c1 ; c1ccc(-c2ccc3c(c2)c2cc(-c4ccccc4)ccc2n3-c2nc(-c3ccc(-c4cccc5cccc(-c6ccccc6)c45)cc3)c3ccccc3n2)cc1 ; c1ccc(-c2cccc3cccc(-c4ccc(-c5nc(-n6c7ccccc7c7cc(-c8ccc9c(c8)c8ccccc8n9-c8ccccc8)ccc76)nc6ccccc56)cc4)c23)cc1]

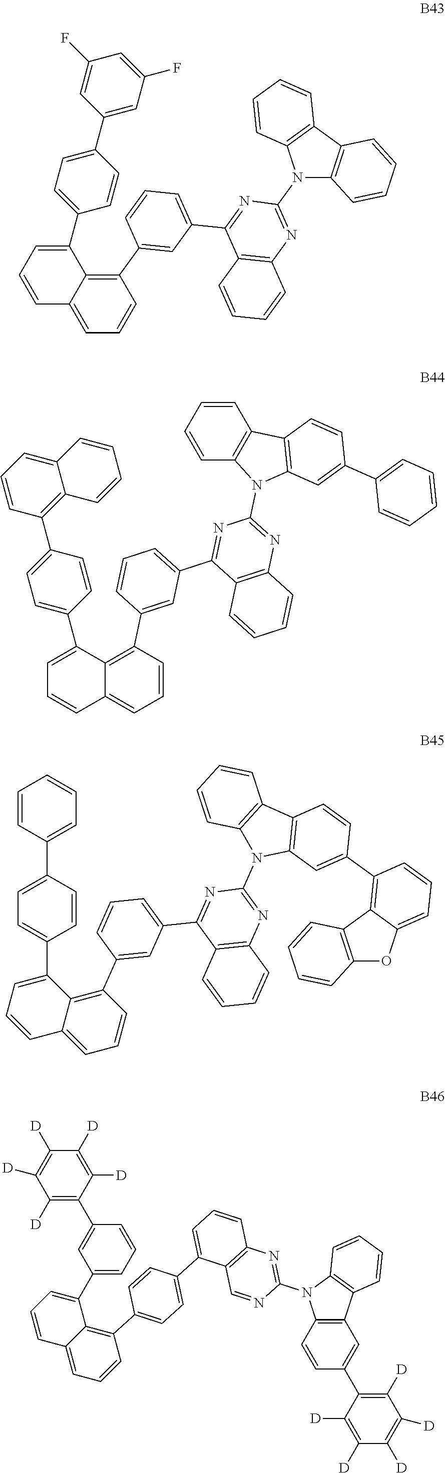 [SMILES: Fc1cc(F)cc(-c2ccc(-c3cccc4cccc(-c5cccc(-c6nc(-n7c8ccccc8c8ccccc87)nc7ccccc67)c5)c34)cc2)c1 ; c1ccc(-c2ccc3c4ccccc4n(-c4nc(-c5cccc(-c6cccc7cccc(-c8ccc(-c9cccc%10ccccc9%10)cc8)c67)c5)c5ccccc5n4)c3c2)cc1 ; c1ccc(-c2ccc(-c3cccc4cccc(-c5cccc(-c6nc(-n7c8ccccc8c8ccc(-c9cccc%10oc%11ccccc%11c9%10)cc87)nc7ccccc67)c5)c34)cc2)cc1 ; [2H]c1c([2H])c([2H])c(-c2cccc(-c3cccc4cccc(-c5ccc(-c6cccc7nc(-n8c9ccccc9c9cc(-c%10c([2H])c([2H])c([2H])c([2H])c%10[2H])ccc98)ncc67)cc5)c34)c2)c([2H])c1[2H]]

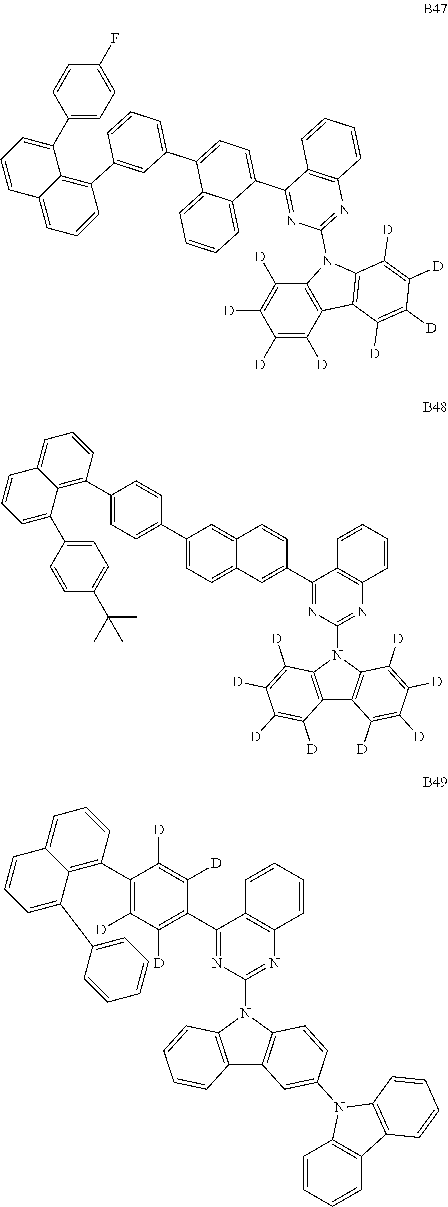 [SMILES: [2H]c1c([2H])c([2H])c2c(c1[2H])c1c([2H])c([2H])c([2H])c([2H])c1n2-c1nc(-c2ccc(-c3cccc(-c4cccc5cccc(-c6ccc(F)cc6)c45)c3)c3ccccc23)c2ccccc2n1 ; [2H]c1c([2H])c([2H])c2c(c1[2H])c1c([2H])c([2H])c([2H])c([2H])c1n2-c1nc(-c2ccc3cc(-c4ccc(-c5cccc6cccc(-c7ccc(C(C)(C)C)cc7)c56)cc4)ccc3c2)c2ccccc2n1 ; [2H]c1c([2H])c(-c2cccc3cccc(-c4ccccc4)c23)c([2H])c([2H])c1-c1nc(-n2c3ccccc3c3cc(-n4c5ccccc5c5ccccc54)ccc32)nc2ccccc12]

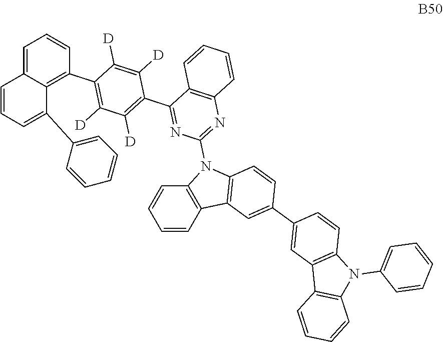 [SMILES: [2H]c1c([2H])c(-c2cccc3cccc(-c4ccccc4)c23)c([2H])c([2H])c1-c1nc(-n2c3ccccc3c3cc(-c4ccc5c(c4)c4ccccc4n5-c4ccccc4)ccc32)nc2ccccc12]

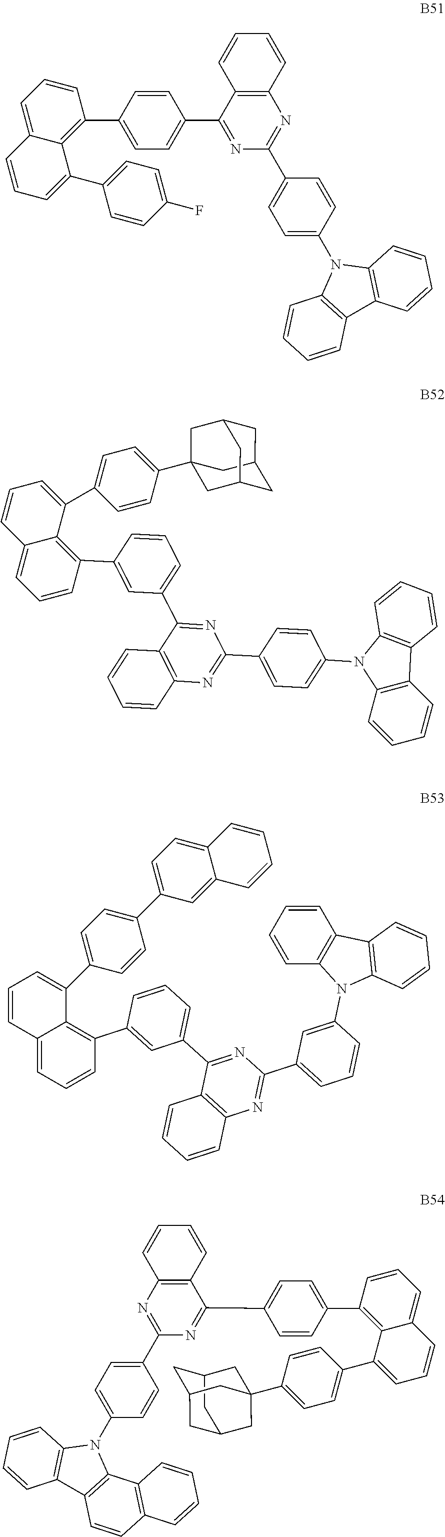 [SMILES: Fc1ccc(-c2cccc3cccc(-c4ccc(-c5nc(-c6ccc(-n7c8ccccc8c8ccccc87)cc6)nc6ccccc56)cc4)c23)cc1 ; c1cc(-c2nc(-c3ccc(-n4c5ccccc5c5ccccc54)cc3)nc3ccccc23)cc(-c2cccc3cccc(-c4ccc(C56CC7CC(CC(C7)C5)C6)cc4)c23)c1 ; c1cc(-c2nc(-c3cccc(-n4c5ccccc5c5ccccc54)c3)nc3ccccc23)cc(-c2cccc3cccc(-c4ccc(-c5ccc6ccccc6c5)cc4)c23)c1 ; c1cc(-c2ccc(-c3nc(-c4ccc(-n5c6ccccc6c6ccc7ccccc7c65)cc4)nc4ccccc34)cc2)c2c(-c3ccc(C45CC6CC(CC(C6)C4)C5)cc3)cccc2c1]

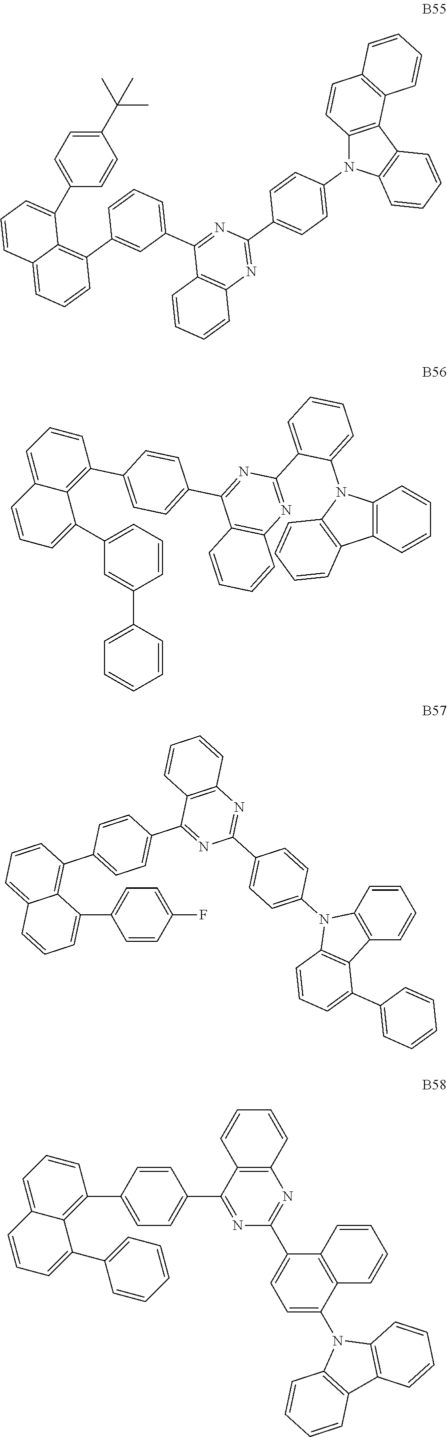 [SMILES: CC(C)(C)c1ccc(-c2cccc3cccc(-c4cccc(-c5nc(-c6ccc(-n7c8ccccc8c8c9ccccc9ccc87)cc6)nc6ccccc56)c4)c23)cc1 ; c1ccc(-c2cccc(-c3cccc4cccc(-c5ccc(-c6nc(-c7ccccc7-n7c8ccccc8c8ccccc87)nc7ccccc67)cc5)c34)c2)cc1 ; Fc1ccc(-c2cccc3cccc(-c4ccc(-c5nc(-c6ccc(-n7c8ccccc8c8c(-c9ccccc9)cccc87)cc6)nc6ccccc56)cc4)c23)cc1 ; c1ccc(-c2cccc3cccc(-c4ccc(-c5nc(-c6ccc(-n7c8ccccc8c8ccccc87)c7ccccc67)nc6ccccc56)cc4)c23)cc1]

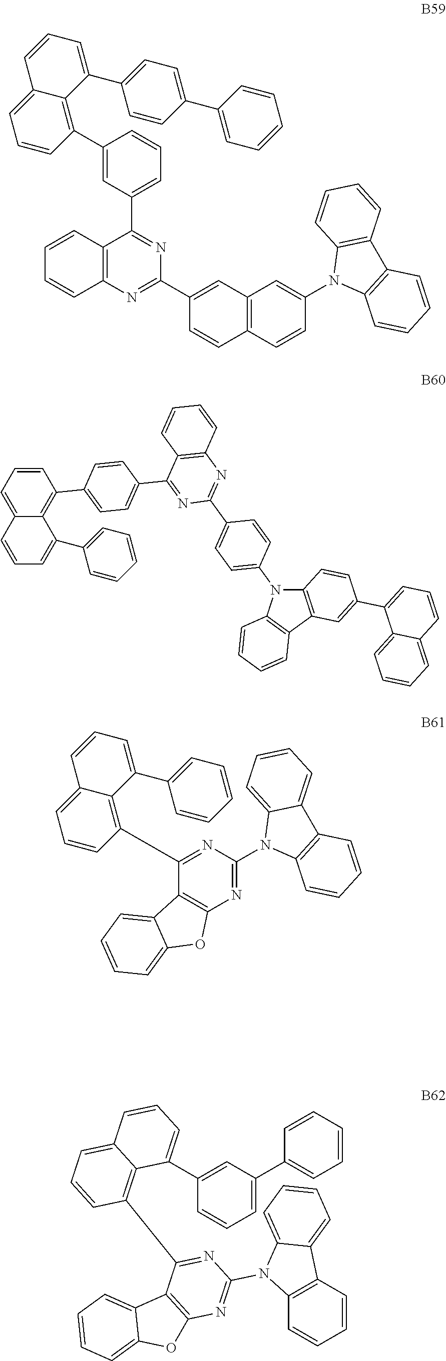 [SMILES: c1ccc(-c2ccc(-c3cccc4cccc(-c5cccc(-c6nc(-c7ccc8ccc(-n9c%10ccccc%10c%10ccccc%109)cc8c7)nc7ccccc67)c5)c34)cc2)cc1 ; c1ccc(-c2cccc3cccc(-c4ccc(-c5nc(-c6ccc(-n7c8ccccc8c8cc(-c9cccc%10ccccc9%10)ccc87)cc6)nc6ccccc56)cc4)c23)cc1 ; c1ccc(-c2cccc3cccc(-c4nc(-n5c6ccccc6c6ccccc65)nc5oc6ccccc6c45)c23)cc1 ; c1ccc(-c2cccc(-c3cccc4cccc(-c5nc(-n6c7ccccc7c7ccccc76)nc6oc7ccccc7c56)c34)c2)cc1]

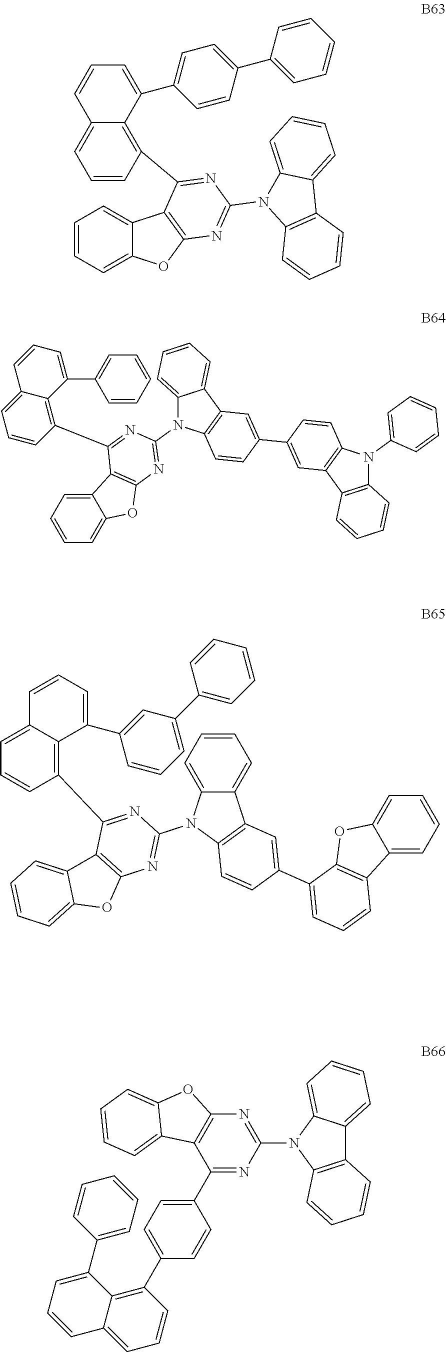 [SMILES: c1ccc(-c2ccc(-c3cccc4cccc(-c5nc(-n6c7ccccc7c7ccccc76)nc6oc7ccccc7c56)c34)cc2)cc1 ; c1ccc(-c2cccc3cccc(-c4nc(-n5c6ccccc6c6cc(-c7ccc8c(c7)c7ccccc7n8-c7ccccc7)ccc65)nc5oc6ccccc6c45)c23)cc1 ; c1ccc(-c2cccc(-c3cccc4cccc(-c5nc(-n6c7ccccc7c7cc(-c8cccc9c8oc8ccccc89)ccc76)nc6oc7ccccc7c56)c34)c2)cc1 ; c1ccc(-c2cccc3cccc(-c4ccc(-c5nc(-n6c7ccccc7c7ccccc76)nc6oc7ccccc7c56)cc4)c23)cc1]

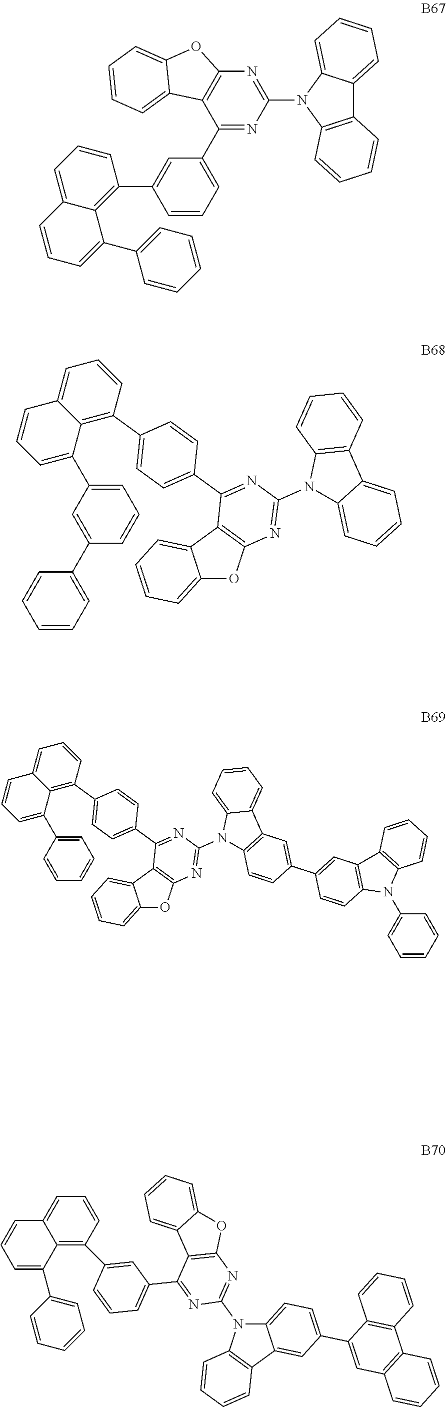 [SMILES: c1ccc(-c2cccc3cccc(-c4cccc(-c5nc(-n6c7ccccc7c7ccccc76)nc6oc7ccccc7c56)c4)c23)cc1 ; c1ccc(-c2cccc(-c3cccc4cccc(-c5ccc(-c6nc(-n7c8ccccc8c8ccccc87)nc7oc8ccccc8c67)cc5)c34)c2)cc1 ; c1ccc(-c2cccc3cccc(-c4ccc(-c5nc(-n6c7ccccc7c7cc(-c8ccc9c(c8)c8ccccc8n9-c8ccccc8)ccc76)nc6oc7ccccc7c56)cc4)c23)cc1 ; c1ccc(-c2cccc3cccc(-c4cccc(-c5nc(-n6c7ccccc7c7cc(-c8cc9ccccc9c9ccccc89)ccc76)nc6oc7ccccc7c56)c4)c23)cc1]

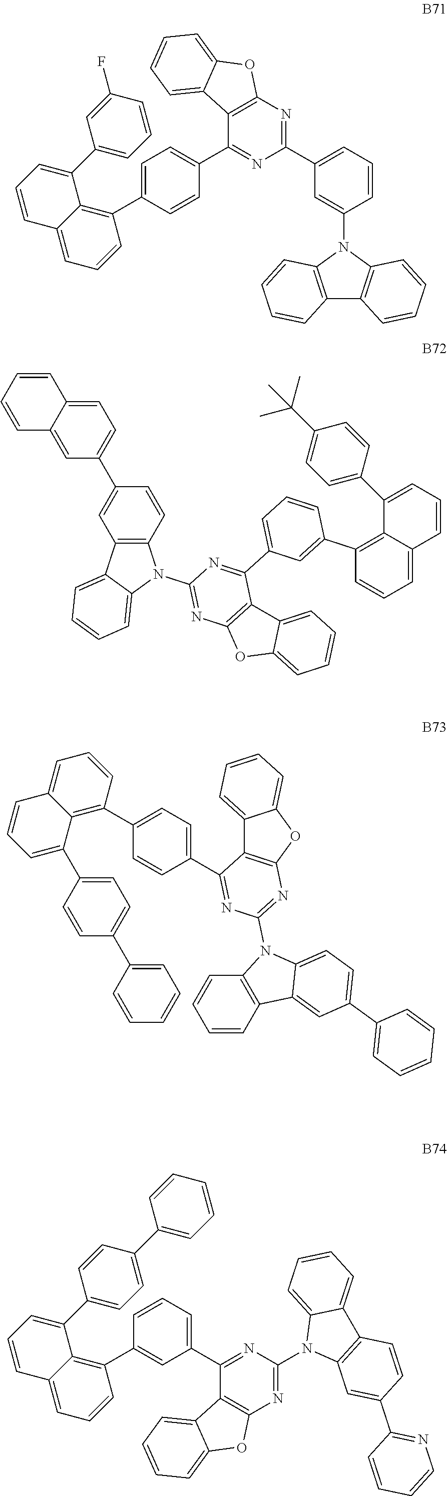 [SMILES: Fc1cccc(-c2cccc3cccc(-c4ccc(-c5nc(-c6cccc(-n7c8ccccc8c8ccccc87)c6)nc6oc7ccccc7c56)cc4)c23)c1 ; CC(C)(C)c1ccc(-c2cccc3cccc(-c4cccc(-c5nc(-n6c7ccccc7c7cc(-c8ccc9ccccc9c8)ccc76)nc6oc7ccccc7c56)c4)c23)cc1 ; c1ccc(-c2ccc(-c3cccc4cccc(-c5ccc(-c6nc(-n7c8ccccc8c8cc(-c9ccccc9)ccc87)nc7oc8ccccc8c67)cc5)c34)cc2)cc1 ; c1ccc(-c2ccc(-c3cccc4cccc(-c5cccc(-c6nc(-n7c8ccccc8c8ccc(-c9ccccn9)cc87)nc7oc8ccccc8c67)c5)c34)cc2)cc1]

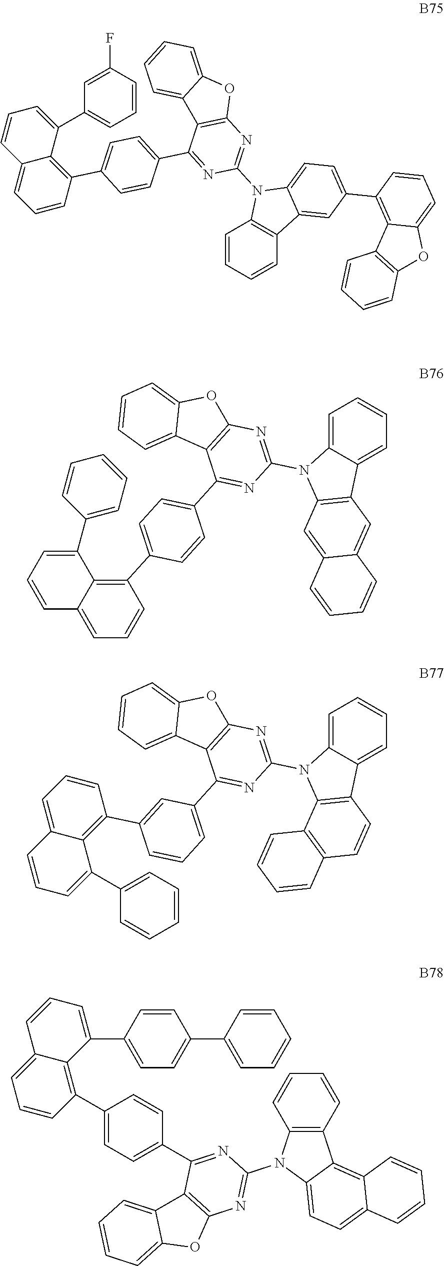 [SMILES: Fc1cccc(-c2cccc3cccc(-c4ccc(-c5nc(-n6c7ccccc7c7cc(-c8cccc9oc%10ccccc%10c89)ccc76)nc6oc7ccccc7c56)cc4)c23)c1 ; c1ccc(-c2cccc3cccc(-c4ccc(-c5nc(-n6c7ccccc7c7cc8ccccc8cc76)nc6oc7ccccc7c56)cc4)c23)cc1 ; c1ccc(-c2cccc3cccc(-c4cccc(-c5nc(-n6c7ccccc7c7ccc8ccccc8c76)nc6oc7ccccc7c56)c4)c23)cc1 ; c1ccc(-c2ccc(-c3cccc4cccc(-c5ccc(-c6nc(-n7c8ccccc8c8c9ccccc9ccc87)nc7oc8ccccc8c67)cc5)c34)cc2)cc1]

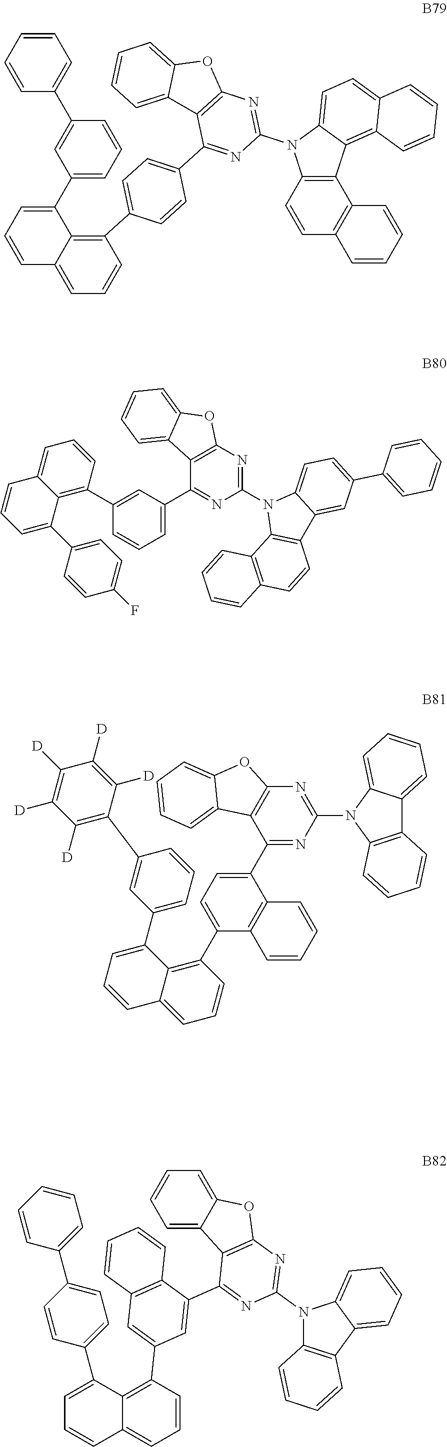 [SMILES: c1ccc(-c2cccc(-c3cccc4cccc(-c5ccc(-c6nc(-n7c8ccc9ccccc9c8c8c9ccccc9ccc87)nc7oc8ccccc8c67)cc5)c34)c2)cc1 ; Fc1ccc(-c2cccc3cccc(-c4cccc(-c5nc(-n6c7ccc(-c8ccccc8)cc7c7ccc8ccccc8c76)nc6oc7ccccc7c56)c4)c23)cc1 ; [2H]c1c([2H])c([2H])c(-c2cccc(-c3cccc4cccc(-c5ccc(-c6nc(-n7c8ccccc8c8ccccc87)nc7oc8ccccc8c67)c6ccccc56)c34)c2)c([2H])c1[2H] ; c1ccc(-c2ccc(-c3cccc4cccc(-c5cc(-c6nc(-n7c8ccccc8c8ccccc87)nc7oc8ccccc8c67)c6ccccc6c5)c34)cc2)cc1]

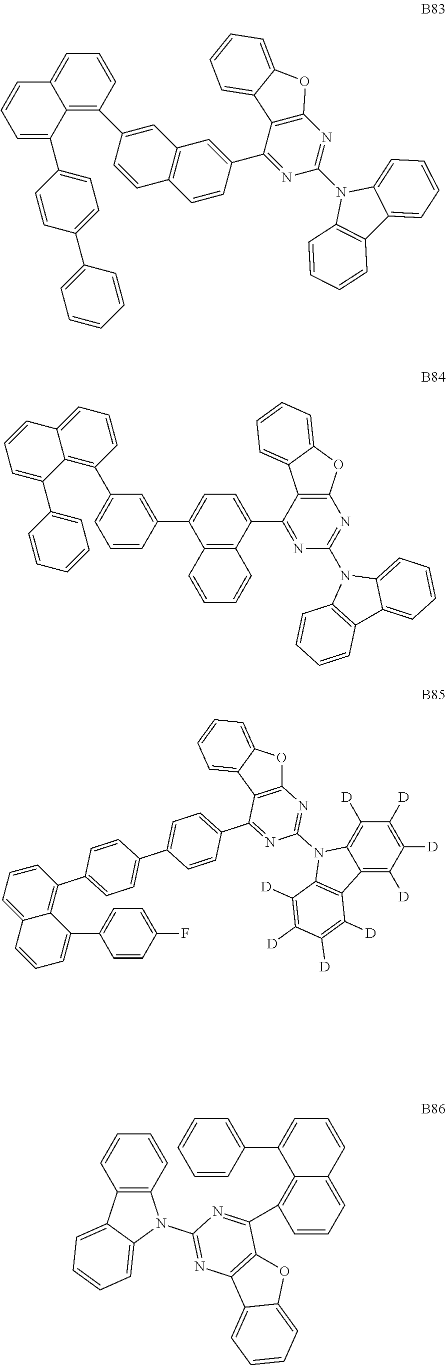 [SMILES: c1ccc(-c2ccc(-c3cccc4cccc(-c5ccc6ccc(-c7nc(-n8c9ccccc9c9ccccc98)nc8oc9ccccc9c78)cc6c5)c34)cc2)cc1 ; c1ccc(-c2cccc3cccc(-c4cccc(-c5ccc(-c6nc(-n7c8ccccc8c8ccccc87)nc7oc8ccccc8c67)c6ccccc56)c4)c23)cc1 ; [2H]c1c([2H])c([2H])c2c(c1[2H])c1c([2H])c([2H])c([2H])c([2H])c1n2-c1nc(-c2ccc(-c3ccc(-c4cccc5cccc(-c6ccc(F)cc6)c45)cc3)cc2)c2c(n1)oc1ccccc12 ; c1ccc(-c2cccc3cccc(-c4nc(-n5c6ccccc6c6ccccc65)nc5c4oc4ccccc45)c23)cc1]

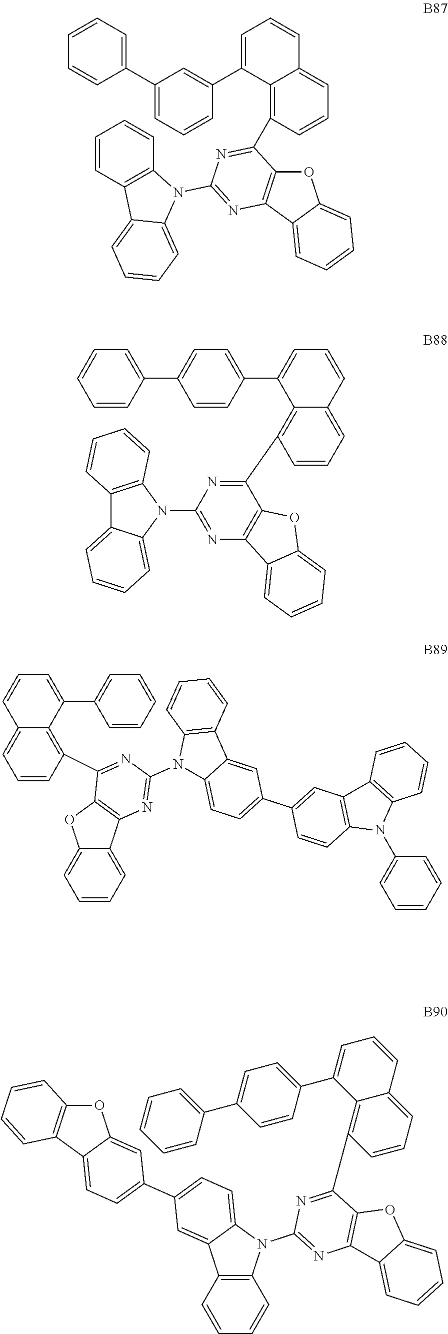 [SMILES: c1ccc(-c2cccc(-c3cccc4cccc(-c5nc(-n6c7ccccc7c7ccccc76)nc6c5oc5ccccc56)c34)c2)cc1 ; c1ccc(-c2ccc(-c3cccc4cccc(-c5nc(-n6c7ccccc7c7ccccc76)nc6c5oc5ccccc56)c34)cc2)cc1 ; c1ccc(-c2cccc3cccc(-c4nc(-n5c6ccccc6c6cc(-c7ccc8c(c7)c7ccccc7n8-c7ccccc7)ccc65)nc5c4oc4ccccc45)c23)cc1 ; c1ccc(-c2ccc(-c3cccc4cccc(-c5nc(-n6c7ccccc7c7cc(-c8ccc9c(c8)oc8ccccc89)ccc76)nc6c5oc5ccccc56)c34)cc2)cc1]

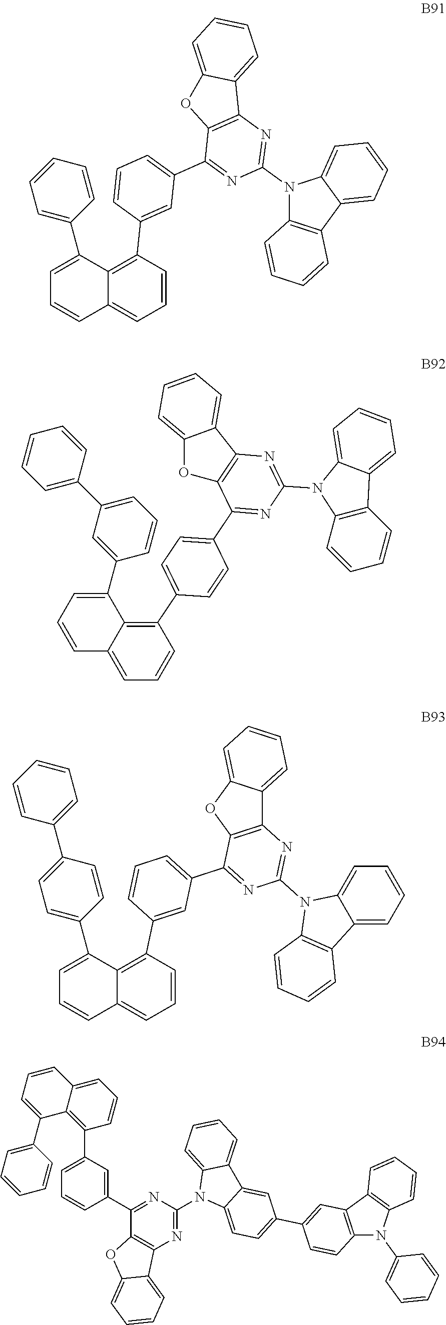 [SMILES: c1ccc(-c2cccc3cccc(-c4cccc(-c5nc(-n6c7ccccc7c7ccccc76)nc6c5oc5ccccc56)c4)c23)cc1 ; c1ccc(-c2cccc(-c3cccc4cccc(-c5ccc(-c6nc(-n7c8ccccc8c8ccccc87)nc7c6oc6ccccc67)cc5)c34)c2)cc1 ; c1ccc(-c2ccc(-c3cccc4cccc(-c5cccc(-c6nc(-n7c8ccccc8c8ccccc87)nc7c6oc6ccccc67)c5)c34)cc2)cc1 ; c1ccc(-c2cccc3cccc(-c4cccc(-c5nc(-n6c7ccccc7c7cc(-c8ccc9c(c8)c8ccccc8n9-c8ccccc8)ccc76)nc6c5oc5ccccc56)c4)c23)cc1]

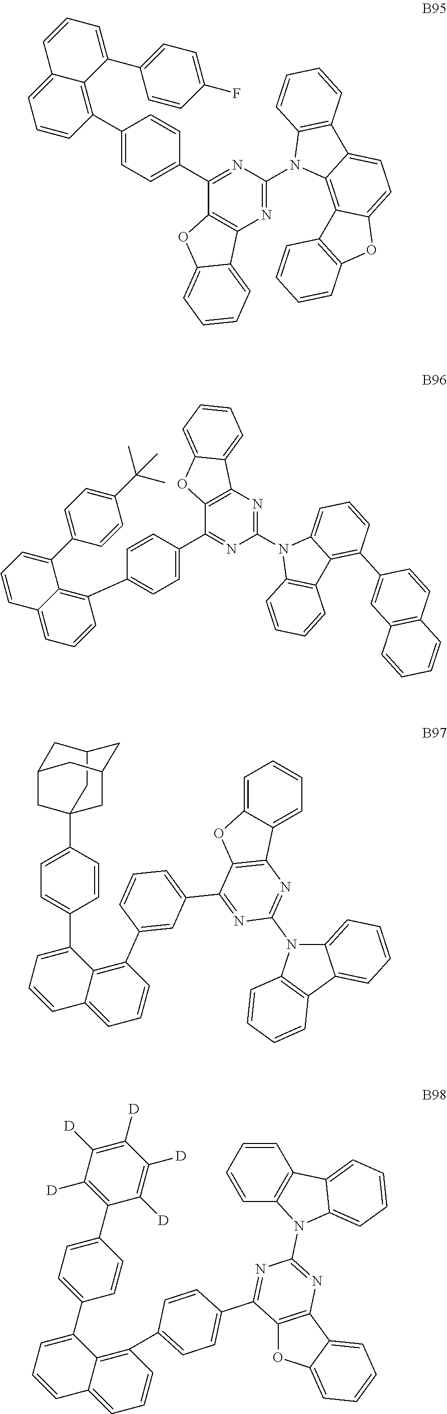 [SMILES: Fc1ccc(-c2cccc3cccc(-c4ccc(-c5nc(-n6c7ccccc7c7ccc8oc9ccccc9c8c76)nc6c5oc5ccccc56)cc4)c23)cc1 ; CC(C)(C)c1ccc(-c2cccc3cccc(-c4ccc(-c5nc(-n6c7ccccc7c7c(-c8ccc9ccccc9c8)cccc76)nc6c5oc5ccccc56)cc4)c23)cc1 ; c1cc(-c2nc(-n3c4ccccc4c4ccccc43)nc3c2oc2ccccc23)cc(-c2cccc3cccc(-c4ccc(C56CC7CC(CC(C7)C5)C6)cc4)c23)c1 ; [2H]c1c([2H])c([2H])c(-c2ccc(-c3cccc4cccc(-c5ccc(-c6nc(-n7c8ccccc8c8ccccc87)nc7c6oc6ccccc67)cc5)c34)cc2)c([2H])c1[2H]]

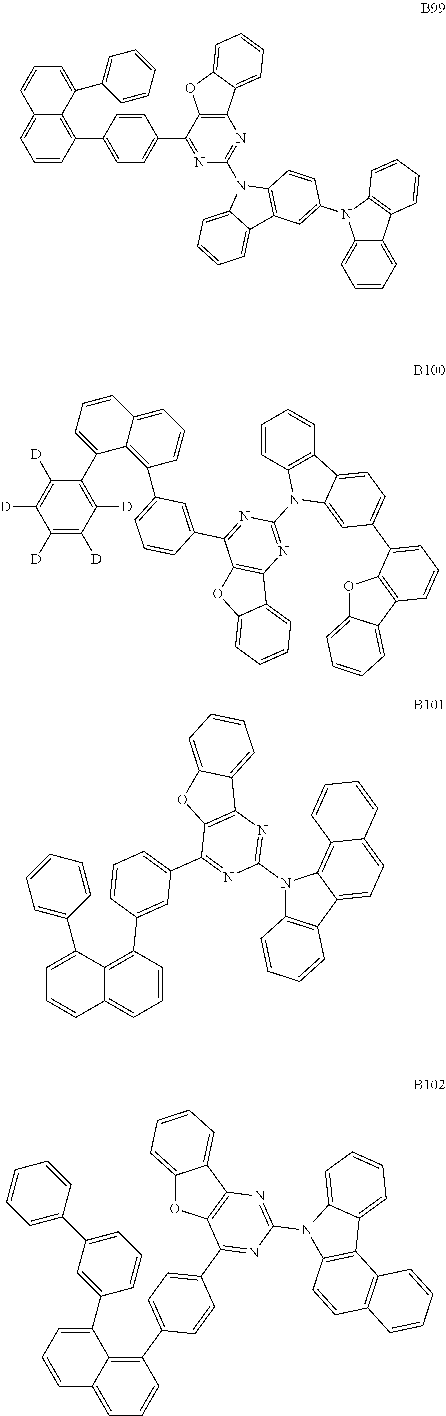 [SMILES: c1ccc(-c2cccc3cccc(-c4ccc(-c5nc(-n6c7ccccc7c7cc(-n8c9ccccc9c9ccccc98)ccc76)nc6c5oc5ccccc56)cc4)c23)cc1 ; [2H]c1c([2H])c([2H])c(-c2cccc3cccc(-c4cccc(-c5nc(-n6c7ccccc7c7ccc(-c8cccc9c8oc8ccccc89)cc76)nc6c5oc5ccccc56)c4)c23)c([2H])c1[2H] ; c1ccc(-c2cccc3cccc(-c4cccc(-c5nc(-n6c7ccccc7c7ccc8ccccc8c76)nc6c5oc5ccccc56)c4)c23)cc1 ; c1ccc(-c2cccc(-c3cccc4cccc(-c5ccc(-c6nc(-n7c8ccccc8c8c9ccccc9ccc87)nc7c6oc6ccccc67)cc5)c34)c2)cc1]

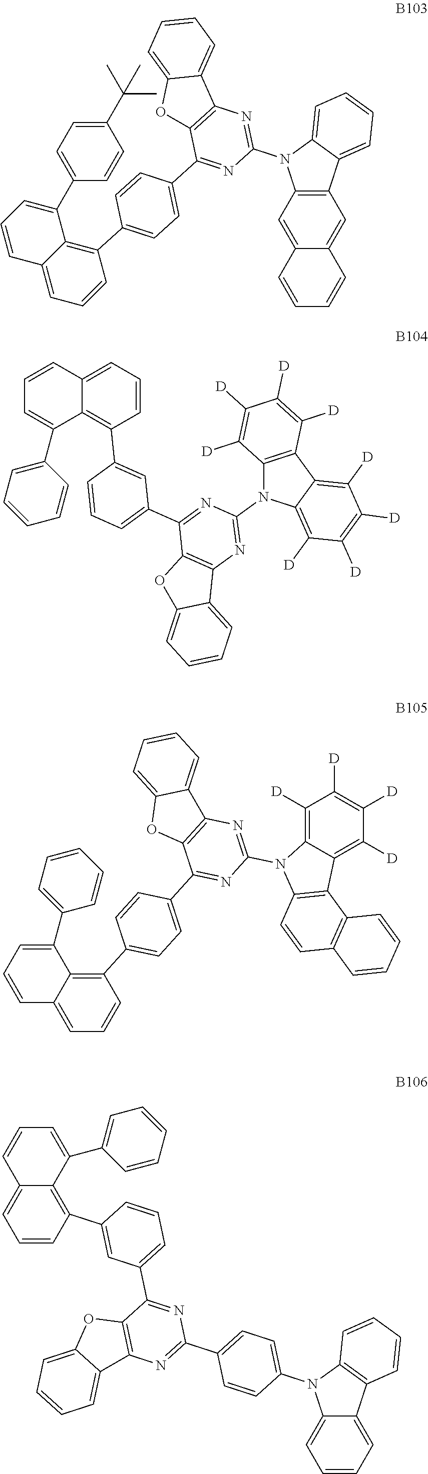 [SMILES: CC(C)(C)c1ccc(-c2cccc3cccc(-c4ccc(-c5nc(-n6c7ccccc7c7cc8ccccc8cc76)nc6c5oc5ccccc56)cc4)c23)cc1 ; [2H]c1c([2H])c([2H])c2c(c1[2H])c1c([2H])c([2H])c([2H])c([2H])c1n2-c1nc(-c2cccc(-c3cccc4cccc(-c5ccccc5)c34)c2)c2oc3ccccc3c2n1 ; [2H]c1c([2H])c([2H])c2c(c1[2H])c1c3ccccc3ccc1n2-c1nc(-c2ccc(-c3cccc4cccc(-c5ccccc5)c34)cc2)c2oc3ccccc3c2n1 ; c1ccc(-c2cccc3cccc(-c4cccc(-c5nc(-c6ccc(-n7c8ccccc8c8ccccc87)cc6)nc6c5oc5ccccc56)c4)c23)cc1]

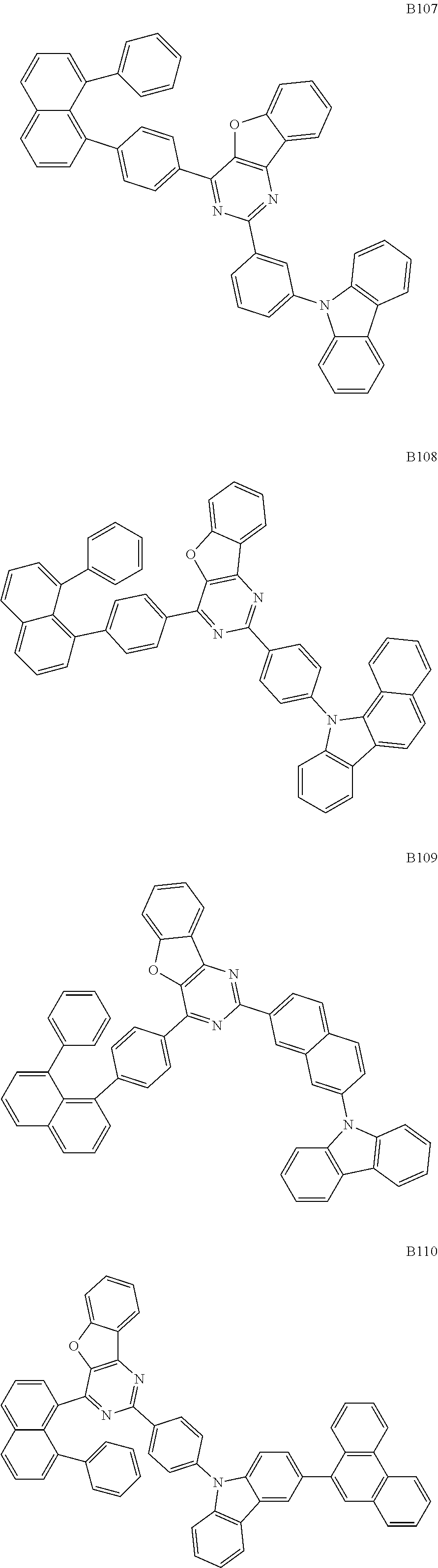 [SMILES: c1ccc(-c2cccc3cccc(-c4ccc(-c5nc(-c6cccc(-n7c8ccccc8c8ccccc87)c6)nc6c5oc5ccccc56)cc4)c23)cc1 ; c1ccc(-c2cccc3cccc(-c4ccc(-c5nc(-c6ccc(-n7c8ccccc8c8ccc9ccccc9c87)cc6)nc6c5oc5ccccc56)cc4)c23)cc1 ; c1ccc(-c2cccc3cccc(-c4ccc(-c5nc(-c6ccc7ccc(-n8c9ccccc9c9ccccc98)cc7c6)nc6c5oc5ccccc56)cc4)c23)cc1 ; c1ccc(-c2cccc3cccc(-c4nc(-c5ccc(-n6c7ccccc7c7cc(-c8cc9ccccc9c9ccccc89)ccc76)cc5)nc5c4oc4ccccc45)c23)cc1]

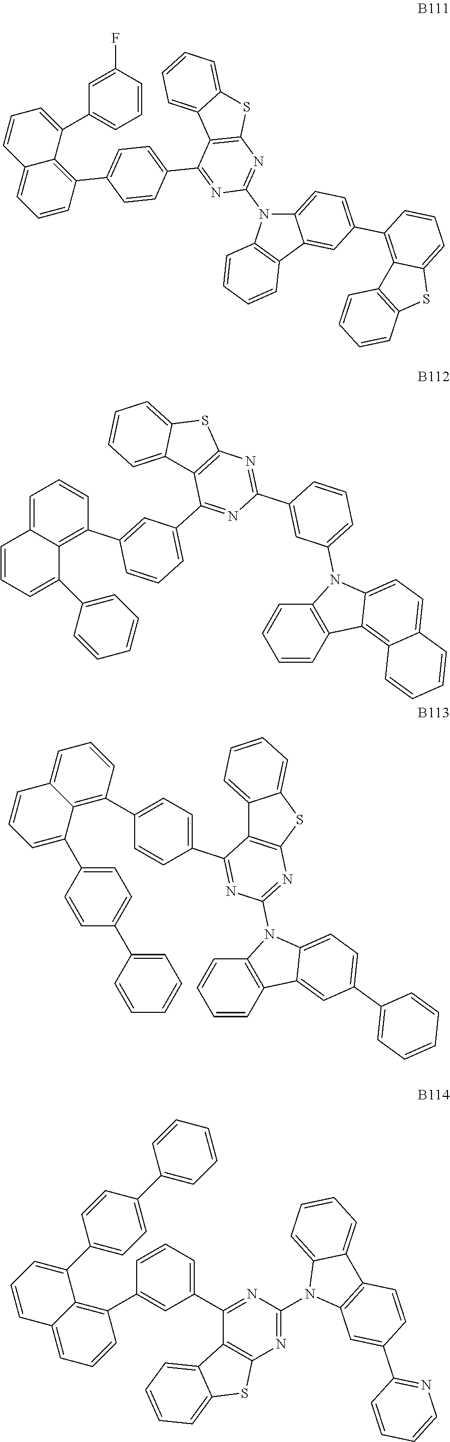 [SMILES: Fc1cccc(-c2cccc3cccc(-c4ccc(-c5nc(-n6c7ccccc7c7cc(-c8cccc9sc%10ccccc%10c89)ccc76)nc6sc7ccccc7c56)cc4)c23)c1 ; c1ccc(-c2cccc3cccc(-c4cccc(-c5nc(-c6cccc(-n7c8ccccc8c8c9ccccc9ccc87)c6)nc6sc7ccccc7c56)c4)c23)cc1 ; c1ccc(-c2ccc(-c3cccc4cccc(-c5ccc(-c6nc(-n7c8ccccc8c8cc(-c9ccccc9)ccc87)nc7sc8ccccc8c67)cc5)c34)cc2)cc1 ; c1ccc(-c2ccc(-c3cccc4cccc(-c5cccc(-c6nc(-n7c8ccccc8c8ccc(-c9ccccn9)cc87)nc7sc8ccccc8c67)c5)c34)cc2)cc1]

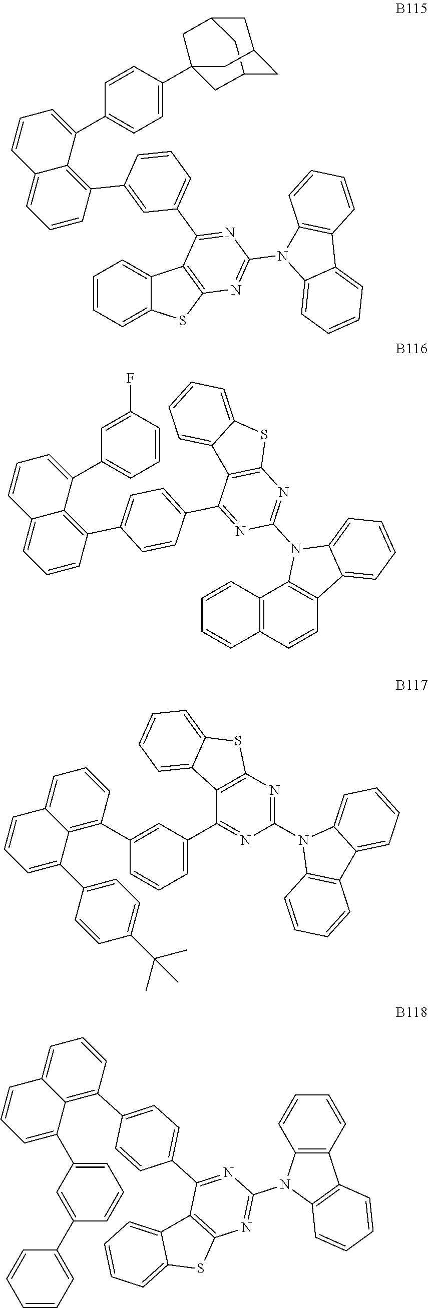 [SMILES: c1cc(-c2cccc3cccc(-c4ccc(C56CC7CC(CC(C7)C5)C6)cc4)c23)cc(-c2nc(-n3c4ccccc4c4ccccc43)nc3sc4ccccc4c23)c1 ; Fc1cccc(-c2cccc3cccc(-c4ccc(-c5nc(-n6c7ccccc7c7ccc8ccccc8c76)nc6sc7ccccc7c56)cc4)c23)c1 ; CC(C)(C)c1ccc(-c2cccc3cccc(-c4cccc(-c5nc(-n6c7ccccc7c7ccccc76)nc6sc7ccccc7c56)c4)c23)cc1 ; c1ccc(-c2cccc(-c3cccc4cccc(-c5ccc(-c6nc(-n7c8ccccc8c8ccccc87)nc7sc8ccccc8c67)cc5)c34)c2)cc1]

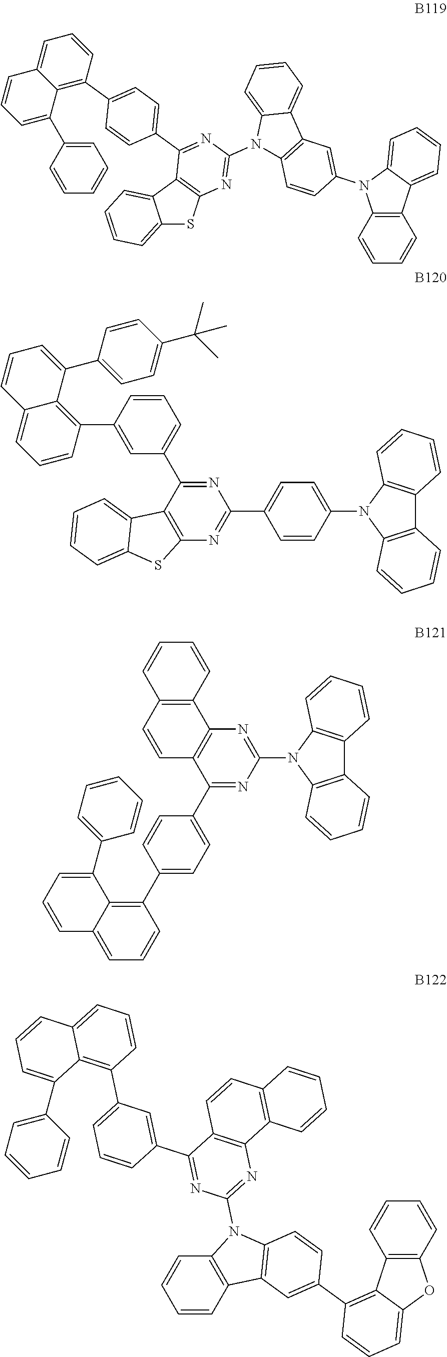

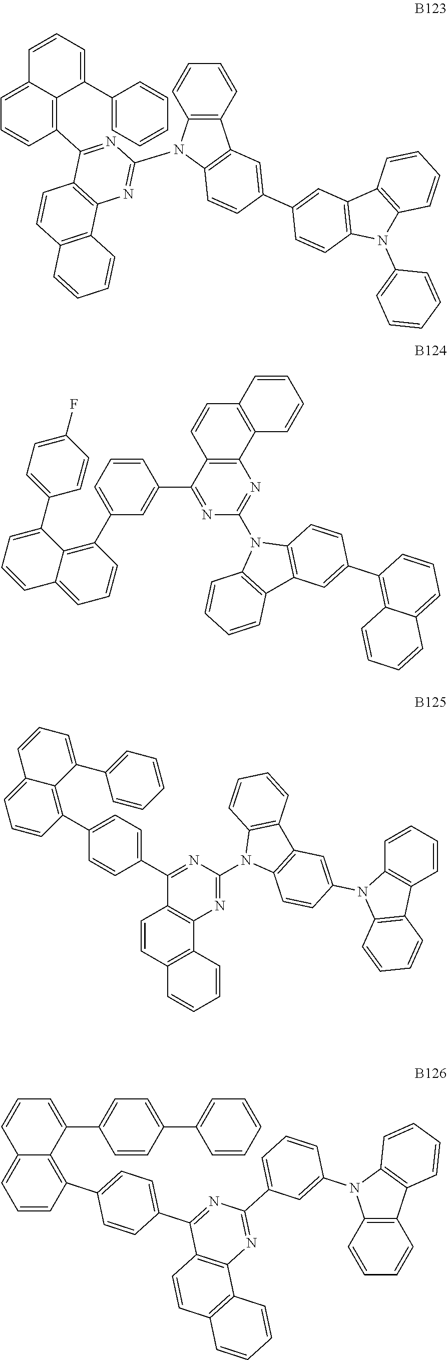 [SMILES: c1ccc(-c2cccc3cccc(-c4nc(-n5c6ccccc6c6cc(-c7ccc8c(c7)c7ccccc7n8-c7ccccc7)ccc65)nc5c4ccc4ccccc45)c23)cc1 ; Fc1ccc(-c2cccc3cccc(-c4cccc(-c5nc(-n6c7ccccc7c7cc(-c8cccc9ccccc89)ccc76)nc6c5ccc5ccccc56)c4)c23)cc1 ; c1ccc(-c2cccc3cccc(-c4ccc(-c5nc(-n6c7ccccc7c7cc(-n8c9ccccc9c9ccccc98)ccc76)nc6c5ccc5ccccc56)cc4)c23)cc1 ; c1ccc(-c2ccc(-c3cccc4cccc(-c5ccc(-c6nc(-c7cccc(-n8c9ccccc9c9ccccc98)c7)nc7c6ccc6ccccc67)cc5)c34)cc2)cc1]

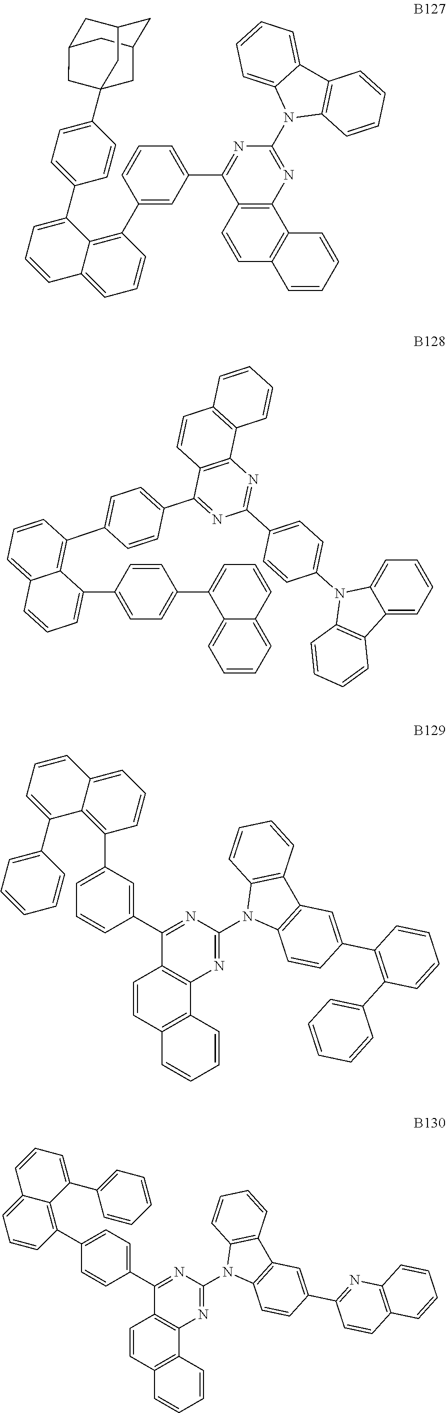 [SMILES: c1cc(-c2nc(-n3c4ccccc4c4ccccc43)nc3c2ccc2ccccc23)cc(-c2cccc3cccc(-c4ccc(C56CC7CC(CC(C7)C5)C6)cc4)c23)c1 ; c1ccc2c(-c3ccc(-c4cccc5cccc(-c6ccc(-c7nc(-c8ccc(-n9c%10ccccc%10c%10ccccc%109)cc8)nc8c7ccc7ccccc78)cc6)c45)cc3)cccc2c1 ; c1ccc(-c2ccccc2-c2ccc3c(c2)c2ccccc2n3-c2nc(-c3cccc(-c4cccc5cccc(-c6ccccc6)c45)c3)c3ccc4ccccc4c3n2)cc1 ; c1ccc(-c2cccc3cccc(-c4ccc(-c5nc(-n6c7ccccc7c7cc(-c8ccc9ccccc9n8)ccc76)nc6c5ccc5ccccc56)cc4)c23)cc1]

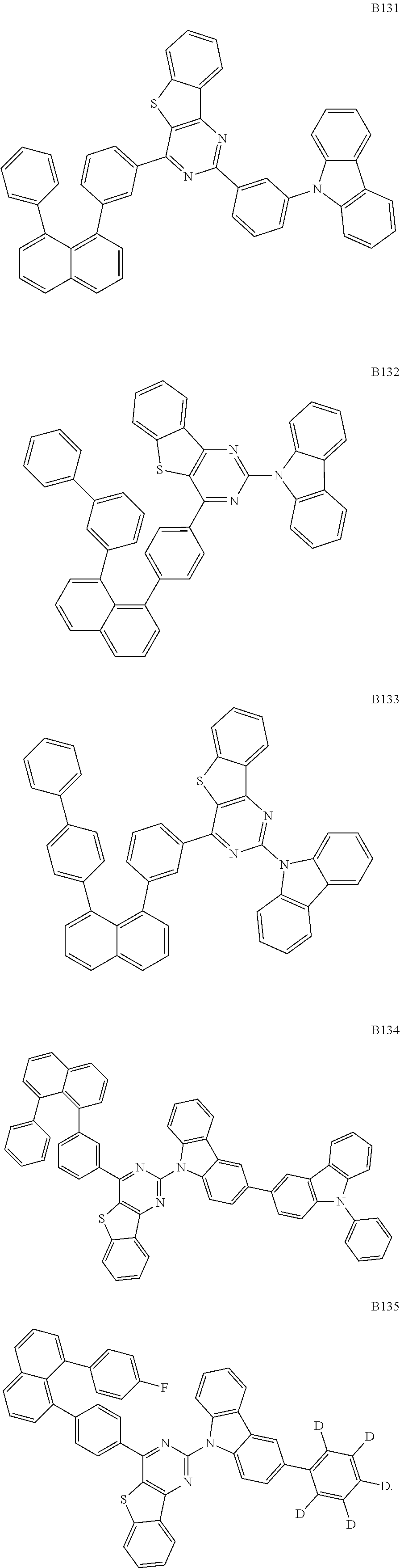

14. An electronic element, comprising an anode and a cathode which are oppositely disposed, and a functional layer disposed between the anode and the cathode, wherein the functional layer comprises the organic compound according to claim 1.

15. The electronic element according to claim 14, wherein the electronic element is an organic electroluminescent device, and the functional layer comprises an organic light-emitting layer comprising the organic compound.

16. An electronic apparatus, comprising the electronic element according to claim 14.
